(19) United States
(12) Patent Application Publication
(10) Pub. No.: US 2025/0051266 A1
(43) Pub. Date: Feb. 13, 2025

(54) Novel GLV-Phenolamide: Biosynthesis and Function in Protecting Plants from Herbivore Attack

(71) Applicant: Max-Planck-Gesellschaft zur Förderung der Wissenschaften e.V., München (DE)

(72) Inventors: Yuechen Bai, Jena (DE); Dapeng Li, Shanghai (CN); Ian T. Baldwin, Berlin (DE); Caiqiong Yang, Jena (DE); Rayko Halitschke, Bucha (DE); Christian Paetz, Jena (DE)

(21) Appl. No.: 18/723,300

(22) PCT Filed: Dec. 22, 2022
(86) PCT No.: PCT/EP2022/087443

§ 371 Date: Jun. 21, 2024

Related U.S. Application Data
(60) Provisional application No. 63/293,193, filed on Dec. 23, 2021.

(30) Foreign Application Priority Data
Dec. 23, 2021 (EP) 21217268.8

(51) Int. Cl. C07C 235/78; A01N 37/42; A01P 15/00; C12N 9/02; C12N 9/04; C12N 9/10; C12N 9/88; C12P 13/02

(52) U.S. Cl. CPC C07C 235/78; A01N 37/42; C12N 9/0006; C12N 9/0071; C12N 9/1029; C12N 9/88; C12P 13/02; A01P 15/00; C12Y 203/01099; C12Y 401/01017; C12Y 403/01024

(57) ABSTRACT

The present invention relates to a compound of general formula (I)

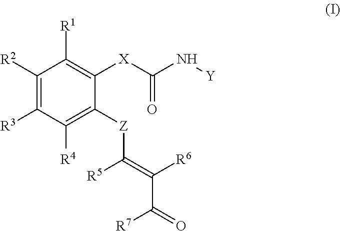

or an enantiomer, diastereomer, stereoisomer, which mediates resistance against leaf- and planthopper pests. The present invention further relates to a method of producing the compound, an enzymatic production method the compound using at least a BBL2 polypeptide, as well as a PPO, AT1, ODC, HPL, PAL, C4H, 4CL, HCT and/or C3H activity. Further envisaged are genetically modified organisms producing the compound, expression cassettes for heterologous expression of the activities, the use of corresponding polypeptides and polynucleotides for the production of the compound, a composition including the compound, as well as uses of the compound for plant protection.

### CROSS-REFERENCE TO RELATED APPLICATIONS

This application is the United States national phase of International Patent Application No. PCT/EP2022/087443 filed Dec. 22, 2022, and claims priority to U.S. Provisional Patent Application No. 63/293,193 filed Dec. 23, 2021, and European Patent Application No. 21217268.8 filed Dec. 23, 2021, the disclosures of which are hereby incorporated by reference in their entireties.

### REFERENCE TO A SEQUENCE LISTING

The Sequence Listing associated with this application is filed in electronic format via Patent Center and is hereby incorporated by reference into the specification in its entirety. The name of XML file containing the Sequence Listing is E38149WO.xml. The size of the XML file is 232,454 bytes and the XML was created on Dec. 21, 2022.

### BACKGROUND OF THE INVENTION

### Field of the Invention

The present invention relates to a compound of general formula (I)

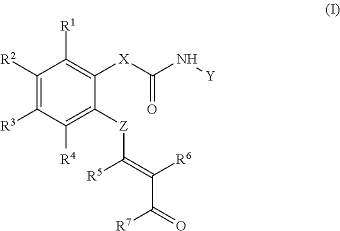

or an enantiomer, diastereomer, stereoisomer, which mediates resistance against leaf- and planthopper pests. The present invention further relates to a method of producing the compound, an enzymatic production method the compound using at least a BBL2 polypeptide, as well as a PPO, AT1, ODC, HPL, PAL, C4H, 4CL, HCT and/or C3H activity. Further envisaged are genetically modified organisms producing the compound, expression cassettes for heterologous expression of the activities, the use of corresponding polypeptides and polynucleotides for the production of the compound, a composition comprising the compound, as well as uses of the compound for plant protection.

### Description of Related Art

Being at the bottom of most terrestrial food chains, plants are continuously attacked by herbivores and pathogens (Kessler and Kalske, Annual Review of Ecology, Evolution, and Systematics 49, 115-138 (2018), Bednarek and Osbourn, Science 324, 746-748 (2009)). Plants provide a variety of resources, such as food, mating and oviposition sites, and shelter for a ma-jority of phytophagous insect species. Host-plant selection by insects involves complex behavioral responses to a variety of physical and chemical characteristics of the host plant that operate at different spatial scales and include long-range olfactory (e.g., plant-derived volatiles perceived by odor receptors) and visual (e.g., plant shape, size, and color) cues and short-range chemotactic and gustatory (e.g., surface metabolites perceived by chemoreceptors) cues (Thorsteinson, Ann. Rev. Entomol. 5, 193-218 (1960)). The physical and chemical characteristics of plants that insects use for host selection depend on the feeding guild and the dietary behavior (e.g., polyphagy or oligophagy) of the insect species (Prokopy and Owens, Entomol Exp Appl 24(3):609-62 (1978))

Insects also can perceive phytohormones. Helicoverpa zea (order Lepidoptera) larvae can, for example, perceive jasmonic acid (JA) (Li et al., Nature 419:712-715 (2002)), which accumulates in the food plants during attack and induces de novo synthesis of plant defense metabolites (Dicke and Baldwin, Trends Plant Sci, 15:167-175, (2010)). Thus, insects may select plants for feeding based on the plant's capacity to produce certain phytohormones.

In some cases insects can suppress the accumulation of plant defense metabolites as a mechanism of food plant selection (Howe and Jander, Annu Rev Plant Biol 59:41-66 (2008)). These suppression mechanisms often are associated with the alteration of phytohormone biosynthesis or signaling-pathways and may involve specific enzymes (e.g., glucose oxidase) produced by the insect (Musser et al., Nature, 416: 599-600 (2002)) or vectored microorganisms (Mayer et al. J Chem Ecol, 34:1045-1049 (2008)).

For insects of the order Hemiptera, the behavioral responses to characteristics of the host plant involve a series of steps including labial dabbing and probing using their piercing mouthparts. These initial probing and feeding attempts also elicit a rapid accumulation of phytohormones, such as JA, and the induced defense metabolites they mediate. When, for example, Nicotiana attenuata plants are rendered JA deficient by silencing the initial committed step of the JA biosynthesis pathway, they are severely attacked in nature by hemipteran leafhoppers of the genus Empoasca.

Research into plant traits that provide resistance against biotic agents such as Empoasca leafhoppers has primarily focused on nonhost resistance for pathogens (Fan et al., Science 331, 1185-1188 (2011); Peart et al., Proc Natl Acad Sci USA 99, 10865-10869 (2002); Sohn et al., New Phytol 193, 58-66 (2012)) and host resistance for herbivores (Agrawal, Science 279, 1201-1202 (1998); Karban and Baldwin, University of Chicago Press, 104-166 (2007)). This difference in em-phasis likely reflects the greater physiological autonomy of herbivores, which are selective in choosing plants to attack, coupled with the challenge of discovering resistance traits of hosts that herbivores refuse to attack. Plants rendered “defenceless” by the abrogation of defense pathways can be attacked by nonhost herbivores in no-choice assays in the laboratory (Muller et al., J Chem Ecol 36, 905-913 (2010); Barth and Jander, Plant J 46, 549-562 (2006)).

However, these assays do not capture the selective procedures by which insects choose their hostplants in nature, limiting the inferences that can be drawn from these laboratory studies about nonhost resistance. Due to the paucity of field studies, the mechanisms and metabolic traits underlying nonhost resistance against herbivores remain largely unknown.

While much is known about plant traits that function in nonhost resistance against pathogens, little is known about nonhost resistance against herbivores, despite its agricultural im-portance. For example Empoasca leafhoppers identify host-plants by eavesdropping on unknown outputs ofjasmonate (JA)-mediated signalling.

There is hence a need for means and methods allowing to improve plant protection against herbivores such as leafhoppers or planthoppers.

### SUMMARY OF THE INVENTION

The present invention addresses this need and provides a compound of general formula (I)

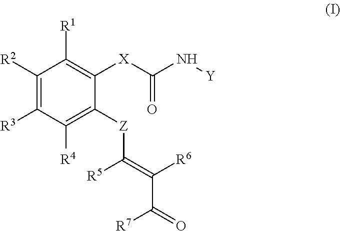

wherein:
R¹, R², R³, and R⁴ are each independently from each other H, OH, (C₁-C₆)-alkyl or (C₁-C₆)-alkoxy;
R⁵, R⁶ and R⁷ are each independently from each other H or (C₁-C₆)-alkyl;
X is straight-chain or branched (C₁-C₈)-alkyl, or straight-chain or branched (C₂-C₅)-alkenyl,
Y is selected from —(CH₂)ₘ—NH₂, —(CH₂)ₙ—NH—(CH₂)ₒ—NH₂, —(CH₂)ₚ—NH—(CH₂)_{q}—NH—(CH₂)ᵣ—NH₂, NH—(CH₂)ₘ—NH₂, NH—(CH₂)ₙ—NH—(CH₂)ₒ—NH₂, and NH—(CH₂)ₚ—NH—(CH₂)_{q}—NH—(CH₂)ᵣ—NH₂, with m, n, o, p, q, and r each being an integer between 1 and 10, or a tyramine ester; and
Z is straight-chain or branched (C₁-C₈)-alkyl, or straight-chain or branched (C₂-C₅)-alkenyl,

or an enantiomer, diastereomer, stereoisomer, or salt thereof.

The inventors surprisingly found that the compound confers resistance to leafhoppers and is likely to provide resistance to all insects that feed by piercing-sucking modes, or oviposit their eggs into leaves.

In a preferred embodiment of said compound two of R¹, R², R³, and R⁴ are OH; R⁵, R⁶ and R⁷ are each H; and X is a straight-chain (C₂-C₅)-alkenyl.

In a preferred embodiment of said compound R¹ and R⁴ are H, R² and R³ are OH, and X is —CH═CH—.

In a further preferred embodiment said compound has the following formula (II):

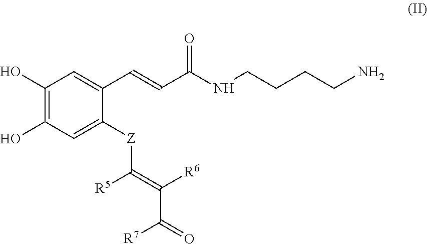

wherein R⁵, R⁶, R⁷ and Z are as defined above;

or is an enantiomer, diastereomer, stereoisomer, or salt thereof.

In yet another preferred embodiment said compound has the formula (III)

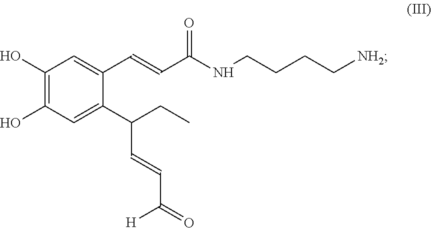 [SMILES: CCC(/C=C/C=O)c1cc(O)c(O)cc1/C=C/C(=O)NCCCCN]

or is an enantiomer, diastereomer, stereoisomer, or salt thereof.

In further aspect the present invention relates to a method of producing the compound according to the invention.

In a preferred embodiment said method is an enzymatic production method using at least a BBL2 (berberine bridge enzyme 2) polypeptide.

In a further preferred embodiment of the method said BBL2 (berberine bridge enzyme 2) polypeptide is
(a) encoded by the polynucleotide having the nucleotide sequence of SEQ ID NO: 1;
(b) encoded by a polynucleotide which is a variant of SEQ ID NO: 1;
(c) encoded by a polynucleotide which is an allelic variant of SEQ ID NO: 1;
(d) encoded by a polynucleotide which is a species homologue of SEQ ID NO: 1;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (a) to (d);
(g) represented by the polypeptide of SEQ ID NO: 2;
(h) represented by a polypeptide fragment of SEQ ID NO: 2 having BBL2 (berberine bridge enzyme 2) function;
(i) represented by a polypeptide domain of SEQ ID NO: 2 having BBL2 (berberine bridge enzyme 2) function;
(j) represented by a polypeptide having an amino acid sequence at least 75%, 80%, 90%, 95%, 97%, 98%, or 99% identical to the amino acid sequence of SEQ ID NO: 2 and having BBL2 (berberine bridge enzyme 2) function; or
(k) represented by a polypeptide being encoded by any one of the polynucleotides specified in (a) to (f).

In a further preferred embodiment of the method said enzymatic production method additionally uses a PPO (polyphenol oxidase) activity or polypeptide, wherein preferably said PPO (polyphenol oxidase) activity or polypeptide is:
(a) encoded by the polynucleotide having the nucleotide sequence of SEQ ID NO: 3 or 5;
(b) encoded by a polynucleotide which is a variant of SEQ ID NO: 3 or 5;
(c) encoded by a polynucleotide which is an allelic variant of SEQ ID NO: 3 or 5;
(d) encoded by a polynucleotide which is a species homologue of SEQ ID NO: 3 or 5;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (a) to (d);
(g) represented by the polypeptide of SEQ ID NO: 4 or 6;
(h) represented by a polypeptide fragment of SEQ ID NO: 4 or 6 having PPO (polyphenol oxidase) activity;
(i) represented by a polypeptide domain of SEQ ID NO: 4 or 6 having PPO (polyphenol oxidase) activity;
(j) represented by a polypeptide having an amino acid sequence at least 75%, 80%, 90%, 95%, 97%, 98%, or 99% identical to the amino acid sequence of SEQ ID NO: 4 or 6 and having PPO (polyphenol oxidase) activity; or
(k) represented by a polypeptide being encoded by any one of the polynucleotides specified in (a) to (f).

In a preferred embodiment said enzymatic production method additionally uses a PPO (polyphenol oxidase) activity or polypeptide, wherein preferably said PPO (polyphenol oxidase) activity or polypeptide is:
(a) encoded by the polynucleotide having the nucleotide sequence of SEQ ID NO: 3 or 5;
(b) encoded by a polynucleotide which is a variant of SEQ ID NO: 3 or 5;
(c) encoded by a polynucleotide which is an allelic variant of SEQ ID NO: 3 or 5;
(d) encoded by a polynucleotide which is a species homologue of SEQ ID NO: 3 or 5;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (a) to (d);
(g) represented by the polypeptide of SEQ ID NO: 4 or 6;
(h) represented by a polypeptide fragment of SEQ ID NO: 4 or 6 having PPO (polyphenol oxidase) activity;
(i) represented by a polypeptide domain of SEQ ID NO: 4 or 6 having PPO (polyphenol oxidase) activity;
(j) represented by a polypeptide having an amino acid sequence at least 75%, 80%, 90%, 95%, 97%, 98%, or 99% identical to the amino acid sequence of SEQ ID NO: 4 or 6 and having PPO (polyphenol oxidase) activity; or
(k) represented by a polypeptide being encoded by any one of the polynucleotides specified in (a) to (f).

In a further preferred embodiment of the method said enzymatic production method additionally uses an AT1 (polyamine hydroxycinnamoyltransferase 1) activity or polypeptide, wherein preferably said AT1 (polyamine hydroxycinnamoyltransferase 1) activity or polypeptide is
(a) encoded by the polynucleotide having the nucleotide sequence of SEQ ID NO: 7;
(b) encoded by a polynucleotide which is a variant of SEQ ID NO: 7;
(c) encoded by a polynucleotide which is an allelic variant of SEQ ID NO: 7;
(d) encoded by a polynucleotide which is a species homologue of SEQ ID NO: 7;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (a) to (d);
(g) represented by the polypeptide of SEQ ID NO: 8;
(h) represented by a polypeptide fragment of SEQ ID NO: 8 having AT1 (polyamine hydroxycinnamoyl-transferase 1) activity;
(i) represented by a polypeptide domain of SEQ ID NO: 8 having AT1 (polyamine hydroxycinnamoyl-transferase 1) activity;
(j) represented by a polypeptide having an amino acid sequence at least 75%, 80%, 90%, 95%, 97%, 98%, or 99% identical to the amino acid sequence of SEQ ID NO: 8 and having AT1 (polyamine hydroxycinnamoyltransferase 1) activity; or
(k) represented by a polypeptide being encoded by any one of the polynucleotides specified in (a) to (f).

In a further preferred embodiment of the method said enzymatic production method additionally uses an ODC (ornithine decarboxylase) activity or polypeptide and/or an HPL (hydroperoxide lyase) activity or polypeptide, wherein preferably
(A) said ODC (ornithine decarboxylase) activity or polypeptide is
(a) encoded by a polynucleotide having the nucleotide sequence of SEQ ID NO: 9 or 11;
(b) encoded by a polynucleotide which is a variant of SEQ ID NO: 9 or 11;
(c) encoded by a polynucleotide which is an allelic variant of SEQ ID NO: 9 or 11;
(d) encoded by a polynucleotide which is a species homologue of SEQ ID NO: 9 or 11;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (a) to (d);
(g) represented by a polypeptide of SEQ ID NO: 10 or 12;
(h) represented by a polypeptide fragment of SEQ ID NO: 10 or 12 having ODC (ornithine decarboxylase) activity;
(i) represented by a polypeptide domain of SEQ ID NO: 10 or 12 having ODC (ornithine decarboxylase) activity;
(j) represented by a polypeptide having an amino acid sequence at least 75%, 80%, 90%, 95%, 97%, 98%, or 99% identical to the amino acid sequence of SEQ ID NO: 10 or 12 and having ODC (ornithine decarboxylase) activity; or
(k) represented by a polypeptide being encoded by any one of the polynucleotides specified in (A) (a) to (f); and
(B) said HPL (hydroperoxide lyase) activity or polypeptide is:
(a) encoded by a polynucleotide having the nucleotide sequence of SEQ ID NO: 13, 15 or 17;
(b) encoded by a polynucleotide which is a variant of SEQ ID NO: 13, 15 or 17;
(c) encoded by a polynucleotide which is an allelic variant of SEQ ID NO: 13, 15 or 17;
(d) encoded by a polynucleotide which is a species homologue of SEQ ID NO: 13, 15 or 17;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (B) (a) to (d);
(g) represented by a polypeptide of SEQ ID NO: 14, 16 or 18;
(h) represented by a polypeptide fragment of SEQ ID NO: 14, 16 or 18 having HPL (hydroperoxide lyase) activity;
(i) represented by a polypeptide domain of SEQ ID NO: 14, 16 or 18 having HPL (hydroperoxide lyase) activity;
(j) represented by a polypeptide having an amino acid sequence at least 75%, 80%, 90%, 95%, 97%, 98%, or 99% identical to the amino acid sequence of SEQ ID NO: 14, 16 or 18 and having HPL (hydroperoxide lyase) activity; or
(k) represented by a polypeptide being encoded by any one of the polynucleotides specified in (B) (a) to (f).

In a further preferred embodiment of the method said enzymatic production method additionally uses a PAL (L-phenylalanine ammonia lyase) activity or polypeptide and/or a C4H (trans-cinnamate 4-hydroxylase) activity or polypeptide and/or an 4CL (4-coumarate:coenzyme A ligase) activity or polypeptide and/or an HCT (Hydroxycinnamoyl-transferase) activity or polypeptide and/or a C3H (coumarate 3-hydroxylase) activity or polypeptide, wherein preferably:
(A) said PAL (L-phenylalanine ammonia lyase) activity or polypeptide is:
(a) encoded by a polynucleotide having the nucleotide sequence of SEQ ID NO: 19, 21, 23 or 25;
(b) encoded by a polynucleotide which is a variant of SEQ ID NO: 19, 21, 23 or 25;
(c) encoded by a polynucleotide which is an allelic variant of SEQ ID NO: 19, 21, 23 or 25;
(d) encoded by a polynucleotide which is a species homologue of SEQ ID NO: 19, 21, 23 or 25;
(e) encoded by a polynucleotide which is at least 75%, 80%, 90%, 95%, 97%, 98%, or 99% identical to the polynucleotide as defined in any one of A (a) to (d);
(f) encoded by a polynucleotide capable of hybridizing under stringent conditions to any one of the polynucleotides specified in A (a) to (d);
(g) represented by a polypeptide of SEQ ID NO: 20, 22, 24 or 26;
(h) represented by a polypeptide fragment of SEQ ID NO: 20, 22, 24 or 26 having PAL (L-phenylalanine ammonia lyase) activity;
(i) represented by a polypeptide domain of SEQ ID NO: 20, 22, 24 or 26 having PAL (L-phenylalanine ammonia lyase) activity;
(j) represented by a polypeptide having an amino acid sequence at least 75%, 80%, 90%, 95%, 97%, 98%, or 99% identical to the amino acid sequence of SEQ ID NO: 20, 22, 24 or 26 and having PAL (L-phenylalanine ammonia lyase) activity; or
(k) represented by a polypeptide being encoded by any one of the polynucleotides specified in A (a) to (f); and
(B) said C4H (trans-cinnamate 4-hydroxylase) activity or polypeptide is:
(a) encoded by a polynucleotide having the nucleotide sequence of SEQ ID NO: 27
(b) encoded by a polynucleotide which is a variant of SEQ ID NO: 27;
(c) encoded by a polynucleotide which is an allelic variant of SEQ ID NO: 27;
(d) encoded by a polynucleotide which is a species homologue of SEQ ID NO: 27; 5 (e) encoded by a polynucleotide which is at least 75%, 80%, 90%, 95%, 97%, 98%, or 99% identical to the polynucleotide as defined in any one of B (a) to (d);
(f) encoded by a polynucleotide capable of hybridizing under stringent conditions to any one of the polynucleotides specified in B (a) to (d);
(g) represented by the polypeptide of SEQ ID NO: 28;
(h) represented by a polypeptide fragment of SEQ ID NO: 28 having C4H (cinnamate 4-hydroxylase) activity;
(i) represented by a polypeptide domain of SEQ ID NO: 28 having C4H (cinnamate 4-hydroxylase) activity;
(j) represented by a polypeptide having an amino acid sequence at least 75%, 80%, 90%, 95%, 97%, 98%, or 99% identical to the amino acid sequence of SEQ ID NO: 28 and having C4H (cinnamate 4-hydroxylase) activity; or
(k) represented by a polypeptide being encoded by any one of the polynucleotides specified in B (a) to (f); and
(C) said 4CL (4-coumarate:coenzyme A ligase) activity or polypeptide is: 20 (a) encoded by a polynucleotide having the nucleotide sequence of SEQ ID NO: 29, 105, 107, 109, 111, 113, 115, 117, 119, 121, 123, 125, 127, 129 or 131;
(b) encoded by a polynucleotide which is a variant of SEQ ID NO: 29, 105, 107, 109, 111, 113, 115, 117, 119, 121, 123, 125, 127, 129 or 131;
(c) encoded by a polynucleotide which is an allelic variant of SEQ ID NO: 29, 105, 107, 109, 111, 113, 115, 117, 119, 121, 123, 125, 127, 129 or 131;
(d) encoded by a polynucleotide which is a species homologue of SEQ ID NO: 29, 105, 107, 109, 111, 113, 115, 117, 119, 121, 123, 125, 127, 129 or 131;
(e) encoded by a polynucleotide which is at least 75%, 80%, 90%, 95%, 97%, 98%, or 99% identical to the polynucleotide as defined in any one of C (a) to (d);
(f) encoded by a polynucleotide capable of hybridizing under stringent conditions to any one of the polynucleotides specified in C (a) to (d);
(g) represented by the polypeptide of SEQ ID NO: 30, 106, 108, 110, 112, 114, 116, 118, 120, 122, 124, 126, 128, 130 or 132;
(h) represented by a polypeptide fragment of SEQ ID NO: 30, 106, 108, 110, 112, 114, 116, 118, 120, 122, 124, 126, 128, 130 or 132 having 4CL (4-coumarate:coenzyme A ligase) activity;
(i) represented by a polypeptide domain of SEQ ID NO: 30, 106, 108, 110, 112, 114, 116, 118, 120, 122, 124, 126, 128, 130 or 132 having 4CL (4-coumarate:coenzyme A ligase) activity;
(j) represented by a polypeptide having an amino acid sequence at least 75%, 80%, 90%, 95%, 97%, 98%, or 99% identical to the amino acid sequence of SEQ ID NO: 30, 106, 108, 110, 112, 114, 116, 118, 120, 122, 124, 126, 128, 130 or 132 and having 4CL (4-coumarate:coenzyme A ligase) activity; or
(k) represented by a polypeptide being encoded by any one of the polynucleotides specified in C (a) to (f); and
(D) said HCT (Hydroxycinnamoyl-transferase) activity or polypeptide is:
(a) encoded by a polynucleotide having the nucleotide sequence of SEQ ID NO: 31, 33, 35, 37, 39, 41, or 43;
(b) encoded by a polynucleotide which is a variant of SEQ ID NO: 31, 33, 35, 37, 39, 41, or 43;
(c) encoded by a polynucleotide which is an allelic variant of SEQ ID NO: 31, 33, 35, 37, 39, 41, or 43;
(d) encoded by a polynucleotide which is a species homologue of SEQ ID NO: 31, 33, 35, 37, 39, 41, or 43;
(e) encoded by a polynucleotide which is at least 75%, 80%, 90%, 95%, 97%, 98%, or 99% identical to the polynucleotide as defined in any one of D (a) to (d);
(f) encoded by a polynucleotide capable of hybridizing under stringent conditions to any one of the polynucleotides specified in D (a) to (d);
(g) represented by a polypeptide of SEQ ID NO: 32, 34, 36, 38, 40, 42 or 44;
(h) represented by a polypeptide fragment of SEQ ID NO: 32, 34, 36, 38, 40, 42 or 44 having HCT (Hydroxycinnamoyl-transferase) activity;
(i) represented by a polypeptide domain of SEQ ID NO: 32, 34, 36, 38, 40, 42 or 44 having HCT (Hydroxycinnamoyl-transferase) activity;
(j) represented by a polypeptide having an amino acid sequence at least 75%, 80%, 90%, 95%, 97%, 98%, or 99% identical to the amino acid sequence of SEQ ID NO: 32, 34, 36, 38, 40, 42 or 44 and having HCT (Hydroxycinnamoyl-transferase) activity; or
(k) represented by a polypeptide being encoded by any one of the polynucleotides specified in D (a) to (f); and
(E) said C3H (coumarate 3-hydroxylase) activity or polypeptide is:
(a) encoded by a polynucleotide having the nucleotide sequence of SEQ ID NO: 45;
(b) encoded by a polynucleotide which is a variant of SEQ ID NO: 45;
(c) encoded by a polynucleotide which is an allelic variant of SEQ ID NO: 45;
(d) encoded by a polynucleotide which is a species homologue of SEQ ID NO: 45;
(e) encoded by a polynucleotide which is at least 75%, 80%, 90%, 95%, 97%, 98%, or 99% identical to the polynucleotide as defined in any one of E (a) to (d);
(f) encoded by a polynucleotide capable of hybridizing under stringent conditions to any one of the polynucleotides specified in E (a) to (d);
(g) represented by a polypeptide of SEQ ID NO: 46;
(h) represented by a polypeptide fragment of SEQ ID NO: 46 having C3H (coumarate 3-hydroxylase) activity;
(i) represented by a polypeptide domain of SEQ ID NO: 46 having C3H (coumarate 3-hydroxylase) activity;
(j) represented by a polypeptide having an amino acid sequence at least 75%, 80%, 90%, 95%, 97%, 98%, or 99% identical to the amino acid sequence of SEQ ID NO: 46 and having C3H (coumarate 3-hydroxylase) activity; or
(k) represented by a polypeptide being encoded by any one of the polynucleotides specified in E (a) to (f).

In a further preferred embodiment said enzymatic production is performed with activities or polypeptides provided in vitro. It is particularly preferred that said method is carried out at a pH of 4.8 or lower, more preferably by using solid phase extraction with argon flow.

In a further preferred embodiment said enzymatic production is performed with activities or polypeptides provided in a living cell, tissue or organism.

In a further preferred embodiment said enzymatic production is performed with and in a genetically modified cell, tissue or organism, wherein said genetic modification allows for the heterologous expression of one or more activities or polypeptides as defined herein above.

In a further aspect the present invention relates to an organism, tissue or cell producing the compound according to the invention, which is genetically modified, wherein said genetic modification allows for the heterologous expression of one or more activities or polypeptides as defined herein.

In a preferred embodiment of said method, organism, tissue or the cell said genetic modification results at least in the expression of a BBL2 (berberine bridge enzyme 2) polypeptide as defined herein.

In a preferred embodiment of said method, organism, tissue or the cell said genetic modification results at least in the expression of a PPO (polyphenol oxidase) activity or polypeptide as defined herein.

In a further preferred embodiment of said method, organism, tissue or the cell said genetic modification results at least in the expression of an AT1(polyamine hydroxycinnamoyl-transferase 1) activity or polypeptide as defined herein.

In a further preferred embodiment of the method, organism, tissue or the cell said genetic modification results at least in the expression of an ODC (ornithine decarboxylase) activity or polypeptide and/or an HPL (hydroperoxide lyase) activity or polypeptide as defined herein.

In yet another preferred embodiment of said method, organism, tissue or the cell said genetic modification results at least in the expression of an PAL (L-phenylalanine ammonia lyase) activity or polypeptide and/or a C4H (trans-cinnamate 4-hydroxylase) activity or polypeptide and/or an 4CL (4-coumarate:coenzyme A ligase) activity or polypeptide or an HCT (Hy-droxycinnamoyltransferase) activity or polypeptide and/or C3H (coumarate 3-hydroxylase) activity or polypeptide as defined herein.

In yet another preferred embodiment of said method, organism, tissue or the cell said expression is conveyed by a native, regulated, tissue specific or constitutive promoter.

It is particularly preferred that said promoter allows for (i) a polycistronic expression of an activity or polypeptide as defined herein, (ii) an individual expression of an activity or polypeptide as defined herein; or (iii) a group-wise expression of groups of at least two activities selected from an activity as defined herein.

It is further particularly preferred that said expression is an overexpression.

In a further preferred embodiment said overexpression is conveyed by a strong regulated or strong constitutive promoter, and/or by the provision of at least a second copy of a genetic element encoding said activity or polypeptide.

In a further specific embodiment of the present invention said enzymatic activity or polypeptide is derived from an organism belonging to the genus Nicotiana. It is particularly preferred that the enzymatic activity or polypeptide is derived from the species Nicotiana attenuata.

It is further preferred that said polynucleotide is comprised in one or more extrachromosomal vectors or plasmids, and/or is integrated in the genome of said organism.

In a further preferred embodiment said genetically modified organism, tissue or cell is eukaryotic. It is particularly preferred that said genetically modified organism is a plant, or wherein said tissue a plant tissue or wherein said cell is a plant cell.

In further preferred embodiments said genetically modified organism belongs to, or the tissue or cell is derived from a higher plant which is attacked by an insect herbivore, preferably a higher pant which is attacked by an insect feeding by lacerate and flush and/or piercing and sucking, more preferably a higher plant of the genus Nicotiana, Solanum, Oryza, Zea, Phaeseolus or Camellia.

In a further aspect the present invention relates to an expression cassette for heterologous expression in a eukaryotic host cell wherein said expression cassette comprises a polynucleotide as defined herein. It is preferred that said host cell is a plant cell.

In a further aspect the present invention relates to a vector or insertion construct comprising the polynucleotide as defined herein or the expression cassette as defined herein.

In yet another aspect the present invention relates to the use of the polypeptide or polynucleotide as defined herein, of the expression cassette as defined herein, or of the vector or insertion construct as defined herein, for the production of the compound of the invention.

In a further aspect the present invention relates to the use of the organism, tissue or cell as defined herein, or of an organism, tissue or cell comprising the expression cassette or the vector or insertion construct as defined herein, for the production of the compound of the invention.

In a further aspect the present invention relates to a composition comprising the compound according to the invention or produced with a method of the invention or produced by an organism, tissue or cell as defined herein. Optionally the composition additionally comprises an acceptable carrier, stabilizer and/or a spreading agent.

In yet another aspect the present invention relates the use of the compound of the invention or produced with a method according to the invention or produced by an organism, tissue or cell as defined herein, or the use of the composition as defined herein for plant protection against an herbivore.

In a preferred embodiment said herbivore is an insect.

In another preferred embodiment said insect is is an insect feeding by lacerate and flush and/or piercing and sucking.

In a further preferred embodiment said insect is a leafhopper or planthopper. It is particularly preferred that said insect is of the family Aphrodinae, Bathysmatophorinae, Cicadellinae, Coelidiinae, Deltocephalinae, Errhomeninae, Euacanthellinae, Eurymelinae, Evacanthinae, Hylicinae, Iassinae, Jascopinae, Ledrinae, Megophthalminae, Mileewinae, Nastlopiinae, Neobalinae, Neocoelidiinae, Nioniinae, Phereurhininae, Portaninae, Signoretiinae, Tartessinae, Typhlocybina, or Ulopinae, more preferably of the genus Empoasca, Circulifer, Nilaparvata, Sogatella, Nephotettix, or Cicadulina.

In a further aspect the present invention relates to the use of the compound of the invention or produced with a method of the invention or produced by an organism, tissue or cell as defined herein or of the composition as defined herein as insecticide. It is preferred that the insecticide is against an insect as define in as defined above.

In yet another preferred embodiment the present invention relates to a method of plant protection comprising contacting a plant or part of a plant with the compound of the invention, or the composition as defined above.

### DESCRIPTION OF THE DRAWINGS

The terms FIG., FIGS., Figure, and Figures are used interchangeably in the specification to refer to the corresponding figures in the drawings.

FIG. 1 A native-insect-guided multi-omics atlas of forward genetic lines of a field-grown N. attenuata MAGIC population highlights deviations from canonical jasmonate signalling for Em-poasca leafhopper nonhost resistance. The figure shows a field plantation of the MAGIC RIL population of native N. attenuata tobacco plants in their native habitat at the WCCER field station, Arizona, USA. Native opportunistic Empoasca leafhopper communities and their feeding damage on leaves are illustrated. Leafhoppers attack these plants in a jasmonate (JA)-dependent manner and preferentially select JA-deficient plants as hosts (images: R. Halitschke, D. Kessler, A. Kessler).

FIG. 2 Schematic of high-throughput phenotyping of phytohormones (1816 samples), 5 transcriptome (350 samples), and metabolome (1706 samples), and Empoasca phenotypes (1907 samples) of 674 MAGIC RILs and parental lines in the field. To remove confounding factors caused by the stochastic nature of insect attack in nature, we mimicked herbivore feeding by immediately applying freshly collected oral secretions (OS) of Manduca sexta larvae to standardized puncture wounds (W) in leaves at standardized leaf positions. This procedure, referred to as W+OS treatment, standardizes the elicitation by precisely timing the initiation of herbivory-elicited responses. The phytohormone and transcriptome datasets were collected 1 h after W+OS treatment and the metabolome dataset after 72 h. PCC, Pearson correlation coefficient.

FIG. 3 Co-association network built from correlations among metabolomes, transcriptomes, phytohormones and SNPs (PCC cutoff P<0.05) and the top 5 most significant SNPs for each gene (small grey circles), metabolites or phytohormones from eQTL or mQTL imputations were retained. Phytohormones and metabolites of different compound classes and JA-related genes are differently labeled. JA-Ile, JA signalling genes (NaAOC, NaMYC2a, NaMYC2b, NaJAZi), NaMYB8 and phenolamides (N-coumaroylputrescine, N-caffeoylputrescine, N-feruloylputrescine) form a central cluster and are labeled.

FIG. 4 Heatmap of coexpressions among metabolites, JA-related genes and phytohormones with Empoasca phenotypes (numbers and area damaged) calculated as pairwise PCC (only significant correlations with P<0.05 are shown with differential labels).

FIG. 5 Reverse genetics coupled with information theory-based unbiased metabolomics reveal an Empoasca-elicited JA-JAZi module regulating induced unknown putrescine-containing phenolamides correlated with Empoasca nonhost resistance. Phenotypes of Empoasca damaged leaves of transgenic N. attenuata lines (n=10) were randomly placed in an open-choice glasshouse environment containing Empoasca leafhoppers (FIG. 26). Representative leaves are shown for each genotype, with insets highlighting the damage of ovJAZi and irMYB8 lines. Different letters indicate significant differences [one-way analysis of variance (ANOVA) followed by Tukey's post hoc multiple comparisons].

FIG. 6 Tissue-specific expression profiles of NaJAZh and NaJAZi in N. attenuata WT (left); heatmap differential labeling depicts the Z-score scaled TPM. Kinetics of relative transcript accumulations of NaJAZh and NaJAZi in leaves of N. attenuata (n=3) in response to continuous E. decipiens and M. sexta feeding with samples harvested at 0 to 24 h after the start of feeding. FLB, flower bud; STI, stigma; COE, corolla early; PED, pedicel; SED, seed; OFL, opening flower; SNP, style; LEC, leaf control; ROT, roots from oral secretion-treated plants; OVA, ovary; NEC, nectary; ANT, anther; STT, stem treated; LET, leaf treated.

FIG. 7 Scatterplots of diversity (Hj) against specialization (6j) indices for specialized metabolomes of leaves (n=4) after 72h of feeding by E. decipiens and M. sexta on four transgenic lines selected from a set of 16 transgenic lines (see FIG. 32 for the complete data set). An increase in metabolome specialization (6j) indicates that, on average, more herbivory/genotype-specific metabolites are produced, whereas an increase in metabolome diversity (Hj) indicates that either qualitatively more metabolites are produced or that quantitatively the global metabolic frequency profile is more uniformly distributed. Differential labelings denote different insects; sym-bols denote different treatments: triangle, insect feeding; circle, untreated control.

FIG. 8 A ranked metabolite specificity (Si) index distribution plot was calculated for each metabolite based on E. decipiens specifically elicited metabolomes from the four transgenic lines shown in FIG. 7 (left) and further linked with a PCC coexpression heatmap among metabolites and E. decipiens number and damage phenotypes (right) for which the data from all reverse-genetic lines shown in FIG. 5 were used to enhance the statistical power of the PCC calculations. Dots are labeled based on compound class annotations. Only significant correlations with P<0.05 are shown with colors.

FIG. 9 In vivo Empoasca choice assays (n=8) conducted by infiltrating synthetic N-coumaroylputrescine (CoP), N-caffeoylputrescine (CP) or N-feruloylputrescine (FP) diluted in 0.1% DMSO solutions into irMYC2 leaves (top) and in vitro Empoasca nonchoice assays (n=3, 25 Em-poasca leafhoppers per replicate) by feeding Empoasca with synthetic CoP, CP or FP diluted in 10% glucose solutions (bottom) revealed that these phenolamides were not affecting leafhopper behavior or performance.

FIG. 10 Elucidating an herbivory-elicited GLV-caffeoylputrescine metabolite and its three-pronged biosynthetic pathways by combining MS/MS structural metabolomics with forward—and reverse-genetics. The figure shows biclustering of 518 idMS/MS spectra constructed from 15 RILs of the field-planting MAGIC population based on shared fragments (NDP-based similarity) and shared neutral losses (NL-based similarity) reveals 7 distinct modules (M) (left) in the molecular network; close-up of module 5 in which putrescine- or caffeoyl-derived phenolamides are enriched (right) harboring an unknown metabolite m/z 347.19 that is directly linked to two isomers of CP (circled in green). CS, N-caffeoylspermidine; CoCS, N′,N″-coumaroyl,caffeoylspermidine; CFS, N′,N″-caffeoyl,feruloylspermidine; DCS, N′, N″-dicaffeoylspermidine; CPD, caffeoylputrescine dimer; CGA, chlorogenic acid; Unk., unknown.

FIG. 11 Elucidating an herbivory-elicited GLV-caffeoylputrescine metabolite and its three-pronged biosynthetic pathways by combining MS/MS structural metabolomics with forward—and reverse-genetics: the figure shows accumulations of m/z 347.19 in Empoasca-elicited EV and irMYC2 lines (top) and MeJA-induced EV, irMYC2, irMYB8, and ovJAZi lines (bottom).

FIG. 12 Elucidating an herbivory-elicited GLV-caffeoylputrescine metabolite and its three-pronged biosynthetic pathways by combining MS/MS structural metabolomics with forward—and reverse-genetics: the figure shows a Manhattan plot for herbivory-induced unknown m/z 347.19 from a mQTL analysis of W+OS elicited leaves from the MAGIC RIL population grown 25 in the glasshouse and extracted with procedures that minimize losses of the phenolamide sector (left). Core JA signalling gene NaMYC2a, phenolamide regulator NaMYB8, CP biosynthetic gene NaAT1 and two unknown biosynthetic candidate genes NaPPO1 and NaPPO2 were imputed in the mQTL analysis (P value cutoffs=10-3) as well as an uncharacterized candidate gene NaBBL2 (P=0.0013). Gene coexpression network constructed using a previously published microarray dataset of irMYB8 plants harvested 1 and 5 h after W+OS elicitation. The phenolamide biosynthetic genes, NaAT1 and NaDH29 were used as baits (diamonds), dots in the center depict genes coexpressed with both baits, whereas the lower dots (NaAT1) and the upper dots (NaDH29) dots depict genes coexpressed with a single bait (right).

FIG. 13 Elucidating an herbivory-elicited GLV-caffeoylputrescine metabolite and its three-pronged biosynthetic pathways by combining MS/MS structural metabolomics with forward—and reverse-genetics: the figure shows virus-induced gene silencing (VIGS) of biosynthetic gene candidates involved in m/z 347.19 production. Silencing NaPPO1, NaPPO2, NaAT1 and NaBBL2 expression abolished the elicitation of m/z 347.19 by W+OS treatment observed in EV control plants (C, untreated controls).

FIG. 14 Elucidating an herbivory-elicited GLV-caffeoylputrescine metabolite and its three-pronged biosynthetic pathways by combining MS/MS structural metabolomics with forward—and reverse-genetics: the figure shows the proposed three-pronged biosynthetic pathway for the Empoasca-elicited m/z 347.19 production, which requires the LOX2-HPL-dependent C6 GLV metabolism, LOX3-dependent and JA-regulated phenylpropanoid metabolism and polyamine metabolism, the outputs of which are putatively conjugated in NaBBL2—and NaPPO1/2-dependent reactions.

FIG. 15 Elucidating an herbivory-elicited GLV-caffeoylputrescine metabolite and its three-pronged biosynthetic pathways by combining MS/MS structural metabolomics with forward—and reverse-genetics: the figure shows scatterplots of metabolite abundance of m/z 347.19 μgainst (Z)-3-hexen-1-ol in MAGIC accessions from glasshouse (top) and m/z 347.19 accumulation after W+OS treatment in leaves of stably transformed EV, asHPL, irLOX2 and irLOX2/3 lines (bottom).

FIG. 16 Structural elucidation, biosynthesis, function and engineering of m/z 347.19 in vitro and in planta: the figure shows In vitro enzymatic assays for m/z 347 production revealed that either NaPPO1 or NaPPO2 can catalyze the condensation of CP and (Z)-3-hexenal (Z3H) to form m/z 347. Extracted ion chromatograms (EIC) for doubly charged CP dimer (m/z 250.13) and m/z 347.19 in E. coli expressing NaPPO1 and NaPPO2 with CP and Z3H or (E)-2-hexenal. Compounds 1, 2, 3, 4 denote isomers of CP dimer; compounds 5, 6 denote isomers of m/z 347.19.

FIG. 17 Structural elucidation, biosynthesis, function and engineering of m/z 347.19 in vitro and in planta: the figure shows proposed enzymatic reactions mediated by NaPPO1 or NaPPO2 for m/z 347.19 synthesis: NaPPO1/2 oxidize CP to caffeoyl quinone putrescine, and activate Z3H for the Michael addition reaction, followed by aromatization to yield m/z 347.19.

FIG. 18 Mortality rates of in vitro Empoasca non-choice assays (n=4, 25 Empoasca leafhoppers per replicate) in which Empoasca were fed for 6 h with either 1 μM m/z 347.19 synthesized by using NaPPO1/NaPPO2 and CP and Z3H and diluted in 10% glucose solution or with 10% glucose as control (top). Empoasca in planta choice assays (n=8) conducted with VIGS plants of EV, PPO1, PPO2 and AT1 (bottom).

FIG. 19 Genes involved in the biosynthesis of m/z 347.19.

FIG. 20 Engineering of biosynthetic pathways for the production of m/z 347.19 in plants. Neither Vicia faba, nor the wild tomato Solanum chilense, accumulates m/z 347.19 in response to elicitation; S. chilense accumulates CP after MeJA elicitation whereas V. faba does not (inserted heatmaps). S. chilense can be engineered to produce m/z 347.19 by expressing NaPPO1, NaPPO2 and NaBBL2 with Z3H infiltrations in MeJA-elicited S. chilense leaves. m/z 347.19 accumulates only in S. chilense expressing NaPPO1/NaPPO2 together with NaBBL2, but not NaPPO1/NaPPO2 alone. V. faba can be engineered to produce m/z 347.19 by expressing NaPPO1, NaPPO2 and NaBBL2 with Z3H and CP infiltrations in leaves. Mortality rates of in vivo Empoasca feeding assays (n=4, 25 Empoasca leafhoppers per replicate) in which Empoasca were fed for 10 h on leaves of reconstituted S. chilense and V. faba, respectively. EIC, extracted ion chromatogram.

FIG. 21 The 2019 field MAGIC population grown at the WCCER field station, Prescott, Arizona, USA. Design of MAGIC population indicating distances between trunk lines (8 in an east-west orientation), splitters (4 on each trunk line) and drippers (18 on each splitter).

FIG. 22 The growth stage of plants during herbivore screening for the MAGIC population. Four RILs were planted with 50 cm spacing around each dripper (water emitter).

FIG. 23 Plant treatment and herbivory screening of MAGIC population. W+OS treat-ment and herbivory screening

FIG. 24 Picture of the MAGIC population by 2019 field team members.

FIG. 25 Multi-omics co-association network annotation. Detailed annotations of the outer layers of the multi-omics co-association network which were not tightly linked to JA signalling (providing additional annotations of the network provided in FIG. 3). Different compound classes, phytohormones, JA-related genes and SNPs are shown with different gray tones.

FIG. 26 The Empoasca choice assay setup in the Isserstedt glasshouse. Different N. attenuata transgenic lines (n=10 plants per genotype) were randomly placed on a glasshouse bench with at least 40 cm distance from each other for an open-choice experimental set-up for Empoasca selection. Leafhoppers were released from populations reared on V. faba plants enclosed in mesh tents located on both sides of the glasshouse bench.

FIG. 27 Transcript abundance of targeted genes in transgenic N. attenuata lines. Relative mRNA abundance of genes of rosette-stage plants of different transgenic lines. For lines transformed with inverted repeat (ir) constructs designed to silence gene expression, transcripts were quantified 1 h after W+OS elicitation of a mature+2 leaf older than the source-sink transi-tion leaf from each line and a size-matched EV plant and untreated control plants (C) of each genotype. For plants transformed with an over-expression (ov) constructs only unelicited control plants were used. Asterisks indicate significant differences between W+OS treatments and untreated controls (C) [Student's t-tests on pairwise differences, *P<0.05, **P<0.01, and ***P<0.001; n=6 plants per treatment/genotype]. N.S., not significant.

FIG. 28 Tissue-specific expression profiles of 13 JAZs in N. attenuata WT. The heatmap 5 labeling depicts the Z-score scaled TPM. FLB, flower bud; STI, stigma; COE, corolla early; PED, pedicel; SED, seed; OFL, opening flower; SNP, style; LEC, leaf control; ROT, roots from plants with OS-treated leaves; OVA, ovary; NEC, nectary; ANT, anther; STT, stem treated; LET, leaf treated.

FIG. 29 Interaction among NaMYC2a/b, NaMYB8 and NaJAZi proteins in Y2H assays. GAL4 DNA-BD-NaMYC2a/b/NaMYB8 and AD-JAZs were co-transformed into yeast. The transformants were grown on QDO (SD-Ade/-His/-Leu/-Trp with 2 mM 3-AT at 30° C.) at the indicated dilutions.

FIG. 30 Setups of Empoasca and Manduca bioassays. Setup of the Empoasca (right) and Manduca (left) feeding experiments in a growth room. Note the clip cages used to constrain Em-poasca attack to particular leaves.

FIG. 31 The Empoasca choice (top) and no-choice (bottom) feeding assays in a growth chamber.

FIG. 32 Metabolome diversity and specialization as it is influenced by insect herbivory in different N. attenuata transgenic lines. Scatterplots of diversity (Hj index) against specialization (δj index) for the specialized metabolome with and without E. decipiens (n=3) and M. sexta (n=4) attack for 72 h on 16 different N. attenuata transgenic lines. This figure provides the complete dataset described in FIG. 7.

FIGS. 33A-J Accumulations of phenolic derivatives and phenolamides in N. attenuata leaves in 16 different transgenic lines in response to continuous feeding from two herbivore species. A simplified pathway reveals the associations among the major phenylpropanoid-quinate and -polyamine conjugates in response to Empoasca or Manduca insect feeding. CGA, Chlorogenic acid; CoP, N-coumaroylputrescine; CP, N-caffeoylputrescine; FP, N-feruloylputrescine; CS, N-caffeoylspermidine; FS, N-feruloylspermidine; DCS, N′, N″-dicaffeoylspermidine; CFS, N′,N″-caffeoyl,feruloylspermidine. Asterisks indicate significant differences between insect treatment and control (Student's t-test, *P<0.05, **P<0.01, ***P<0.001; n=3 to 4 plants per treat-ment/genotype). FIG. 33 A) overview, FIG. 33B) CGA, FIG. 33C) Phenylalanine, FIG. 33D) CoP, FIG. 33E) CS, FIG. 33F) CP, FIG. 33G) FS, FIG. 33H) FP, FIG. 33I) DCS and FIG. 33J) CFS.

FIG. 34 Standard curves of CoP, CP and FP. Standard curves for CoP, CP and FP were constructed by preparing synthetic CoP, CP and FP and used for the quantification of the phenolamide levels in leaves and insect diets. LC-MS conditions are provided in the Examples.

FIG. 35 The annotated MS/MS spectrum and structure for the unknown metabolite m/z 347.19. IdMS/MS of m/z 347.19 was annotated by highlighting neutral losses of putrescine and C₆H80 and the corresponding structure of m/z 347.19 as shown on the right.

FIG. 36 Correlations between herbivory-elicited accumulations of m/z 347.19 and the accumulations of jasmonates and Empoasca phenotypes in the MAGIC RIL population grown in the 2019 field season. Pairwise Pearson correlation coefficient (PCC) was conducted using amounts of m/z 347.19 quantified 72 h after W+OS treatment of leaves (1706 samples) with levels of different jasmonates quantified 1 h after W+OS treatment of leaves (1816 samples) and Empoasca phenotypes (1907 samples) from the MAGIC RIL population grown at the Arizona field station. Samples of replicates are averaged. Significant correlations with P value <0.05 are depicted.

FIG. 37 Transcript accumulations of m/z 347.19 biosynthetic genes in N. attenuata leaves in different transgenic lines in response to OS-treatments. Transcript accumulations of m/z 347.19 biosynthetic genes in WT and irMYB8 lines in response to OS-treatment of leaves after 1 h and 5 h, respectively. *P<0.05, **P<0.01, ***P<0.001.

FIG. 38 Detailed kinetics of transcript accumulations of the same biosynthetic genes in WT plants in response to OS-treatment of leaves. *P<0.05, **P<0.01, ***P<0.001.

FIG. 39 Transcript abundance of m/z 347.19 biosynthetic genes in EV and irMYC2 lines 7 h after OS-treatment as quantified by RNAseq and expressed as fragments per kilobase of transcript per million reads sequenced (FPKM). Asterisks indicate significant differences compared to untreated control plants (Two-way ANOVA followed by post hoc multiple comparisons, *P<0.05, **P<0.01, ***P<0.001; n=3 plants per treatment/genotype). N.S., not significant.

FIG. 40 Silencing efficiency of NaAT1, NaPPO1, NaPPO2 and NaBBL2 in VIGS plants. Relative mRNA abundance of NaAT1, NaPPO1, NaPPO2 and NaBBL2 in the rosette-stage of N. attenuata VIGS plants, respectively. Asterisks indicate significant differences between W+OS treatments and untreated controls (C) [Student's t-tests on pairwise differences, *P<0.05, **P<0.01, and ***P<0.001; n=8 to 11 plants per treatment/genotype]. N.S., not significant.

FIG. 41 Accumulation of phenolamides in N. attenuata leaves in different Virus-induced gene silencing (VIGS) plants in response to OS-treatment. Silencing NaPPO1 and NaPPO2 does not significantly alter herbivory-induced phenolamide accumulations comparing with EV. CoP, N-coumaroylputrescine; FP, N-feruloylputrescine; CS, N-caffeoylspermidine; FS, N-feruloylspermidine; CoCS, N′,N″-coumaroyl,caffeoylspermidine; CFS, N′,N″-caffeoyl,feruloylspermidine; DCS, N′, N″-dicaffeoylspermidine; DFS, N′, N″-diferuloylspermidine. Different letters indicate significant differences [Two-way ANOVA followed by Tukey's post hoc multiple comparisons; n=4 to 8 plants per treatment/genotype].

FIG. 42 Correlations between herbivory-elicited m/z 347.19 and volatile organic compounds (VOC8) in a MAGIC RIL population. The Pearson correlation coefficients (PCC) were calculated among the accumulation of m/z 347.19 at 72 h after W+OS treatment of leaves and the release of VOCs during the 24 h following W+OS treatment in the 650 MAGIC RILs grown in the glasshouse. The dashed line indicates the significant PCC correlation threshold with a P value <0.05.

FIG. 43 SDS-PAGE analysis of recombinant NaPPO1 protein during the purification process. SDS-PAGE separations of NaPPO1. Lanes: 1, sample of un-induced control (1 mL); 2, sample of induced overnight fraction (1 mL); 3, sample of insoluble fraction (5 mL); 4, sample of soluble fraction; 5-8, samples collected after gradient rinsing with 40 mM (10 mL), 80 mM (1 mL), 250 mM (1 mL), and 250 mM (1 mL) imidazole buffers. Arrows indicate the purified NaPPO1 protein.

FIG. 44 SDS-PAGE analysis of recombinant NaPPO2 protein during the purification process. SDS-PAGE separations of NaPPO2. Lanes: 1, sample of un-induced control (1 mL); 2, sample of induced overnight fraction (1 mL); 3, sample of insoluble fraction (5 mL); 4, sample of soluble fraction; 5-8, samples collected after gradient rinsing with 40 mM (10 mL), 80 mM (1 mL), 250 mM (1 mL), and 250 mM (1 mL) imidazole buffers. Arrows indicate the purified NaPPO2 protein.

FIG. 45 SDS-PAGE analysis of recombinant NaBBL2. Lanes: 1, sample of un-induced control (1 mL); 2, sample of induced overnight fraction (1 mL); 3, sample of insoluble fraction (5 mL); 4, sample of soluble fraction; 5-8, samples collected after gradient rinsing with 40 mM (10 mL), 80 mM (1 mL), 250 mM (1 mL), and 250 mM (1 mL) imidazole buffers. Arrows indicate the purified NaBBL2 protein.

FIG. 46 Comparison of the MS/MS spectra of in vitro produced m/z 347.19 with Em-poasca-elicited m/z 347.19 in leaves. The MS/MS spectrum of in vitro produced m/z 347.19 is the top spectrum (black) and compared with the MS/MS spectrum of OS-elicited m/z 347.19 in N. attenuata leaves (red: bottom spectrum). The MS/MS spectra were obtained with MS/MS data acquisition targeting m/z 347.19 using an 8 Da mass isolation window and a collision energy of 25 eV. The corresponding extracted ion chromatograms (EIC) for m/z 347.19 (25 min LC method) are at the left.

FIG. 47 Comparison of the MS/MS spectra of in vitro produced unknown m/z 250.132 with a N-caffeoylputrescine standard. The MS/MS spectrum of the in vitro produced unknown with m/z 250.132 is shown (top spectrum). The unknown doubly charged m/z 250.132 (C₂₆H₃₆N₄O₆²⁺) is annotated as a N-caffeoylputrescine dimer comparing with the MS/MS spectrum of N-caffeoylputrescine standards (bottom spectrum).

FIG. 48 NaBBL2 is not required for the in vitro production of m/z 347.19. EICs for m/z 347.19 in E. coli expressing NaPPO1 and NaPPO2 and NaBBL2 with CP and (Z)-3-hexenal added to the media. Arrows indicate two isomers of m/z 347.19 produced in the in vitro enzymatic assay.

FIG. 49 Michaelis-Menten kinetics of PPO1 and BBL2 with different concentrations of caffeoylputrescine and (Z)-3-hexenal. Steady-state kinetics of caffeoylputrescine or (Z)-3-hexenal conversion to CPH catalyzed by PPO1 purified from E. coli B121 expressing PPO1. Error bars represent the standard error of the means (SEM) from three independent measurements of reaction velocity. The kinetic constants, apparent Km and Vmax (Table 2), and their standard errors were inferred using nonlinear Michaelis-Menten curve-fitting in GraphPad Prism (v. 9.0d). Substrate concentration for (Z)-3-hexenal was 1 mM.

FIG. 50 Michaelis-Menten kinetics of PPO2 and BBL2 with different concentrations of caffeoylputrescine and (Z)-3-hexenal. Steady-state kinetics of caffeoylputrescine or (Z)-3-hexenal conversion to CPH catalyzed by PPO2 purified from E. coli B121 expressing PPO2. Error bars represent the standard error of the means (SEM) from three independent measurements of reaction velocity. The kinetic constants, apparent Km and Vmax (Table 2), and their standard errors were inferred using nonlinear Michaelis-Menten curve-fitting in GraphPad Prism (v. 9.0d). Substrate concentration for (Z)-3-hexenal was 1 mM.

FIG. 51 Michaelis-Menten kinetics of PPO1 and BBL2 with different concentrations of caffeoylputrescine and (Z)-3-hexenal. Steady-state kinetics of caffeoylputrescine or (Z)-3-hexenal conversion to CPH catalyzed by PPO1 purified from E. coli B121 expressing PPO1. Error bars represent the standard error of the means (SEM) from three independent measurements of reaction velocity. The kinetic constants, apparent Km and Vmax (see Table 2), and their standard errors were inferred using nonlinear Michaelis-Menten curve-fitting in GraphPad Prism (v. 9.0d). Substrate concentration for caffeoylputrescine was 80 μM.

FIG. 52 Michaelis-Menten kinetics of PPO2 and BBL2 with different concentrations of caffeoylputrescine and (Z)-3-hexenal. Steady-state kinetics of caffeoylputrescine or (Z)-3-hexenal conversion to CPH catalyzed by PPO2 purified from E. coli B121 expressing PPO2. Error bars represent the standard error of the means (SEM) from three independent measurements of reaction velocity. The kinetic constants, apparent Km and Vmax (Table 2), and their standard errors were inferred using nonlinear Michaelis-Menten curve-fitting in GraphPad Prism (v. 9.0d). Substrate concentration for (Z)-3-hexenal was 1 mM.Substrate concentration for caffeoylputrescine was 80 μM.

FIG. 53 Substrate specificity of NaPPO1 and NaPPO2 enzymes. Overlay of total ion chromatograms (TIC) of enzyme assays for authentic chlorogenic acid (CGA std), N-coumaroylputrescine (CoP std), (Z)-3-hexenal or (E)-2-hexenal with purified NaPPO1/NaPPO2 from E. coli. Compounds 1 and 2 denote CGA and CoP; 3 and 4 denote contamination peaks from (Z)-3-hexenal and CoP standards, respectively.

FIG. 54 ¹H NMR spectrum and chemical shifts of m/z 347.19. The ¹H-NMR spectrum of the enzyme product of m/z 347.19 in 700 μL MeOH-d₃, spiked with 0.1% formic acid, with water presaturation, 32 scans (left), and ¹H and ¹³C chemical shifts of m/z 347.19 (right).

FIG. 55 Estimation of half-life of m/z 347.19. Different time acquisitions of NMR spectra for purified m/z 347.19 are shown. The half-life time of m/z 347.19 as estimated from the signal for position 2 at 6 ppm is approximately 22 h in acidified methanol-d₃ (0.1% formic acid) at room temperature in darkness.

FIG. 56 Accumulation of m/z 347.19, CP and biosynthetic enzymes across different plant species. Accumulations of MeJA-induced m/z 347.19 in closely-related Nicotiana species. Asterisks indicate significant differences among lanolin plus wounding (W) or MeJA treatments and lanolin controls (C) [Student's t-tests on pairwise differences, *P<0.05, **P<0.01, and ***P<0.001; n=6 plants per treatment/genotype]. At, N. attenuata; Ac, N. acuminata; Li, N. linearis; Mi, N. miersii; Ob, N. obtusifolia; Pa, N. pauciflora; Sp, N. spegazzinii;

FIG. 57 Phylogenetic tree of sequenced plant genomes created using the Taxonomy Common Tree from NCBI. The heatmap labels are scaled by the percentage of amino acid identity of the top BLAST (blastp) hit by querying NaAT1, NaPPO1, NaPPO2 and NaBBL2 homologues of the respective plant proteomes (left), and the heatmap on the right represents the presence and absence of the MeJA-induced caffeoylputrescine and m/z 347.19 in different species (intensity threshold >500).

FIG. 58 Localization of NaPPO1 and NaPPOl in N. attenuata. (A) ‘thylakoid transfer domain’ (Joy et al., Plant Physiol 107, 1083-1089 (1995)) of plastid PPOs was identified in amino acid sequence of NaPPO1 and NaPPO2 by N-terminal amino acid sequence alignment (thylakoid transfer domain labeled with red rectangle). Alignment of the N-terminal amino acid sequence of NaPPO1 and NaPPO2 with those of plastid polyphenol oxidase (PPO) precursors [Antirrhinum majus aureusidin synthase (AmAS1; AB044884.1), PPO of Vitis vinifer, BAA08234.1, PPO of Solanum tuberosum, AAA85122.1, PPO of Phytolacca americana (PAP1), BAA08234.1].

FIG. 59 Subcellular localization of 35S::NaPPO1-GFP or 35S:p:NaPPO2-GFP, GFP was fused to the C terminal of open reading frame of NaPPOland NaPPO2, transiently expressed in N. attenuata leaves. Control: uninfected plants, scale bar: 20 pm.

FIG. 60 Proposed mechanism of nonhost resistance in N. attenuata μgainst Empoasca leafhoppers. A model of in vivo enzymatic reactions that produce m/z 347.19 is proposed. NaPPO1 and NaPPO2 are localized in the plastid and hence physically separated from caffeoylputrescine (CP) which is likely produced in the cytosol. Upon Empoasca damage, an Em-poasca-elicited jasmonate signalling pathway is activated, in which JA-Ile or other JA derivatives recruit NaJAZi and releases its interaction with NaMYC2 and NaMYB8 to activate CP production. NaPPO1 or NaPPO2 condenses CP with PPO-transported (Z)-3-hexenal from the plastid in reactions proposed in FIG. 17 with the assistance of NaBBL2, and m/z 347.19 is accumulated in the central vacuole, given its relative stability in the weak acetic conditions that commonly charac-terize vacuoles. Alternatively, chloroplasts with their cargoes of GLVs and PPOs may be phago-cytized in response to Empoasca attack and transported into the central vacuole to produce m/z 347.19 with the CP present in the central vacuole.

FIG. 61 Amino acid sequence alignment of NaBBL2 with other characterized berberine bridge enzymes. Red boxes highlight the residues (His and Cys) of berberine bridge enzymes involved in bicovalent attachment of FAD. Cys residue was found to be mutated in NaBBL2 enzymes (C₁₇₃ to G173 mutation). Alignment was performed using MUSCLE algorithm. Accessions numbers: N. attenuata BBL2 (XM_019396928), E. californica BBE (CAK49173), P. somniferum BBE (XP_001394039), C. sativa THCA synthase (AMQ48632), C. sativa CBDA synthase (A6P6V9), C. ja-ponica BBL (BAJ40864).

### DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated nu-merical value of +20%, preferably +15%, more preferably ±10%, and even more preferably ±5

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method, procedure or use there is no time or time interval coherence between the steps, i.e. the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect a compound of general formula (I)

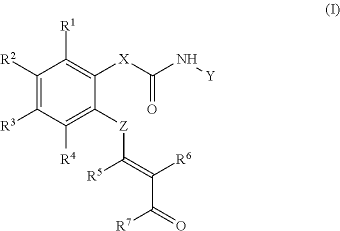

wherein:
R¹, R², R³, and R⁴ are each independently from each other H, OH, (C₁-C₆)-alkyl or (C₁-C₆)-alkoxy;
R⁵, R⁶ and R⁷ are each independently from each other H or (C₁-C₆)-alkyl;
X is straight-chain or branched (C₁-C₈)-alkyl, or straight-chain or branched (C₂-C₈)-alkenyl
Y is selected from —(CH₂)ₘ—NH₂, —(CH₂)ₙ—NH—(CH₂)ₒ—NH₂, —(CH₂)ₚ—NH—(CH₂)_{q}—NH—(CH₂)ᵣ—NH₂, NH—(CH₂)ₘ—NH₂, NH—(CH₂)ₙ—NH—(CH₂)ₒ—NH₂, and NH—(CH₂)ₚ—NH—(CH₂)_{q}—NH—(CH₂)ᵣ—NH₂, with m, n, o, p, q, and r each being an integer between 1 and 10, or a tyramine ester; and
Z is straight-chain or branched (C₁-C₈)-alkyl, or straight-chain or branched (C₂-C₈)-alkenyl,

or an enantiomer, diastereomer, stereoisomer, or salt thereof.

In a further preferred embodiment the present invention relates to the compound of general formula (I) wherein:
two of R¹, R², R³, and R⁴ are OH; and the remaining of R¹, R², R³, and R⁴ are each independently from each other H, OH, (C₁-C₆)-alkyl or (C₁-C₆)-alkoxy;
R⁵, R⁶ and R⁷ are each H;
X is a straight-chain (C₂-C₅)-alkenyl;
Y is selected from —(CH₂)ₘ—NH₂, —(CH₂)ₙ—NH—(CH₂)ₒ—NH₂, —(CH₂)ₚ—NH—(CH₂)_{q}—NH—(CH₂)ᵣ—NH₂, NH—(CH₂)ₘ—NH₂, NH—(CH₂)ₙ—NH—(CH₂)ₒ—NH₂, and NH—(CH₂)ₚ—NH—(CH₂)_{q}—NH—(CH₂)ᵣ—NH₂, with m, n, o, p, q, and r each being an integer between 1 and 10, or a tyramine ester; and
Z is straight-chain or branched (C₁-C₈)-alkyl, or straight-chain or branched (C₂-C₅)-alkenyl,

or an enantiomer, diastereomer, stereoisomer, or salt thereof.

In a further preferred embodiment the present invention relates to the compound of general formula (I) wherein:
R¹ and R⁴ are H;
R² and R³ are OH,
R⁵, R⁶ and R⁷ are each H;
X is X is —CH═CH—;
Y is selected from —(CH₂)ₘ—NH₂, —(CH₂)ₙ—NH—(CH₂)ₒ—NH₂, —(CH₂)ₚ—NH—(CH₂)_{q}—NH—(CH₂)ᵣ—NH₂, NH—(CH₂)ₘ—NH₂, NH—(CH₂)ₙ—NH—(CH₂)ₒ—NH₂, and NH—(CH₂)ₚ—NH—(CH₂)_{q}—NH—(CH₂)ᵣ—NH₂, with m, n, o, p, q, and r each being an integer between 1 and 10, or a tyramine ester; and
Z is straight-chain or branched (C₁-C₈)-alkyl, or straight-chain or branched (C₂-C₅)-alkenyl,

or an enantiomer, diastereomer, stereoisomer, or salt thereof

In one embodiment, X is a straight-chain (C₂-C₈)-alkenyl.

In one embodiment, X is a straight-chain (C₂-C₆)-alkenyl.

In one embodiment, X is a straight-chain (C₂-C₄)-alkenyl.

In one embodiment, X is —CH═CH—.

In one embodiment, Y is selected from —(CH₂)ₘ—NH₂, —(CH₂)ₙ—NH—(CH₂)ₒ—NH₂, —(CH₂)ₚ—NH—(CH₂)_{q}—NH—(CH₂)ᵣ—NH₂, with m, n, o, p, q, and r each being an integer between 1 and 3.

In one embodiment, Y is selected from NH—(CH₂)ₘ—NH₂, NH—(CH₂)ₙ—NH—(CH₂)ₒ—NH₂, and NH—(CH₂)ₚ—NH—(CH₂)_{q}—NH—(CH₂)ᵣ—NH₂, with m, n, o, p, q, and r each being an integer between 1 and 10, or a tyramine ester.

In one embodiment, Y is selected from —(CH₂)ₘ—NH₂, —(CH₂)ₙ—NH—(CH₂)ₒ—NH₂, —(CH₂)ₚ—NH—(CH₂)_{q}—NH—(CH₂)ᵣ—NH₂, with m, n, o, p, q, and r each being 1.

In one embodiment, Y is selected from NH—(CH₂)ₘ—NH₂, NH—(CH₂)ₙ—NH—(CH₂)ₒ—NH₂, and NH—(CH₂)ₚ—NH—(CH₂)_{q}—NH—(CH₂)ᵣ—NH₂, with m, n, o, p, q, and r each being 1, or a tyramine ester.

In one embodiment, compound (I) is of general formula (Ia)

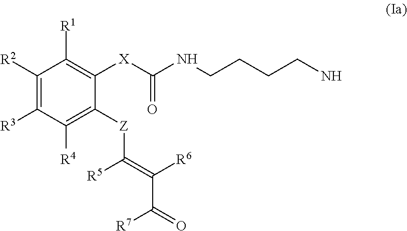

wherein R¹, R², R³, R⁴, R⁵, R⁶, R⁷, X and Z are defined as for formula (I) above, or an enantiomer, diastereomer, stereoisomer, or salt thereof.

In one embodiment, at least one of R¹, R², R³, and R⁴ in formula (Ia) is OH; and the remaining of R¹, R², R³, and R⁴ are each independently from each other H, OH, (C₁-C₆)-alkyl or (C₁-C₆)-alkoxy. Preferably, in formula (Ia) R¹ and R⁴ are H and R² and R³ are OH.

In one embodiment, R⁵ and R⁶ in formula (Ia) are both H.

In one embodiment, R⁵, R⁶ and R⁷ in formula (Ia) are each H.

In one embodiment, X in formula (Ia) is straight-chain (C₂-C₆)-alkenyl.

In one embodiment, X in formula (Ia) is —CH═CH—.

In one embodiment, Z in formula (Ia) is straight-chain or branched (C₁-C₈)-alkyl.

In one embodiment, compound (1) is of general formula (Ib)

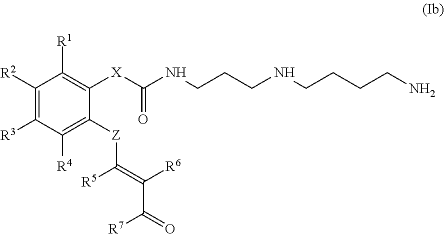

wherein R¹, R², R³, R⁴, R⁵, R⁶ and R⁷, X and Z are defined as for formula (1) above,

or an enantiomer, diastereomer, stereoisomer, or salt thereof.

In one embodiment, at least one of R¹, R², R³, and R⁴ in formula (Ib) is OH; and the remaining of R¹, R², R³, and R⁴ are each independently from each other H, OH, (C₁-C₆)-alkyl or (C₁-C₆)-alkoxy. Preferably, in formula (Ib) R¹ and R⁴ are H and R² and R³ are OH.

In one embodiment, R⁵ and R⁶ in formula (Ib) are both H.

In one embodiment, R⁵, R⁶ and R⁷ in formula (Ib) are each H.

In one embodiment, X in formula (Ib) is straight-chain (C₂-C₆)-alkenyl.

In one embodiment, X in formula (Ib) is —CH═CH—.

In one embodiment, Z in formula (Ib) is straight-chain or branched (C₁-C₈)-alkyl.

In one embodiment, compound (I) is of general formula (Ic)

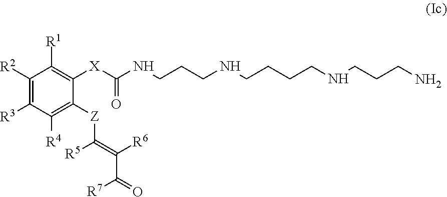

wherein R¹, R², R³, R⁴, R⁵, R⁶ and R⁷, X and Z are defined as for formula (I) above, or an enantiomer, diastereomer, stereoisomer, or salt thereof.

In one embodiment, at least one of R¹, R², R³, and R⁴ in formula (Ic) is OH; and the remaining of R¹, R², R³, and R⁴ are each independently from each other H, OH, (C₁-C₆)-alkyl or (C₁-C₆)-alkoxy. Preferably, in formula (Ic) R¹ and R⁴ are H and R² and R³ are OH.

In one embodiment, R⁵ and R⁶ in formula (Ic) are both H.

In one embodiment, R⁵, R⁶ and R⁷ in formula (Ic) are each H.

In one embodiment, X in formula (Ic) is straight-chain (C₂-C₆)-alkenyl.

In one embodiment, X in formula (Ic) is —CH═CH—.

In one embodiment, Z in formula (Ic) is straight-chain or branched (C₁-C₈)-alkyl.

In one embodiment, compound (I) is of general formula (Id)

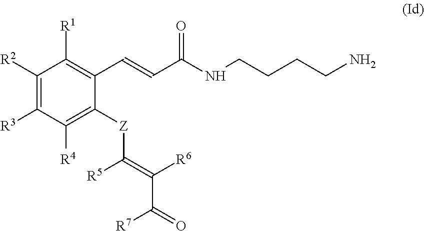

wherein R¹, R², R³, R⁴, R⁵, R⁶ and R⁷, and Z are defined as for formula (I) above, or an enantiomer, diastereomer, stereoisomer, or salt thereof.

In one embodiment, at least one of R¹, R², R³, and R⁴ in formula (Id) is OH; and the remaining of R¹, R², R³, and R⁴ are each independently from each other H, OH, (C₁-C₆)-alkyl or (C₁-C₆)-alkoxy. Preferably, in formula (Id) R¹ and R⁴ are H and R² and R³ are OH.

In one embodiment, R⁵ and R⁶ in formula (Id) are both H.

In one embodiment, R⁵, R⁶ and R⁷ in formula (Id) are each H.

In one embodiment, X in formula (Id) is straight-chain (C₂-C₆)-alkenyl.

In one embodiment, X in formula (Id) is —CH═CH—.

In one embodiment, Z in formula (Id) is straight-chain or branched (C₁-C₈)-alkyl.

In one embodiment, at least one of R¹, R², R³, and R⁴ in formula (I), (Ia), (Ib), (Ic) or (Id) is OH; and the remaining of R¹, R², R³, and R⁴ are each independently from each other H, OH, or (C₁-C₆)-alkoxy.

In one embodiment, at least two of R¹, R², R³, and R⁴ in formula (1), (Ia), (Ib), (Ic) or (Id) are OH; and the remaining of R¹, R², R³, and R⁴ are each independently from each other H, OH, (C₁-C₆)-alkyl or (C₁-C₆)-alkoxy.

In one embodiment, at least two of R¹, R², R³, and R⁴ in formula (1), (Ia), (Ib), (Ic) or (Id) are OH; and the remaining of R¹, R², R³, and R⁴ are each independently from each other H, OH, or (C₁-C₆)-alkoxy.

In one embodiment, two of R¹, R², R³, and R⁴ in formula (1), (Ia), (Ib), (Ic) or (Id) are OH; and the remaining two of R¹, R², R³, and R⁴ are each H. Preferably, R¹ and R⁴ are H and R² and R³ are OH.

In a further particularly preferred embodiment the present invention relates to a compound of formula (II)

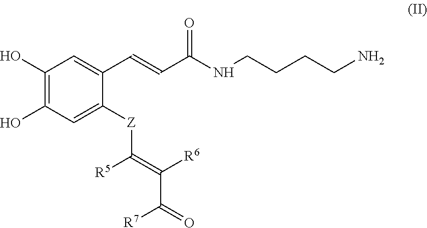

wherein:
R⁵, R⁶ and R⁷ are each independently from each other H or (C₁-C₆)-alkyl;
and Z is straight-chain or branched (C₁-C₈)-alkyl, or straight-chain or branched (C₂—C₈)-alkenyl.

or an enantiomer, diastereomer, stereoisomer, or salt thereof.

In one embodiment, X is —CH═CH—, R¹ is OH, R², R³ and R⁴ are H. In one embodiment X is —CH═CH—, R² is OH, R¹, R³ and R⁴ are H. In one embodiment X is —CH═CH—, R³ is OH, R¹, R² and R⁴ are H. In one embodiment X is —CH═CH—, R⁴ is OH, R¹, R² and R³ are H.

In one embodiment X is —CH═CH—, R¹ and R⁴ are H, R² is —OCH₃ and R³ is OH.

In one embodiment X is —CH═CH—, R¹ and R⁴ are H, R² and R³ are OH.

In one embodiment X is —CH═CH—, R¹ is H, R³ is OH, R² and R⁴ are —OCH₃.

In one embodiment Y is —(CH₂)₄NH₂. In one embodiment Y is —(CH₂)₃—NH—(CH₂)₄—NH₂. In one embodiment Y is —(CH₂)₃—NH—(CH₂)₄—NH₂. In one embodiment Y is —(CH₂)₃—NH—(CH₂)₄—NH—(CH₂)₃—NH₂.

In one embodiment R⁵ and R⁶ are H, Z is (C₁-C₆)-alkyl. In one embodiment R⁵ and R⁶ are H, Z is C₂-alkyl. In one embodiment R⁵ and R⁶ are H, Z is C₃-alkyl. In one embodiment R⁵ and R⁶ are H, Z is C₄-alkyl. In one embodiment R⁵ and R⁶ are H, Z is Cs-alkyl. In one embodiment R⁵ and R⁶ are H, Z is C₆-alkyl. In one embodiment R⁵ and R⁶ are H, Z is CH₂. In one embodiment R⁵ and R⁶ are H, Z is CH₂CH₂. In one embodiment R⁵ and R⁶ are H, Z is CH(CH₃). In one embodiment R⁵ and R⁶ are H, Z is CH₂CH₂CH₂. In one embodiment R⁵ and R⁶ are H, Z is CH(CH₃)CH₂. In one embodiment R⁵ and R⁶ are H, Z is CH₂CH(CH₃). In one embodiment R⁵ and R⁶ are H, Z is CH(CH₂CH₃). In one embodiment R⁵ and R⁶ are H, Z is C(CH₃)₂. In one embodiment R⁵ and R⁶ are H, Z is CH₂CH₂CH₂CH₂.

In one embodiment R⁵ and R⁶ are H, Z is CH(CH₃)CH₂CH₂. In one embodiment R⁵ and R⁶ are H, Z is CH₂CH(CH₃)CH₂. In one embodiment R⁵ and R⁶ are H, Z is CH₂CH₂CH(CH₃). In one embodiment R⁵ and R⁶ are H, Z is C(CH₃)₂CH₂. In one embodiment R⁵ and R⁶ are H, Z is CH₂C(CH₃)₂. In one embodiment R⁵ and R⁶ are H, Z is CH₂CH(CH₂CH₃). In one embodiment R⁵ and R⁶ are H, Z is CH(CH₂CH₃)CH₂. Preferably, in the above embodiments, wherein R⁵ and R⁶ are H, R⁷ is also H.

In another embodiment X is —CH═CH—, R¹ is OH, R², R³ and R⁴ are H and Y is —(CH₂)₄NH₂. In one embodiment X is —CH═CH—, R² is OH, R¹, R³ and R⁴ are H and Y is —(CH₂)₄NH₂. In one embodiment X is —CH═CH—, R³ is OH, R¹, R² and R⁴ are H and Y is —(CH₂)₄NH₂. In one embodiment X is —CH═CH—, R⁴ is OH, R¹, R² and R³ are H and Y is —(CH₂)₄NH₂. In one embodiment X is —CH═CH—, R¹ and R4 are H, R² is —OCH₃ and R³ is OH and Y is —(CH₂)₄NH₂. In one embodiment X is —CH═CH—, R¹ and R⁴ are H, R² and R³ are OH and Y is —(CH₂)₄NH₂. In one embodiment X is —CH═CH—, R¹ is H, R³ is OH, R² and R⁴ are —OCH₃ and Y is —(CH₂)₄NH₂.

In a further embodiment X is —CH═CH—, R¹ is OH, R², R³ and R⁴ are H and Y is —(CH₂)₃—NH—(CH₂)₄—NH₂. In one embodiment X is —CH═CH—, R² is OH, R¹, R³ and R⁴ are H and Y is —(CH₂)₃—NH—(CH₂)₄—NH₂. In one embodiment X is —CH═CH—, R³ is OH, R¹, R² and R⁴ are H and Y is —(CH₂)₃—NH—(CH₂)₄—NH₂. In one embodiment X is —CH═CH—, R⁴ is OH, R¹, R² and R³ are H and Y is —(CH₂)₃—NH—(CH₂)₄—NH₂. In one embodiment X is —CH═CH—, R¹ and R⁴ are H, R² is —OCH₃ and R³ is OH and Y is —(CH₂)₃—NH—(CH₂)₄—NH₂. In one embodiment X is —CH═CH—, R¹ and R⁴ are H, R² and R³ are OH and Y is —(CH₂)₃—NH—(CH₂)₄—NH₂. In one embodiment X is —CH═CH—, R¹ is H, R³ is OH, R² and R⁴ are —OCH₃ and Y is —(CH₂)₃—NH—(CH₂)₄—NH₂.

In a further embodiment X is —CH═CH—, R¹ is OH, R², R³ and R⁴ are H and Y is —(CH₂)₃—NH—(CH₂)₄—NH—(CH₂)₃—NH₂. In one embodiment X is —CH═CH—, R² is OH, R¹, R³ and R⁴ are H and Y is —(CH₂)₃—NH—(CH₂)₄—NH—(CH₂)₃—NH₂. In one embodiment X is —CH═CH—, R³ is OH, R¹, R² and R⁴ are H and Y is —(CH₂)₃—NH—(CH₂)₄—NH—(CH₂)₃—NH₂. In one embodiment X is —CH═CH—, R⁴ is OH, R¹, R² and R³ are H and Y is —(CH₂)₃—NH—(CH₂)₄—NH—(CH₂)₃—NH₂. In one embodiment X is —CH═CH—, R¹ and R⁴ are H, R² is —OCH₃ and R³ is OH and Y is —(CH₂)₃—NH—(CH₂)₄—NH—(CH₂)₃—NH₂. In one embodiment X is —CH═CH—, R¹ and R⁴ are H, R² and R³ are OH and Y is —(CH₂)₃—NH—(CH₂)₄—NH—(CH₂)₃—NH₂. In one embodiment X is —CH═CH—, R¹ is H, R³ is OH, R² and R⁴ are —OCH₃ and Y is —(CH₂)₃—NH—(CH₂)₄—NH—(CH₂)₃—NH₂.

In a further particularly preferred embodiment the compound is of general formula (I) or an enantiomer, diastereomer, stereoisomer, or salt thereof, wherein X is —CH═CH—, R¹ is OH, R², R³ and R⁴ are H, Y is —(CH₂)₄NH₂, R⁵, R⁶ and R⁷ are H, and Z is (C₁-C₆)-alkyl. In a further particularly preferred embodiment X is —CH═CH—, R² is OH, R¹, R³ and R⁴ are H, Y is —(CH₂)₄NH₂, R⁵, R⁶ and R⁷ are H, and Z is (C₁-C₆)-alkyl. In a further particularly preferred embodiment X is —CH═CH—, R³ is OH, R¹, R² and R⁴ are H, Y is —(CH₂)₄NH₂, R⁵, R⁶ and R⁷ are H, and Z is (C₁-C₆)-alkyl. In a further particularly preferred embodiment X is —CH═CH—, R⁴ is OH, R¹, R² and R³ are H, Y is —(CH₂)₄NH₂, R⁵, R⁶ and R⁷ are H, and Z is (C₁-C₆)-alkyl. In a further particularly preferred embodiment X is —CH═CH—, R¹ and R⁴ are H, R² is —OCH₃ and R³ is OH, Y is —(CH₂)₄NH₂, R⁵, R⁶ and R⁷ are H, and Z is (C₁-C₆)-alkyl.

In a further particularly preferred embodiment X is —CH═CH—, R¹ and R⁴ are H, R² and R³ are OH, Y is —(CH₂)₄NH₂, R⁵, R⁶ and R⁷ are H, and Z is (C₁-C₆)-alkyl. In a further particularly preferred embodiment X is —CH═CH—, R¹ is H, R³ is OH, R² and R⁴ are —OCH₃, Y is —(CH₂)₄NH₂, R⁵, R⁶ and R⁷ are H, and Z is (C₁-C₆)-alkyl.

In a further particularly preferred embodiment the compound is of general formula (I) or an enantiomer, diastereomer, stereoisomer, or salt thereof, wherein X is —CH═CH—, R¹ is OH, R², R³ and R⁴ are H, Y is —(CH₂)₃—NH—(CH₂)₄—NH₂, R⁵, R⁶ and R⁷ are H, and Z is (C₁-C₆)-alkyl. In a further particularly preferred embodiment X is —CH═CH—, R² is OH, R¹, R³ and R⁴ are H, Y is —(CH₂)₃—NH—(CH₂)₄—NH₂, R⁵, R⁶ and R⁷ are H, and Z is (C₁-C₆)-alkyl. In a further particularly preferred embodiment X is —CH═CH—, R³ is OH, R¹, R² and R⁴ are H, Y is —(CH₂)₃—NH—(CH₂)₄—NH₂, R⁵, R⁶ and R⁷ are H, and Z is (C₁-C₆)-alkyl. In a further particularly preferred embodiment X is —CH═CH—, R⁴ is OH, R¹, R² and R³ are H, Y is —(CH₂)₃—NH—(CH₂)₄—NH₂, R⁵, R⁶ and R⁷ are H, and Z is (C₁-C₆)-alkyl. In a further particularly preferred embodiment X is —CH═CH—, R¹ and R⁴ are H, R² is —OCH₃ and R³ is OH, Y is —(CH₂)₃—NH—(CH₂)₄—NH₂, R⁵, R⁶ and R⁷ are H, and Z is (C₁-C₆)-alkyl. In a further particularly preferred embodiment X is —CH═CH—, R¹ and R⁴ are H, R² and R³ are OH, Y is —(CH₂)₃—NH—(CH₂)₄—NH₂, R⁵, R⁶ and R⁷ are H, and Z is (C₁-C₆)-alkyl. In a further particularly preferred embodiment X is —CH═CH—, R¹ is H, R³ is OH, R² and R⁴ are —OCH₃, Y is —(CH₂)₃—NH—(CH₂)₄—NH₂, R⁵, R⁶ and R⁷ are H, and Z is (C₁-C₆)-alkyl.

In a further particularly preferred embodiment the compound is of general formula (I) or an enantiomer, diastereomer, stereoisomer, or salt thereof, wherein X is —CH═CH—, R¹ is OH, R², R³ and R⁴ are H, Y is —(CH₂)₃—NH—(CH₂)₄—NH—(CH₂)₃—NH₂, R⁵, R⁶ and R⁷ are H, and Z is (C₁-C₆)-alkyl. In a further particularly preferred embodiment X is —CH═CH—, R² is OH, R¹, R³ and R⁴ are H, Y is —(CH₂)₃—NH—(CH₂)₄—NH—(CH₂)₃—NH₂, R⁵, R⁶ and R⁷ are H, and Z is (C₁-C₆)-alkyl. In a further particularly preferred embodiment X is —CH═CH—, R³ is OH, R¹, R² and R⁴ are H, Y is —(CH₂)₃—NH—(CH₂)₄—NH—(CH₂)₃—NH₂, R⁵, R⁶ and R⁷ are H, and Z is (C₁-C₆)-alkyl. In a further particularly preferred embodiment X is —CH═CH—, R⁴ is OH, R¹, R² and R³ are H, Y is —(CH₂)₃—NH—(CH₂)₄—NH—(CH₂)₃—NH₂, R⁵, R⁶ and R⁷ are H, and Z is (C₁-C₆)-alkyl. In a further particularly preferred embodiment X is —CH═CH—, R¹ and R⁴ are H, R² is —OCH₃ and R³ is OH, Y is —(CH₂)₃—NH—(CH₂)₄—NH—(CH₂)₃—NH₂, R⁵, R⁶ and R⁷ are H, and Z is (C₁-C₆)-alkyl. In a further particularly preferred embodiment X is —CH═CH—, R¹ and R⁴ are H, R² and R³ are OH, Y is —(CH₂)₃—NH—(CH₂)₄—NH—(CH₂)₃—NH₂, R⁵, R⁶ and R⁷ are H, and Z is (C₁-C₆)-alkyl. In a further particularly preferred embodiment X is —CH═CH—, R¹ is H, R³ is OH, R² and R⁴ are —OCH₃, Y is —(CH₂)₃—NH—(CH₂)₄—NH—(CH₂)₃—NH₂, R⁵, R⁶ and R⁷ are H, and Z is (C₁-C₆)-alkyl.

In a further particularly preferred embodiment the compound is of general formula (I) or an enantiomer, diastereomer, stereoisomer, or salt thereof, wherein X is —CH═CH—, R¹ is OH, R², R³ and R⁴ are H, Y is —(CH₂)₄NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R² is OH, R¹, R³ and R⁴ are H, Y is —(CH₂)₄NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R³ is OH, R¹, R² and R⁴ are H, Y is —(CH₂)₄NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R⁴ is OH, R¹, R² and R³ are H, Y is —(CH₂)₄NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ and R⁴ are H, R² is —OCH₃ and R³ is OH, Y is —(CH₂)₄NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ and R⁴ are H, R² and R³ are OH, Y is —(CH₂)₄NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ is H, R³ is OH, R² and R⁴ are —OCH₃, Y is —(CH₂)₄NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂.

In a further particularly preferred embodiment the compound is of general formula (1) or an enantiomer, diastereomer, stereoisomer, or salt thereof, wherein X is —CH═CH—, R¹ is OH, R², R³ and R⁴ are H, Y is —(CH₂)₃—NH—(CH₂)₄—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R² is OH, R¹, R³ and R⁴ are H, Y is —(CH₂)₃—NH—(CH₂)₄—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R³ is OH, R¹, R² and R⁴ are H, Y is —(CH₂)₃—NH—(CH₂)₄—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R⁴ is OH, R¹, R² and R³ are H, Y is —(CH₂)₃—NH—(CH₂)₄—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ and R⁴ are H, R² is —OCH₃ and R³ is OH, Y is —(CH₂)₃—NH—(CH₂)₄—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ and R⁴ are H, R² and R³ are OH, Y is —(CH₂)₃—NH—(CH₂)₄—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ is H, R³ is OH, R² and R⁴ are —OCH₃, Y is —(CH₂)₃—NH—(CH₂)₄—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂.

In a further particularly preferred embodiment the compound is of general formula (1) or an enantiomer, diastereomer, stereoisomer, or salt thereof, wherein X is —CH═CH—, R¹ is OH, R², R³ and R⁴ are H, Y is —(CH₂)₃—NH—(CH₂)₄—NH—(CH₂)₃—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R² is OH, R¹, R³ and R⁴ are H, Y is —(CH₂)₃—NH—(CH₂)₄—NH—(CH₂)₃—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R³ is OH, R¹, R² and R⁴ are H, Y is —(CH₂)₃—NH—(CH₂)₄—NH—(CH₂)₃—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R⁴ is OH, R¹, R² and R³ are H, Y is —(CH₂)₃—NH—(CH₂)₄—NH—(CH₂)₃—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ and R⁴ are H, R² is —OCH₃ and R³ is OH, Y is —(CH₂)₃—NH—(CH₂)₄—NH—(CH₂)₃—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ and R⁴ are H, R² and R³ are OH, Y is —(CH₂)₃—NH—(CH₂)₄—NH—(CH₂)₃—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂. In a further particularly preferred embodiment X is —CH═CH—, R¹ is H, R³ is OH, R² and R⁴ are —OCH₃, Y is —(CH₂)₃—NH—(CH₂)₄—NH—(CH₂)₃—NH₂, R⁵, R⁶ and R⁷ are H, and Z is C₃-alkyl, wherein the C₃-alkyl is selected from CH₂CH₂CH₂, CH(CH₃)CH₂, CH₂CH(CH₃), CH(CH₂CH₃), and C(CH₃)₂.

In the most preferred embodiment the present invention relates to a compound having the following formula (III):

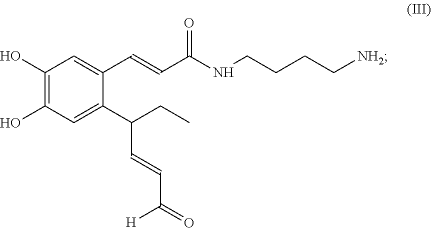 [SMILES: CCC(/C=C/C=O)c1cc(O)c(O)cc1/C=C/C(=O)NCCCCN]

or an enantiomer, diastereomer, stereoisomer, or salt thereof.

In certain specific embodiments of the invention in the compounds as defined above, alkyl and alkenyl chains, i.e. the straight-chain or branched (C₁-C₈)-alkyl of X, the straight-chain or branched (C₂-C₅)-alkenyl of X, the straight-chain or branched (C₁-C₈)-alkyl of Z and the straight-chain or branched (C₂-C₅)-alkenyl of Z may optionally be substituted with suitable substituents. Suitable substituents include, but are not limited to OH, F, Cl, Br or CN.

In a further aspect the present invention relates to a method of producing the compound of the invention as defined herein above, e.g. the compound of formula (I), preferably the compound of formula (II) and more preferably the compound of formula (III). The term “producing the compound of the invention” as used herein means that the compound of the invention is generated by any suitable procedure, in any suitable amount and in any suitable purity. Such procedure may, for example, be based on components being present in an in vitro environment, or being present in an in vivo environment.

The production is preferably an enzymatic production method. The term “enzymatic production method” as used herein means that one or more reactants or precursors of the compound according to the invention are modified, transformed, combined or otherwise altered into one or more products, finally yielding a compound according to the invention, with the assistance of one or more enzymes and optionally additional factors such as energy carriers, co-factors, reducing equivalents, ions etc. and under specific conditions, e.g. a specific pH, a certain ion concentration, in a specific environment, e.g. an aqueous environment etc. The enzymatic production method may involve one, two, three, 4, 5, 6, 7, 8, 9, 10 or more enzymatic conversion, transformation or modification steps. The method may further comprise steps, which are non-enzymatic, e.g. allow for the correct positioning of a compound, allow for an interaction of two or more polypeptides or allow for a recruiting of polypeptides or activities to a complex or site of reaction. The order of these steps may vary. Typically, the order of the conversion, transformation or modification steps follows a certain chemical logic staring with simple reactants and ending with complex products. The method may in certain embodiments make use of a precursor or reactant which may be the product of another enzymatic production method or enzymatic activity or which may be the product of a non-enzymatic synthesis or be provided de novo. The presence, identity and amount of such precursor(s) may have an influence on the overall number of steps in the enzymatic production method. For example, a precursor or reactant may be provided which requires only enzymatic steps 4, 5, 6 etc. in order to yield a compound according to the invention since said precursor or reactant is identical or similar to the product of enzymatic steps 1 through 3; or a precursor may be provided which requires enzymatic steps 8, 9 and 10 in order to yield a compound according to the invention since said precursor or reactant is identical or similar to the product of enzymatic steps 1 through 7. Further, different precursors or reactants for different enzymatic steps may be provided simultaneously or sequentially during the product method, e.g. 2, 3, 4, 5 or more different precursors or reactants may be provided in order to yield a compound according to the invention. In certain embodiments, the presence of enzymatic activities may be made dependent on the presence of a precursor or reactant in the environment where the production method is performed.

In specific embodiments precursors, reactants or substrates may be provided to the site of reaction, e.g. a living cell or organism, preferably a plant as defined herein, from an external site. Such precursors, reactants or substrates may, for example, be provided in pre- or pro-form, which require one or more metabolic steps in order to yield a form which can be used within the method of the present invention. These steps may preferably be performed by standard conversion processes of a living cells, in particular of plant cell or plant.

In a further specific embodiment said precursor, reactant or substrate may be provided to a living cell, e.g. a plant cell as defined herein, in form of a lipophilic ester, e.g. as methyl ester.

Such esters may be removed by suitable enzymes such as esterases. Esterases are typically present in a living cell, e.g. in a higher plant cell.

The precursors, reactants or substrates may, for example, be provided to a living cell, e.g. a plant as defined herein, via spraying techniques (further information may be derived, for example from Daware and Lokhande, International Journal of Innovations in Engineering and Science, 4, 8 (2019). In further embodiments, they may be mixed with a surfactant or spreading agent, e.g. as defined herein below.

In a preferred embodiment, the enzymatic production method using at least a BBL2 (berberine bridge enzyme 2) polypeptide. The term “polypeptide” as used herein refers to a continuous and unbranched peptide chain of a certain length. In contrast thereto a “peptide” relates to any type of amino acid sequence comprising more than 2 amino acids or functional derivatives thereof. Furthermore, the peptide may be combined with further chemical moieties or function-alities. A polypeptide may, for example, have a length of more than 20 to 50 amino acids. The term “protein” as used herein relates to an arrangement of one or more polypeptides. Accordingly, a protein may comprise or consist of one polypeptide and thus by synonymous to polypeptide. In other embodiments, a protein may comprise 2 or more polypeptides which may be organized in units or subunits of a higher order structure in the form of a protein. The term “activity” as used herein relates to a polypeptide, preferably an enzyme, fulfilling a certain biological function, preferably an enzymatic function. In certain embodiments, the activity may be an enzymatic function which converts a reactant into a product.

A “BBL2 (berberine bridge enzyme 2) polypeptide” relates to a BBL2 polypeptide or fulfilling the biologic function of a BBL2 polypeptide. The berberine bridge enzymes have been described as FAD-linked oxidases, which have a special C-terminal structural element adjacent to the substrate binding region. Typically, a FAD binding module is formed by the N- and C- terminal parts of the protein. There is a substrate binding module that, in collaboration with isoalloxazine ring of FAD, disposes the environment for efficient substrate binding and oxidation.

Without wishing to be bound by theory, it is assumed that BBL2 functions within the context of the present invention as a non-catalytic protein in the enzymatic production method as described herein. This function may be similar to a non-catalytic chalcone isomerase-like (CHIL) in flavonoid metabolism. It is further assumed that BBL2 interacts with the reactive PPOactivated N-caffeoylputrescine (CP) or a derivative thereof as a reaction intermediate to stabilize it in the cell environment and avoids conversion to a by-product that cannot react with (Z)-3-hexenal to form a compound according to the invention, e.g. CPH (caffeoylputrescine-3-hexenal compound). As such, BBL2 is believed to allow for more efficient channelling of substrates among active enzymes, e.g. in the form of a metabolon. Metabolons typically facilitate channelling of labile and toxic intermediates and increase local substrate concentrations and prevent undesired metabolic cross-talk. Such dynamic assembly and disassembly permits rapid reorganization of metabolic profiles in response to environmental challenges and are thought to involve scaffolding proteins. Accordingly, a scaffolding function devoted to BBL2 could possibly allow for a dynamic assembly and disassembly of a metabolon channelling reaction intermediates and maximizing catalytic efficiency toward the production of a compound according to the invention, e.g. CPH.

Preferably BBL2 is (a) encoded by the polynucleotide having the nucleotide sequence of SEQ ID NO: 1; (b) encoded by a polynucleotide which is a variant of SEQ ID NO: 1; (c) encoded by a polynucleotide which is an allelic variant of SEQ ID NO: 1; (d) encoded by a polynucleotide which is a species homologue of SEQ ID NO: 1; (e) encoded by a polynucleotide which is at least 75%, 80%, 90%, 95%, 97%, 98%, or 99% identical to the polynucleotide as defined in any one of (a) to (d); (f) encoded by a polynucleotide capable of hybridizing under stringent conditions to any one of the polynucleotides specified in (a) to (d); (g) represented by the polypeptide of SEQ ID NO: 2; (h) represented by a polypeptide fragment of SEQ ID NO: 2 having BBL2 (berberine bridge enzyme 2) function; (i) represented by a polypeptide domain of SEQ ID NO: 2 having BBL2 (berberine bridge enzyme 2) function (j) represented by a polypeptide having an amino acid sequence at least 75%, 80%, 90%, 95%, 97%, 98%, or 99% identical to the amino acid sequence of SEQ ID NO: 2 and having BBL2 (berberine bridge enzyme 2) function or (k) represented by a polypeptide being encoded by any one of the polynucleotides specified in (a) to (f).

The term “polynucleotide” as used herein relates to a nucleic acid or nucleic acid molecule as known to the person skilled in the art, e. g. a DNA, RNA, single stranded DNA, cDNA, or derivatives thereof. The nucleic acid can further be linear or circular. Preferably, the term refers to a DNA molecule.

The term “allelic variant” as used herein refers to a variant polynucleotide which exists in two or more different allele forms at a particular locus.

As used herein, a “fragment”, “variant” or “homologue” of a polynucleotide relates to polynucleotide which comprises or consists of a nucleotide sequence which has at least 75%, preferably 80%, 85%, 90%, 91%, 92%, 93%, 94%, 95%, 96%, 97%, 98%, 99% or nucleotide sequence identity to nucleotide sequence of a reference polynucleotide (e. g. that of SEQ ID NO: 1). In certain embodiments fragments, variants and homologues may encode a polypeptide being capable of performing one, more or all function(s) performed by the corresponding reference 5 polypeptide (e.g. that of SEQ ID NO: 2).

Similarly, a “fragment”, “variant” or “homologue” of a polypeptide relates to polypeptide which comprises or consists of an amino acid sequence which has at least 75%, preferably 80%, 85%, 90%, 91%, 92%, 93%, 94%, 95%, 96%, 97%, 98%, 99% or more amino acid sequence identity to the amino acid sequence of a reference polypeptide (e. g. that of SEQ ID NO: 2). In certain embodiments fragments, variants and homologues of a reference polypeptide may be capable of performing one, more or all function(s) performed by the reference polypeptide (e.g. that of SEQ ID NO: 2).

The term “species homologue” relates to a homologous, i.e. highly similar, polynucleotide or amino acid sequence which is derived from a different species than the reference sequence (e.g. SEQ ID NO: 1 or 2). Such a high similarity is typically a strong evidence that two sequences are related by evolutionary changes from a common ancestral sequence. A species homologue is typically a functional homologue, i.e. not only the nucleotide or amino acid sequence of the homologue is similar to the reference sequence, but also the function of the corresponding polypeptide, e.g. an enzymatic activity, is similar or identical to the function of a reference polypeptide.

The term “stringent conditions” or “stringent hybridization conditions” as used herein refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 g/m) denatured, sheared salmon sperm DNA, followed by washing the filters in 0.1×SSC at about 65° C. Changes in the stringency of hybridization and signal detection are primarily accomplished through the manipulation of formamide concentration (lower per-centages of formamide result in lowered stringency); salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization may be carried out at higher salt concentrations (e. g. with 5×SSC). Further variations in the above conditions may be accomplished through the inclusion and/or substitution of alternate blocking reagents used to suppress background in hybridization experiments. Typical blocking reagents to be used in the context of the present invention include Denhardt's reagent, BLOTTO, heparin or denatured salmon sperm DNA. The inclusion of specific blocking reagents may require modification of the hybridization conditions described above, due to problems with compatibility.

By a nucleic acid having a nucleotide sequence at least, for example, 95% “identical” to a reference nucleotide sequence of the present invention, it is intended that the nucleotide sequence of the polynucleotide is identical to the reference sequence except that the nucleotide sequence may include up to five point mutations per each 100 nucleotides of the reference nucleotide sequence encoding the polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence or any fragment as described herein. Whether any particular nucleic acid molecule is at least 75%, 80%, 85%, 86%, 87%, 88%, 89%, 90%, 91%, 92%, 93%, 94%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al., 1990, Comp. App. Biosci. 6: 237-245. In a nucleotide sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=l, Gap Penalty=5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage may then be subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence. Whether any particular polypeptide is at least at least 75%, 80%, 85%, 86%, 87%, 88%, 89%, 90%, 91%, 92%, 93%, 94%, 95%, 96%, 97%, 98% or 99% identical to, for instance, an amino acid sequence of the present invention can be determined conventionally by using known computer programs. A preferred method for determining the best overall match between a query sequence (a sequence of the present invention) and a subject sequence, also referred to as a global sequence alignment, can be determined using the FASTDB computer program based on the algorithm of Brutlag et al., 1990, Comp. App. Biosci. 6: 237-245. In an amino acid sequences alignment the query and subject sequences are both amino acid sequences. The result of said global sequence alignment is given in percent identity.

Preferred parameters used in a FASTDB amino acid alignment are: Matrix=PAM 0, k-tuple=2, Mismatch Penalty=l, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned may be determined by the results of the FASTDB sequence alignment.

This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N—and C-terminal residues of the subject sequence.

According to specific embodiments alternative local sequence identity algorithms may be used for sequence alignment and comparison purposes. For example, the algortihm of Smith and Waterman, 1981, Adv. Appl. Math. 2:482, the sequence identity alignment algorithm of Needle-man and Wunsch, 1970, J. Mol. Biol. 48:443, the search for similarity method of Pearson and Lipman, 1988, Proc. Nat. Acad. Sci. U.S.A. 85:2444, or computerized implementations of these algorithms such as multiple sequence alignment tools Clustal Omega (https://www.ebi.ac.uk/Tools/msa/clustalo/), T-coffee/M-coffee (http://tcoffee.crg.cat/), BLAST (https://blast.ncbi.nlm.nih.gov), FASTA (https://www.ebi.ac.uk/Tools/sss/fasta/) or the like, preferably using the default settings, may be employed. A further envisaged example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360. Another example of a useful algorithm is the BLAST algorithm, described in Altschul et al., 1990, J. Mol. Biol. 215:403-410 or the WU-BLAST-2 program. WU-BLAST-2 uses several search parameters, most of which are set to the default values. An additional useful algorithm is gapped BLAST which uses BLOSUM-62 substitution scores.

In a further embodiment the enzymatic production method additionally uses a PPO (polyphenol oxidase) activity or polypeptide. It is preferred that the enzymatic production method uses a BBL polypeptide in combination with a PPO activity or polypeptide.

A “PPO (polyphenol oxidase) activity or polypeptide” relates to a PPO polypeptide or fulfilling the enzymatic or biologic function of a PPO polypeptide. The polyphenol oxidase enzymes typically accept monophenols and/or o-diphenols as substrates. The enzymes have been described as catalyzing the o-hydroxylation of monophenol molecules in which the benzene ring contains a single hydroxyl substituent to o-diphenols. They further catalyse the oxidation of o-diphenols to produce o-quinones. Further, PPO was found to catalyse the polymerization of o-quinones to produce polyphenols.

In particularly preferred embodiments, said PPO (polyphenol oxidase) activity or polypeptide is: (a) encoded by the polynucleotide having the nucleotide sequence of SEQ ID NO: 3 or 5;(b) encoded by a polynucleotide which is a variant of SEQ ID NO: 3 or 5; (c) encoded by a polynucleotide which is an allelic variant of SEQ ID NO: 3 or 5; (d) encoded by a polynucleotide which is a species homologue of SEQ ID NO: 3 or 5; (e) encoded by a polynucleotide which is at least 75%, 80%, 90%, 95%, 97%, 98%, or 99% identical to the polynucleotide as defined in any one of (a) to (d); (f) encoded by a polynucleotide capable of hybridizing under stringent conditions to any one of the polynucleotides specified in (a) to (d); (g) represented by the polypeptide of SEQ ID NO: 4 or 6; (h) represented by a polypeptide fragment of SEQ ID NO: 4 or 6 having PPO (polyphenol oxidase) activity; (i) represented by a polypeptide domain of SEQ ID NO: 4 or 6 having PPO (polyphenol oxidase) activity; (j) represented by a polypeptide having an amino acid sequence at least 75%, 80%, 90%, 95%, 97%, 98%, or 99% identical to the amino acid sequence of SEQ ID NO: 4 or 6 and having PPO (polyphenol oxidase) activity; or (k) represented by a polypeptide being encoded by any one of the polynucleotides specified in (a) to (f).

In a further embodiment the enzymatic production method additionally uses an AT1 (polyamine hydroxycinnamoyltransferase 1) activity or polypeptide. It is preferred that the enzymatic production method uses a BBL polypeptide in combination with a PPO activity or polypeptide and an AT1 activity or polypeptide.

An “AT1 (polyamine hydroxycinnamoyltransferase 1) activity or polypeptide” relates to an AT1 polypeptide or fulfilling the enzymatic or biologic function of an AT1 polypeptide. The polyamine hydroxycinnamoyltransferase has been described as enzyme that catalyzes the transfer of an acyl from, e.g. p-coumaryol-CoA to a polyamine such as agmatine or putrescine. It has been shown that it can use feruloyl-CoA, caffeoyl-CoA and sinapoyl-CoA as acyl donors.

In particularly preferred embodiments, said AT1 (polyamine hydroxycinnamoyl-transferase 1) activity or polypeptide is: (a) encoded by the polynucleotide having the nucleotide sequence of SEQ ID NO: 7; (b) encoded by a polynucleotide which is a variant of SEQ ID NO: 7; (c) encoded by a polynucleotide which is an allelic variant of SEQ ID NO: 7; (d) encoded by a polynucleotide which is a species homologue of SEQ ID NO: 7; (e) encoded by a polynucleotide which is at least 75%, 80%, 90%, 95%, 97%, 98%, or 99% identical to the polynucleotide as defined in any one of (a) to (d); (f) encoded by a polynucleotide capable of hybridizing under stringent conditions to any one of the polynucleotides specified in (a) to (d); (g) represented by the polypeptide of SEQ ID NO: 8; (h) represented by a polypeptide fragment of SEQ ID NO: 8 having AT1 (polyamine hydroxycinnamoyltransferase 1) activity; (i) represented by a polypeptide domain of SEQ ID NO: 8 having AT1 (polyamine hydroxycinnamoyltransferase 1) activity; (j) represented by a polypeptide having an amino acid sequence at least 75%, 80%, 90%, 95%, 97%, 98%, or 99% identical to the amino acid sequence of SEQ ID NO: 8 and having AT1 (polyamine hydroxycinnamoyl-transferase 1) activity; or (k) represented by a polypeptide being encoded by any one of the polynucleotides specified in (a) to (f).

In a further embodiment the enzymatic production method additionally uses an ODC (ornithine decarboxylase) activity or polypeptide. It is preferred that the enzymatic production method uses a BBL polypeptide in combination with a PPO activity or polypeptide and an AT1 activity or polypeptide and/or an ODC activity or polypeptide.

An “ODC (ornithine decarboxylase) activity or polypeptide” relates to an ODC polypeptide or fulfilling the enzymatic or biologic function of an ODC polypeptide. The ornithine decarboxylase has been described as enzyme that catalyzes the decarboxylation of ornithine to form putrescine. This decarboxylation reaction catalyzed by ornithine decarboxylase is the first and committed step in the synthesis of polyamines such as putrescine, spermidine and spermine.

In particularly preferred embodiments, said ODC (ornithine decarboxylase) activity or polypeptide is: (a) encoded by a polynucleotide having the nucleotide sequence of SEQ ID NO: 9 or 11; (b) encoded by a polynucleotide which is a variant of SEQ ID NO: 9 or 11; (c) encoded by a polynucleotide which is an allelic variant of SEQ ID NO: 9 or 11; (d) encoded by a polynucleotide which is a species homologue of SEQ ID NO: 9 or 11; (e) encoded by a polynucleotide which is at least 75%, 80%, 90%, 95%, 97%, 98%, or 99% identical to the polynucleotide as defined in any one of (a) to (d); (f) encoded by a polynucleotide capable of hybridizing under stringent conditions to any one of the polynucleotides specified in (a) to (d); (g) represented by a polypeptide of SEQ ID NO: 10 or 12; (h) represented by a polypeptide fragment of SEQ ID NO: 10 or 12 having ODC (ornithine decarboxylase) activity; (i) represented by a polypeptide domain of SEQ ID NO: 10 or 12 having ODC (ornithine decarboxylase) activity; (j) represented by a polypeptide having an amino acid sequence at least 75%, 80%, 90%, 95%, 97%, 98%, or 99% identical to the amino acid sequence of SEQ ID NO: 10 or 12 and having ODC (ornithine decarboxylase) activity; or (k) represented by a polypeptide being encoded by any one of the polynucleotides specified in (a) to (f).

In a further embodiment the enzymatic production method additionally uses an HPL (hydroperoxide lyase) activity or polypeptide. It is preferred that the enzymatic production method uses a BBL polypeptide in combination with a PPO activity or polypeptide and an AT1 activity or polypeptide and/or an ODC activity or polypeptide and/or an HPL activity or polypeptide. In further embodiments the enzymatic production method uses a BBL polypeptide in combination with a PPO and ODC and HPL activity or polypeptide; or a BBL polypeptide in combination with a PPO and AT1 and HPL activity; or a BBL polypeptide in combination with an a PPO and AT1 and ODC and HPL activity.

An “HPL (hydroperoxide lyase) activity or polypeptide” relates to an HPL polypeptide or fulfilling the enzymatic or biologic function of a HPL polypeptide. The hydroperoxide lyase has been described as catalyzing the cleavage of C—C bonds in the hydroperoxides of fatty acids.

In particularly preferred embodiments, said HPL (hydroperoxide lyase) activity or polypeptide is: (a) encoded by a polynucleotide having the nucleotide sequence of SEQ ID NO: 13, 15 or 17; (b) encoded by a polynucleotide which is a variant of SEQ ID NO: 13, 15 or 17; (c) encoded by a polynucleotide which is an allelic variant of SEQ ID NO: 13, 15 or 17; (d) encoded by a polynucleotide which is a species homologue of SEQ ID NO: 13, 15 or 17; (e) encoded by a polynucleotide which is at least 75%, 80%, 90%, 95%, 97%, 98%, or 99% identical to the polynucleotide as defined in any one of (a) to (d); (f) encoded by a polynucleotide capable of hybridizing under stringent conditions to any one of the polynucleotides specified in (a) to (d); (g) represented by a polypeptide of SEQ ID NO: 14, 16 or 18; (h) represented by a polypeptide fragment of SEQ ID NO: 14, 16 or 18 having HPL (hydroperoxide lyase) activity; (i) represented by a polypeptide domain of SEQ ID NO: 14, 16 or 18 having HPL (hydroperoxide lyase) activity; (j) represented by a polypeptide having an amino acid sequence at least 75%, 80%, 90%, 95%, 97%, 98%, or 99% identical to the amino acid sequence of SEQ ID NO: 14, 16 or 18 and having HPL (hydroperoxide lyase) activity; or (k) represented by a polypeptide being encoded by any one of the polynucleotides specified in (a) to (f).

In a further embodiment the enzymatic production method additionally uses a PAL (L-phenylalanine ammonia lyase) activity or polypeptide. It is preferred that the enzymatic production method uses a BBL polypeptide in combination with a PPO activity or polypeptide and an AT1 activity or polypeptide and/or an ODC activity or polypeptide and/or an HPL activity or polypeptide and/or a PAL activity or polypeptide. In further embodiments the enzymatic production method uses a BBL polypeptide in combination with a PPO and ODC and PAL activity or polypeptide, or a BBL polypeptide in combination with a PPO and AT1 and ODC and PAL activity or polypeptide; or a BBL polypeptide in combination with a PPO and ODC and HPL and PAL activity; or a BBL polypeptide in combination with a PPO and AT1 and ODC and HPL and PAL activity.

A “PAL (L-phenylalanine ammonia lyase) activity or polypeptide” relates to a PAL polypeptide or fulfilling the enzymatic or biologic function of a PAL polypeptide. L-phenylalanine ammonia lyase was found to catalyze a reaction converting L-phenylalanine to ammonia and trans-cinnamic acid. Phenylalanine ammonia lyase (PAL) is the first and committed step in the phenyl propanoid pathway and is involved in the biosynthesis of polyphenol compounds such as flavonoids, phenylpropanoids, and lignin in plants.

In particularly preferred embodiments, said PAL (L-phenylalanine ammonia lyase) activity or polypeptide is: (a) encoded by a polynucleotide having the nucleotide sequence of SEQ ID NO: 19, 21, 23 or 25;(b) encoded by a polynucleotide which is a variant of SEQ ID NO: 19, 21, 23 or 25;(c) encoded by a polynucleotide which is an allelic variant of SEQ ID NO: 19, 21, 23 or 25;(d) encoded by a polynucleotide which is a species homologue of SEQ ID NO: 19, 21, 23 or 25;(e) encoded by a polynucleotide which is at least 75%, 80%, 90%, 95%, 97%, 98%, or 99% identical to the polynucleotide as defined in any one of A (a) to (d);(f) encoded by a polynucleotide capable of hybridizing under stringent conditions to any one of the polynucleotides specified in (a) to (d);(g) represented by a polypeptide of SEQ ID NO: 20, 22, 24 or 26; (g) represented by a polypeptide of SEQ ID NO: 20, 22, 24 or 26; (h) represented by a polypeptide fragment of SEQ ID NO: 20, 22, 24 or 26 having PAL (L-phenylalanine ammonia lyase) activity; (i) represented by a polypeptide domain of SEQ ID NO: 20, 22, 24 or 26 having PAL (L-phenylalanine ammonia lyase) activity; (j) represented by a polypeptide having an amino acid sequence at least 75%, 80%, 90%, 95%, 97%, 98%, or 99% identical to the amino acid sequence of SEQ ID NO: 20, 22, 24 or 26 and having PAL (L-phenylalanine ammonia lyase) activity; or (k) represented by a polypeptide being encoded by any one of the polynucleotides specified in (a) to (f).

In a further embodiment the enzymatic production method additionally uses a C4H (trans-cinnamate 4-hydroxylase) activity or polypeptide. It is preferred that the enzymatic production method uses a BBL polypeptide in combination with a PPO activity or polypeptide and an AT1 activity or polypeptide and/or an ODC activity or polypeptide and/or an HPL activity or polypeptide and/or a PAL activity or polypeptide and/or a C4H activity or polypeptide. In further embodiments the enzymatic production method uses a BBL polypeptide in combination with a PPO and ODC and HPL and C4H activity or polypeptide, or a BBL polypeptide in combination with a PPO and AT1 and ODC and HPL and PAL and C4H activity or polypeptide; or a BBL polypeptide in combination with a PPO and ODC and HPL and PAL and C4H activity; or a BBL polypeptide in combination with a PPO and AT1 and ODC and HPL and PAL and C4H activity or polypeptide.

A “C4H (trans-cinnamate 4-hydroxylase) activity or polypeptide” relates to a C4H polypeptide or fulfilling the enzymatic or biologic function of a C4H polypeptide. Trans-cinnamate 4-hydroxylase was described as catalyzing a reaction converting trans-cinnamic acid (CA) to p-couma-ric acid (COA) in the phenylpropanoid/lignin biosynthesis pathway of plants.

In particularly preferred embodiments, said C4H (L-phenylalanine ammonia lyase) activity or polypeptide is: (a) encoded by a polynucleotide having the nucleotide sequence of SEQ ID NO: 27; (b) encoded by a polynucleotide which is a variant of SEQ ID NO: 27; (c) encoded by a polynucleotide which is an allelic variant of SEQ ID NO: 27; (d) encoded by a polynucleotide which is a species homologue of SEQ ID NO: 27; (e) encoded by a polynucleotide which is at least 75%, 80%, 90%, 95%, 97%, 98%, or 99% identical to the polynucleotide as defined in any one of B (a) to (d); (f) encoded by a polynucleotide capable of hybridizing under stringent conditions to any one of the polynucleotides specified in (a) to (d); (g) represented by the polypeptide of SEQ ID NO: 28; (h) represented by a polypeptide fragment of SEQ ID NO: 28 having C4H (cinnamate 4-hydroxylase) activity; (i) represented by a polypeptide domain of SEQ ID NO: 28 having C4H (cinnamate 4-hydroxylase) activity; (j) represented by a polypeptide having an amino acid sequence at least 75%, 80%, 90%, 95%, 97%, 98%, or 99% identical to the amino acid sequence of SEQ ID NO: 28 and having C4H (cinnamate 4-hydroxylase) activity; or (k) represented by a polypeptide being encoded by any one of the polynucleotides specified in (a) to (f).

In a further embodiment the enzymatic production method additionally uses a 4CL (4-coumarate:coenzyme A ligase) activity or polypeptide. It is preferred that the enzymatic production method uses a BBL polypeptide in combination with a PPO activity or polypeptide and an AT1 activity or polypeptide and/or an ODC activity or polypeptide and/or an HPL activity or polypeptide and/or a PAL activity or polypeptide and/or a C4H activity or polypeptide and/or a 4CL activity or polypeptide. In further embodiments the enzymatic production method uses a BBL polypeptide in combination with a PPO and ODC and HPL and PAL and C4H and 4CL activity or polypeptide, or a BBL polypeptide in combination with a PPO and AT1 and ODC and HPL and PAL and C4H and 4CL activity or polypeptide; or a BBL polypeptide in combination with a PPO and ODC and HPL and PAL and C4H and 4CL activity; or a BBL polypeptide in combination with a PPO and AT1 and ODC and HPL and PAL and C4H and 4CL activity; or a BBL polypeptide in combination with a PPO and HPL and PAL and 4CL activity.

A “4CL (4-coumarate:coenzyme A ligase) activity or polypeptide” relates to an 4CL polypeptide or fulfilling the enzymatic or biologic function of a C4H polypeptide.

4-coumarate:coenzyme A ligase is a ligase which specifically forms carbon-sulfur bonds as acid-thiol ligases. It catalyzes the formation of hydroxycinnamates CoA esters, and plays an es-sential role at the divergence point from general phenylpropanoid metabolism to major branch pathway of coumarin.

In particularly preferred embodiments, said 4CL (4-coumarate:coenzyme A ligase) activity or polypeptide is: (a) encoded by a polynucleotide having the nucleotide sequence of SEQ ID NO: 29, 105, 107, 109, 111, 113, 115, 117, 119, 121, 123, 125, 127, 129 or 131; (b) encoded by a polynucleotide which is a variant of SEQ ID NO: 29, 105, 107, 109, 111, 113, 115, 117, 119, 121, 123, 125, 127, 129 or 131; (c) encoded by a polynucleotide which is an allelic variant of SEQ ID NO: 29, 105, 107, 109, 111, 113, 115, 117, 119, 121, 123, 125, 127, 129 or 131; (d) encoded by a polynucleotide which is a species homologue of SEQ ID NO: 29, 105, 107, 109, 111, 113, 115, 117, 119, 121, 123, 125, 127, 129 or 131; (e) encoded by a polynucleotide which is at least 75%, 80%, 90%, 95%, 97%, 98%, or 99% identical to the polynucleotide as defined in any one of (a) to (d); (f) encoded by a polynucleotide capable of hybridizing under stringent conditions to any one of the polynucleotides specified in C (a) to (d); (g) represented by the polypeptide of SEQ ID NO: 30, 106, 108, 110, 112, 114, 116, 118, 120, 122, 124, 126, 128, 130 or 132; (h) represented by a polypeptide fragment of SEQ ID NO: 30, 106, 108, 110, 112, 114, 116, 118, 120, 122, 124, 126, 128, 130 or 132 having 4CL (4-coumarate:coenzyme A ligase) activity; (i) represented by a polypeptide domain of SEQ ID NO: 30, 106, 108, 110, 112, 114, 116, 118, 120, 122, 124, 126, 128, 130 or 132 having 4CL (4-coumarate:coenzyme A ligase) activity;(j) represented by a polypeptide having an amino acid sequence at least 75%, 80%, 90%, 95%, 97%, 98%, or 99% identical to the amino acid sequence of SEQ ID NO: 30, 106, 108, 110, 112, 114, 116, 118, 120, 122, 124, 126, 128, 130 or 132 and having 4CL (4-coumarate:coenzyme A ligase) activity; or (k) represented by a polypeptide being encoded by any one of the polynucleotides specified in (a) to (f).

In a further embodiment the enzymatic production method additionally uses a HCT (Hy-droxycinnamoyltransferase) activity or polypeptide. It is preferred that the enzymatic production method uses a BBL polypeptide in combination with a PPO activity or polypeptide and an AT1 activity or polypeptide and/or an ODC activity or polypeptide and/or an HPL activity or polypeptide and/or a PAL activity or polypeptide and/or a C4H activity or polypeptide and/or a 4CL activity or polypeptide and/or a HCT activity or polypeptide. In further embodiments the enzymatic production method uses a BBL polypeptide in combination with a PPO and ODC and HPL 10 and PAL and C4H and 4CL activity or polypeptide, or a BBL polypeptide in combination with a PPO and AT1 and ODC and HPL and PAL and C4H and 4CL and a HCT activity or polypeptide; or a BBL polypeptide in combination with a PPO and ODC and HPL and PAL and C4H and 4CL and HCT activity; or a BBL polypeptide in combination with a PPO and AT1 and ODC and HPL and PAL and C4H and HCT activity; or a BBL polypeptide in combination with a PPO and HPL and PAL and C4H and 4CL and HCT activity.

A “HCT (Hydroxycinnamoyl-transferase) activity or polypeptide” relates to anHCT polypeptide or fulfilling the enzymatic or biologic function of a HCT polypeptide. Hydroxycinnamoyl-transferase, which is also known as shikimate hydroxycinnamoyltransferase participates in phenylpropanoid biosynthesis. It uses 4-coumaroyl-CoA and shikimate as substrate.

In particularly preferred embodiments, said HCT (Hydroxycinnamoyl-transferase) activity or polypeptide is: (a) encoded by a polynucleotide having the nucleotide sequence of SEQ ID NO: 31, 33, 35, 37, 39, 41, or 43; (b) encoded by a polynucleotide which is a variant of SEQ ID NO: 31, 33, 35, 37, 39, 41, or 43; (c) encoded by a polynucleotide which is an allelic variant of SEQ ID NO: 31, 33, 35, 37, 39, 41, or 43; (d) encoded by a polynucleotide which is a species homologue of 25 SEQ ID NO: 31, 33, 35, 37, 39, 41, or 43 (e) encoded by a polynucleotide which is at least 75%, 80%, 90%, 95%, 97%, 98%, or 99% identical to the polynucleotide as defined in any one of (a) to (d); (f) encoded by a polynucleotide capable of hybridizing under stringent conditions to any one of the polynucleotides specified in (a) to (d); (g) represented by a polypeptide of SEQ ID NO: 32, 34, 36, 38, 40, 42 or 44; (h) represented by a polypeptide fragment of SEQ ID NO: 32, 34, 36, 38, 40, 42 or 44 having HCT (Hydroxycinnamoyl-transferase) activity; (i) represented by a polypeptide domain of SEQ ID NO: 32, 34, 36, 38, 40, 42 or 44 having HCT (Hydroxycinnamoyl-transferase) activity; (j) represented by a polypeptide having an amino acid sequence at least 75%, 80%, 90%, 95%, 97%, 98%, or 99% identical to the amino acid sequence of SEQ ID NO: 32, 34, 36, 38, 40, 42 or 44 and having HCT (Hydroxycinnamoyl-transferase) activity; or (k) represented by a polypeptide being encoded by any one of the polynucleotides specified in (a) to (f).

In a further embodiment the enzymatic production method additionally uses a C3H (coumarate 3-hydroxylase) activity or polypeptide. It is preferred that the enzymatic production method uses a BBL polypeptide in combination with a PPO activity or polypeptide and an AT1 activity or polypeptide and/or an ODC activity or polypeptide and/or an HPL activity or polypeptide and/or a PAL activity or polypeptide and/or a C4H activity or polypeptide and/or a 4CL activity or polypeptide and/or a HCT activity or polypeptide and/or a C3H activity or polypeptide In further embodiments the enzymatic production method uses a BBL polypeptide in combination with a PPO and ODC and HPL and PAL and C4H and 4CL activity or polypeptide, or a BBL polypeptide in combination with a PPO and AT1 and ODC and HPL and PAL and C4H and 4CL and HCT and a C3H activity or polypeptide; or a BBL polypeptide in combination with a PPO and ODC and HPL and PAL and C4H and 4CL and HCT and C3H activity or polypeptide activity; or a BBL polypeptide in combination with a PPO and AT1 and HPL and PAL and C4H and 4CL and HCT and C3H activity or polypeptide activity; or a BBL polypeptide in combination with a PPO and HPL and PAL and 4CL and HCT and C3H activity or polypeptide activity.

A “C3H (coumarate 3-hydroxylase) activity or polypeptide” relates to aC3H polypeptide or fulfilling the enzymatic or biologic function of a C3H polypeptide. Coumarate 3-hydroxylases was found to catalyzing the direct 3-hydroxylation of 4-coumarate to caffeate in lignin biosynthesis.

In particularly preferred embodiments, said C3H (coumarate 3-hydroxylase) activity or polypeptide is: (a) encoded by a polynucleotide having the nucleotide sequence of SEQ ID NO: 45;(b) encoded by a polynucleotide which is a variant of SEQ ID NO: 45;(c) encoded by a polynucleotide which is an allelic variant of SEQ ID NO: 45;(d) encoded by a polynucleotide which is a species homologue of SEQ ID NO: 45;(e) encoded by a polynucleotide which is at least 75%, 80%, 90%, 95%, 97%, 98%, or 99% identical to the polynucleotide as defined in any one of E (a) to (d); (f) encoded by a polynucleotide capable of hybridizing under stringent conditions to any one of the polynucleotides specified in (a) to (d);(g) represented by a polypeptide of SEQ ID NO: 46; (h) represented by a polypeptide fragment of SEQ ID NO: 46 having C3H (coumarate 3-hydroxylase) activity;(i) represented by a polypeptide domain of SEQ ID NO: 46 having C3H (coumarate 3-hydroxylase) activity; (j) represented by a polypeptide having an amino acid sequence at least 75%, 80%, 90%, 95%, 97%, 98%, or 99% identical to the amino acid sequence of SEQ ID NO: 46 and having C3H (coumarate 3-hydroxylase) activity; or (k) represented by a polypeptide being encoded by any one of the polynucleotides specified in (a) to (f).

According to the invention the enzymatic production may be performed in any environment or context which suitably leads to a compound as defined herein. The environment may be an in vitro environment or an in vivo environment. The term “in vitro” as used herein means that the production is performed with biological molecules, e.g. activities or polypeptides as defined herein, and optionally with additional factors such as energy carriers, co-factors, reducing equivalents, ions etc. outside of their normal biological context, e.g. in a tube or reactor or reaction vessel or the like. In an in vitro environment typically no cells or tissues are present.

The environment may alternatively be an “in vivo” environment. In such an environment biological molecules may be provided in a cellular context, preferably in living cell, tissue or organism. This may include the provision of activities or polypeptides as mentioned produced by a cell or a group of cells, or the provision of additional factors such as energy carriers, co-factors, reducing equivalents, ions etc. by the cell or cellular environment. In certain embodiments, one or more of these components may be provided from the outside of the cell, tissue or organism, e.g. via a culture medium, injections or the like. The in vivo environment may be a homologous or natural environment, i.e. an environment where the biological molecules etc. are provided in their natural context. Alternatively, the in vivo environment is a heterologous environment. The term “heterologous” as used herein means that at least one activity or polypeptide required for the enzymatic production or a sequence encoding this activity or polypeptide or any other additional factor such as energy carriers, co-factors is not normally or naturally found in the producing cell, tissue or organism, but is introduced into said cell, tissue or organism or has been modified in the producing cell, tissue or organism. The heterologous environment may, for example, be a higher plant species into which one or more activities or polypeptides required for the enzymatic production, or sequences encoding the one or more activities or polypeptides, have been introduced, e.g. via genetic engineering. In other embodiments, the heterologous environment may be a microbiologic cell, e.g. a bacterial or fungal cell, which comprises one or more activities or polypeptides required for the enzymatic production or sequences encoding the one or more activities or polypeptides that have been introduced via, e.g. genetic engineering or transformation into the cell.

In specific preferred embodiments the enzymatic production according to the invention is performed with activities or polypeptides provided an in vitro environment under any suitable conditions. The activities or polypeptides required for the production may comprise those defined herein, e.g. a BBL2 polypeptide and a PPO, AT1, ODC, HPL, PAL, C4H, 4CL, HCT and/or C3H polypeptide or activity. One or more of these activities or polypeptides may be provided in any suitable purity, concentration or amount. The one or more activities or polypeptides may, for example, be obtained, extracted and/or purified according to suitable protocols known to the skilled person. For example, the one or more activities or polypeptides required for the production of a compound according to the invention may be expressed from vectors or plasmids in suitable microbes, i.e. bacterial expression systems, preferably by using suitable overexpression promoters. The expression may be performed under suitable temperature conditions, e.g. at 37° C. or 16° C. Subsequently the bacterial cells may be collected, lysed and homogenized. Supernatant may be incubated with suitable resins or on suitable columns to purify and separate polypeptide fractions. In a particularly preferred embodiment the production is performed as described in the Examples, in particular in Example 16.

According to certain embodiments, the in vitro enzymatic production may be performed with different groups or combinations or selections of activities or polypeptides. The grouping may typically be defined according to the step or advancement in the overall pathway leading to a compound according to the present invention, e.g. as depicted in FIG. 19. According to the selection of activities or polypeptides specific starting metabolites or compounds may be required. For example, if a group of activities or polypeptides comprising PPO and BBL2 is selected, a starting metabolite or starting compound such as N-Caffeoylputrescine may be required and accordingly be provided in a suitable amount, purity or concentration in the in vitro environment.

In addition a further reactant such as (Z)-3-hexenal (Z3H) may be provided.

For example, if a group of activities or polypeptides comprising AT1, ODC HPL, PPO and BBL2 is selected, a starting metabolite or starting compound such as putrescine, spermine or spermidine may be required and accordingly be provided in a suitable amount, purity or concentration in the in vitro environment. In addition a further reactant such as Caffeoyl-CoA may be provided.

For example, if a group of activities or polypeptides comprising PAL, C4H, 4CL, HCT, C3H AT1, ODC, HPL, PPO and BBL2 is selected, a starting metabolite or starting compound such as phenylalanine may be required and accordingly be provided in a suitable amount, purity or concentration in the in vitro environment. In addition a further reactant may be provided.

In addition to the above examples the present invention further envisages also any other selection or grouping of polypeptides or activities as defined herein, as well as the addition of any further suitable reaction, starting compound or metabolite yield a compound of the invention. Also envisaged is the use of subgroupings of the activities or polypeptides as defined herein to yield a certain intermediate product and the transfer of the intermediate to a different environment comprising a different or further subgrouping of the activities or polypeptides as defined herein. In other embodiments, the activities or polypeptides may be provided in a combined form, e.g. all groupings or subgroupings are introduced into the in vitro environment in combination, or they may be provided consecutively, e.g. a second, third, fourth etc. activity is provided after a specific time period since the previous provision has passed etc.

In further embodiments, the production method and/or its efficiency may be controlled by sample taking after a certain period of time. Such samples may subsequently be tested in order to identify certain compounds and/or in order to measure the amount of such compounds, e.g. an intermediate or a compound according to the invention as defined herein. For such a testing for example mass spectroscopy means and methods may be employed.

The production method may be performed with any suitable buffer, e.g. an acetate buffer. It is particularly preferred that the in vitro production method is performed under conditions of a pH of about 4.8 or lower, e.g. a pH 4.7, 4.6, 4.5, 4.4., 4.3, 4.2, 4.1, 4.0, 3.9, 3.8, 3.7, 3.6, 3.5, 3.4, 3.3, 3.2, 3.1, 3.0 etc. or any value in between the mentioned valued or any lower value Particularly preferred is a pH of 4.8. Such a pH may be obtained in a >80 mM acetate buffer. In additional embodiments the method is performed a temperature of about 5 to 15° C., preferably at about 6 to 12° C., more preferably at about 7 to 10° C., most preferably at about 8° C. In further embodiments, the method is performed for any suitable period of time, preferably for 24 to 72 h, more preferably for about 48 h. Further details may be derived from the Examples, e.g. from Example 16 or 17. Activities or polypeptides may alternatively be purchased from any suitable supplier.

In further preferred embodiments, the production method is carried out by using or involves the use of a solid phase extraction (SPE) approach. It is particularly preferred that the SPE uses an argon flow.

The term “production” as used in the context of an in vivo production refers to the generation or synthesis of a compound of the invention by a suitable living cell or tissue or organism.

The synthesized compound may further be accumulated by said cell, tissue or organism. The term “accumulate” or “accumulation” means that the synthesized compound is stored intracellularly and/or is excreted into the surrounding in both cases leading to an overall increase of the compound concentration in comparison to a natural orwildtype cell or organisms which, forexample, does not comprise or express an activity or polypeptide as defined herein, e.g. a BBL2, PPO, AT1, ODC, HPL, PAL, C4H, 4CL, HCT and/or C3H activity.

For producing the compound of the invention in vivo the present invention envisages in certain embodiments a heterologous production in a suitable microbial cell or organism. Examples of suitable microbial cells or organisms include prokaryotic or eukaryotic expression hosts.

For example, a microbial cell or organism may be a bacterium, e.g. a bacterium of the genus Klebsiella, Clostridium, Bacillus, Arthobacter, Streptomyces, Corynebacterium, Erwinia, Xanthomonas, Lactobacillus, Caldicellulosiruptor, Pseudomonas, Alcanivorax, Brevibacterium, Bifidobacterium, Escherichia, or Staphylococcus. Preferably, the bacterium may be of the genus Escherichia such, more preferably the bacterium is Escherichia coli. Also envisaged is the employment of fungi, e.g. of the genus Aspergillus, Candida, Saccharomyces, Ustilago, Cryptococcus, Fusarium, Rhizopus, Magnaporthe, Komagataella, Trichderma, Penicillium, Acremonium, Mucor, Alternaria, Botrytis, Endothia, Rhizoctonia, Sclerotinia, Klyveromyces, Torulopsis, Sporotrichum, Geotrichum, Verticillium, Botryosphaeria, Trichothecium, Hansenula, Schizosaccharomyces, Brettanomyces, or Neurospora. Preferred is the employment of Candida and Saccharomyces.

In specific embodiment, the method envisages growing said microbial cell or organism in a culture medium. The term “growing in a culture medium” as used herein refers to the use of any suitable means and methods known to the person skilled in the art, which allows the growth of the cell or organism as defined herein and which is suitable for the synthesis and/or accumulation of the compound of the invention in said cell or organism. The culture medium may, for example, be adapted to the growth pattern of the organism, e.g. comprise a carbon source or, in case of autotrophic organisms lack a carbon source.

In specific embodiments for E. coli and related organisms media such as Terrific Broth (TB), Luria-Bertani Medium (LB), or M9 minimal medium may be used. The skilled person would further be aware of other media which are suitable for E. coli, also envisaged herein, as well as their preparation, e.g. from suitable literature sources or databases. Typically, the TB medium may comprise in a 1 liter unit 12 g Bacto-tryptone, 24 g Bacto yeast extract, 4 mL Glycerol, add distilled water ad 900 ml, which is autoclaved and subsequently completed with the addition of 100 mL sterile 0.17M KH₂PO₄ and 0.72M K₂HPO₄. Typically, the LB medium may comprise in a 1 liter unit Typically, the LB medium may comprise in a 1 liter unit 10 g Bacto-tryptone, 5 g yeast extract, 10 g NaCl, distilled water ad 1000 ml, which is subsequently autoclaved. Typically, a M9 minimal medium in a 1 liter unit may comprise 880 ml sterile water, 100 ml M9 salts stock solution, 1 ml autoclaved 1 M MgSO₄, 0.1 ml autoclaved 1 M CaCl₂ and 20 ml 20% glucose (sterile), wherein the M9 salts stock solution (10×) comprises 60 g Na2HPO₄×7 H2O, 30 g KH₂PO₄, 5 g NaCl, 10 g NH₄Cl to which water ad 1000 ml is added and which is subsequently autoclaved.

The cultivation may be carried out as batch process or in a continuous fermentation process. Preferably, the cell or organism is grown in the presence of a precursor such as phenylalanine or other suitable amino acids. Methods for carrying out batch or continuous fermentation processes are well known to the person skilled in the art and are described in the literature, e.g. in Li et al., Microb Cell Fact, 2015, 14 (83). The culturing may be carried out under specific temperature conditions, e.g. between 15° C. and 37° C., preferably between 20° C. and 30° C. or 15° C. and 30° C., more preferably between 20° C. and 30° C. and most preferably at about 24° C. In another embodiment, the culturing may be carried out at pH range of pH 3 to 4.8. The fermentation period may vary in dependence on the dimension of the fermentation approach, the medium used, the organism used etc. In certain embodiments of the present invention, a fermentation period of about 6 to 72 h may be used, preferably a fermentation period of about 10 to 24 h, more preferably a fermentation period of 12, 14, 16, 18 or 20 h or any value in between the mentioned values. The fermentation may start with a freshly inoculated culture, e.g. coming from a solid medium or a frozen stock. It is preferred that the fermentation starts with pre-culture, e.g. an overnight culture. Cells may accordingly be transferred to the main culture at a specific OD₆₀₀ value, e.g. at an OD₆₀₀ of 0.5 to 0.8, preferably at 0.6. If a batch fermentation is performed, cells may be cultivated in glucose-limited defined minimal medium at high densities, the formation may be stopped at a specific OD₆₀₀ value of about 1 to 100, preferably of about 100. Further details may be derived from suitable literature sources such as Li et al., Microb Cell Fact, 2015, 14 (83).

In further specific embodiments, the culture medium may comprise additional substances. An example of such an additional substance is an antibiotic, e.g. tetracycline, ampicillin, kanamycin. Such antibiotics may be used as selection instruments for extrachromosomal elements comprising a corresponding resistance cassette, or as inducers for corresponding regulated promoters, e.g. as defined herein below. They may be used in any suitable concentration, e.g. in a suitable concentration range of 50 to 400 μg/ml in the case of ampicillin such as 50, 100, 150 μg/ml, or in a suitable range of 25 to 50 μg/ml in the case of kanamycin, such as 25 or 50 μg/ml. Further details would be known to the skilled person, or can be derived from suitable literature sources.

For producing the compound of the invention in vivo the present invention alternatively envisages in certain embodiments a heterologous production in a suitable eukaryotic, preferably higher eukaryotic cell or organism, or in a tissue of said organism, preferably an organism, cell or tissue as defined herein below.

The in vivo production of the compound of the present invention a higher eukaryotic cell or organism such as a plant cell, plant tissue or a plant organism may in embodiment be performed in order to accumulate said compound in said plant cell or tissue with the aim of subsequently extracting it, e.g. with the assistance of suitable plant material extraction methods, preferably as mentioned in the Examples. In an alternative embodiment, the in vivo production of the compound of the present invention in a higher eukaryotic cell or organism such as a plant cell, plant tissue or a plant organism may be performed in order to increase the resistance of the producing cell, tissue or organism against an insect herbivore, e.g. by a herbivore normally attacking the producing plant, e.g. as defined herein. The corresponding producing cell, tissue or organism may hence be protected against such attacks allowing for an increase in harvest and yield.

For a heterologous production in a suitable cell, tissue or organism said cell, tissue or organism may be genetically modified. This genetic modification allows for the expression or heterologous expression of one or more activities or polypeptides as defined herein. For example, the genetic modification allows for the expression of a BBL2 polypeptide and a PPO, AT1, ODC, HPL, PAL, C4H, 4CL, HCT and/or C3H activity or polypeptide. The genetic modification may additionally also allow for the expression or heterologous expression of any additional factor, element, polypeptide or activities which is required for a production method according to the present invention or which facilitates accessory steps such as transport, accumulation, import, export, excretion, stabilization, pre- or pro-precursor modification, energy supply, reactant supply etc.

The term “genetically modified” or “genetic modification” as used herein means that a cell, tissue or organism is altered by any suitable genetic means and methods known to the skilled person in order to produce a compound of the invention. Similarly, the term “cell, tissue or organism which is genetically modified” as used herein means that a cell, tissue or organism has 15 been modified or altered by any suitable genetic means and methods known to the skilled person such that it synthesizes a compound according to the invention. The cell, tissue or organism may further also accumulate and/or excrete or export said compound. The term also includes the modification of an already genetically modified cell or organism, e.g. as starting cell or organism.

In the present invention a cell, tissue or organism is genetically modified to express one or activities or polypeptides as defined herein. These activities or polypeptides are typically linked to the phenylpropanoid pathway, the polyamine pathway, the green leaf volatiles (GLV) pathway and/or the caffeoylputrescine-hexenal (CPH) pathway, e.g. as depicted in FIG. 19. The present invention envisages further activities such as transporting activities, export or import activities, secretion or excretion activities, activities leading to precursors or intermediates transport, accumulation activities, pre- or pro-precursor modification activities, activities involved in energy supply or reactant supply etc.

Methods for genetically modifying organisms are known to the person skilled in the art and are described in the literature. They comprise commonly used methods for introducing genetic elements or material into a cell, tissue or organism so as to be contained in the cells, integrated into the chromosome or extrachromosomally (see, e.g. Pfeifer et al., Science 291, 1790-1792 (2001); Wang et al., Appl Microbiol Biotechnol 77 (2007)), or the removal or destruction, or modification, of genetic elements or sequences present in the genome or the organism (see, e.g.

Peiru et al., Microbial Biotechnology (2008) 1(6), 476-486; Zhang et al., Biotechnol Prog. 28(1), 52-59 (2012)).

The term “genetic element” as used herein means any molecular unit which is able to transport genetic information. It accordingly relates to a gene, preferably to a chimeric gene, a foreign gene, a transgene or a codon-optimized gene. The term “gene” refers to a nucleic acid molecule or fragment that expresses a specific protein, preferably it refers to nucleic acid molecules including regulatory sequences preceding (5′ non-coding sequences) and following (3′ non-coding sequences) the coding sequence. The term “chimeric gene” refers to any gene that is not a native gene, comprising regulatory and coding sequences that are not found together in nature.

Accordingly, a chimeric gene may comprise regulatory sequences and coding sequences that are derived from different sources, or regulatory sequences and coding sequences derived from the same source, but arranged in a manner different than that found in nature. According to the present invention a “foreign gene” refers to a gene not normally found in the organism or cell but that is introduced into said organism or cell by gene transfer, or has been modified in the organism to correspond to said foreign gene. Foreign genes can comprise native genes inserted into a non-native organism, or chimeric genes. The term “transgene” refers to a gene that has been introduced into the genome by a transformation procedure.

The term “coding sequence” refers to a DNA sequence which codes for a specific amino acid sequence. The term “regulatory sequence” refers to a nucleotide sequence located up-stream (5′ non-coding sequences), within, or downstream (3′ non-coding sequences) of a coding sequence, and which influences the transcription, RNA processing or stability, or translation of the associated coding sequence. Regulatory sequences may include promoters, enhancers, translation leader sequences, polyadenylation recognition sequences, RNA processing sites, effector binding sites and stem-loop structures.

The term “promoter” refers to a DNA sequence capable of controlling the expression of a coding sequence or functional RNA. Typically, a coding sequence is located 3′ to a promoter sequence. Promoters may be derived from the organism in which the expression takes place, may be derived from a native or from a foreign gene, or be composed of different elements derived from different promoters found in nature, e.g. in different taxonomic groups or classes, or even comprise synthetic DNA segments. It is understood by a person skilled in the art that different promoters may direct the expression of a gene at different stages of development, or in response to different environmental or physiological conditions. Promoters that cause a gene to be expressed in most genetic backgrounds and/or at most times are commonly referred to as “constitutive promoters”. Typically, since the exact boundaries of regulatory sequences have not been completely defined, DNA fragments of different lengths may have identical promoter activity. On the other hand, promoters that cause a gene to be expressed in specific contexts only, e.g. based on the presence of specific factors, growth stages, temperatures, pH or the presence of specific metabolites etc. are understood as “regulated promoters”. The promoters are typically operably linked to the coding sequences to be expressed as defined herein. The term “native promoter” as used herein relates to a promoter which is operably linked to a coding sequences to be expressed in a wildtype situation.

The term “3′ non-coding sequences” refers to DNA sequences located downstream of a coding sequence. This may include sequences encoding regulatory signals capable of affecting mRNA processing or gene expression. The 3′ region can influence the transcription, i.e. the presence of RNA transcripts, the RNA processing or stability, or translation of the associated coding sequence. The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. The term “mRNA” refers to messenger RNA that can be translated into protein by the cell.

The term “operably linked” refers to the association of nucleic acid sequences on a single nucleic acid fragment so that the function of one is affected by the other. In the context of a promoter the term means that a coding sequence is rendered capable of affecting the expression of that coding sequence, i.e., the coding sequence is under the transcriptional control of the promoter. Such a control may affect one gene or open reading frame (monocistronic), or it may affect a group of genes or open reading frames

In a particularly preferred embodiment the genetic modification results at least in the expression of a BBL2 (berberine bridge enzyme 2) polypeptide, preferably as defined herein above. For example, the polynucleotide having the nucleotide sequence of SEQ ID NO: 1, or a variant thereof, or species homologue thereof, or a polynucleotide which is 75%, 80%, 90%, 95%, 97%, 98%, or 99% identical to the polynucleotide of SEQ ID NO: 1, may be introduced into a producing cell, tissue or organism as genetic element. The introduction may be performed with a suitable promoter as defined herein allowing for an overexpression of BBL2.

In a particularly preferred embodiment the genetic modification results additionally in the expression of a PPO (polyphenol oxidase) activity or polypeptide, preferably as defined herein above. For example, the polynucleotide having the nucleotide sequence of SEQ ID NO: 3 or 5, or a variant thereof, or species homologue thereof, or a polynucleotide which is 75%, 80%, 90%, 95%, 97%, 98%, or 99% identical to the polynucleotide of SEQ ID NO: 3 or 5, may be introduced into a producing cell, tissue or organism as genetic element. The introduction may be performed, for example, with a suitable promoter as defined herein allowing for an overexpression of PPO.

In a further particularly preferred embodiment the genetic modification results additionally in the expression of an AT1 (polyamine hydroxycinnamoyltransferase 1) activity or polypeptide, preferably as defined herein above. For example, the polynucleotide having the nucleotide sequence of SEQ ID NO: 7, or a variant thereof, or species homologue thereof, or a polynucleotide which is 75%, 80%, 90%, 95%, 97%, 98%, or 99% identical to the polynucleotide of SEQ ID NO: 7, may be introduced into a producing cell, tissue or organism as genetic element. The introduction may be performed, for example, with a suitable promoter as defined herein allowing for an over-expression of AT1.

In a further particularly preferred embodiment the genetic modification results additionally in the expression of an ODC (ornithine decarboxylase) activity or polypeptide and/or an HPL (hydroperoxide lyase) activity or polypeptide, preferably as defined herein above. For example, the polynucleotide having the nucleotide sequence of SEQ ID NO: 9 or 11; and/or 13, 15 or 17 or a variant thereof, or species homologue thereof, or a polynucleotide which is 75%, 80%, 90%, 95%, 97%, 98%, or 99% identical to the polynucleotide of SEQ ID NO: 9 or 11; and/or 13, 15 or 17, may be introduced into a producing cell, tissue or organism as genetic element. The introduction may be performed, for example, with a suitable promoter as defined herein allowing for an overexpression of ODC and/or an HPL.

In a further particularly preferred embodiment the genetic modification results additionally in the expression of an PAL (L-phenylalanine ammonia lyase) activity or polypeptide and/or a C4H (trans-cinnamate 4-hydroxylase) activity or polypeptide and/or an 4CL (4-coumarate:coenzyme A ligase) activity or polypeptide and/or an HCT (Hydroxycinnamoyl-transferase) activity and/or a C3H (coumarate 3-hydroxylase) activity or polypeptide, preferably as defined herein above. For example, the polynucleotide having the nucleotide sequence of SEQ ID NO: 19, 21, 23 or 25; and/or 27, and/or 29, 105, 107, 109, 111, 113, 115, 117, 119, 121, 123, 125, 127, 129 or 131 and/or 31, 33, 35, 37, 39, 41, or 43; and/or 45, or a variant thereof, or species homologue thereof, or a polynucleotide which is 75%, 80%, 90%, 95%, 97%, 98%, or 99% identical to the polynucleotide of SEQ ID NO: 19, 21, 23 or 25; and/or 27, and/or 29, 105, 107, 109, 111, 113, 115, 117, 119, 121, 123, 125, 127, 129 or 131 and/or 31, 33, 35, 37, 39, 41, or 43; and/or 45, may be introduced into a producing cell, tissue or organism as genetic element. The introduction may be performed, for example, with a suitable promoter as defined herein allowing for an overexpression of PAL, C4H, 4CL and/or HCT.

The introduction may be controlled with suitable methods, e.g. sequencing or PCR ampli-fications. In further embodiments, the expression of BBL2 and optionally of PPO, and further optionally of AT1, and further optionally of ODC and/or HPL, and further optionally of PAL, C4H, 4CL, HCT and/or C3H may be tested with extraction and functional assays as known to the skilled person or described in the Examples.

The expression is typically controlled or conveyed by a native, regulated, tissue specific or constitutive promoter as defined herein. For example, a native promoter may be employed in situations in which the genetically modified organism is taxonomically related to the organism of origin of the heterologous genes or genetic elements, or if a similar usage of promoters or transcription initiation structures is known for these organisms. Also, in case a similar genomic GC-content is known for the genetically modified organism and the organism of origin of the heterologous genes or genetic elements, the use of a native promoter may be envisaged. Alternatively, all or some of the native promoter structures or regions associated with any of the above characterized genes or genetic elements allowing for the heterologous expression of activities or polypeptides according to the invention may be replaced or partially replaced or modified to result in regulated or constitutive promoter function. Examples or regulated promoters which can be used in the context of the present invention include inducible promoters. Accordingly, the regulation may be made dependent on extracellular factors such as temperature, the presence of certain metabolites or small molecules etc. Suitable examples include the tetracyclin inducible promoter PTet. Examples of suitable constitutive promoters include gltA, yfgF or glyA. Also envisaged are the E. coli promoters lac, trp, phoA, araBAD, rha and tac. Further envisaged are tissue specific promoters, i.e. promoter which are only active in certain tissues or after certain developmental steps. Such a promoter may, for example, be used for tissue specific expression of the activities or polypeptides as mentioned above, finally yielding a tissue specific accumulation of the compound of the invention only in subset of tissues of an organism, e.g. only in tissues exposed to herbivore attack such as leafs or flowers.

In preferred embodiments, the promoter allows for a polycistronic expression of an activity or polypeptide as defined above. The term “polycistronic” as used herein refers to sequences which encode for multiple different polypeptides or activities. In case of polycistronic transcription, a control may be provided by the organization concept of an “operon” which is understood to represent a functioning unit of genomic DNA containing a cluster of genes under the control of a single promoter. The genes of an operon are typically transcribed together into an mRNA strand and are either translated together, or undergo trans-splicing to create monocistronic mRNAs that are translated separately, e.g. several strands of mRNA that each encode a single gene product. In such a scenario the genes contained in the operon are typically expressed together, or they are not expressed. Hence, several genes are typically co-transcribed in an operon.

In general, expression of prokaryotic operons leads to the generation of polycistronic mRNAs.

Typically, an operon comprises three genetic component, (i) a promoter, i.e. an sequence which enables a gene to be transcribed, e.g. as defined herein (ii) an operator, which is a segment that can be bound by a repressor, which may obstruct the transcription of genes; and (iii) structural genes that are co-expressed. In addition a regulatory gene may be present which encodes a repressor protein capable of binding to the operator sequence. Polycistronic arrangement may also be present in eukaryotes, e.g. higher eukaryotes. If a polycistronic transcription takes place, there may be intercistronic or intergenic regions between open reading frames. Such intercistronic regions may comprise ribosomal binding sites, e.g. comprising Shine-Dalgarno sequences or other functional elements having an influence on the transcription and/or translation.

Operons may, in certain embodiments, comprise all coding sequences for BBL2, PPO, AT1, ODC, HPL, PAL, C4H, 4CL, HCT and C3H, or any subgroup thereof, e.g. BBL2 and PPO, or BBL2 and PPO and AT1, or ODC and HPL, or ODC and HPL and PAL, or C4H and 4CL, HCT and C3H etc.

In further preferred embodiments, the promoter allows for a polycistronic expression an individual expression of an activity or polypeptide as of an activity or polypeptide as defined above. For example each of BBL2, PPO, AT1, ODC, HPL, PAL, C4H, 4CL, HCT and C3H may be provided with its own promoter. The promoter may, in certain embodiments, be regulable. Also different regulable promoters for different activities may be envisaged, e.g. leading to a differential expression pattern for the activities, e.g. according to production order or necessity.

In yet another preferred embodiments a group-wise expression of groups of at least two activities selected from an activity as defined above is envisaged. For example, groups of BBL2 and PPO, AT1 and ODC, HPL and PAL, C4H and 4CL, HCT and C3H, or any other combination of 2, 3, 4, 5, 6, 7, 8 or more of the activities may be expressed. The group-wise expression may be implemented with an operon structure as defined above, or differentially regulable promoters as defined above.

In a further preferred embodiment, said expression as mentioned herein above is an overexpression. The term “overexpression” relates to the accumulation of more transcripts and in particular of more polypeptides and activities than upon the expression of a native copy of the genetic element which gives rise to said polypeptide or activity in the context of the organism of origin. In further, alternative embodiments, the term may also refer to the accumulation of more transcripts and in particular of more polypeptides or activities than upon the expression of typical, moderately expressed housekeeping genes such as actin, GAPDH or ubiquitin.

In preferred embodiments, the overexpression as mentioned above may lead to an increase in the transcription rate of a gene of about 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% or any value in between these values in comparison to the corresponding wildtype or native transcription (without modification or over-expression) in the context of the organism of origin. In preferred embodiments, such increase of in the transcription rate of a gene may be provided for at least one, or more than one, e.g. 2, 3, 4, 5, 6, 7, 8 or all of the genes or genetic elements encoding BBL2, PPO, AT1, ODC, HPL, PAL, C4H, 4CL, HCT and/or C3H; or of SEQ ID NO: 1, 3, 5, 7, 9, 11, 13, 15, 17, 19, 21, 23, 25, 27, 29, 31, 33, 35, 37, 39, 41, 43, 45, 105, 107, 109, 111, 113, 115, 117, 119, 121, 123, 125, 127, 129, 131, or homologous sequences thereof as defined herein.

In yet another preferred embodiment, the overexpression may lead to an increase in the amount of polypeptide encoded by the over-expressed gene of about 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% or any value in between these values in comparison to the corresponding wildtype or original amount of polypeptide (without modification or over-expression) in the context of the organism of origin. In preferred embodiments, such increase in the amount polypeptide encoded by the over-expressed gene may be provided for at least one, or more than one, e.g. 2, 3, 4, 5, 6, 7, 8 or all of the polypeptides of BBL2, PPO, AT1, ODC, HPL, PAL, C4H, 4CL, HCT and/or C3H; or of SEQ ID NO: 2, 4, 6, 8, 10, 12, 14, 16, 18, 20, 22, 24, 26, 28, 30, 32, 34, 36, 38, 40, 42, 44, 46, 106, 108, 110, 112, 114, 116, 118, 120, 122, 124, 126, 128, 130, 132, or homologous sequences thereof as defined herein.

In a particularly preferred embodiment, at least the gene coding for the BBL2 and PPO 10 activity or polypeptide, is over-expressed. Such an overexpression may lead to an increase in the amount of transcript and/or polypeptide encoded by the over-expressed gene of about 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% or any value in between these values in comparison to the corresponding wildtype or original amount of polypeptide (without modification or over-expression) in the context of the organism of origin.

An overexpression as defined herein above may, in one embodiment, be conveyed by the usage of promoters as defined herein above. Promoters envisaged by the present invention, which may be used for the overexpression of genes as described herein, may either be constitutive promoters, or regulated promoters. In preferred embodiments, the promoters are heterologous promoters or synthetic promoters, e.g. a strong heterologous promoter, or a regulated heterologous promoter. In particularly preferred embodiments, strong regulated or strong constitutive promoters are used. Examples of such strong constitutive or strong regulated promoters include T7 promoter (constitutive E. coli), rhaP (inducible E. coli) or, for example, S35 promoters for plants. Further suitable plant promoter which are envisaged herein can be derived from Plant Prom, a database of plant promoter sequences (accessed on Dec. 21, 2021 at http://www.soft-berry.com/berry.phtml?topic=plantprom&group=data&subgroup=plantprom), or PlantPromot-erdb an alternative plant promoter database (accessed on Dec. 21, 2021 at https://ppdb.agr.gifu-u.ac.jp/ppdb/cgi-bin/index.cgi).

Alternatively, the overexpression as defined herein above, may, in other embodiments, be conveyed by at least a second copy of the genetic element encoding the activity or polypeptide. For example, a gene or genetic element may be present in a single cell with 2, 3, 4, 5, 6, 7, 8, 9, 10, 15, 20, 25, 30, 35, 40, 45, 50, 100, 200, 300, 400, 500 or more than 500, or any value in between the mentioned values, copies. These copies may either be provided in the genome of an organism, or on a non-genomic plasmid or vector. In case of a plasmid or vector, the number and compatibility of the gene copies may be defined and regulated by the form and nature of the plasmid or vectors origin or ori. Examples of suitable bacterial origins are derived from plasmids such as pBR322, pUC, pSC101 or p15A. Plasmids or vectors envisaged by the present invention may thus be derived from these plasmids, or modified versions thereof.

An expression or overexpression as defined herein above may, in a further embodiment, be conveyed by an optimization of the codon-usage, e.g. by an adaptation of the codon usage of a gene or genetic element as defined herein above to the codon usage of the genes which are transcribed or expressed most often in the target organism or cell, or which are most highly expressed (in comparison to housekeeping gene, e.g. as defined herein above). Examples of such codon-usage of highly expressed genes may comprise the codon-usage of a group of the 5, 10, 15, 20, 25 or 30 or more most highly expressed genes of the organism in which the expression takes place.

In a further particularly preferred embodiment one or more enzymatic activities or polypeptides used for the production of a compound according to the invention, e.g one or more of BBL2, PPO, AT1, ODC, HPL, PAL, C4H, 4CL, HCT and C3H as defined herein, is derived from an organism belonging to the genus Nicotiana. The genus typically comprises at least the species Nicotiana acaulis, Nicotiana acuminata, Nicotiana africana, Nicotiana alata, Nicotiana ameghinoi, Nicotiana amplexicaulis, Nicotiana arentsii, Nicotiana attenuata, Nicotiana azambujae, Nicotiana benavidesii, Nicotiana benthamiana, Nicotiana bonariensis, Nicotiana burbidgeae, Nicotiana cav-icola, Nicotiana clevelandii, Nicotiana cordifolia, Nicotiana corymbosa, Nicotiana cutleri, Nicotiana debneyi, Nicotiana excelsior, Nicotiana exigua, Nicotiana forgetiana, Nicotiana fragrans, Nicotiana glauca Graham, Nicotiana glutinosa, Nicotiana goodspeedii, Nicotiana gossei, Nicotiana hesperis, Nicotiana heterantha, Nicotiana ingulba, Nicotiana kawakamii, Nicotiana knightiana, Nicotiana langsdorffii, Nicotiana linearis, Nicotiana longibracteata, Nicotiana longiflora, Nicotiana maritima, Nicotiana megalosiphon, Nicotiana miersii, Nicotiana mutabilis, Nicotiana nesophila, Nicotiana noctiflora, Nicotiana nudicaulis, Nicotiana occidentalis, Nicotiana obtusifolia, Nicotiana otophora, Nicotiana paa, Nicotiana palmeri, Nicotiana paniculata, Nicotiana pauciflora, Nicotiana petuniodes, Nicotiana plumbaginifolia, Nicotiana quadrivalvis, Nicotiana raimondii, Nicotiana re-panda, Nicotiana rosulata, Nicotiana rotundifolia, Nicotiana rustica, Nicotiana setchellii, Nicotiana simulans, Nicotiana solanifolia, Nicotiana spegazzinii, Nicotiana stenocarpa, Nicotiana stock-tonii, Nicotiana suaveolens, Nicotiana sylvestris, Nicotiana tabacum, Nicotiana thrysiflora, Nicotiana tomentosa, Nicotiana tomentosiformis, Nicotiana trigonophylla, Nicotiana truncata, Nicotiana umbratica, Nicotiana undulata, Nicotiana velutina, Nicotiana wigandioides, Nicotiana wutt-kei. It is particularly preferred that the one or more enzymatic activities or polypeptides used for the production of a compound according to the invention, e.g. one or more of BBL2, PPO, AT1, ODC, HPL, PAL, C4H, 4CL, HCT and C3H as defined herein, is derived from an organism belonging to the species Nicotiana attenuate.

In a further embodiment, the polynucleotide as defined herein, e.g. encoding BBL2, PPO, AT1, ODC, HPL, PAL, C4H, 4CL, HCT or C3H is comprised in one or more extrachromosomal vectors or plasmids, and/or is integrated in the genome of said organism or is comprised in an expression cassette, preferably for heterologous expression in a eukaryotic host cell, more preferably for expression in a plant cell.

The present invention accordingly also, in further aspects, relates to a vector or insertion construct or an expression cassette comprising sad polynucleotide as defined herein.

A suitable vector may be, for example, be a plasmid, a BAC or a phage vector, preferably a plasmid. The term “plasmid” refers to any plasmid suitable for transformation of bacteria, fungi or higher eukaryotes such as plants, or any other suitable host organism according to the present invention, known to the person skilled in the art and in particular to any plasmid suitable for expression of proteins in bacteria, e.g. E. coli, fungi or higher eukaryotes such as plants, e.g. plasmids which are capable of autonomous replication. Polynucleotides according to the present invention, e.g. as defined herein above, may be joined to a vector containing a selectable marker for propagation in a host. Generally, a plasmid vector is introduced in a precipitate, such as a calcium phosphate precipitate, or in a complex with a charged lipid. If the vector is a phage, it may be packaged in vitro using an appropriate packaging cell line and then transduced into host cells. Preferably, the vectors will include at least one selectable marker. Such markers include, for instance tetracycline, kanamycin or ampicillin resistance genes for culturing in E. coli and other bacteria. Vectors preferred for use in bacteria include, but are not limited to, pQE70, pQE60 and pQE9, available from QIAGEN, Inc.; pBluescript vectors, Phagescript vectors, pNH8A, pNH16a, pNH18A, pNH46A, available from Stratagene Cloning Systems, Inc.; pKK223-3, pKK233-3, pDR540, pRIT5 available from Pharmacia Biotech, Inc., and pET vectors available from No-vagen. Vectors preferred for use in plants include, but are not limited to pB1121, pCAMBIA, pEarleyGate 201, pPZP100, pBI221, pCAMBIA1381Xb, pEarleyGate 202, pPZP101pBINPLUS, pCAMBIA1381Xc, pEarleyGate 203, pPZP102, pBin19, pCAMBIA1381Z, pEarleyGate 204, pPZP111, pCAMBIA0105.1R, pCAMBIA1390, pEarleyGate 205, pPZP112, pCAMBIA0305.1, pKANNIBAL, pHANNIBAL, pGreenll, pGreen. Othersuitable vectorswould be known to the person skilled in the art, or can be derived from suitable literature sources such as ChenBiotechnol Adv. 30(5), 1102-1107 (2012).

Introduction of a vector as defined herein above into a cell, tissue or organism according to the present invention can be effected by any suitable technique, e.g. calcium phosphate transfection, DEAE-dextran mediated transfection, cationic lipid-mediated transfection, electro-poration, chemical transformation, transduction, Agrobacterium based transformation or particle gun based transformation.

For a genomic integration, one, two or more copies of a genetic element as mentioned above may be introduced into the cell or organism via an “insertion construct” and thereby be placed in the chromosome. The integration site may be either in vicinity of an original copy (if present), or, preferably, at any suitable location, e.g. at a different location. The insertion may advantageously be preselected via the choice of homologous flanks which are necessary for the integration. The insertion site may accordingly be determined according to known features of the genome, e.g. transcription activity of chromosomal regions, potential distance to the first copy (original gene), orientation of the first copy (original gene), the presence of further inserted genes etc. In certain embodiments, the insertion construct may comprise one copy of a genetic element, e.g. a polynucleotide encoding BBL2, PPO, AT1, ODC, HPL, PAL, C4H, 4CL, HCT and/or C3H, or additional copies may be provided in tandem repeat forms. For example an insertion construct may comprise BBL2, PPO, AT1, ODC, HPL, PAL, C4H, 4CL, HCT or C3H alone, or BBL2, PPO, AT1, ODC, HPL, PAL, C4H, 4CL, HCT and C3H as group, or any subgroup thereof. In further embodiments, the present invention envisages the use of non-tandem repeats. In certain embodiments, the copies of the genetic element may be kept as different and/or remote as possible.

Such differences may be based on the use of different promoters, the modification of genomic flanks of the genes, or, in specific embodiments, the modification of the nucleotide sequence of the second or further copy vs. the first copy (original version) of a gene, or a third copy vs. a second copy and/or vs. a first copy (original version) of a gene etc.

An “expression cassette” as used herein relates to is a polynucleotide construct comprising a gene or genetic element and a regulatory sequence to be expressed by a transfected cell.

The expression cassette typically directs a cell's machinery to generate RNA and proteins. The expression cassette may, in certain embodiments, be designed for modular cloning of protein-encoding sequences. In further embodiments, the expression cassette more be composed of more than one gene or genetic element, e.g. a polynucleotide encoding BBL2, PPO, AT1, ODC, HPL, PAL, C4H, 4CL, HCT and C3H as group, or any subgroup thereof; as well as sequences controlling their expression. For example, the expression cassette may comprises a promoter sequence, an open reading frame, and a 3′ untranslated region, optionally comprising, e.g. in eukaryotes, a polyadenylation site.

In a further central aspect the present invention relates to an organism, tissue or cell producing the compound according to the present invention. The organism, tissue or cell may be genetically modified, preferably as defined herein above, e.g. by the introduction of a vector or plasmid, an insertion construct or an expression cassette according to the present invention.

A further, preferably envisaged possibility for the genetic modification of a cell, organism or tissue (group of cells) is a molecular modification of the genome, preferably using a genomic editing system.

“Genomic editing systems” advantageously allow to provide genomic modifications without the necessity of inserting antibiotics resistance cassettes or any additional selection marker.

Such genomic editing approaches may, for example, be based on the use of the CRISPR/Cas system, a TALEN-system, or a zinc finger nuclease (ZFN)-system.

Particularly preferred is the use of the CRISPR (Clustered Regularly Interspaced Short Palindromic Repeats)/Cas system. CRISPR/Cas can be utilized to reduce expression of specific genes (or groups or similar genes) or to edit genomic sequences. This is typically achieved through the expression of single stranded RNA in addition to a CRISPR gene or nuclease. The technique typically relies on the expression of a CRISPR gene such as Cas9, or other similar genes in addition to an RNA guide sequences (see, for example, Cong et al., Science, 339 (6121), 819-823 (2013)).

Double stranded cleavage may accordingly be targeted to specific sequences using the expression of appropriate flanking RNA guide sequences, which may be provide as one component of the multicomponent system, e.g. together with Cas9 or a similar functionality. In a preferred embodiment RNA guide sequences and CRISPR gene expression (e.g. Cas9) may be included as part of an expression construct.

The term “TALEN-system” relates to the use of TALEN, i.e. the Transcription Activator-Like Effector Nuclease, which is an artificial restriction enzyme, generated by fusing the TAL effector DNA binding domain to a DNA cleavage domain. TAL effectors are proteins which are typically secreted by Xanthomonas bacteria or related species, or which are derived therefrom and have been modified. The DNA binding domain of the TAL effector may comprise a highly conserved sequence, e.g. of about 33-34 amino acid sequence with the exception of the 12th and 13th amino acids which are highly variable (Repeat Variable Diresidue or RVD) and typically show a strong correlation with specific nucleotide recognition. The TALEN DNA cleavage domain may be derived from suitable nucleases. For example, the DNA cleavage domain from the Fokl endonuclease or from Fokl endonuclease variants may be used to construct hybrid nucleases. TALENs may preferably be provided as separate entities due to the peculiarities of the Fokl domain, which functions as a dimer. TALENs or TALEN components may preferably be engineered or modified in order to target any desired DNA sequence. Such engineering may be carried out according to suitable methodologies, e.g. Zhang et al., Nature Biotechnology, 1-6 (2011), or Reyon et al., Nature Biotechnology, 30, 460-465 (2012).

The term “zinc finger nuclease (ZFN)-system” as used herein refers to a system of artificial restriction enzymes, which are typically generated by fusing a zinc finger DNA-binding domain to a DNA-cleavage domain. Zinc finger domains may preferably be engineered or modified in order to target any desired DNA sequence. Such engineering methods would be known to the skilled person or can be derived from suitable literature sources such as Bae et al., Nat Biotechnol, 21, 275-80 (2003); Wright et al., Nature Protocols, 1, 1637-1652 (2006)) Typically, the non-specific cleavage domain from type Ils restriction endonucleases, e.g. from Fokl, may be used as the cleavage domain in ZFNs. Since this cleavage domain dimerizes in order to cleave DNA a pair of ZFNs is typically required to target non-palindromic DNA sites. ZFNs envisaged by the present invention may further comprise a fusion of the non-specific cleavage to the C-terminus of each zinc finger domain. For instance, in order to allow two cleavage domains to dimerize and cleave DNA, two individual ZFNs are typically required to bind opposite strands of DNA with C-termini provided in a specific distance. It is to be understood that linker sequences between the zinc finger domain and the cleavage domain may requires the 5′ terminus of each binding site to be separated by about 5 to 7 bp. The present invention envisages any suitable ZNF form or variant, e.g. classical Fokl fusions, or optimized version of the Fokl, as well as enzymes with modified dimerization interfaces, improved binding functionality or variants, which are able to provide heterodimeric species.

According to preferred embodiments of the present invention said genetic modification allows or the expression of one or more activities or polypeptides as defined herein, e.g. BBL2, PPO, AT1, ODC, HPL, PAL, C4H, 4CL, HCT and/or C3H. The expression may be a heterologous expression as defined herein, preferably an overexpression as defined herein. The genetic modification of the cell, organism or tissue is a genetic modification as define herein above in detail. In particular, the genetic modification may result at least in the expression of a BBL2 (berberine bridge enzyme 2) polypeptide as defined above, additionally in the expression of a PPO (polyphenol oxidase) activity or polypeptide as defined above, additionally in the expression of an AT1 (polyamine hydroxycinnamoyltransferase 1) activity or polypeptide as defined above, additionally in the expression of an ODC (ornithine decarboxylase) activity or polypeptide and/or an HPL (hydroperoxide lyase) activity or polypeptide as defined above, and additionally in the expression of an PAL (L-phenylalanine ammonia lyase) activity or polypeptide and/or a C4H (trans-cinnamate 4-hydroxylase) activity or polypeptide and/or an 4CL (4-coumarate:coenzyme A ligase) activity or polypeptide and/or an HCT (Hydroxycinnamoyl-transferase) activity or polypeptide and/or C3H (coumarate 3-hydroxylase) activity or polypeptide as defined above.

In a preferred embodiment said genetically modified organism, tissue or cell is prokaryotic, e.g. a bacterium or fungus, preferablya bacterium of the genus Klebsiella, Clostridium, Bacillus, Arthobacter, Streptomyces, Corynebacterium, Erwinia, Xanthomonas, Lactobacillus, Caldicellulosiruptor, Pseudomonas, Alcanivorax, Brevibacterium, Bifidobacterium, Escherichia, or Staphylococcus, more preferably Escherichia coli; or fungus of the genus Aspergillus, Candida, Saccharomyces, Ustilago, Cryptococcus, Fusarium, Rhizopus, Magnaporthe, Komagataella, Trichderma, Penicillium, Acremonium, Mucor, Alternaria, Botrytis, Endothia, Rhizoctonia, Sclerotinia, Klyveromyces, Torulopsis, Sporotrichum, Geotrichum, Verticillium, Botryosphaeria, Trichothecium, Hansenula, Schizosaccharomyces, Brettanomyces, or Neurospora, preferably Candida or Saccharomyces.

It is further preferred that said genetically modified organism, tissue or cell is eukaryotic.

It is more preferred that the genetically modified organism is a plant, or that said tissue is a plant tissue or that said cell is a plant cell. In a further particularly preferred embodiment said genetically modified organism is a higher plant which is attacked by an insect herbivore, or said genetically modified tissue belongs to a higher plant which is attacked by an insect herbivore, or said genetically modified cell is a higher plant cell, wherein said plant is attacked by an insect herbi-vore.

The term “insect herbivore” as used herein relates to an insect which is anatomically and physiologically adapted to eating plant material, such as foliage for the main component of its diet. As a result of their plant diet, herbivorous insects typically have mouthparts adapted to rasping or grinding. It is particularly preferred that said insect herbivore is an insect which feeds by lacerate and flush and/or by piercing and sucking.

In specific embodiments said plant is a higher pant which is attacked by an insect feeding by lacerate and flush and/or piercing and sucking. The feeding mechanism in “lacerate—and-flush” insects typically involves the repeated insertion and withdrawal of a stylet into a plant tissue. “Piercing and sucking” means that plant cells are pierced with a stylet, liquified and saliva is then used to suck ruptured cell content with a sucking mouthpiece. Further information may be derived from suitable textbooks such as Strong et al., Insects on Plants, Harvard University Press (1984).

In further embodiments the plant is a plant which is attacked by an insect which oviposits its eggs into leaves of said plant. Non-liming examples of such insects include bagworms, aphids, mealybugs, cicadas, earworms, beetles, lace bugs, leafhoppers, and scales.

In further preferred embodiments said herbivore insect is a leafhopper or planthopper. Even more preferably, the insect belongs to the family of Aphrodinae, Bathysmatophorinae, Cicadellinae, Coelidiinae, Deltocephalinae, Errhomeninae, Euacanthellinae, Eurymelinae, Evacanthinae, Hylicinae, Iassinae, Jascopinae, Ledrinae, Megophthalminae, Mileewinae, Nastlopiinae, Neobalinae, Neocoelidiinae, Nioniinae, Phereurhininae, Portaninae, Signoretiinae, Tartessinae, Typhlocybina, or Ulopinae. Even more preferably the insect is of the genus Empoasca, Circulifer, Nilaparvata, Sogatella, Nephotettix, or Cicadulina. In a specific embodiment said plant is thus a higher pant which is attacked by a leafhopper or planthoppfer, more preferably by an insect belonging to the family of Aphrodinae, Bathysmatophorinae, Cicadellinae, Coelidiinae, Deltocephalinae, Errhomeninae, Euacanthellinae, Eurymelinae, Evacanthinae, Hylicinae, lassinae, Jascopinae, Ledrinae, Megophthalminae, Mileewinae, Nastlopiinae, Neobalinae, Neocoelidiinae, Nioniinae, Phereurhininae, Portaninae, Signoretiinae, Tartessinae, Typhlocybina, or Ulopinae, or even more preferably belonging to the genus Empoasca, Circulifer, Nilaparvata, Sogatella, Nephotettix, or Cicadulina.

In more preferred embodiment said higher plant is crop plant which is attacked by an insect feeding by lacerate and flush and/or piercing and sucking.

In the most preferred embodiment, said higher plant is of the genus Nicotiana, Solanum, Oryza, Zea, Phaeseolus or Camellia.

In a further aspect the present invention relates to the use of the polypeptide or polynucleotide as defined herein above, of the expression cassette as defined herein above or of the vector or insertion construct as defined herein above, for the production of the compound according to the invention, e.g. as defined herein above.

In a further aspect the present invention relates a composition comprising the compound according to the invention, as defined herein above, or produced with a method as defined herein above or produced by an organism, tissue or cell as defined herein above, preferably a genetically modified organism, tissue or cell. The composition may preferably be an agricultural composition. The term “agricultural composition” as used herein refers to a composition which is suitable, e.g. comprises effective concentrations and amounts of ingredients such as the compound of the invention as defined herein, for use in agriculture or horticulture. The composition may further, in specific embodiments, be for usage on or in plants in non-agricultural environ-ments.

In one embodiment, the composition may be designed for use in or on or at the locus of a plant. Typically, the usage may be in the leaf or flowering zone of a plant, alternatively also in the root zone. The term “locus of a plant” is to be understood as any type of environment, soil, area or material where the plant is growing or intended to grow. Preferably, the term relates to soil on which a plant is growing.

“Effective amounts” or “effective concentrations” of compounds as defined herein may be determined according to suitable in vitro and in vivo testings known to the skilled person.

These amounts and concentrations may be adjusted to the locus, plant species or variety, soil, climate conditions or any other suitable parameter which may have an influence on herbivore attacks.

The composition may comprise a suitable carrier, preferably an agrochemical charrier.

The term “agrochemical carrier” as used herein is a substance or composition which facilitates the delivery and/or release of agrochemicals (including a compound as defined herein) in their field of use, in particular on or into plants. Examples of suitable agrochemical carriers include solid carriers such as mineral earths e.g. silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers. Further suitable examples of carriers include fumed silica or precipitated silica, which may, for instance, be used in solid formulations as flow aid, anti-caking aid, milling aid and as carrier for liquid active ingredients. Additional examples of suitable carriers are microparticles, for instance microparticles which stick to plant leaves and release their content over a certain period of time. In specific embodiments, such agrochemical carriers may be composite gel microparticles that can be used to deliver plant-protection active principles, e.g. as described in U.S. Pat. No. 6,180,141; or compositions comprising at least one phytoactive compound and an encapsulating adjuvant, wherein the adjuvant comprises a fungal cell or a fragment thereof, e.g. as described in WO 2005/102045; or carrier granules, coated with a lipophilic tackifier on the surface, wherein the carrier granule adheres to the surface of plants, grasses and weeds, e.g. as disclosed in US 2007/0280981. In further specific embodiments, such carriers may include specific, strongly binding molecules which assure that the carrier sticks to the plant until its content is completely delivered. For instance, the carrier may be or comprise cellulose binding domains (CBDs) have been described as useful agents for attachment of molecular species to cellulose (see U.S. Pat. No. 6,124,117); or direct fusions between a CBD and an enzyme; or a multifunctional fusion protein which may be used for delivery of encapsulated agents, wherein the multifunctional fusion proteins may consist of a first binding domain which is a carbohydrate binding domain and a second binding domain, wherein either the first binding domain or the second binding domain can bind to a microparticle (see also WO 03/031477). Further suitable examples of carriers include bifunctional fusion proteins consisting of a CBD and an anti-RR6 antibody fragment binding to a microparticle. In another specific embodiment the carrier may be an active ingredient carrier granule that adheres to the surface of plants using a moisture-active coating, for instance including gum arabic, guar gum, gum karaya, gum tragacanth and locust bean gum. Upon application of the granule onto a plant surface, water from precipitation, irrigation, dew, co-application with the granules from special application equipment, or guttation water from the plant itself may provide sufficient moisture for adher-ence of the granule to the plant surface (see also US 2007/0280981).

In further embodiments, the composition may comprise or may additionally comprise a stabilizer. The term “stabilizer” as used herein means any suitable compound or composition which allows to reduce the reactivity of the compound according to the invention and/or which prevents or slows down reactive portions of the composition according to the invention from interacting with each other and/or from autocatalytic polymerization. In preferred embodiments, the stabilizer is a compound or composition which is capable of lowering the pH and/or maintaining a low pH, more preferably a pH of 4.8 or below. In further embodiments, the stabilizer may be detergent such as a zwitterionic detergent or the like.

In further embodiments, the composition may comprise or may additionally comprise a spreading agent. The term “spreading agent” as used herein refers to a substance, the addition of which to a liquid or semi-solid composition, leads to an enlargement of the surface covered by the composition. Spreading agents typically act by e.g. reducing the surface tension of a composition or by increasing the wettability of the treated surface. As a result, it facilitates the uniform distribution of the composition and its ingredients, and also the formation of a uniform film on the treated surface, e.g. a plant leaf. The spreading agent typically suppresses the tendency of compositions with a high surface tension (such as e.g. aqueous solutions) from forming droplets on the treated surface which would lead to spot-like concentration of the substances present in the composition. Suitable examples of spreading agents to be used in the context of the present invention include oils or fats such as short-chain fatty acids (C6 to C10), e.g. coconut oil and ba-bassu oil, plant butters, squalene, medium-length fatty acid chains (C12 to C16) or with high lec-ithin or squalene content e.g. avocado oil, sesame oil, grapeseed oil or amaranth oil, or long-chain fatty acids (C18 to C24), e.g. evening primrose oil, borage oil, hemp oil and wild rose oil. A further suitable class of spreading agents is the class of hydrophobins, i.e. small, cysteine-rich proteins with a length of about 100-150 amino acids which occur in nature only in filamentous fungi.

In further embodiments, composition composition according to the present invention may comprise an adjuvant. An “adjuvant” as used refers means a compound which aids or mod-ifies the action of the principal ingredient of a composition, e.g. a compound according to the present invention. Adjuvants may can act as wetting agents, stickers, perpetrators or activators. In specific embodiments, they may be used for spraying.

An example of an adjuvant envisaged by the present invention is a surfactant. “Surfac-tants” are surface-active agents which are typically amphiphilic and comprise a hydrophobic and hydrophilic group. They may be anionic, cationic, nonionic or amphoteric. Examples include LAS, LES, CTAC, DODAC, APEO, FAEO and AEO.

The composition according to the present invention may further comprise additional active ingredients. For example, the composition may comprise for example at least one pesticidal compound. For example, the composition may additionally comprise at least one herbicidal compound and/or at least one fungicidal compound and/or at least one insecticidal compound. Suitable examples of herbicidal compounds include, but are not limited to 2,4-dichlorophenoxy acetic acid, aminopyralid, atrazine, clopyralid, dicamba, glufosinate ammonium, fluazifop, fluroxy-pyr, glyphosate, imazapyr, imazapic, imazamox, linuron, 2-methyl-4-chlorophenoxyacetic acid, metolachlor, paraquat, pendimethalin, picloram, triclopyr, flazasulfuron or metsulfuron-methyl. Suitable examples of fungicidal compounds include, but are not limited to carbendazim, thi-ophanate, thiabendazole, flusilazole, azoxystrobin, difenoconazole, kasugamycin,or isoprothi-olane. Suitable examples of insecticidal compounds include, but are not limited to contact insec-ticides such as cephate, carbaryl, fipronil, pyrethrins, pyrethroids such as bifenthrin, cyfluthrin, cypermethrin, deltamethrin, lambda-cyhalothin, permethrin, es-fenvalerate, tefluthrin or tralo-methrin, as well as fipronil or spinosad; or a surfactant insecticide comprising, for example, a neonicotinoid-based compound and a silicone-based surfactant. Also envisaged are inhibitors of P450s such as piperniyl butoxide which is a general P450 inhibitor. These inhibitors may be used in combination with a contact or surfactant insecticide as mentioned above.

In particularly preferred embodiments, the composition is designed or prepared for plant protection, specifically for plant protection against an herbivore, e.g. as defined herein. The composition may, for example, be formulated in any suitable wayto allow for a application on a plant, e.g. a higher plant, preferably a crop plant as defined herein. The composition may, for example, be formulated to comprise an adjuvant, spreading agent and/or surfactant which allow for an efficient application, e.g. via spraying or any other suitable method.

In a further aspect the present invention relates to the use of the compound according to the invention, as defined herein above, or produced with a method as defined herein above or produced by an organism, tissue or cell as defined herein above, preferably a genetically modified organism, tissue or cell, or of the composition as defined herein above for plant protection. In a preferred embodiment the present invention relates to the use of the compound according to the invention, as defined herein above, or produced with a method as defined herein above or produced by an organism, tissue or cell as defined herein above, preferably a genetically modified organism, tissue or cell, or of the composition as defined herein above is for plant protection against an herbivore.

It is preferred that said herbivore is an herbivore insect as defined above, preferably a leafhopper or planthopper, more preferably belonging to the family of Aphrodinae, Bathysmatophorinae, Cicadellinae, Coelidiinae, Deltocephalinae, Errhomeninae, Euacanthellinae, Eurymelinae, Evacanthinae, Hylicinae, Iassinae, Jascopinae, Ledrinae, Megophthalminae, Mileewinae, Nastlopiinae, Neobalinae, Neocoelidiinae, Nioniinae, Phereurhininae, Portaninae, Signoretiinae, Tartessinae, Typhlocybina, or Ulopinae, even more preferably belonging to the genus Empoasca, Circulifer, Nilaparvata, Sogatella, Nephotettix, or Cicadulina.

The compound may accordingly be provided by said organism, tissue or cell. It may be provided any suitable amount, concentration, or form as known to the skilled person. The compound may, for example, be accumulated in said organism, tissue or cell and thereby contribute to protection of said organism, tissue or cell against an herbivore. Alternatively, the compound may be excreted or exported or otherwise be delivered to the outside of said organism, cell, or tissue and subsequently be used for different purposes, e.g. as ingredient in a plant protection composition.

In a further aspect the present invention relates to the use of the compound according to the invention, as defined herein above, or produced with a method as defined herein above or produced by an organism, tissue or cell as defined herein above, preferably a genetically modified organism, tissue or cell, or of the composition as defined herein above as insecticide. The insecticide may be employed in any suitable context or environment or on or in any plant considered suitable by the skilled person. It is preferred that said insecticide is a specific insecticide against an insect herbivore, more preferably against an insect herbivore which feeds by lacerate and flush and/or by piercing and sucking, even more preferably against a leafhopper or planthopper, e.g. belonging to the family of Aphrodinae, Bathysmatophorinae, Cicadellinae, Coelidiinae, Deltocephalinae, Errhomeninae, Euacanthellinae, Eurymelinae, Evacanthinae, Hylicinae, Iassinae, Jascopinae, Ledrinae, Megophthalminae, Mileewinae, Nastlopiinae, Neobalinae, Neocoelidiinae, Nioniinae, Phereurhininae, Portaninae, Signoretiinae, Tartessinae, Typhlocybina, or Ulopinae, most preferably against an insect of the genus Empoasca, Circulifer, Nilaparvata, Sogatella, Nephotettix, or Cicadulina.

In a further aspect, the present invention relates to the use of an organism, tissue or cell as defined herein, or an organism, tissue or cell comprising the expression cassette as defined, or the vector or insertion construct as defined herein, for the production of the compound according to the invention. The compound may accordingly be provided by said organism, tissue or cell. It may be provided any suitable amount, concentration, or form as known to the skilled person. The compound may, for example, be accumulated in said organism, tissue or cell and thereby contribute to protection of said organism, tissue or cell against an herbivore. Alternatively, the compound may be excreted or exported or otherwise be delivered to the outside of said organism, cell, or tissue and subsequently be used for different purposes, e.g. as ingredient in a plant protection composition.

In a further embodiment, the present invention relates to the use of an organism as defined herein, e.g. an organism comprising the expression cassette as defined herein, or being genetically modified in order to express the compound of the invention, for agricultural, horti-cultural or ornamental production. Preferably, said organism is a higher plant, e.g. a crop plant, which is used for the production of food products, or it may a plant which is employed for horti-cultural purposes or as ornamental plant. By producing a compound of the invention said organisms, e.g. plants, are protected against herbivore attacks and can thus produce food products, or be marketed as horticultural or ornamental plants.

A use according to the invention may a continued use or a period use. A periodic use may, for example, imply a use over a certain period of time, e.g. 1, 2, 3, 4, 5, 6, 7 or more day(s), 1, 2, 3, 4, 5, 6, 7, 8, 9, 10 or more week(s), 1, 2, 3, 4, 5, 6, 7, 8, 9, 10, 11, 12 or more month(s), or 1, 2, 3, 4, 5, 6, 7, 8, 9, 10 or more years or any time period in between the mentioned values. The use may be based on the employment of any suitable amount, concentration, or form of a compound or composition or according to the invention.

In a final aspect the present invention relates to a method of plant protection comprising contacting a plant or part of a plant with the compound according to the invention or the composition as defined herein. The method may comprise the following steps preparation of a compound or composition according to the present invention for the envisaged applications form, e.g. by formulation as spray etc.; optionally packaging into suitable vessels, e.g. for transport or storage, or direct use in application device; spreading of compound or composition to targets, e.g. higher plants in a field, which are susceptible to herbivore attacks, via suitable application techniques. Alternatively, the method may be based on the employment of previously prepared material, e.g. propagation material which has been contacted with said compound or composition at a different site than the locus of a plant.

The method may be performed one or more than one times, e.g. 2, 3, 4, 5, 6, 7, 8 times within a specific time period such as week, a month or a year.

Said application step may involve, for example, the contacting of compounds according to the invention or compositions thereof with plants or plant propagation material, e.g. seeds. This may include spraying, dressing, coating, pelleting, dusting or soaking application methods. Alternatively, the compound or composition according to the present invention may be applied with ground machines or aircrafts or unmanned aerial vehicles (UAV) such as drones.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

### General Strategy

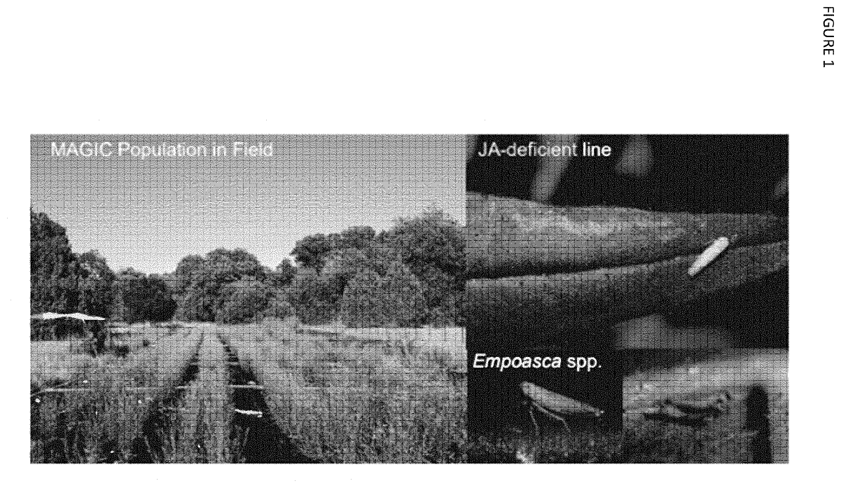

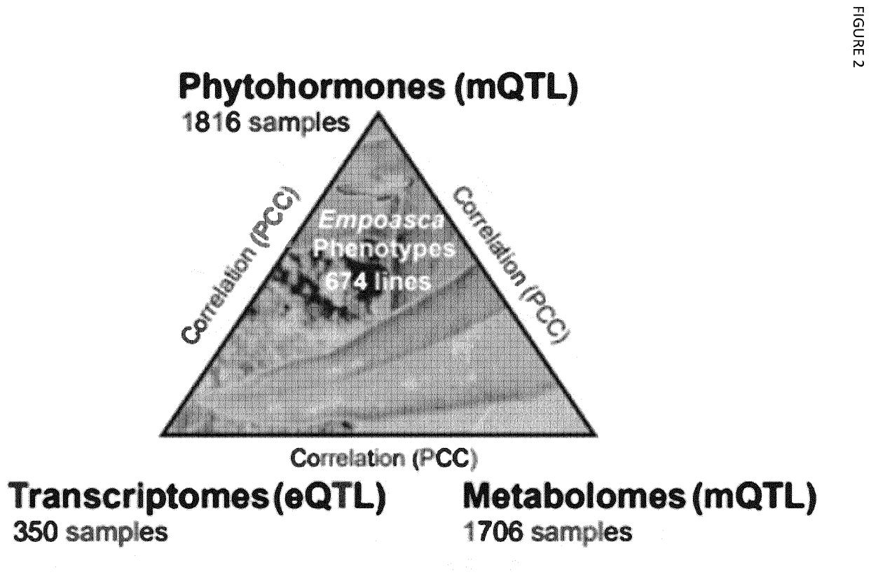

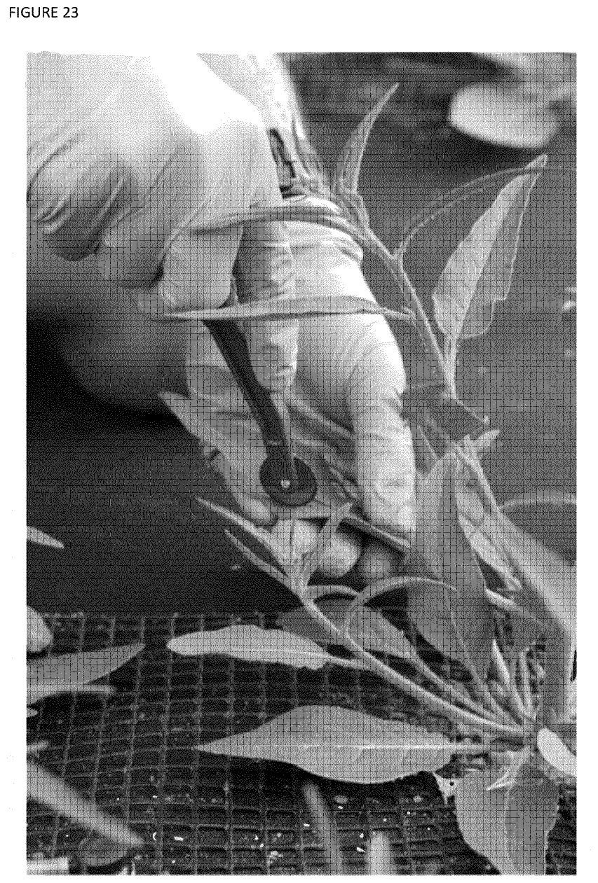

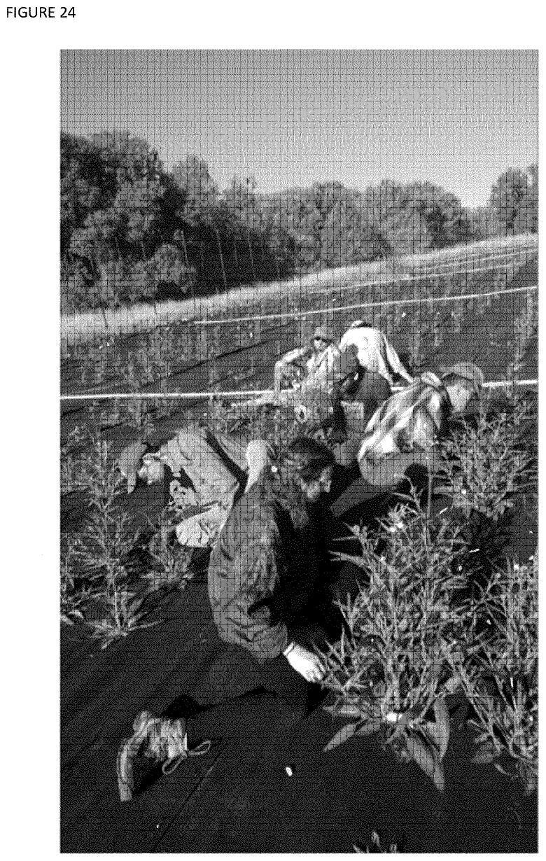

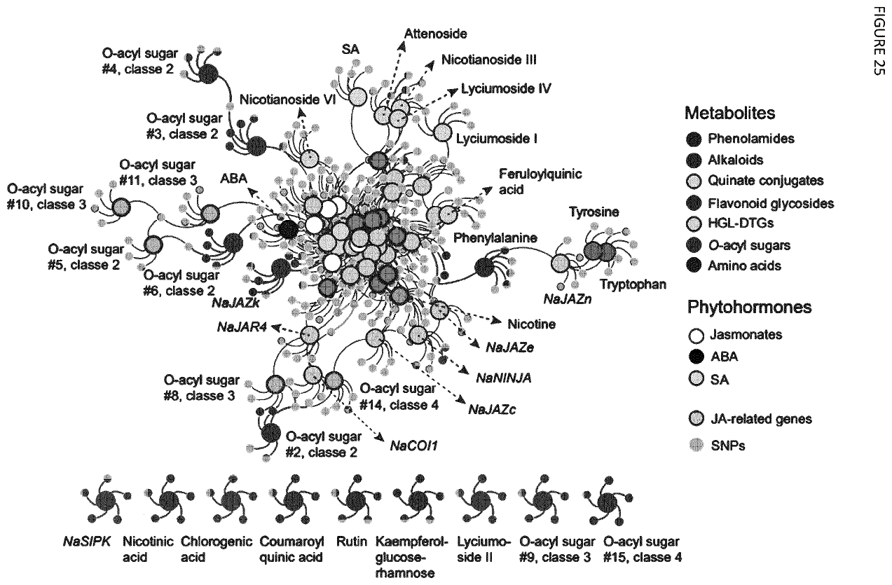

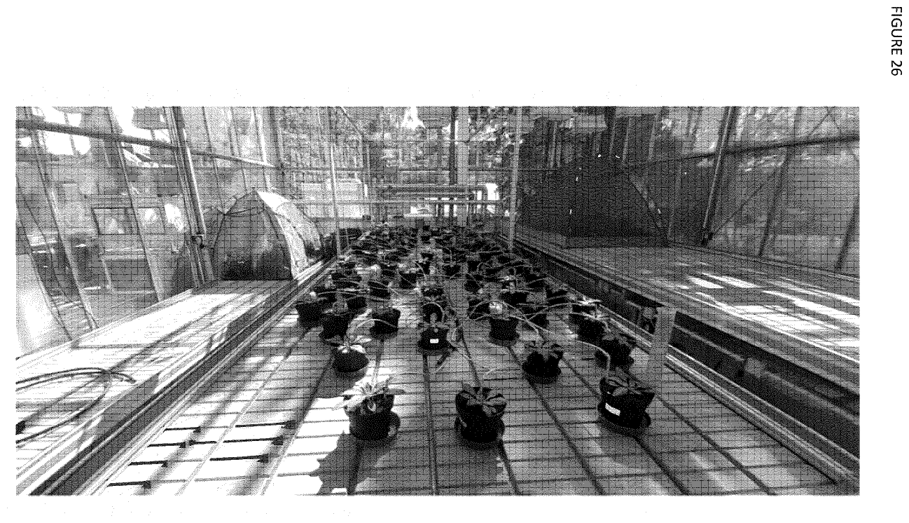

To uncover the JA-elicited nonhost resistance traits of N. attenuata, we adopted a forward genetic strategy. A replicated population of 650 recombinant inbred lines (RILs) from a 26-parent Multiparent Advanced Generation Inter-Cross (MAGIC) population was planted into a native habitat in Arizona (FIG. 1 and FIGS. 21 and 22). In this setting, Empoasca leafhoppers are abundant and damage their native host cucumbers (Cucurbita foetidissima). JA-deficient N. attenuata lines were attacked by the leafhoppers (Kallenbach et al., Proc Natl Acad Sci USA 109, E1548-1557 (2012)) at rates that varied within the MAGIC populations (FIG. 1). The Empoasca attack levels in 1907 individual plants of 674 RILs and parental lines of the field-grown MAGIC population and constructed a multi-omics dataset were quantified based on high-throughput analyses of phytohormones, transcriptomes, and metabolomes. This multi-omics dataset was produced from leaves elicited by a simulated herbivory treatment. Herbivore attack by treating standardized puncture wounds with oral secretions of Manduca sexta larvae (W+OS) was mimicked, to remove confounding factors caused by the stochastic nature of insect attack in nature and capture transiently expressed genes and metabolites in these samples from field-grown plants (FIG. 2 and FIGS. 23 and 24).

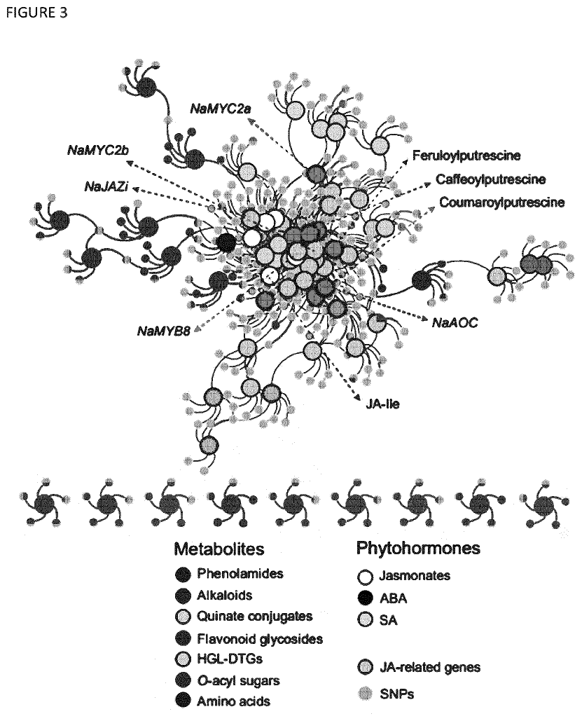

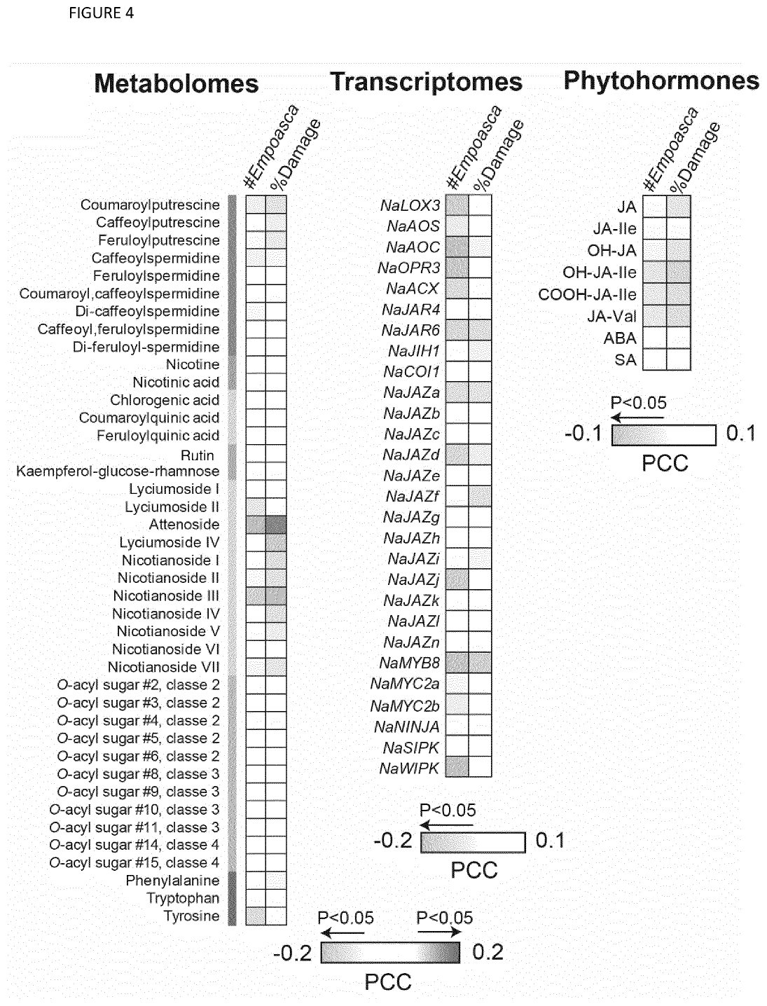

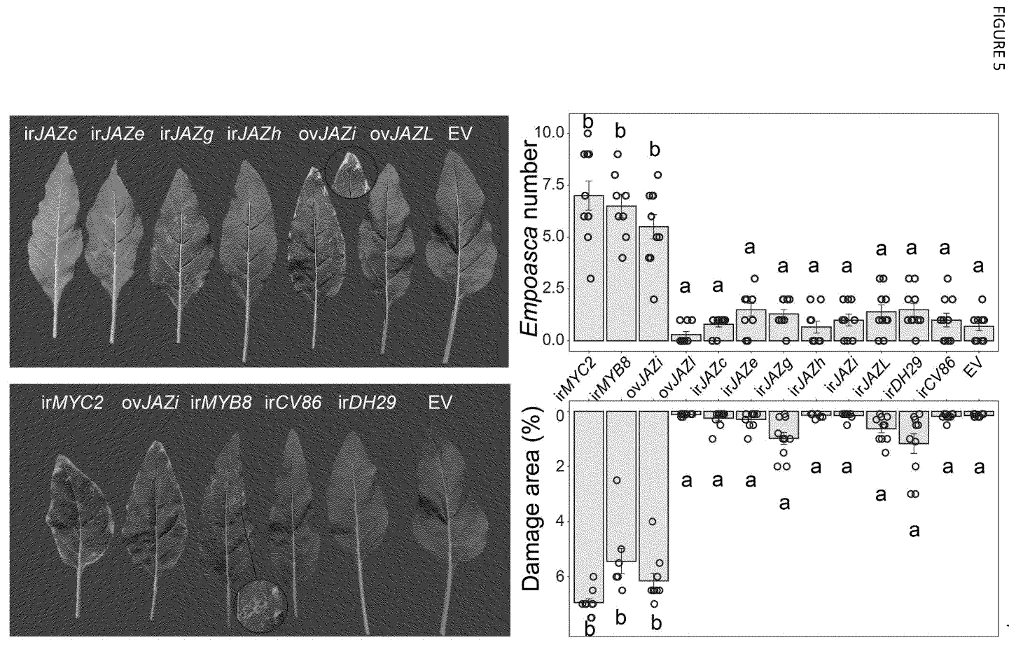

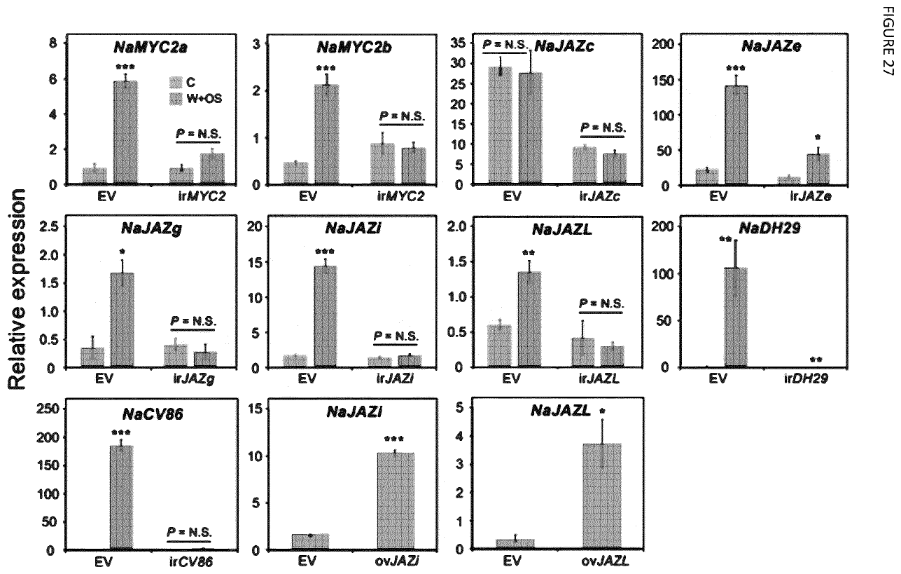

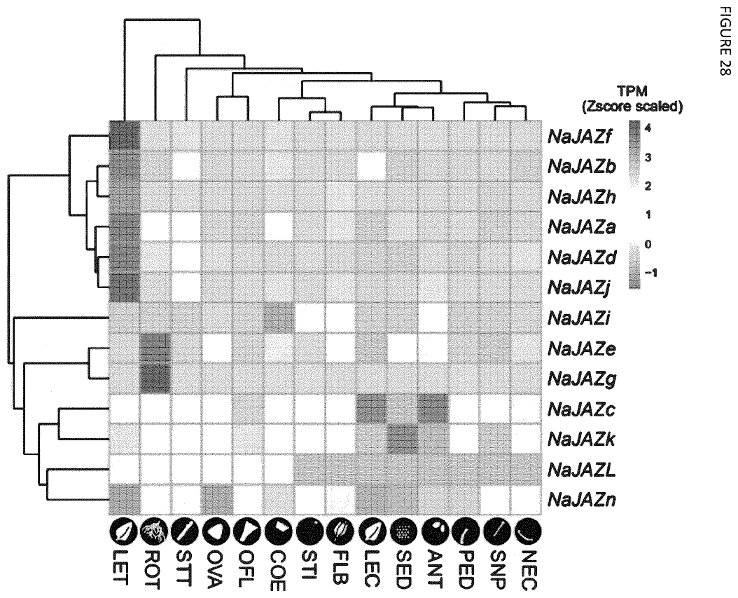

To analyse associations among the genetic and metabolic responses of this multi-omics dataset, the focus was set on JA signalling-related genes and used previously acquired knowledge of N. attenuata leaf chemistry (Li et al., Proc Natl Acad Sci USA 112, E4147-4155 (2015), Li, S. et al., Proc Natl Acad Sci USA 113, E7610-E7618 (2016)) to construct a co-association network of the JA-dependent module. This network considered not only the correlations among metabolites, phytohormones and gene expressions, but also the shared SNPs, inferred from metabolic quan-titative trait loci (mQTL) or expression QTL (eQTL) analyses for each of these components (FIG. 3, FIG. 25). This co-association network revealed that JAs and JA-related genes nucleated by the JA-regulated phenolamide master transcription factor regulator, NaMYB8 (Onkokesung et al., Plant Physiol 158, 389-407 (2012)), formed a genetic hub that clustered with induced phenolamides, such as N-coumaroylputrescine (CoP), N-caffeoylputrescine (CP), N-feruloylputrescine (FP), and malonylated 17-HGL-DTGs (FIG. 3). More peripheral to this hub were glycosylated 17-HGL-DTG precursors such as lyciumoside 1, lyciumoside IV, attenoside and nicotianoside III and other specialized metabolites such as nicotine, acylsugars and flavonoids (FIG. 25). A NaJAZi gene clustered centrally to JAs but distant from NaJAR4 and NaCO1l, suggesting the engagement of noncanoni-cal JA signalling. To further resolve the components in the co-association network responsible for Empoasca susceptibility, we conducted a pairwise correlational analysis among the-omics datasets and Empoasca abundance and leaf area damaged in the RILs of the MAGIC populations (FIG. 4). The putrescine-derived phenolamides and malonylated 17-HGL-DTGs were negatively correlated with the Empoasca numbers and damage, whereas glycosylated 17-HGL-DTG precursors were positively correlated. JA-related genes, NaMYB8, NaLOX3, NaAOC, NaOPR3, NaJAR6 and NaWIPK, exhibited the highest negative correlation scores with the Empoasca numbers and damage. There was considerable heterogeneity in the expression of the JA-related family of JAZ genes, with the expression of NaJAZa, NaJAZd, NaJAZf, NaJAZi and NaJAZj being negatively correlated with the Empoasca numbers and damage; the expression of JAZ genes known to mediate M. sexta defense responses, such as NaJAZh (Oh et al., Plant Physiol 159, 769-788 (2012)), showed no significant correlations. JA, but not the canonical mediator of JA signalling, JA-Ile, was negatively correlated, and negative correlations were observed for the hydroxylated and carbox-ylated JAs (OH-JA, OH-JA-Ile and COOH-JA-Ile) and JA-valine conjugates (JA-Val). These results suggested that a JA signalling sector was likely involved in the JA-dependent Empoasca leafhopper resistance response.

### Example 2

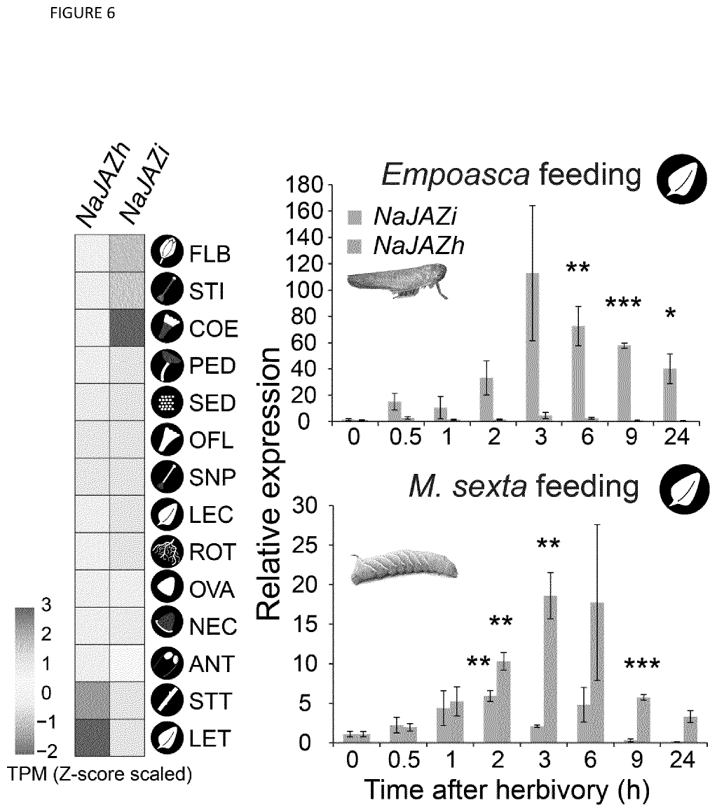

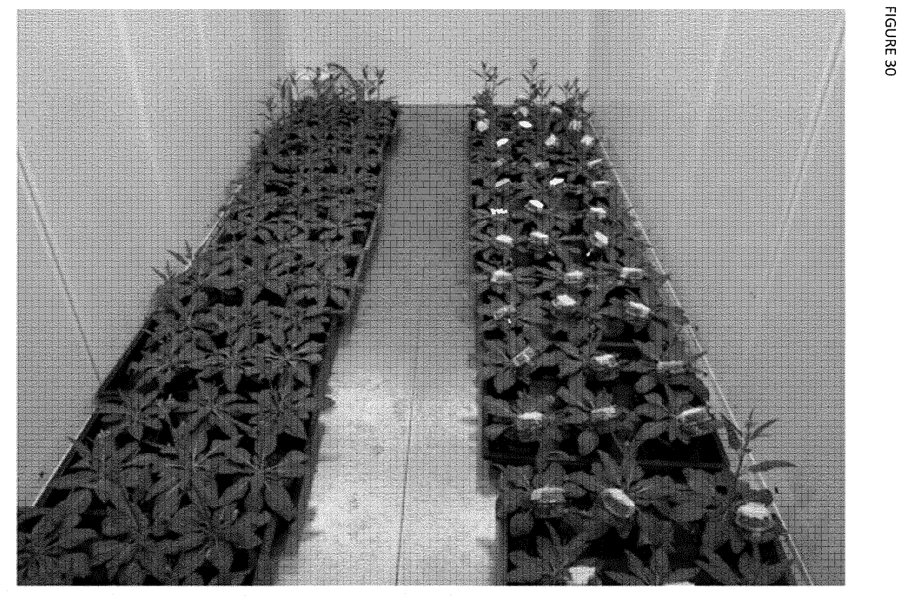

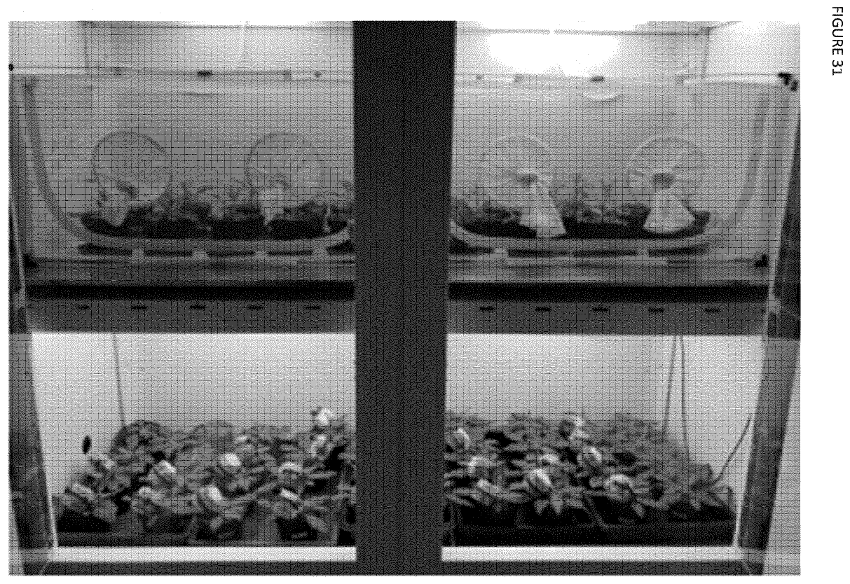

An elicited JA-JAZi module regulates Empoasca resistance To further disentangle the intricacies of the Empoasca-elicited JA signalling sector and its regulated downstream metabolic signatures responsible for Empoasca resistance, a reverse genetics approach to examine the involvement of phenolamides was adopted. Isogenic lines of N. attenuata plants individually RNAi-silenced (ir: inverted repeat) or overexpressed (ov) in different JAZ genes and NaMYC2 to evaluate JA signalling-deficiency, in NaMYB8, the phenolamide master TF regulator, as well as in DH29 and CV86, which catalyze spermidine conjugation steps in phenolamide biosynthesis, were screened in a glasshouse open-choice screening experiment using laboratory colonies of Empoasca decipiens (FIG. 5 and FIGS. 26 and 27). Nymphs and adult E. decipiens preferentially selected irMYC2, irMYB8 and ovJAZi plants for feeding and reproduction, in contrast to the other transgenic lines which were only slightly damaged by a few probing events (FIG. 5). Diverse JAZ proteins allow the JA signalling cascade to regulate an array of metabolic and 15 developmental traits in different tissues at different times (Chini et al., Curr Opin Plant Biol 33, 147-156 (2016), Major et al., New Phytol 215, 1533-1547 (2017)) and thereby contextualize responses and optimize fitness. The modularity of the JA-JAZi sector may provide specific responses relevant to Empoasca leafhoppers. A tissue-wide transcriptomics analysis of all JAZ genes in the N. attenuata genome revealed that NaJAZi is highly expressed in flower tissues and not respon-sive to M. sexta attack in leaves (Li et al., Proc Natl Acad Sci USA 114, E7205-E7214 (2017)), whereas NaJAZh showed the opposite pattern (FIG. 6 and FIG. 28). These patterns were confirmed by exposing leaves to E. decipiens and M. sexta attack and monitoring the kinetics of the JAZ transcript accumulations: M. sexta feeding elicited NaJAZh transcripts in leaves, whereas Em-poasca feeding elicited NaJAZi transcript in leaves (FIG. 6). Y2H assays revealed that NaJAZi interacts with NaMYC2a, whereas NaMYB8 interacts with NaMYC2b (FIG. 29). These data reveal that a MYC2-MYB8-JAZi JA sector is engaged in Empoasca resistance in leaves.

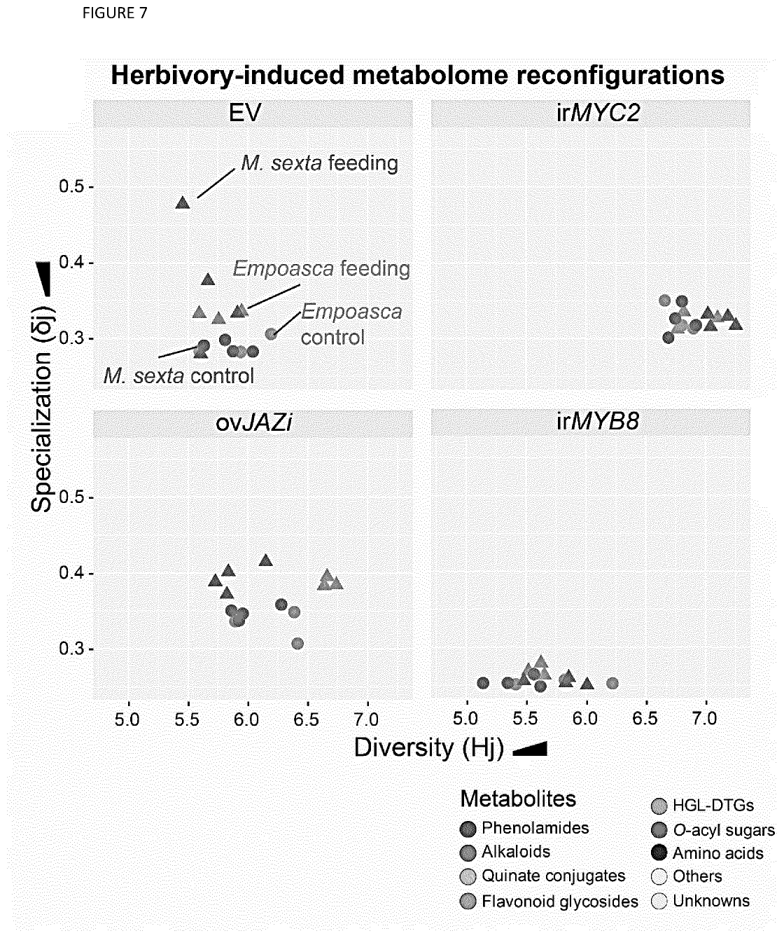

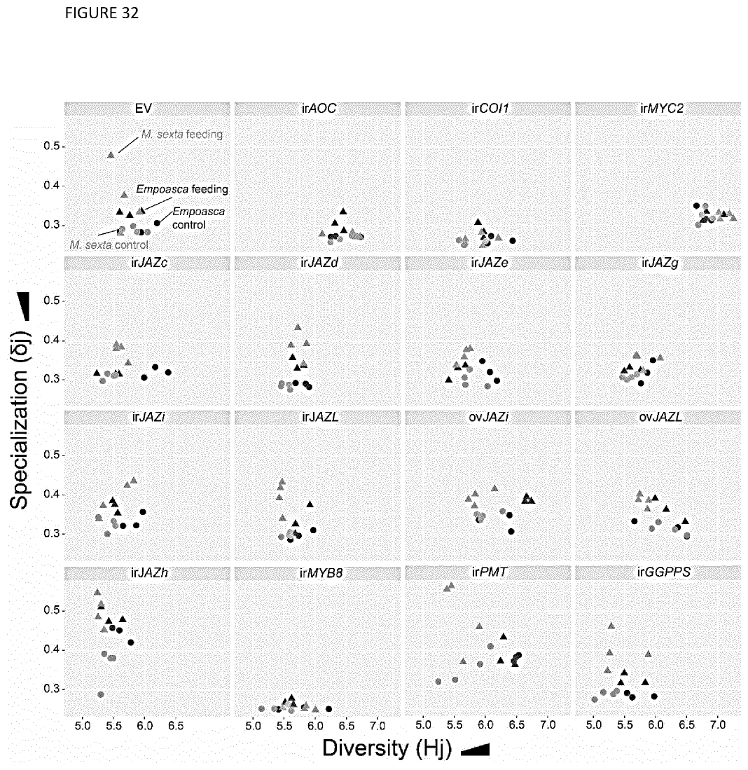

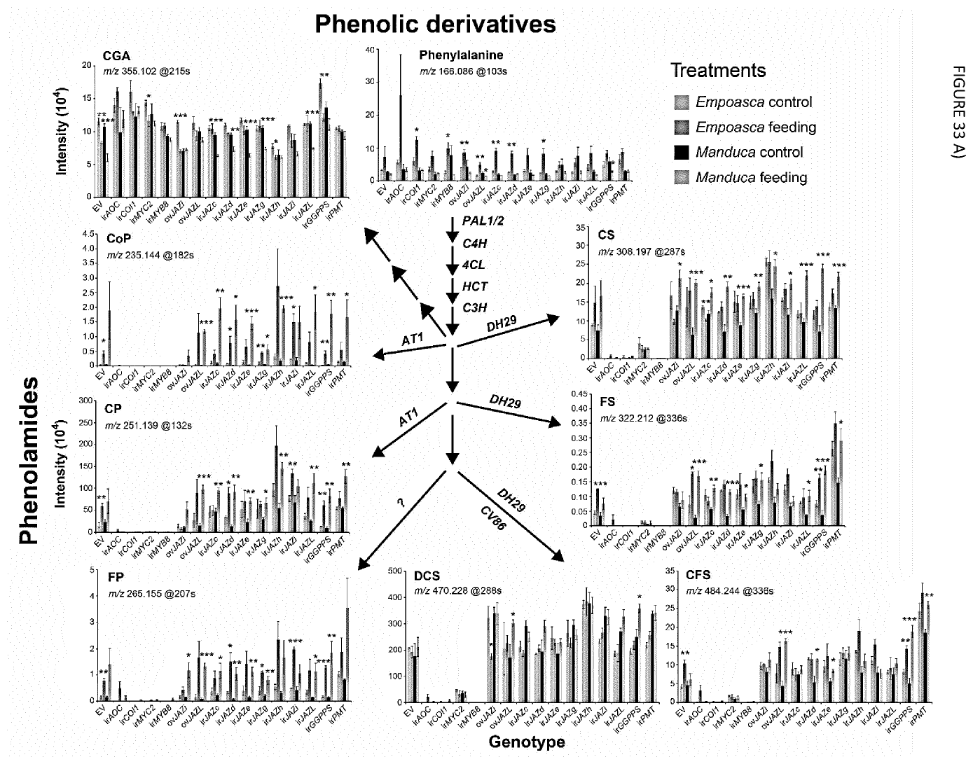

To identify the metabolites elicited by this JA sector, either E. decipiens adults and nymphs or M. sexta larvae on leaves of rosette-stage plants of JA signalling-deficient transgenic lines (FIG. 5) were reared as well as on irAOC and irCOl1 plants, on irGGPPS, which are deficient in 17-HGL-DTGs accumulations and on irPMT plants which are impaired in nicotine accumulations (FIGS. 30 and 31). An analytical and computational workflow (Li et al., Proc Natl Acad Sci USA 112, E4147-4155 (2015), Li et al., Proc Natl Acad Sci USA 113, E7610-E7618 (2016), Li et al., Sci Adv 6, eaaz0381 (2020)) was used to collect high-resolution indiscriminant (data-independent) MS/MS spectra (termed idMS/MS) from these leaf extracts. Metabolome specialization was quantified (6 index), metabolome diversity (Hj index) and metabolic specificity of individual metabolites (Si 10 index) using an information theoryframework (Li et al., Sci Adv 6, eaaz0381 (2020), Li and Gaque-rel, Annu Rev Plant Biol 72, 867-891 (2021)). In the dimensions of information theory-processed metabolome specialization and diversity, M. sexta attack elicited overall higher metabolome plasticity resulting in higher δj scores than did the attack by E. decipiens. The different transgenic lines showed distinct trajectories of metabolome plasticity reprogrammed by the attack of the two insect species (FIG. 7 and FIG. 32).

Focusing on transgenic lines preferred by Empoasca, it was noticed that the distinct signatures of metabolome specialization elicited by herbivore attack were weaker in irMYC2 and irMYB8 plants (FIG. 7 and FIG. 32). This suggested that MYC2 is a master regulator of metabolome plasticity in response to insect attack and that MYB8-dependent herbivory-induced phenolamides comprise the metabolic sector responsible for the increases in metabolome specialization.

The separation of the trajectories of metabolome changes elicited by Empoasca and Manduca attack in the ovJAZi lines, rather than their abolishment, further pointed to a small set of metabolites elicited by Empoasca attack, regulated by NaJAZi, and potentially involved in Empoasca resistance.

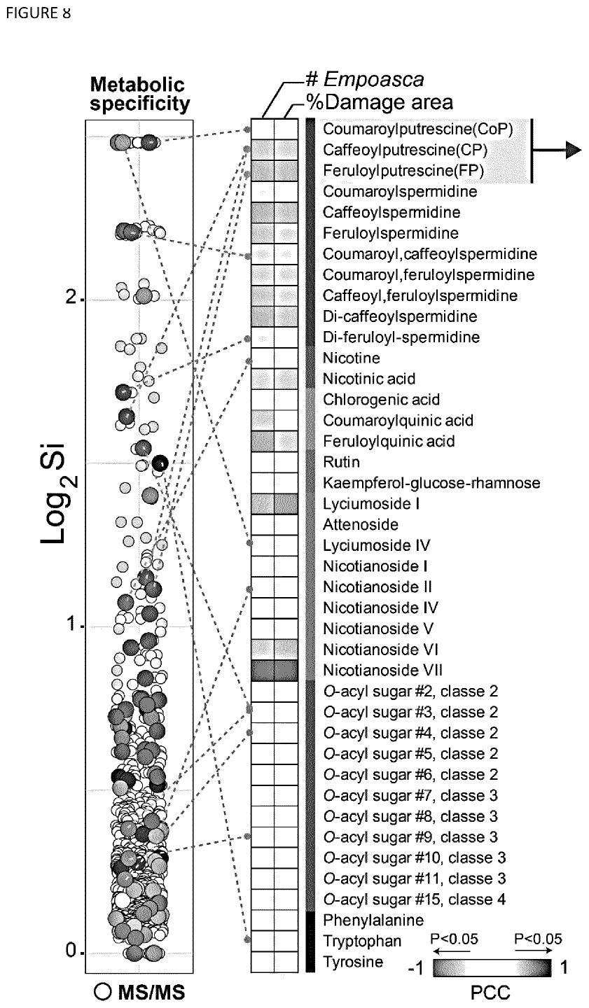

To identify these metabolites, Si scores were ranked for metabolite specificity calculated for each MS/MS spectrum from the E. decipiens-elicited metabolomes from the 4 transgenic lines and linked the Si scores with coexpression heatmaps derived from correlations calculated among individual metabolites and Empoasca numbers and damage using the global variance generated from all reverse-genetics lines used in the feeding experiment (FIG. 8, FIG. 32). Phenolamides ranked at the top of the metabolic specificity Si scores with the putrescine-derived metabolites among the highest and these were negatively correlated with Empoasca numbers and damage, in contrast to particular 17-HGL-DTGs, quinate conjugates and nicotinic acid which showed positive correlations (FIG. 8).

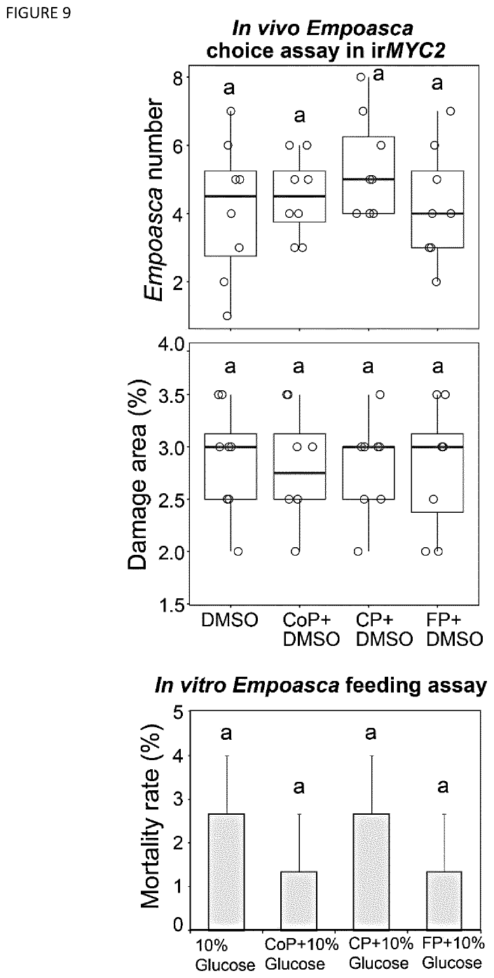

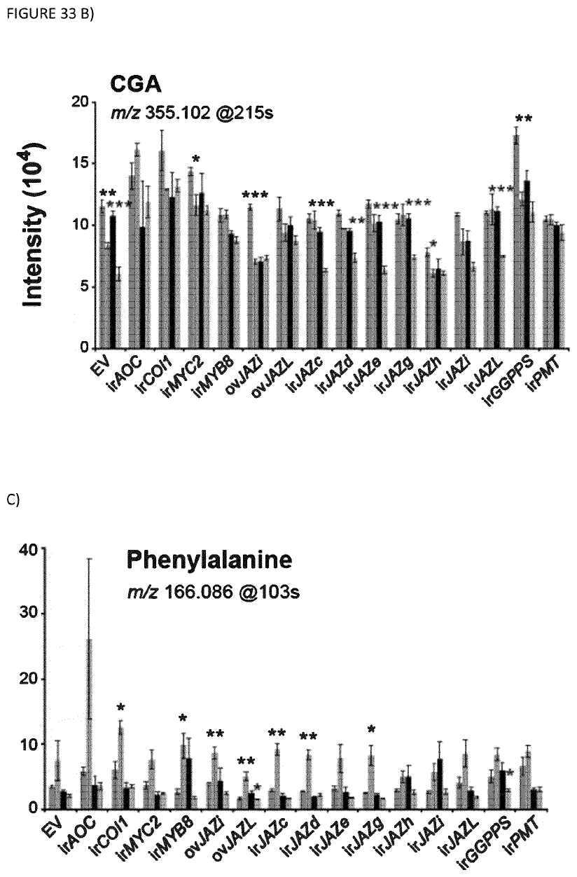

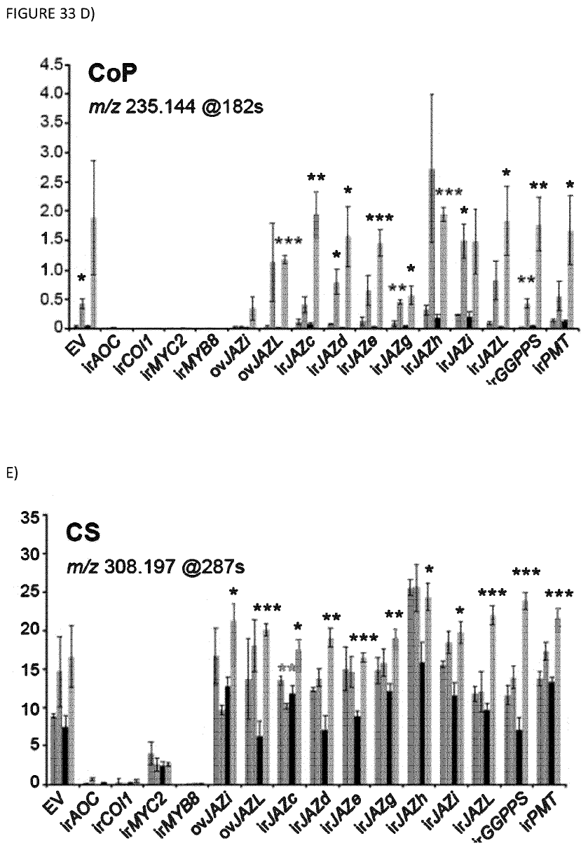

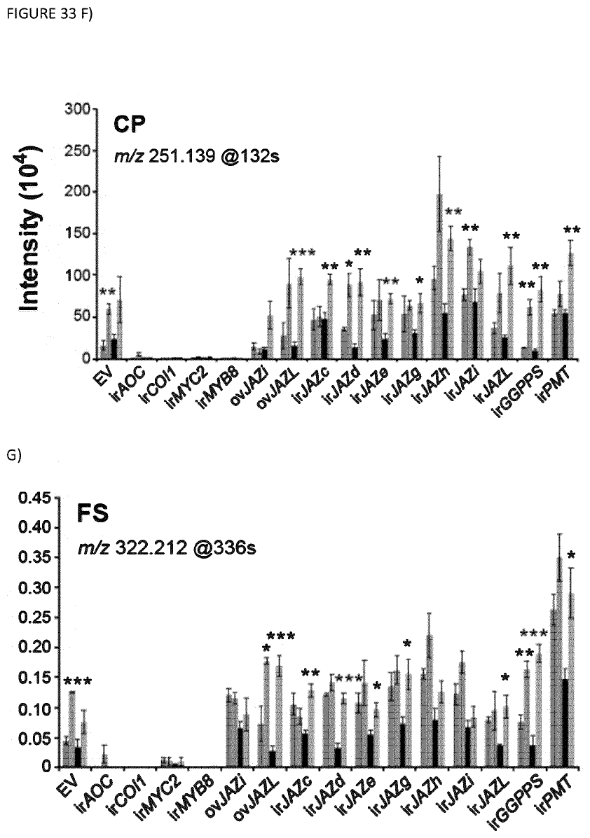

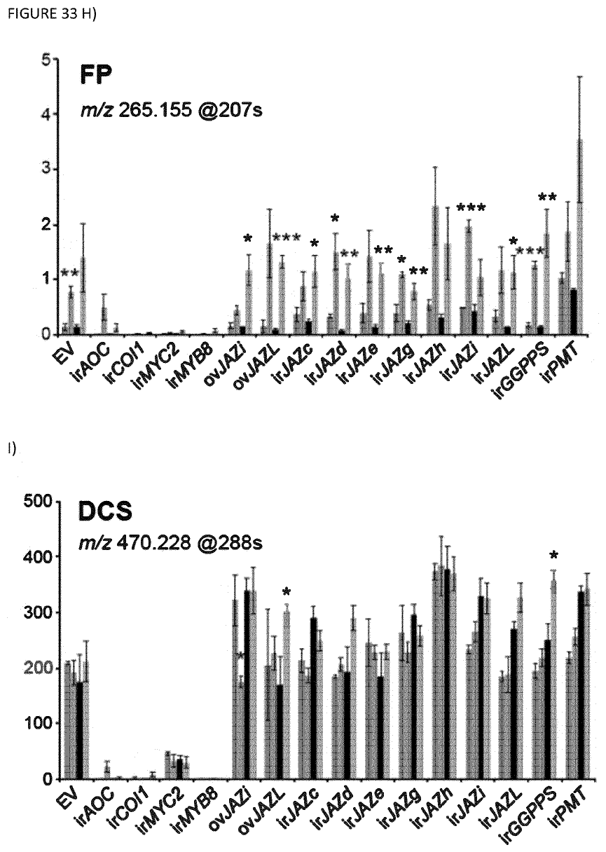

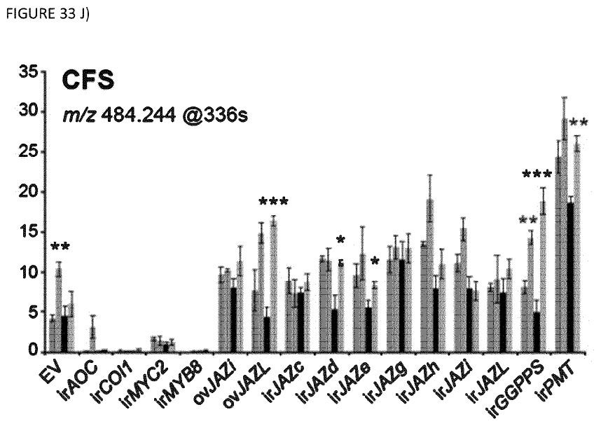

The putrescine-derived phenolamides, CoP, CP and FP were reduced in irAOC, irCOll, irMYC2 and irMYB8 lines, and selectively decreased in ovJAZi plants damaged by Empoasca feeding, but not by Manduca feeding, while other spermidine-derived phenolamides showed similar responses to the attack of the two herbivore species in ovJAZi plants (FIGS. 33A-J). The spermi-dine-derived metabolites could be excluded as mediators of Empoasca nonhost resistance based on the lack of responses of leafhoppers to the irDH29 and irCV86 lines (FIG. 5). To further explore the involvement of CoP, CP and FP, in vivo Empoasca choice assays were conducted by individually infiltrating physiologically relevant concentrations of synthetic CoP (7 μM), CP (100 μM) and FP (10 μM) into leaves of irMYC2 plants which were devoid of elicited phenolamides (FIG. 9 and FIG. 34). However, these infiltrations did not alter the preference of Empoasca for irMYC2 plants.

In vitro Empoasca direct feeding assays conducted with individual compounds at physiologically relevant concentrations in glucose solutions revealed no significant changes in mortality rates of E. decipiens compared to those fed on glucose controls (FIG. 9). These data suggested that CoP, CP and FP were not directly responsible for Empoasca resistance and that other yet-unknown putrescine-derived phenolamide metabolites were responsible.

### Example 3

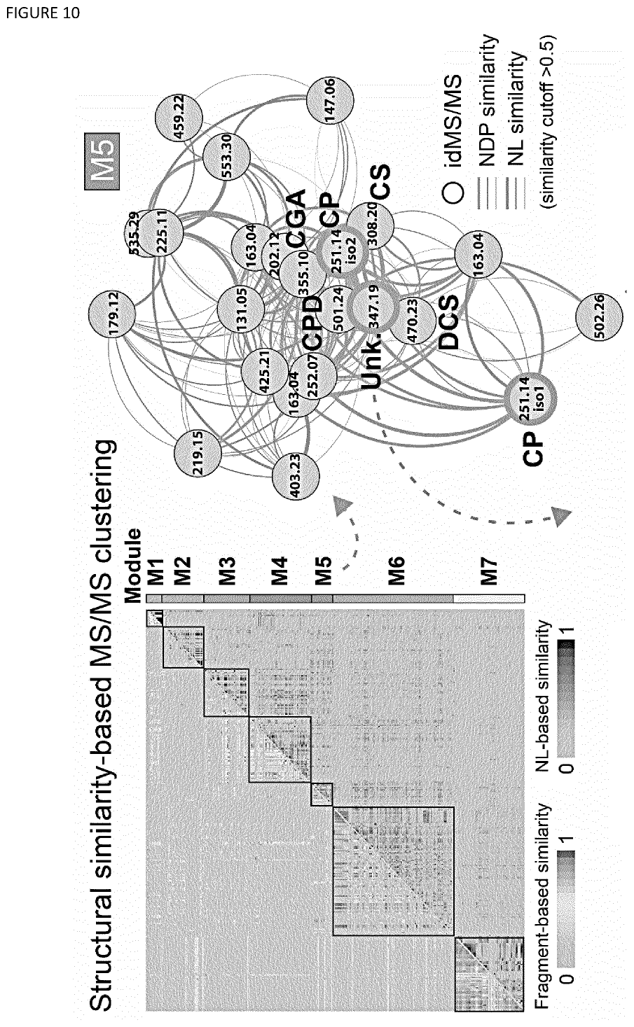

Multi-omics reveals the defense and its 3-pronged pathway Leaves of herbivore-attacked N. attenuata plants grown in the glasshouse accumulate a variety of putrescine- and spermidine-derived phenolamides (Li et al., Proc Natl Acad Sci USA 112, E4147-4155 (2015), Onkokesung et al., Plant Physiol 158, 389-407 (2012)). 15 RILs were selected from the field-based multi-omics dataset of the MAGIC population (FIG. 1), that accumulated high levels of structurally diverse OS-induced phenolamides, to construct idMS/MS and identify the structures of putrescine-derived phenolamides; this effort resulted in 518 non-redundant idMS/MS spectra (FIG. 10). A biclustering analysis was performed to cluster spectra according to fragment (normalized dot product) and neutral loss-based similarities, which resulted in seven modules (FIG. 10). Module 5, particularly enriched in phenolamide-related compounds containing caffeoyl or putrescine moieties, was further mapped onto a molecular network (FIG. 10). An unknown compound at m/z 347.196 ([M+H]+, C₁₉H₂₇N₂O₄+) occupied the first layer of directly-linked network neighbors for the two isomers of CP (m/z 251.14) due to their shared neutral losses of putrescine of A88.10 Da and fragment peak at m/z 163.04 (C9H703+) corresponding to the caffeoyl moiety (FIG. 10 and FIG. 35). The idMS/MS for a fragment peak at m/z 259.09 (C15H1504+), resulting from the loss of putrescine of the molecular ion, showed that it further fragmented to m/z 163.04 with a neutral loss of 96.055 Da (C6H80). This implied that the unknown m/z 347.19 is a CP derivative decorated with a C6H80 residue on the aromatic ring of the caffeoyl moiety (FIG. 35).

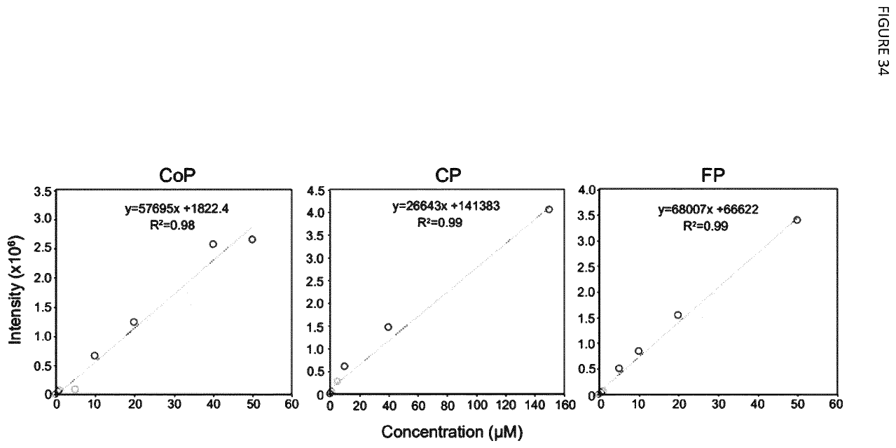

To test whether the unknown m/z 347.19 metabolite is regulated by the specific JA-JAZi module, the co-association network and co-expression analysis for induced m/z 347.19 μgainst Empoasca numbers and damage and JAs in the field-planted MAGIC population was explored (FIG. 1). The m/z 347.19 was negatively correlated with Empoasca damage but positively correlated with JA, JA-Ile and JA-Val (FIG. 36). In a next step the Empoasca-induced metabolomes of JA-deficient transgenic lines were mined (FIG. 2). However, with similar computational workflows, it was not possible to identify this compound in the dataset described earlier (FIG. 2). Extensive experimentation revealed that field-planted RILs elicited more of this unknown compound than did glasshouse-grown RILs (Data 2) and that seemingly minor differences in leaf sampling and extraction technique, specifically the simultaneous exposure of leaves to aluminum foil and liquid N2 and unfavorable pH conditions (Table 1), resulted in the loss of this unstable phenolamide in plants grown and sampled under glasshouse conditions.

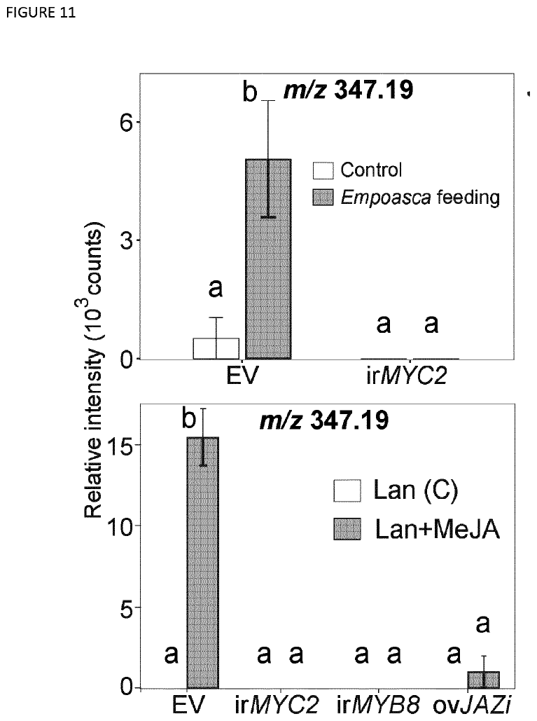

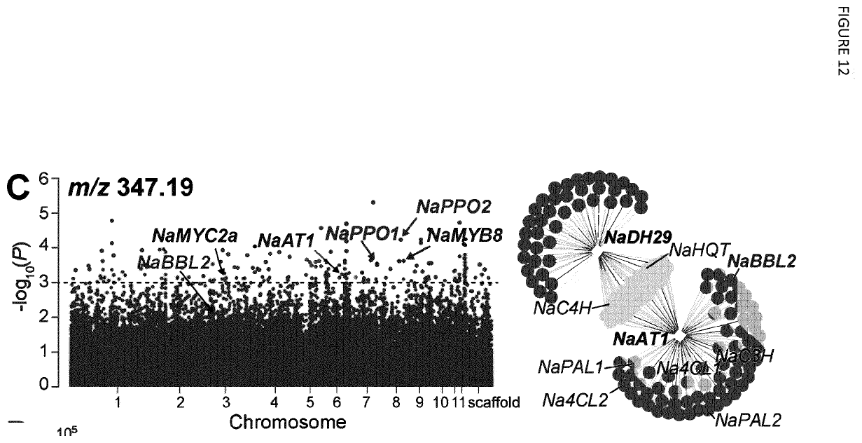

Empoasca was reared on irMYC2 plants again and, by optimizing extraction conditions, found that Empoasca feeding strongly elicited m/z 347.19 accumulations in EV plants; these accumulations were abolished in irMYC2 lines (FIG. 11). During this extraction optimization effort, it was realized that the Empoasca leafhopper elicitation procedure could be replaced with the experimentally more tractable elicitations by larval oral secretions or MeJA. Consistently, MeJA-induced production of m/z 347.19 was hampered in irMYC2, but also in irMYB8 and ovJAZi lines (FIG. 11). These results revealed that the unknown m/z 347.19 is regulated by the JA-JAZi-MYC2-MYB8 signalling sector which is likely responsible for Empoasca resistance.

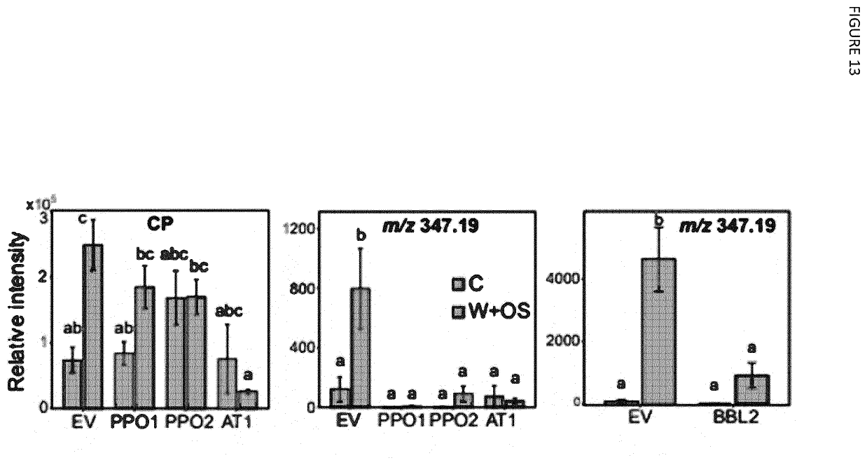

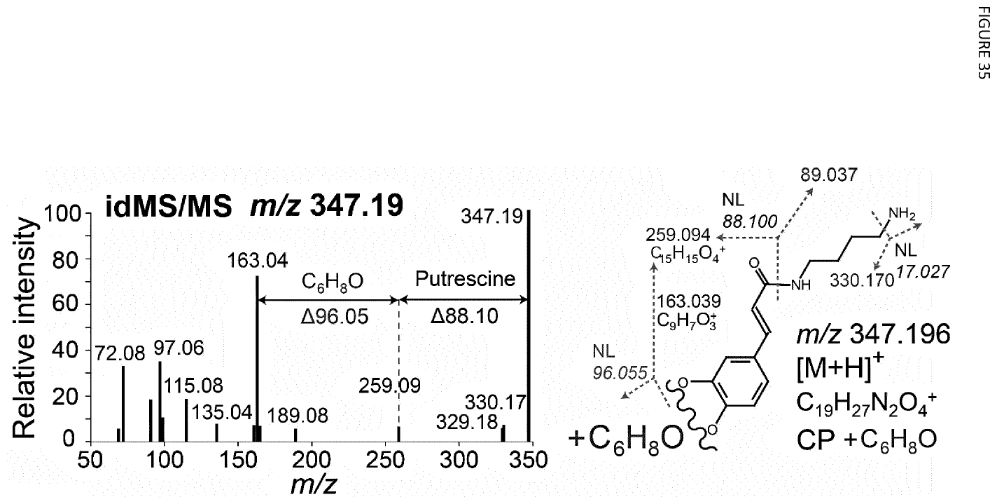

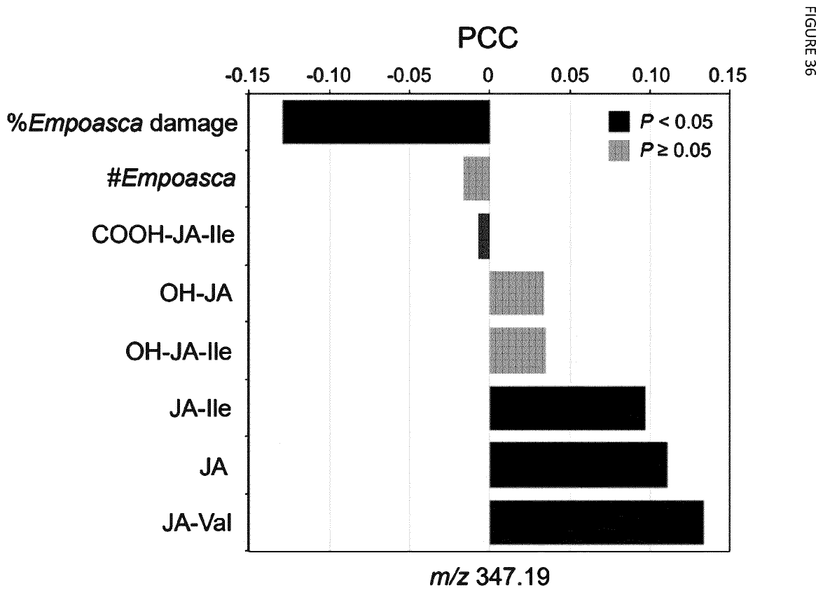

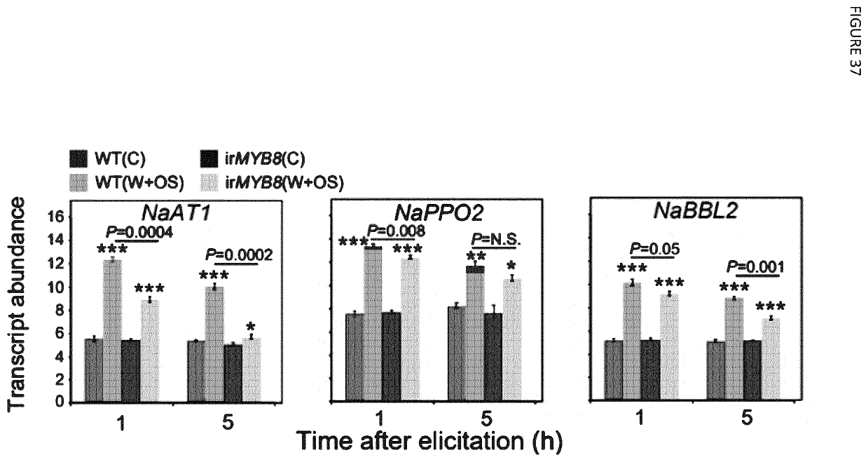

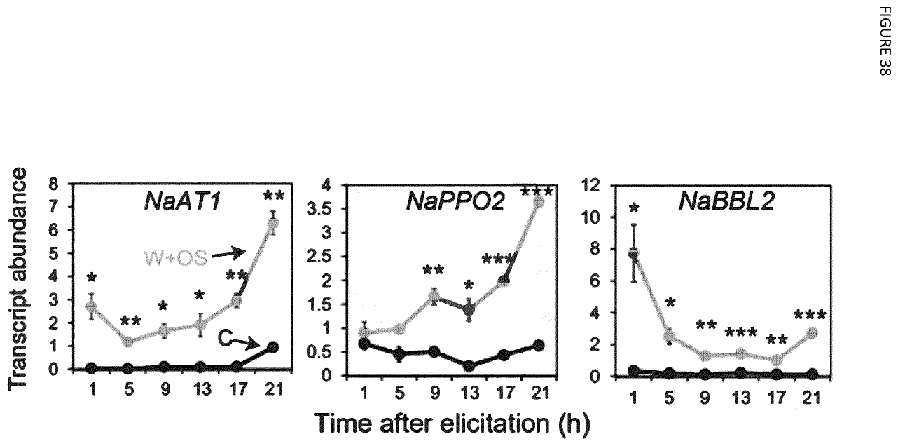

To investigate the biosynthetic origins of m/z 347.19, OS-elicited leaves of the entire MAGIC RIL population grown under glasshouse condition and phenolamide-permissive conditions were extracted, and an mQTL analysis was conducted (FIG. 12). The analysis imputed a series of genes (P's<10-3) known to be involved in CP regulation and biosynthesis, including NaMYC2a, NaMYB8 and NaAT1 which encodes a hydroxycinnamoyl-coenzyme A: putrescine acyltransferase responsible for CP biosynthesis (Onkokesung et al., Plant Physiol 158, 389-407 (2012)), as well as two polyphenol oxidases, NaPPO1 and NaPPO2, located on chromosome 7 and 8, respectively (FIG. 12). Coexpression analyses of a microarray data set of irMYB8 lines (Schafer et al., J Integr Plant Biol 59, 844-850 (2017)) using NaAT1 and NaDH29 as baits revealed a cluster of genes in the NaAT1 groupings known to be involved in the biosynthesis of CP, including NaPAL1, NaPAL2, Na4CL1 and NaC3H. A berberine bridge enzyme-like gene, NaBBL2 was highly coexpressed with NaAT1 and decreased in its induced expressions in irMYB8 lines (FIG. 12 and FIG. 37). The mQTL data set was revisited and it was found that NaBBL2, located on chromosome 3, was associated with m/z 347.19 (FIG. 12), albeit at reduced statistical significance (P=0.0013). Time-resolved microarray data Kim et al., PLoS One 6, e26214 (2011)) of herbivory-elicited expression of NaAT1, NaPPO2 and NaBBL2 revealed that NaAT1 and NaPPO2 showed similar induction patterns in N. attenuata WT whereas NaBBL2 was highly induced at 1 h and retained its induction at later time points albeit at reduced levels (FIG. 38). Moreover, NaAT1, NaPPO1, NaPPO2 and NaBBL2 were reduced in their inductions in herbivory-elicited RNAseq transcriptome dataset of irMYC2 lines (FIG. 39).

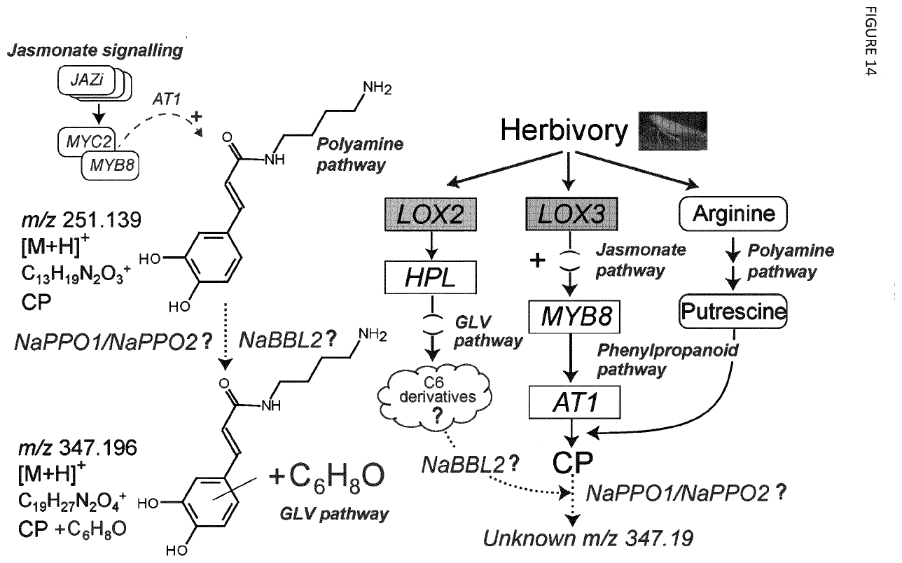

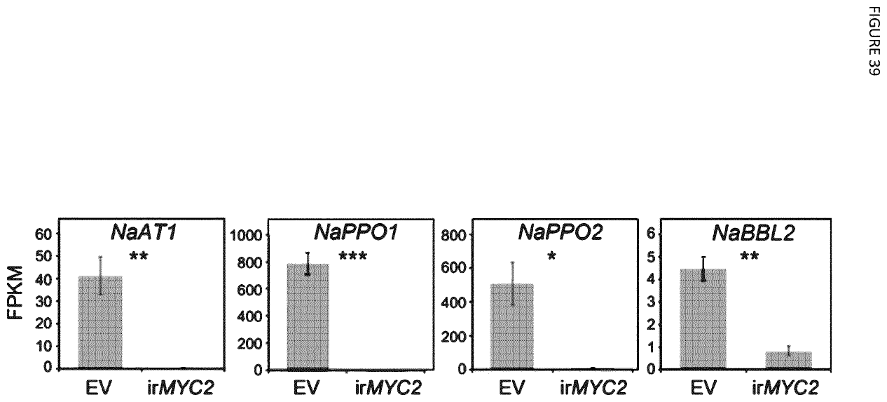

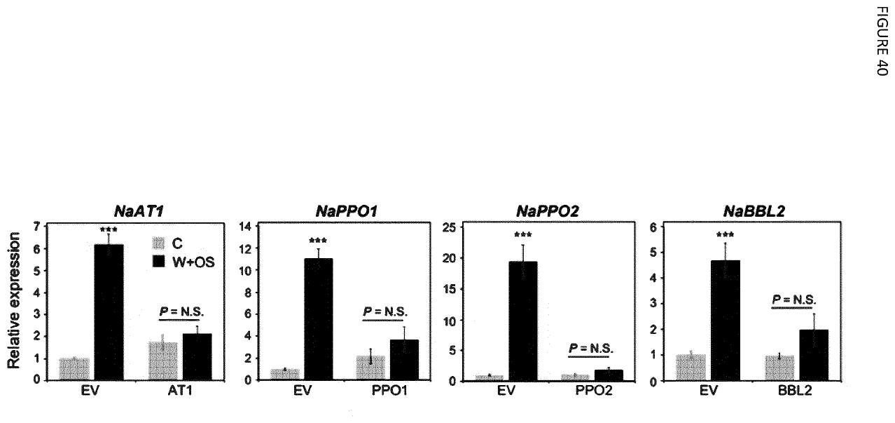

Although the number and order of the biosynthetic steps for m/z 347.19 accumulations remained elusive, it was hypothesized that possible oxidation and acylation reactions were likely required. The further steps thus focused on oxidases and acyltransferases collectively imputed from the multi-omics analysis. Candidate genes included three acyltransferases, NaACT1/2/3, three polyphenol oxidases, NaPPO1/2/3 and a BBL gene, NaBBL2. The in vivo functions of the candidate genes as well as NaAT1 as positive controls were evaluated by silencing their expression in N. attenuata using virus induced gene silencing (VIGS). Consistent with a previous analysis, VIGS of NaAT1 abolished m/z 347.19 accumulation (Li et al., Proc Natl Acad Sci USA 112, E4147-4155 (2015)), and NaPPO1, NaPPO2 and NaBBL2 truncated the elicitation of m/z 347.19 (FIG. 13 and FIG. 40). Untargeted metabolomics analysis of the VIGS-silenced plants revealed that silencing NaPPO1 and NaPPO2 truncated m/z 347.19 accumulations without changes in other phenolamides (FIG. 13 and FIG. 41). These results suggest that NaPPO1, NaPPO2 and NaBBL2 are required for the in vivo production of m/z 347.19.

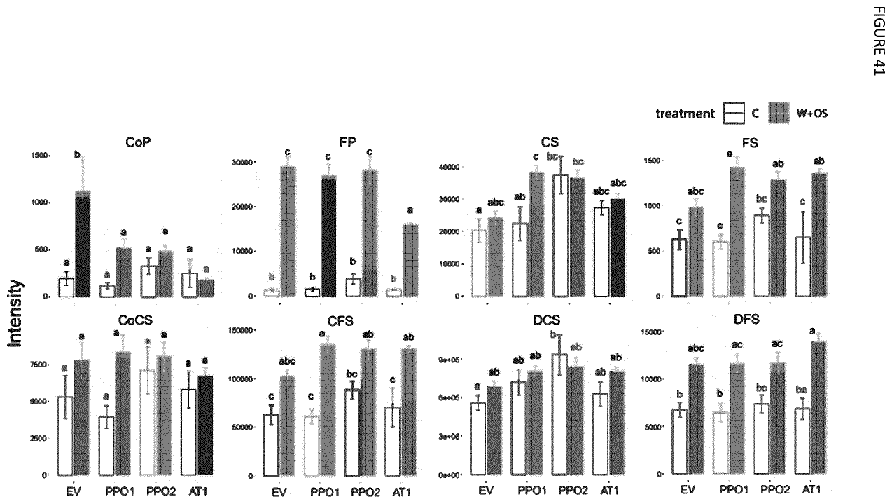

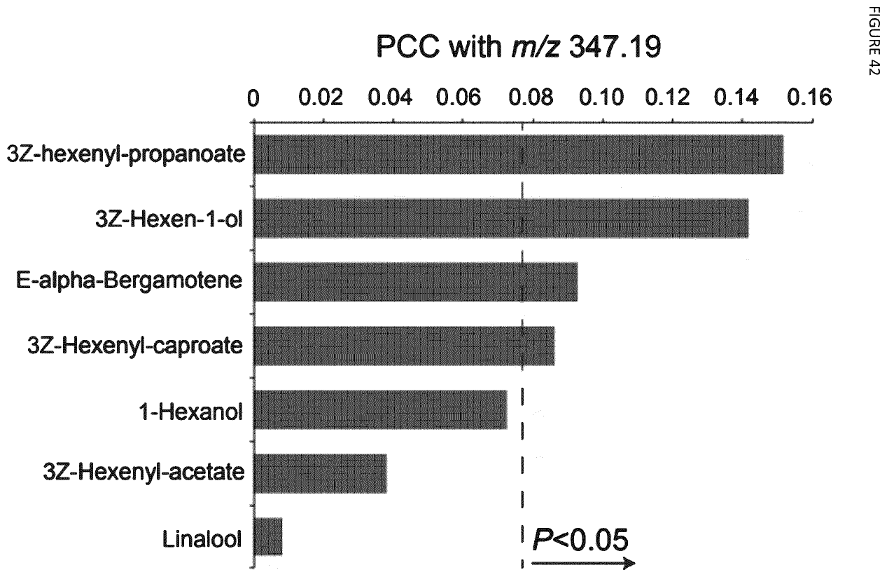

Previous analysis suggested that the additional C6H80 residue of m/z 347.19 was produced from the fatty acid oxylipin cascade, which converts C18 polyunsaturated fatty acids released from biological membranes during stresses, wounding and herbivory (Matsui, Curr Opin Plant Biol 9, 274-280 (2006)) to produce green leaf volatiles (GLVs) enriched in reactive C6 derivatives (Li et al., Proc Natl Acad Sci USA 112, E4147-4155 (2015)). However, the origins of the C6 metabolite and the biochemical reaction involved for the formation of m/z 347.19 remained unknown. To clarify this, the herbivory-elicited GLVs was measured in the same glasshouse-grown MAGIC RIL population used forthe imputation (FIG. 3) and a correlational analysis with herbivory-elicited m/z 347.19 accumulations was conducted. (Z)-3-hexenal-derived volatiles, such as (Z)-3-hexenyl-propanoate and (Z)-3-hexenol, were the most significantly positively correlated metabolites with m/z 347.19, while 1-hexanol, linalool or other elicited volatiles were not (FIG. 14 and FIG. 42).

The C6 aldehydes, with their molecular formula of C6H80, are the most reactive aldehydes produced from the GLV pathway and have been hypothesized (Li et al., Proc Natl Acad Sci USA 112, E4147-4155 (2015)) to be the missing substrates for the biosynthesis of m/z 347.19.

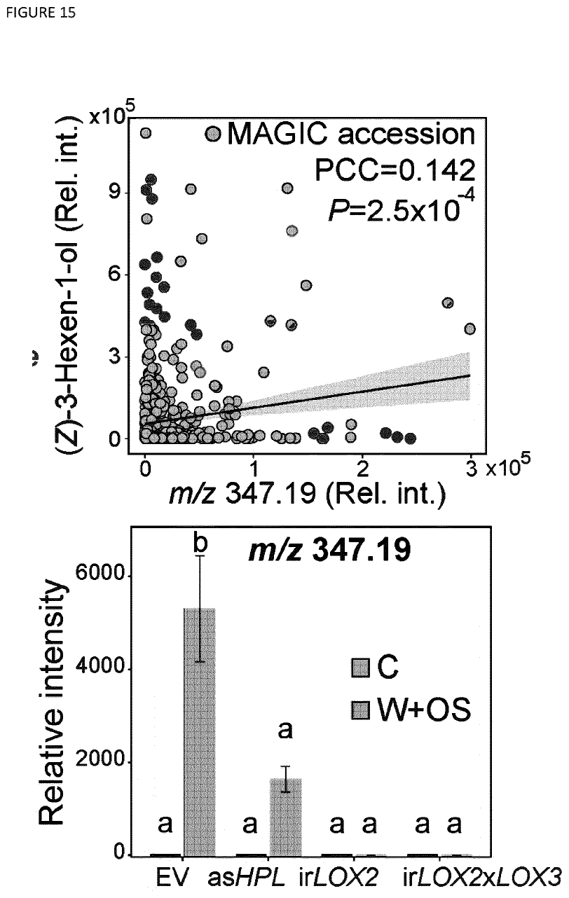

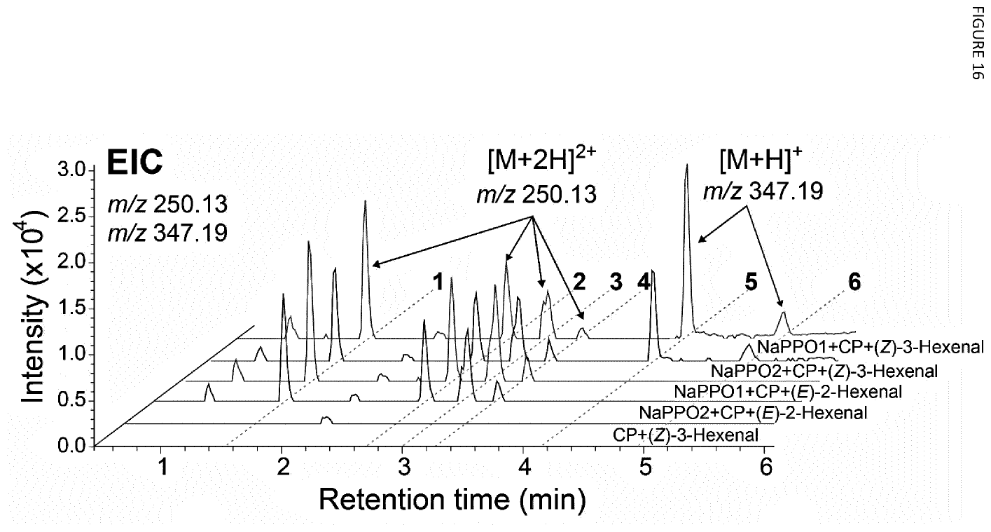

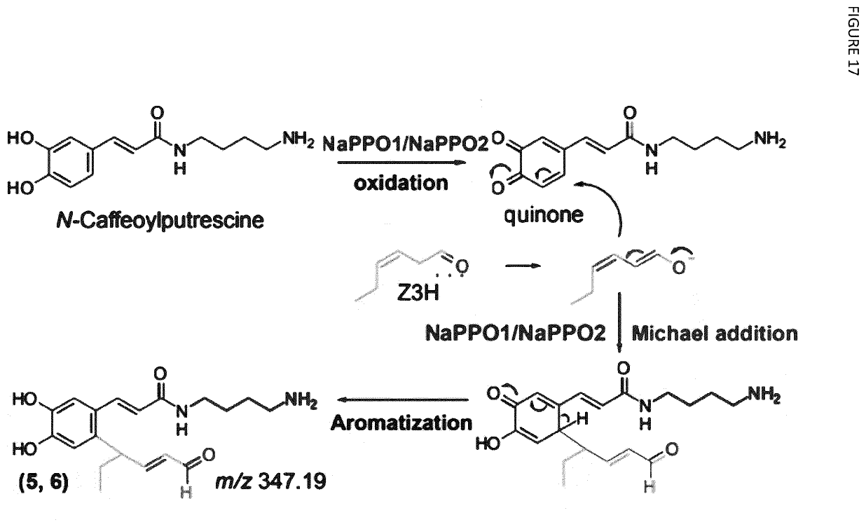

Consistent with previous analysis (Allmann et al., Plant Cell Environ 33, 2028-2040 (2010)) stably silencing LIPOXYGENASE2 (irLOX2) in N. attenuata, which controls the first committed step in the GLV pathway, abolishes C6 aldehydes production and total GLV emissions, and stably silenced crosses of irLOX2 and irLOX3 (irLOX2xirLOX3), completely eliminated m/z 347.19 production (Li et al., Proc Natl Acad Sci USA 112, E4147-4155 (2015)) (FIG. 15). Silencing NaHPL (with an antisense construct: asHPL), which catalyzes the formation of initial C6 GLV products of (Z)-3-hexenal, and its isomer (E)-2-hexenal, results in considerable time-dependent reductions of GLVs in N. attenuata (Joo et al., Plant Cell Environ 42, 972-982 (2019)), and reduced m/z 347.19 accumulations (to about 1/3 of WT levels: FIG. 15). From these results, it was surmised that in response to Empoasca probing, m/z 347.19 is produced by a three-pronged metabolic pathway, composed of the LOX2/HPL-GLV pathway, the LOX3-JA regulated phenylpropanoid pathway and the polyamine pathway, which are condensed by NaPPO1, NaPPO2 and NaBBL2 using CP and (Z)-3-hexenal or (E)-2-hexenal to produce m/z 347.19 (FIG. 14).

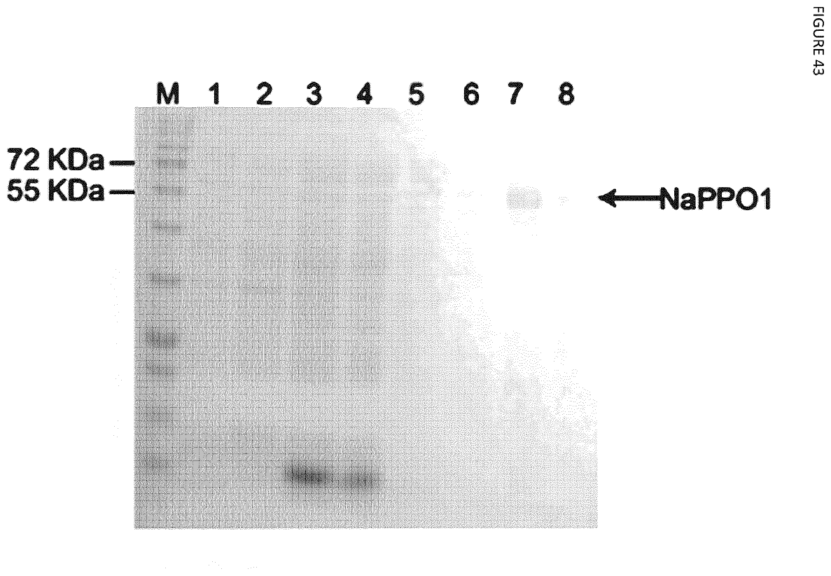

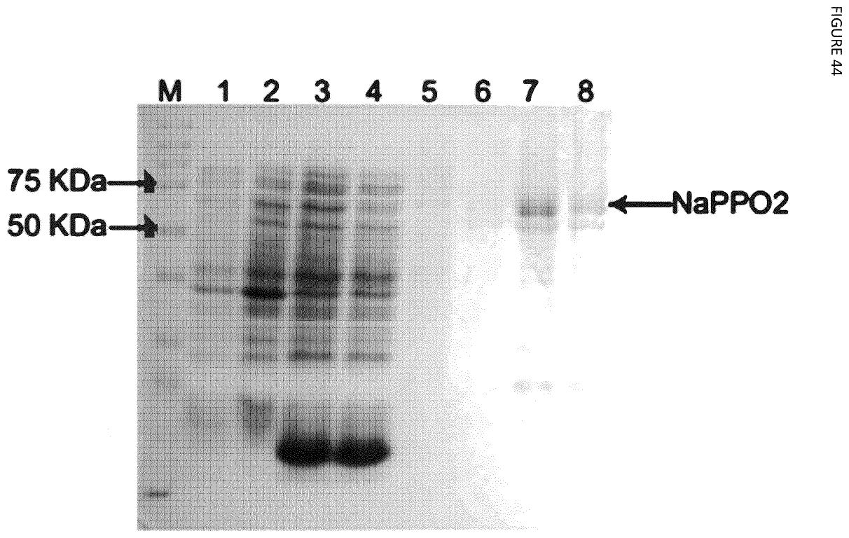

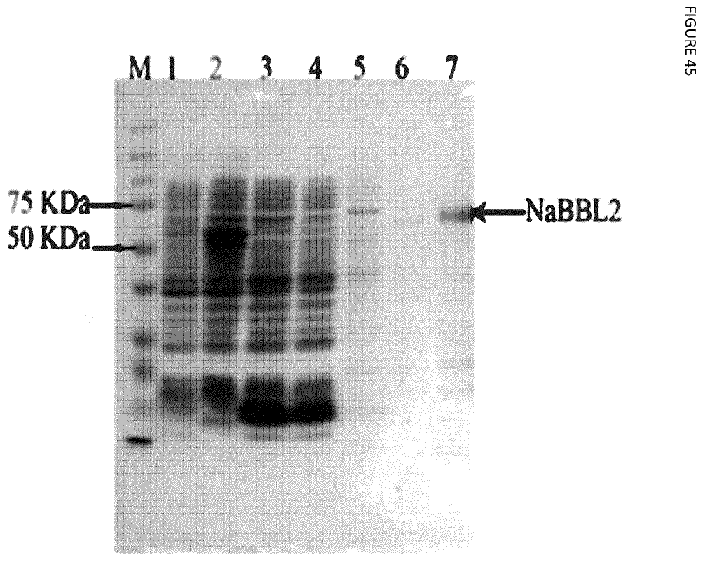

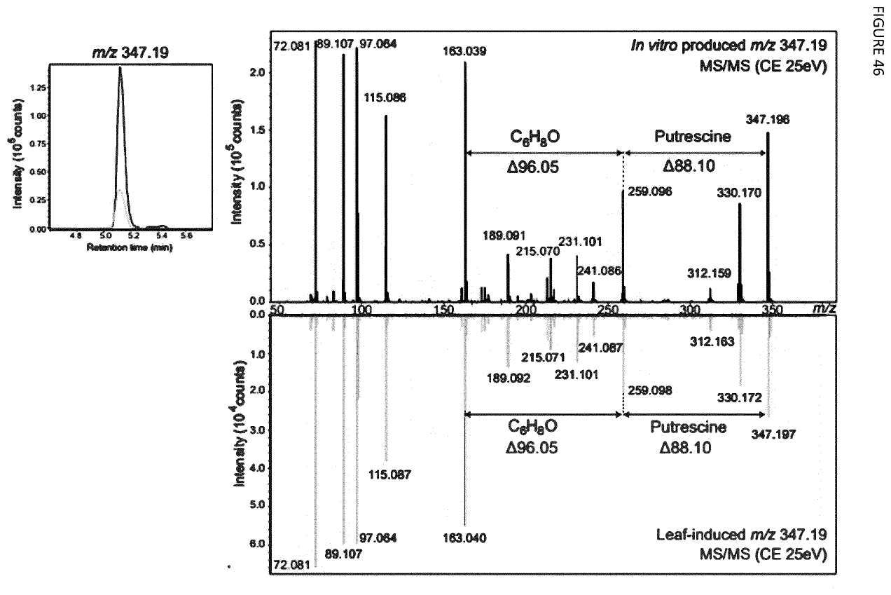

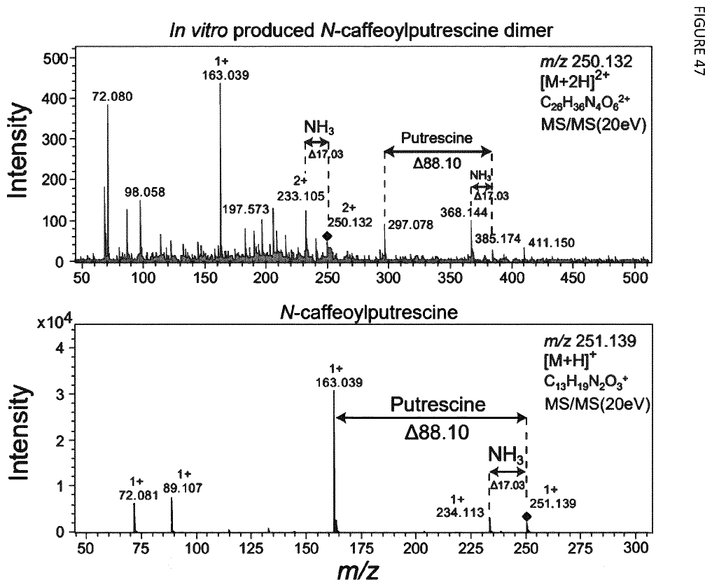

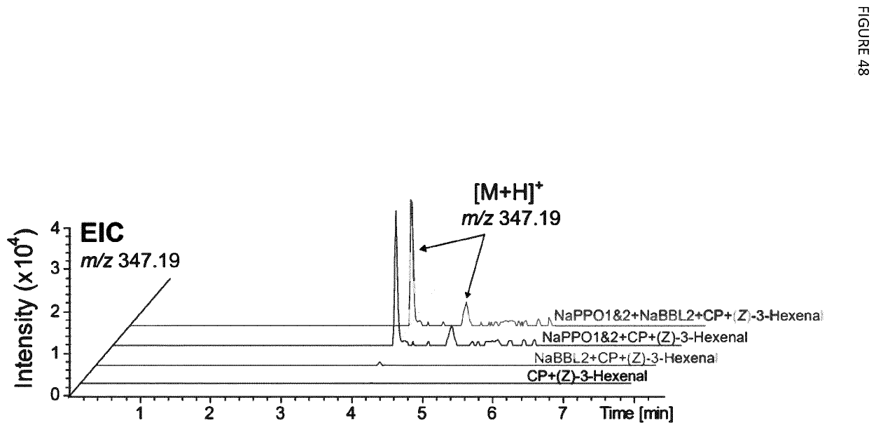

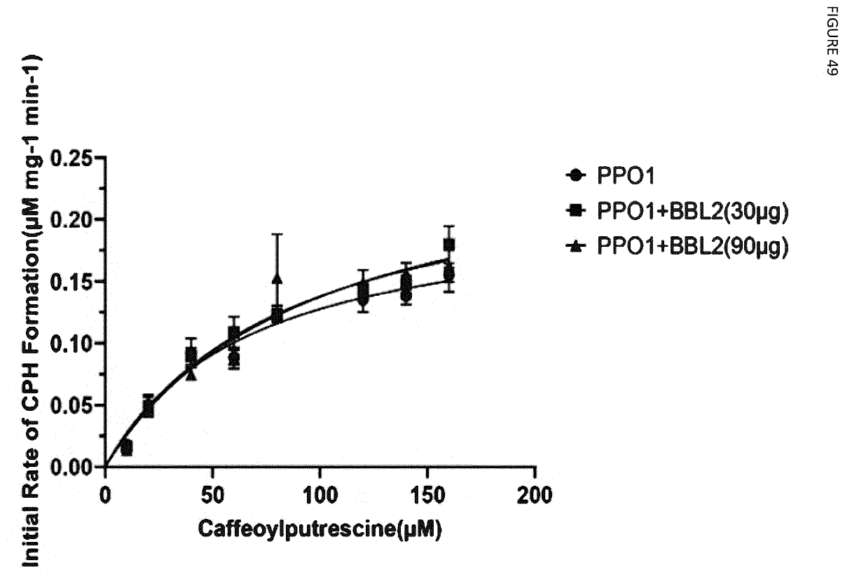

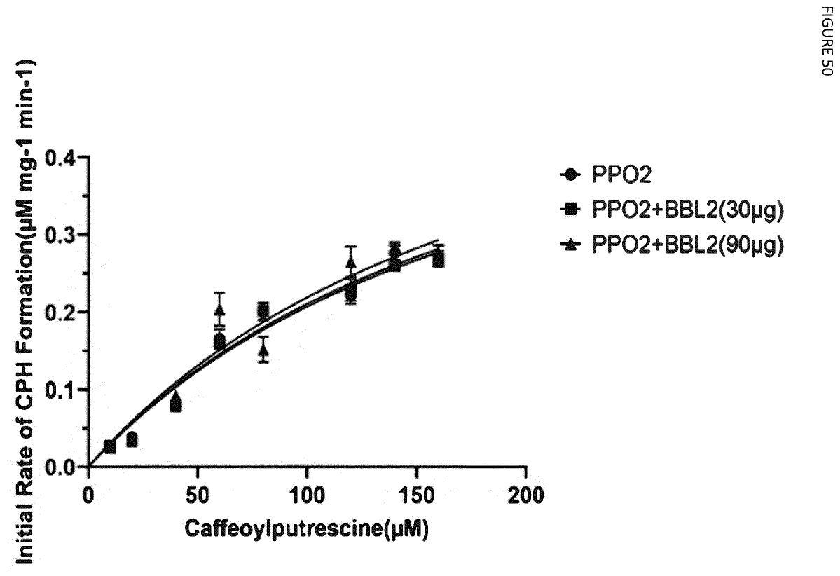

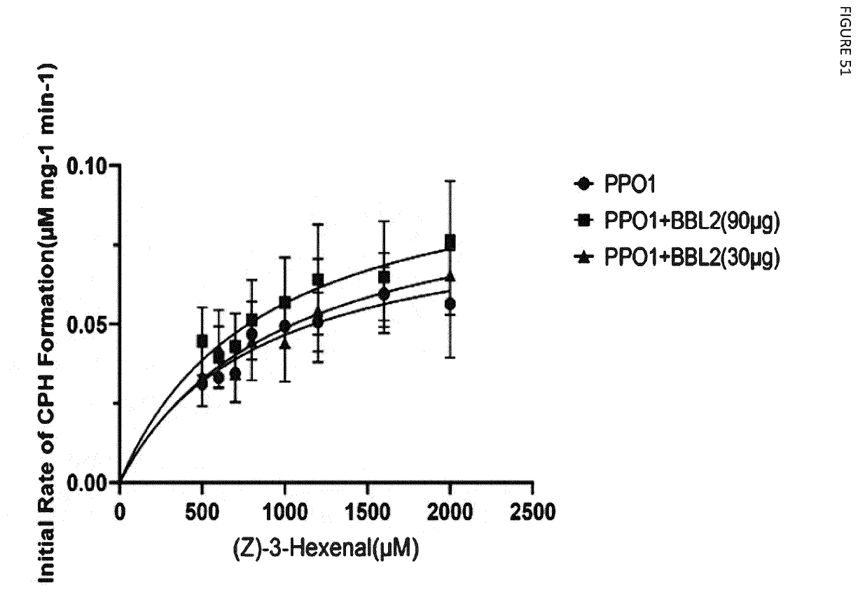

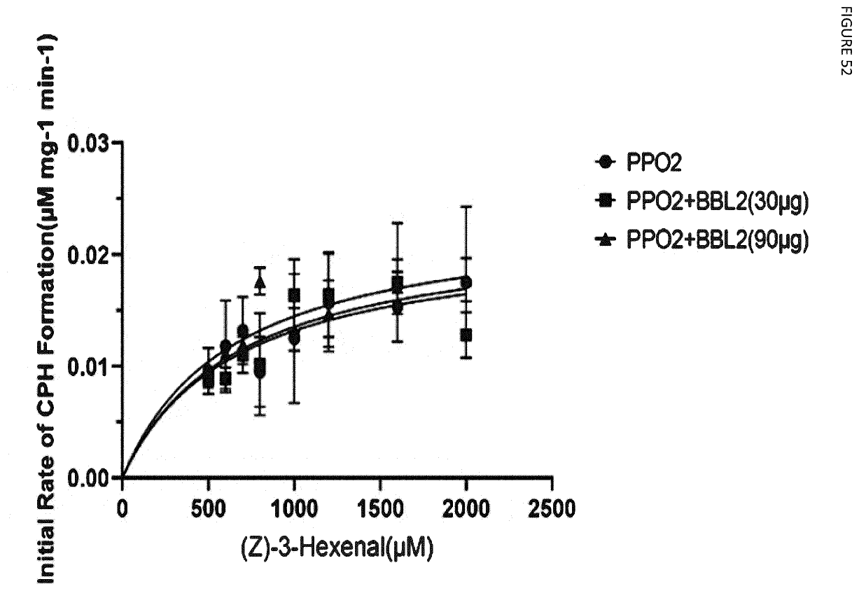

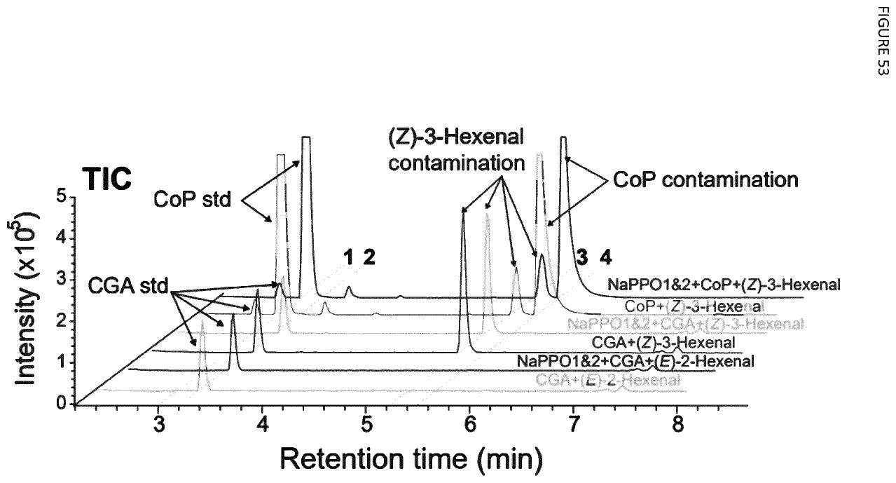

To test this hypothesis, purified NaPPO1, NaPPO2 and NaBBL2 proteins with N-terminal hexahistidine tags after expression in Escherichia coli were isolated (FIGS. 44-45). Incubation of either NaPPO1 or NaPPO2 (NaPPO1/2) with CP and (Z)-3-hexenal yielded a m/z 347.19 peak with identical MS/MS spectrum and retention time as the m/z 347.19 induced in N. attenuata leaves (FIG. 46). Additionally, by-products of doubly charged CP dimers at m/z 250.13 ([M+2H]2+, C26H36N4O62+) which are not detected in oral secretion-induced leaves of N. attenuata WT (Data 2) were also produced in vitro (FIG. 16 and FIG. 47). These doubly charged CP dimers were only produced when the quantities of the (Z)-3-hexenal substrate were lower than those of the CP substrate in vitro. NaPPO1/2 showed little-to-no activity when incubated with CP and (E)-2-hexenal (FIG. 16). NaBBL2 alone could not use CP and (Z)-3-hexenal as the substrates to produce m/z 347.19, and under in vitro conditions, the addition of NaBBL2 in the presence of NaPPO1/2, CP and (Z)-3-hexenal did not significantly increase the production of m/z 347.19 (FIGS. 48-52 and Table 2). As PPOs have broad substrate specificities that can accept both hydroxy benzenes and/or ortho-dihydroxylated benzenes as substrates (McLarin and Leung, S Crit Rev Biochem Mol Biol 55, 274-308 (2020)) the substrate specificities of NaPPO1 and NaPPO2 were explored by incubating NaPPO1/2 and (Z)-3-hexenal with CoP or with chlorogenic acid (CGA) which contains aromatic dihydroxylation as CP. However, no new products were found, indicating that CoP and CGA were not accepted as substrates by NaPPO1/2 (FIG. 53). Together, these data reveal that NaPPO1/2 accepts CP and (Z)-3-hexenal as substrates to produce m/z 347.19 in vitro.

### Example 4

### Biosynthetic Logic of the Reactive m/z 347.19 Chemistry

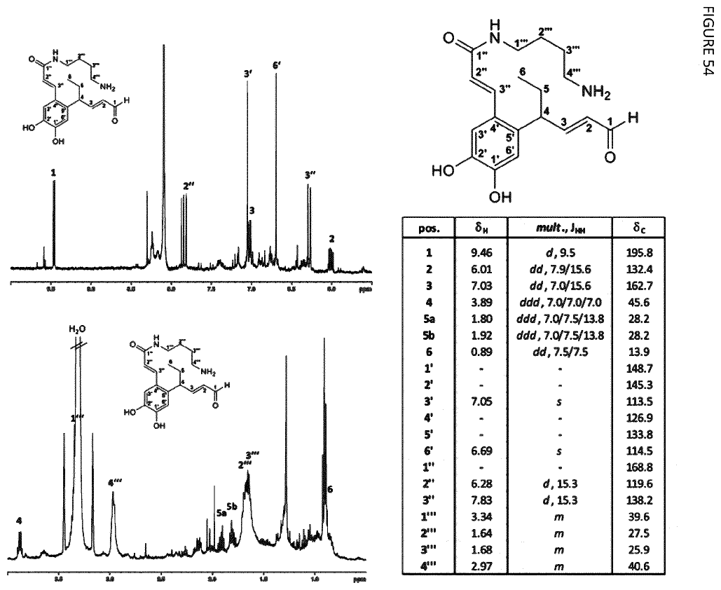

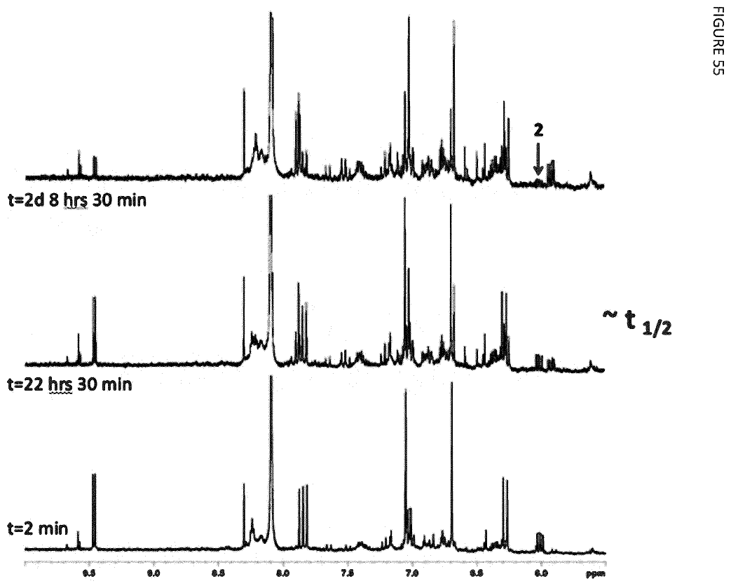

To elucidate the chemical structure of m/z 347.19, it was attempted to isolate and purify the m/z 347.19 using induced N. attenuata leaf material and enzyme assay-derived products. However, several attempts failed due to the instability of m/z 347.19. While relatively stable in ammonium-acetate buffer (pH 4.8), when concentrated either by rotatory evaporation or freeze drying, m/z 347.19 rapidly decomposed (Table 1). These observations indicate that m/z 347.19 is reactive and unstable at high pH. The purification procedures for m/z 347.19 were modified to produce large quantities from enzymatic assays under weak acidic conditions and purified m/z 347.19 using solid phase extraction under argon atmospheres. The purified m/z 347.19 was then subjected to NMR analysis, which elucidated its structural identity as a CP-5-(Z)-3-hexenal compound (hereafter named CPH) (FIG. 54). CPH's half-life is only about 22h in acidified methanol-d3 (0.1% formic acid) at room temperature in darkness (FIG. 55). CPH contains both a reactive moiety of a, 3-unsaturated aldehyde derived from (Z)-3-hexenal, which is electrophilic, and an amine feature of the caffeoylputrescine, which is nucleophilic.

CPH results from the biochemical union of so-called “direct” (CP) and “indirect” ((Z)-3-hexenal) defense metabolism and the hypothesis that CPH was the metabolic trait underlying Empoasca nonhost resistance was formulated. In suggestion were two possible mechanisms of action: that the rapid polymerization of the electrophilic and nucleophilic groups could occlude the mouthparts of probing Empoasca leafhoppers; that the a, 3-unsaturated aldehyde may function as a protein-crosslinker that disables Empoasca proteins (29). A three-step biosynthetic mechanism for the production of CPH was proposed: NaPPO1/2 oxidizes CP to the corresponding caffeoyl quinone derivative, and activates (Z)-3-hexenal for a Michael addition reaction, the product of which is aromatized to form CPH (FIG. 17).

### CPH is Responsible for Empoasca Resistance

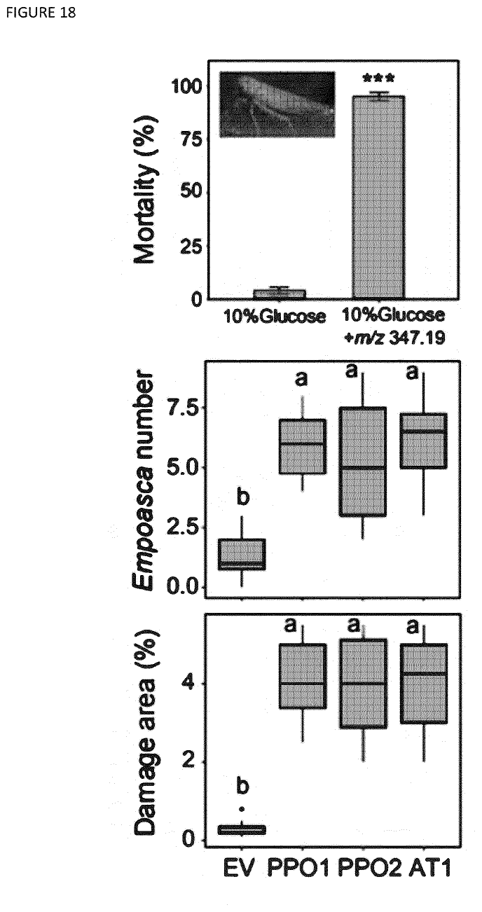

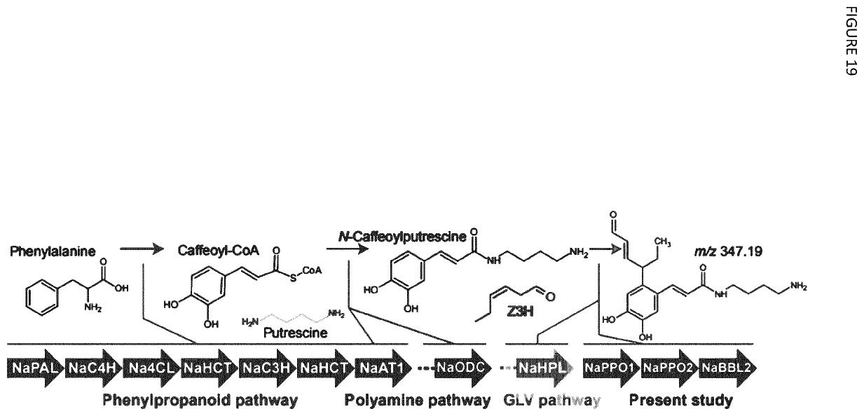

To test if CPH is responsible for Empoasca resistance, E. decipiens were fed with physiologically relevant concentrations of 1 μM (estimated from field-collected elicited leaves) of NMR-confirmed CPH in diets containing 10% glucose in vitro. After 6 h of feeding, the CPH treatment caused almost 100% percent mortality of E. decipiens, contrasting to leafhopper growth on control diets (P=3×10-8, Student's t-test) (FIG. 18). NaAT1 expression was silenced in N. attenuata plants using VIGS, which disrupted the production of both CP and the CPH (Li et al., Proc Natl Acad Sci USA 112, E4147-4155 (2015)) (FIG. 13). In vivo choice assays revealed that NaAT1-silenced plants received significantly more Empoasca damage and higher Empoasca numbers than did EV plants (FIG. 18). Similarly, silencing of either NaPPO1 or NaPPO2 in plants abolished only the accumulation of CPH in plants, without significant alterations in other phenolamide pools (FIG. 13 and FIG. 41) and resulted in a clear Empoasca feeding preference (P=3.8×10-6 and P=1.5×10-3, respectively, Student's t-test) and greater Empoasca damage (P=1.1×10-7 and P=3.5×10-6, respectively, Student's t-test) compared to EV plants (FIG. 18). Together, these in vitro and in vivo results suggest that CPH in N. attenuata is responsible for the plant's Empoasca nonhost resistance.

NaBBL2 is required to engineer CPH biosynthesis in crop species

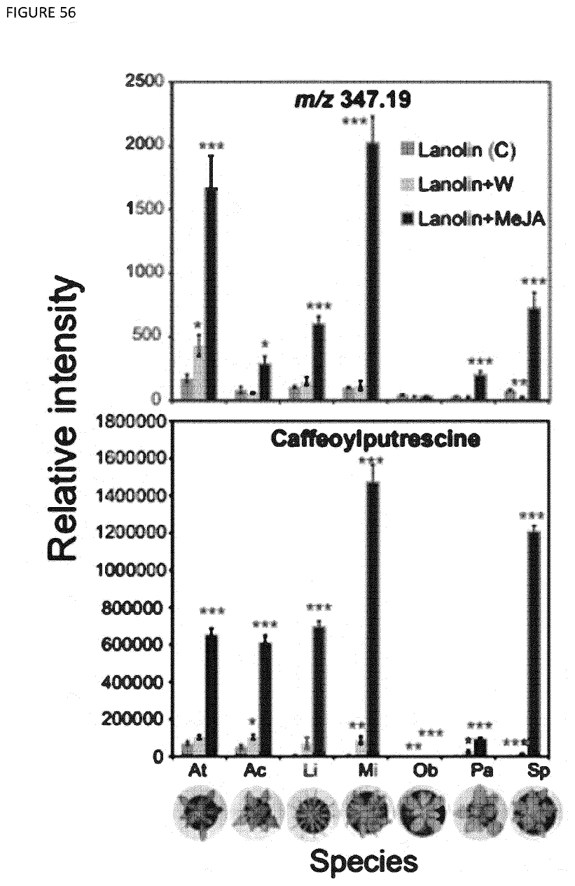

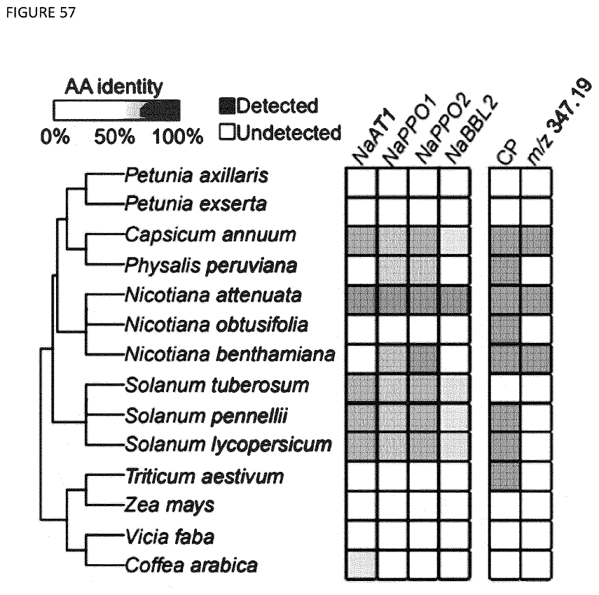

The discovery of CPH and its biosynthetic pathways underlying nonhost resistance offers a framework for the engineering of CPH biosynthesis in crop plants as a means of optimizing a plant's endogenous metabolism for defense against the attack of devastating leafhopper pests, the diseases they vector, and other nonhost pests of crops. It was investigated whether CPH is widely found in Solanaceae and other plant taxa. Metabolic profiling of N. attenuata's close rel-atives, revealed that 6 of 7 Nicotiana species induced CPH in a coordinated fashion with CP when elicited by MeJA (FIG. 56). Exactly 13 different taxa were selected from different plant families including several crop species and compared amino acid identities of NaAT1, NaPPO1, NaPPO2 and NaBBL2 with their closest homologues using BLAST searches of the NCBI sequence database (FIG. 57): 5 Solanaceae taxa of the 13 species examined contain all orthologues of the four protein sequences. Moreover, MeJA-induced CP in 8 species was detected, including 7 Solanaceae species and wheat, while only 2 species, Capsicum annuum and Nicotiana benthamiana, other than N. attenuata, produced CPH (FIG. 57). These results suggest that CPH production may be re-stricted to the Solanaceae.

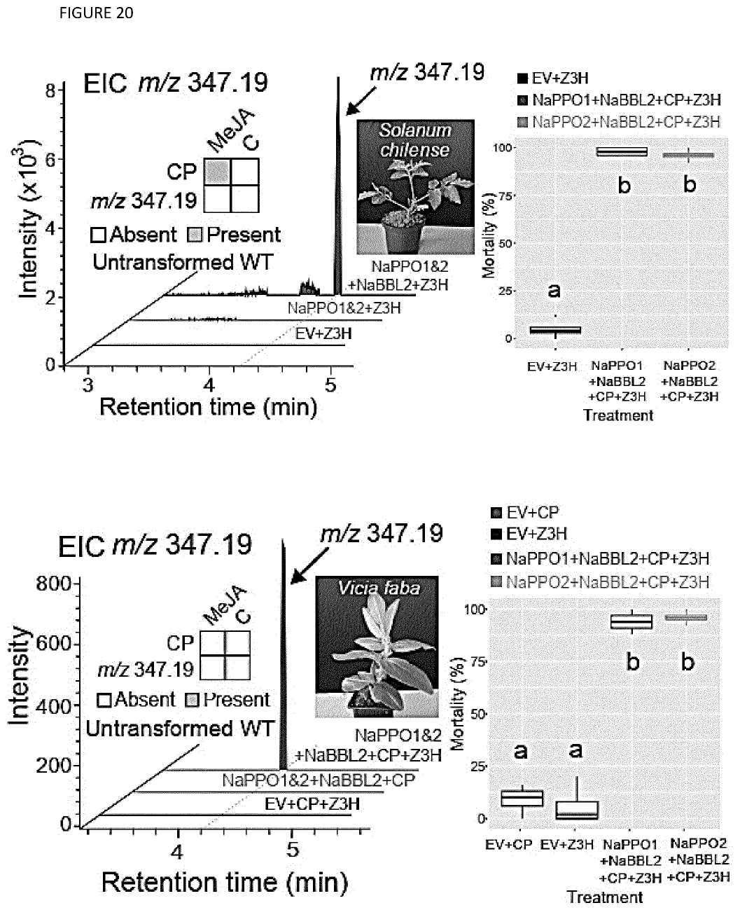

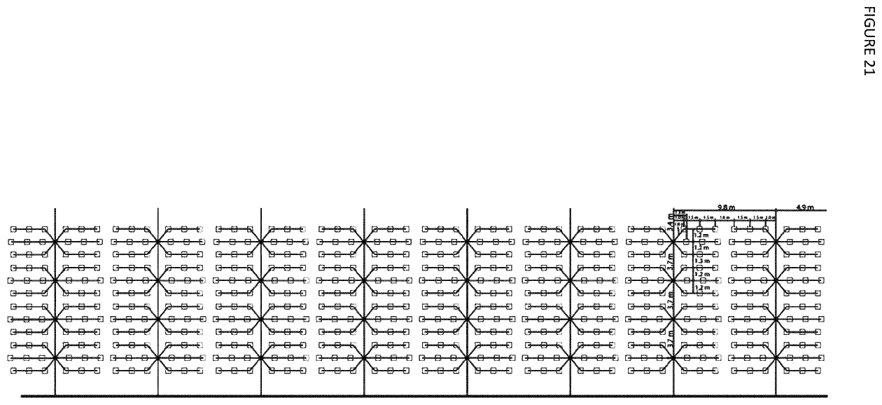

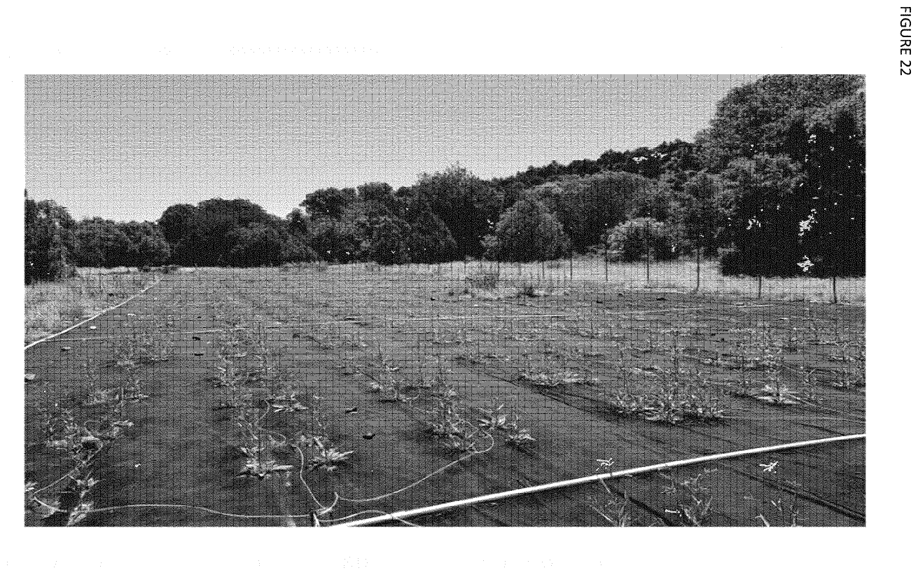

Synthetic biology has enabled the transfer of metabolic pathways among taxa due to shared cofactors and metabolism (30-33). It was attempted to reconstitute the complete CPH pathway in vivo (FIG. 20).

Vicia faba and Solanum chilense were selected for Agrobacterium-mediated transient expression of the CPH pathway for several reasons: Neither species accumulated CPH in untreated and MeJA treated tissues. V. faba is an ideal hostplant for Empoasca rearing. CoP, CP, or FP do not accumulate in V. faba, whereas CP levels are induced by MeJA treatment of S. chilense, providing an internal precursor for CPH production (FIG. 20). Moreover, both are readily transformed and likely to produce correctly folded active proteins with which to test biochemical activities of CPH biosynthetic genes in planta.

NaPPO1 or NaPPO2were transciently co-expressed together with (Z)-3-hexenal and CP leaf infiltrations in V. faba or without CP infiltrations in S. chilense. However, it was not to detect any CPH in either species (FIG. 20). PPOs are generally localized to plastids, which would physically separate them from their phenolic substrates, which are known to be localized in vacuoles (34).

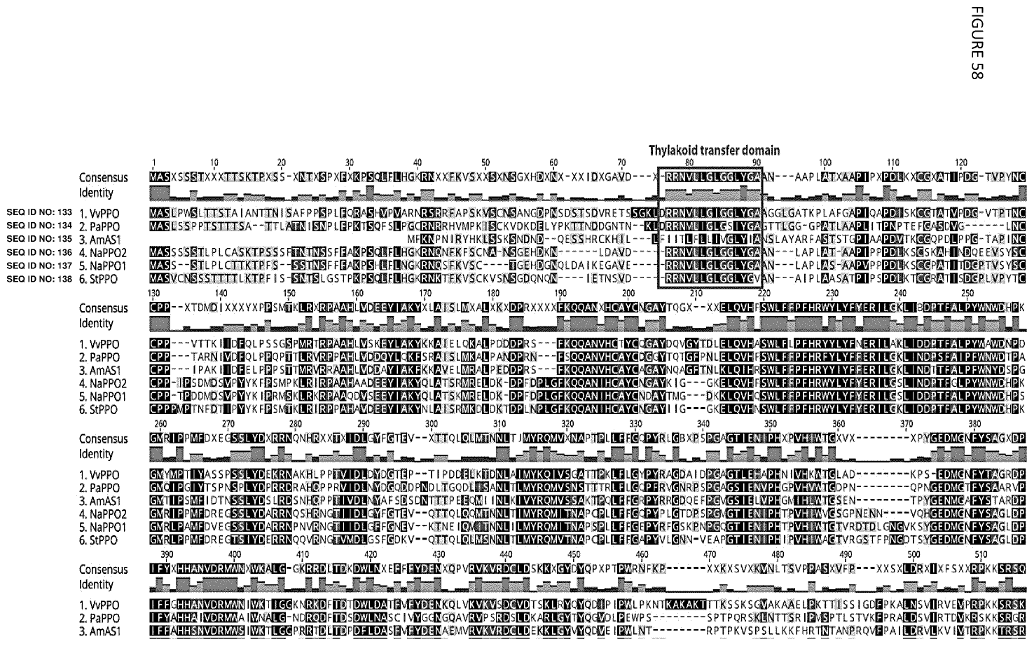

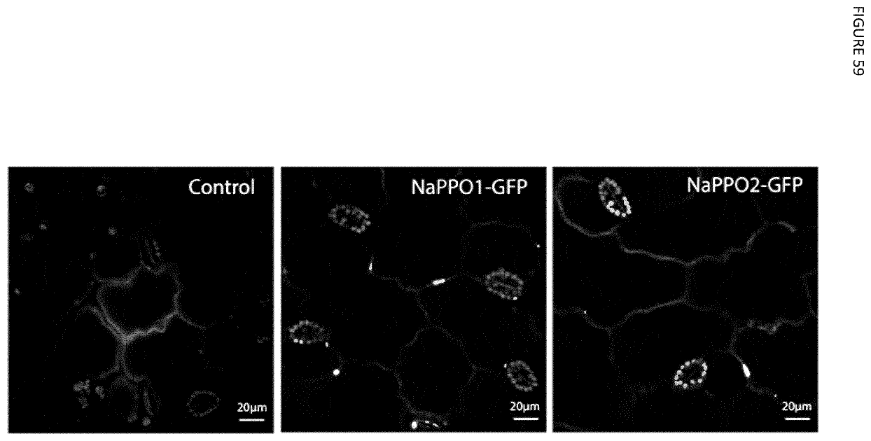

In N. attenuata, a thylakoid transfer domain was identified in both NaPPO1 and NaPPO2 N-terminal sequences (FIG. 58). Transient expression of GFP-tagged NaPPO1 and NaPPO2 in N. attenuata leaves confirmed that both NaPPO1 and NaPPO2 are plastid-localized (FIG. 59).

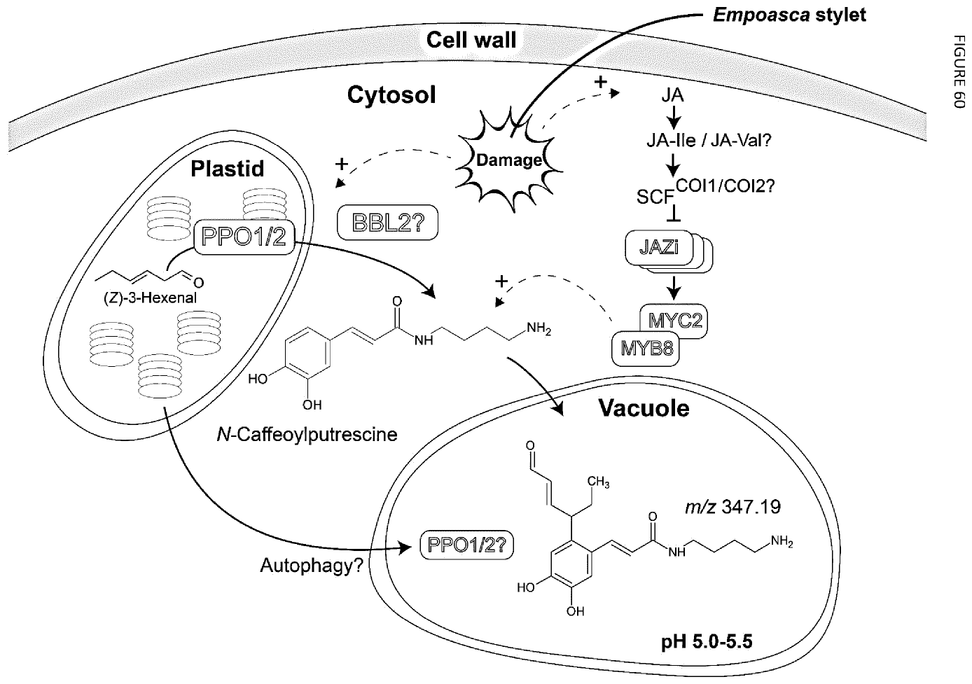

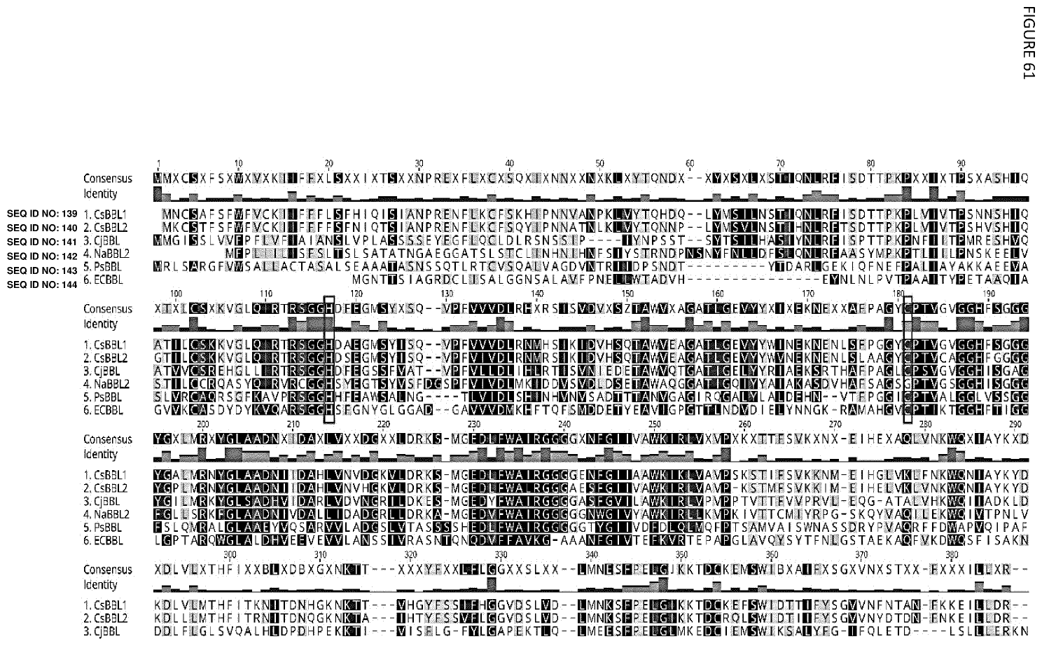

The three-pronged pathway proposal for CPH biosynthesis is hence challenged by the separate enzymatic localizations of the different components: CP (likely vacuolar or cytosolic: (34, 35)); GLVs, JAs, and PPOs (plastidial: (34, 36)). This challenge was reminiscent of nicotine biosynthesis which requires a BBL gene to join the mitochondria-localized pyridine ring derived from nicotinic acid with the pyrrolidine ring derived from the peroxisome-localized N-methylpyrrolin-ium cation to produce nicotine (37). It was hypothesized that NaBBL2 is required for the production of CPH in vivo and to test this hypothesis, NaBBL2 along with NaPPO1 or NaPPO2 were expressed in S. chilense plants. One day after Agrobacterium infiltration, S. chilense plants were treated with MeJA to induce CP production and 3 days later, the leaves were infiltrated with (Z)-3-hexenal. After 6 h, the leaves were harvested for LC-MS analysis and found that the leaves had accumulated substantial quantities of CPH (FIG. 20). For V. faba plants, NaBBL2 along with NaPPO1 or NaPPO2 was expressed; 3 days after Agrobacterium infiltration, the leaves were infiltrated with both CP and (Z)-3-hexenal, and harvested leaves for LC-MS analysis after 6h. Again, CPH accumulated (FIG. 20). From these results, it was inferred that NaBBL2, while not being required for in vitro synthesis, is required for in vivo CPH biosynthesis. Additional work is required to evaluate if NaBBL2 is playing a role in solving the localization challenge, which could have other possible solutions (FIGS. 60 and 61 and Supplementary Materials). Finally, Empoasca feeding trials were conducted on the CPH engineered V. faba and S. chilense plants and it was observed that these Empoasca host crop plants became lethal hostplants for Empoasca (FIG. 20).

This mechanistic analysis of Empoasca nonhost resistance provides another example of the innovative chemical solutions that native plants have evolved to solve their ecological challenges (Rajniak et al., Nature 525, 376-379 (2015)). The natural history-driven multi-omics framework that were employed for the discovery of CPH and its marriage with synthetic biology approaches highlight how readily the results of millions of years of innovation by natural selection can be transferred to crop plants to catalyze the next, greener and ecologically more nuanced revolution in plant protection (S. M. Cook et al., Annu Rev Entomol 52, 375-400 (2007)) and do-mestication (Zhu et al., Cell 172, 249-261 e212 (2018), Sanchez-Perez et al., Science 364, 1095-1098 (2019), Szymanski et al., Nat Genet 52, 1111-1121 (2020)). Crop plants face challenges not substantially different from those of native plants, being constantly tested by an herbivore com-munity that challenges the host/nonhost distinction. In a world of climate change and globally homogenized herbivore communities, opportunistic associations will dominate natural and man-made ecosystems. Insight into how native plants have coped with opportunistic associations will help to design crops more resilient in the face of unknown stresses as the world's climate changes (Xu and Weng, Advanced Genetics 1, e10022 (2020)).

### Example 5

Materials & Methods Summary

Two replicates of the 650 RILs from a 26-parent MAGIC population and their 26 parental 5 lines were planted at the WCCER field station, Prescott, Arizona, USA. To elicit a standardized herbivory response, leaves of all RILs, which were in the early-flowering stage were wounded and immediately treated with diluted Manduca sexta oral secretions (W+OS) or left untreated (control) and harvested on dry ice at 1 h and 72h. One week after metabolite sampling, all plants of the field population were screened for natural Empoasca leafhopper numbers and damage; these leafhoppers had opportunistically sampled the N. attenuata plants from neighboring native cu-cumber hostplants. The mQTL and eQTL mapping between SNPs and the relative abundance of each compound or transcript using a set of 646 RILs of the MAGIC population was done with the R package software GAPIT using General linear models (GLM). The multi-omics co-association network was built from correlations among metabolomes, transcriptomes, phytohormones and SNPs. For Empoasca choice assays, transgenic lines of N. attenuata at the early-rosette growth stage were randomly placed in an open-choice glasshouse environment containing Empoasca leafhoppers reared on bean plants in the MPI-CE glasshouse in Isserstedt, Germany. Yeast two-hybrid and qRT-PCR were used for characterizing Empoasca-induced jasmonate signalling genes. Compound-specific idMS/MS was constructed using UHPLC-ESI/qTOF-MS for idMS/MS acquisition and rule-based computational approaches for idMS/MS assembly. Metabolome diversity and specialization and metabolic specificity was calculated using information theory by consider-ing the Shannon entropy of the idMS/MS frequency distributions. In vivo Empoasca choice and in vitro Empoasca feeding assays were conducted by infiltrating synthetic caffeoylputrescine (CP), coumaroylputrescine (CoP) or feruloylputrescine (FP) into irMYC2a/2b leaves or by feeding Em-poasca with the compounds diluted in 10% glucose solutions. 15 RILs, which induced putrescine-containing phenolamides after OS elicitation and accumulated a diverse set of known and unknown phenolamides, were used to construct idMS/MS for MS/MS structural metabolomics analysis. MS/MS similarity scoring, bi-clustering and molecular networking were used to identify the unknown m/z 347.19 metabolite. OS-induced volatile emissions were collected using polydimethylsiloxane (PDMS) tubing from 650 MAGIC RILs planted in the main MPI-CE glasshouse and analyzed bythermal desorption-gas chromatography-mass spectrometry (TD-GC-MS). NaPPO1/2 and NaBBL2 genes were elucidated by combining mQTL analysis for herbivory-induced unknown m/z 347.19 and transcriptomics analysis of the microarray and RNAseq datasets of OS-induced kinetics of WT and irMYC2a/2b and irMYB8 lines. The candidate genes were functionally vali-dated by virus-induced gene silencing (VIGS) and in vitro enzymatic assays using E. coli expressed NaPPO1, NaPPO2 and NaBBL2 with CP and (Z)-3-hexenal. The CPH (CP-5-(Z)-3-hexenal) chemical structure was characterized by NMR. CPH's resistance function against Empoasca was tested by in vitro non-choice assays with synthesized CPH or by in planta choice assays conducted with VIGS plants of EV, NaPPO1, NaPPO2 and NaAT1. The biosynthetic pathway of CPH was reconstituted in Vicia faba and Solanum chilense by transient coexpressing NaPPO1, NaPPO2 and NaBBL2 with CP and (Z)-3-hexenal leaf infiltrations. The nonhost resistance function of CPH against Em-poasca was further evaluated with the CPH engineered V. faba and S. chilense plants.

### Example 6

### Plant and Material Method, Metabolite Extraction

Plant and material method: Nicotiana attenuata inbred for 31 generations (originally collected at the DI ranch in southwestern Utah, USA) was used as the wild type (WT) genetic background for all experiments and transformations. Plants were grown as previously described (Krugel et al., Chemoecology 12, 177-183 (2002)) with a day/night cycle of 16 h (26-28° C.)/8 h (22-24° C.) in a glasshouse at the Max Planck Institute for Chemical Ecology (MPI-CE), Jena, Germany. VIGS experiments were carried out in a climate chamber (York) with growth conditions at the start of the experiments being 22/22° C. 16/8 h light/dark at 65% relative humidity with low light levels, approximately 100 μmol m−2 s−1 for 2 d after inoculation, after which, light levels were returned to normal high light levels (400-1000 μmol m−2 s−1 PAR) (Saedler and Baldwin, J Exp Bot 55, 151-157 (2004)). Empoasca choice assays were performed in a plant growth chamber (Percival Scientific) with a constant temperature of 242C and 16 h day/8 h night light regime, 70% relative humidity and 100% light intensity (350 μmol s−1 m−2 PAR).To prevent Empoasca infesta-tions in the main glasshouse facility of the MPI-CE, we performed large-scale choice assays in a separate glasshouse facility located in Isserstedt, Germany, approximately 7 km from the main glasshouse facility. The choice assays were performed under natural light conditions, a constant temperature of 242C, and 70% relative humidity.

Metabolite extraction: Approximately 100 mg of ground leaf tissue was weighed and extracted as follows using extraction buffers containing 80% methanol. One milliliter of extraction buffer [50 mM acetate buffer (pH 4.8) containing 80% methanol] per 100 mg of tissue was added, and samples were homogenized in a ball mill (Genogrinder 2000; SPEX CertiPrep) for 45 s at a rate of 1× and at 1100 strokes per minute. Homogenized samples were centrifuged at 16,000×g at 4° C. for 30 min, and 800 μL supernatants were transferred into 1.5-mL microcentrifuge tubes and re-centrifuged as before. Supernatants of 600 μL were transferred to 2-mLglass vials for MS-based metabolomics.

### Example 7

### Magic 2019 Field Experiments, Insect Collections and Treatments

Magic 2019 field experiments: Two replicates of the 650 recombinant inbred lines (RILs) of the MAGIC population as well as the 26 parental lines that were used for the breeding of the MAGIC RIL population (Ray et al., Plant J. 99, 414-425 (2019) were planted at the Walnut Creek Center for Education and Research (WCCER) in Prescott, Arizona, USA. Prior to the planting, a drip watering system was installed for watering the MAGIC population plants. The field plot consisted of 8 main 1″ diameter trunk lines, each containing 4 splitters of irrigation lines, each consisting of 18 drippers, with 4 plants being watered by each dripper. The 4 plants at each dripper were planted 50 cm apart and the 4-plant clusters were planted 150 cm apart. The watering system and general planting layout are depicted in FIG. 21, with vertical lines indicating the 8 main trunk water lines. Smoke-treated N. attenuata seeds were germinated in Jiffy-7© peat pellets which had been hydrated with a native soil extract to provide plants with an appropriate micro-biome during germination (Santhanam et al., Proc Natl Acad Sci USA 112, E5013-E5020 (2015)).

To elicit a standardized herbivore-specific response in a kinetically defined manner, leaves were wounded with three rows of puncture wounds on each side of the midrib with a fabric pattern wheel (Dritz, Spartanburg, SC) and immediately treated with 20 μL of 1:5 diluted Manduca sexta oral secretions (W+OS) or left untreated (control) (McCloud and Baldwin, Planta 203, 430-435 (1997)). On every sampled plant, the first three stem leaves were treated with W+OS and sampled at 1 and 72h; control leaves were sampled at Oh. All leaf samples were immediately frozen on dry ice and transported on dry-ice in dry-ice shipping containers by airplane and stored at the MPI-CE in −80° C. freezers. One week after metabolite sampling, the field team counted the number of Empoasca spp. on each plant and visually estimated the proportion of leaf area damaged by leafhopper feeding for each plant of the MAGIC population.

Insect collections and treatments: A laboratory colony of Empoasca decipiens was estab-lished from insects collected on Buddleja (butterfly lilac) plants grown in the surroundings of the MPI-CE, Jena, Germany. Insects were reared on Vicia fabae in four mesh tents in a glasshouse in Isserstedt, Germany. The tents (220×90×110 cm, Tatonka, Germany) were covered entirely with mesh, allowing for air exchange. To screen genetic and metabolic responses specific to leafhopper attack, 25 adult leafhoppers were caged in 50-mL plastic containers (Huhtamaki) on leaves of different transgenic lines at the rosette stage (FIGS. 30 and 31). Empty 50-mL plastic containers were placed on EV plant leaves as controls. For metabolomics analysis, leaves were treated for 72 h, the plastic containers together with leaves and insects were removed from the plants, and the leafhoppers were returned to the colony. For the quantification of transcript abundances, samples were collected at 0, 0.5, 1, 2, 3, 6, 9 and 24 h, respectively. Harvested leaves were immediately frozen in liquid nitrogen and stored in −80° C. freezers until use. Control leaves were harvested and similarly stored.

M. sexta eggs were from an in-house colony maintained by Department of Evolutionary Neuroethology at MPI-CE, Jena, Germany, as previously described (Koenig et al., Insect Biochem Mol Biol 66, 51-63 (2015)). The M. sexta feeding assays were conducted by placing newly hatched M. sexta on fully-expanded rosette stage leaves.

For methyl jasmonate (MeJA) treatments, MeJA (Sigma, Catalogue #: 392707-25ML) was dissolved in heat-liquefied lanolin (502C) at a concentration of 7.5 mg mL-1. Pure lanolin was used as a negative control. The abaxial side of the bases of the first three fully elongated leaves (four weeks after potting) was treated with 20 μL lanolin paste containing 150 μg MeJA (Lan+MeJA), with 20 μL of lanolin plus wounding treatment (Lan+W), or with 20 μL of pure lanolin as control (Baldwin et al., J Chem Ecol 22, 61-74 (1996)). Leaves were harvested (midveins were removed) at 72 h after treatment, flash-frozen in liquid nitrogen, and stored at −80° C. until use.

### Example 8

### Empoasca Choice Experiments and Transgenic Lines

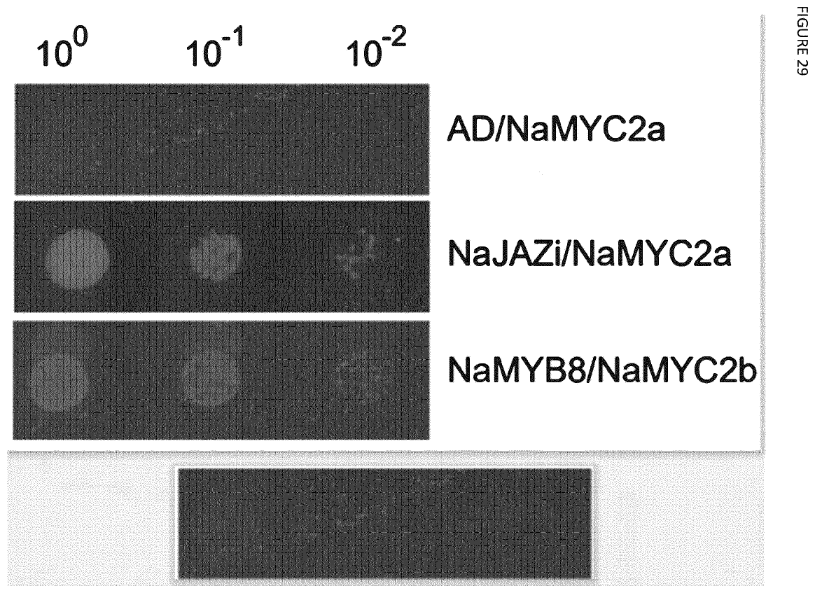

Empoasca choice experiments: N. attenuata plants were germinated and grown in the main glasshouse facility at the MPI-CE and transferred to the Isserstedt glasshouse at the early rosette stage. After transferring plants to the Isserstedt glasshouse, plants were acclimated for at least two days before experiments were initiated. Plants were randomly placed on the table in a distance of at least 40 cm from each other. The E. decipiens colony tents were placed on two tables adjacent to the N. attenuata plants (FIG. 26) and ca. 400 E. decipiens individuals were re-1o leased into the glasshouse. After two weeks, the damaged area was visually estimated as the percentage of the total leaf area of each leaf, as previously described (Kallenbach et al., Proc Natl Acad Sci USA 109, E1548-E1557 (2012)). Average leaf damage was calculated for three different leaf types: rosette leaves, the first (oldest) three stem leaves and all younger stem leaves. We also counted the number of Empoasca individuals on each plant. Each genotype was represented by 10 replicate plants.

The Empoasca choice assays forthe silenced PPO1, PPO2 and BBL2 plants were performed in tents placed in a growth chamber (Percival Scientific) (FIG. 31). The silenced PPO1, PPO2 and BBL2 plants were transfer into the tents from the glasshouse, when plants were in the early elongation stages of growth. After transferring plants into the tents, plants were acclimated for at least two days before being exposed to E. decipiens attack. Plants were randomly placed in the tent at a distance of at least 15 cm from each other; each tent contained four genotypes: EV, PPO1, PPO2 and BBL2 silenced plants. 50 E. decipiens adults were released into each tent. After one week, the damaged area was visually estimated and quantified as the percentage of the total leaf area of each leaf, as previously described. We also counted the number of Empoasca individuals on each plant in the tent. Each genotype was represented by 8 replicate plants.

Generation of transgenic lines: NaMYC2 (irMYC2, A-17-110-2-2)/JAZi (irJAZi, A-17-013-2)/JAZc (irJAZc, A-09-220-4-1)/JAZe (irJAZe, A-09-250-7)/JAZg (irJAZg, A-19-070-5)/JAZL (irJAZL, A-18-029)/DH29 (irDH29, A-06-051-1)/CV86 (irCV86, A-06-022-6)-silenced lines and NaJAZi (ovJAZi, A-17-007-1)/JAZL (ovJAZL, A-18-042)-overexpressing lines were generated by the published Agrobacterium tumefaciens-mediated transformation method (Krugel et al., Chemoecology 12, 177-183 (2002)) using pSOL8 binary vectors containing the inverted repeat fragment of the NaMYC2 (LOC109232914, LOC109205493), NaJAZi (LOC109240311), NaJAZc (LOC109233155), NaJAZe (LOC109223947), NaJAZL (LOC109220335), NaJAZg (LOC109219395), NaDH29 (LOC109206371) or NaCV86 (LOC109206370) sequences. NaJAZi and NaJAZL overexpressing lines were constructed by using pSOL9 vectors with NaJAZi (LOC109240311) and NaJAZL (LOC109220335) sequences, respectively. Homozygous T₂ diploid plants harboring single insertions were used in all studies. The number of insertion copies, as well as the fidelity of the insertion (over-reads and truncations), were evaluated by NanoString analysis (He et al., Proc Natl Acad Sci USA 116, 14651-14660 (2019)). In brief, DNA extracted from individual transgenic plants was used for NanoString nCounter© to detect specific regions of the inserted fragments according to the designed probes. To validate the single-copy-number insertions inferred from the selective marker resistance segregation rates, a published transformed line with a single-copy insertion (irAGO8), was used as a positive control (Pradhan et al., Plant Physiol 175, 927-946 (2017)).

The following four previously characterized transgenic lines were also used in this study: irAOC (A-07-457-1) plants silenced in the expression of the NaAOC gene (Kallenbach et al., Proc Natl Acad Sci USA 109, E1548-E1557 (2012)), irCOl1(A-04-249-A-1) plants silenced in the expression of the NaCOIl1 gene (Paschold, et al., Plant J 51, 79-91 (2007)), irMYB8 (A-07-810-2) plants silenced in the expression of the NaMYB8 gene (Onkokesung et al., Plant Physiol 158, 389-407 (2012)), irJAZh (A-09-368-7) plants silenced in the expression of the NaJAZh gene (Oh et al., Plant Physiol 159, 769-788 (2012)), irPMT (A-03-108) plants silenced in the expression of the NaPMT gene (Steppuhn et al., PLoS Biol 2, E217 (2004)), irGGPPS (A-08-231) plants silenced in the expression of the NaGGPPS gene (Heiling et al., Plant Cell 22, 273-292 (2010)), asHPL (A-247) plants silenced in the expression of the NaHPL gene (Halitschke et al., Plant J 40, 35-46 (2004)), and irLOX2 (A-04-52-2) and crosses of irLOX2 and LOX3 (A07-707-2) plants silenced in the expression of NaLOX2 and both NaLOX2 and NaLOX3 gene, respectively (Allmann et al., Plant Cell Environ 33, 2028-2040 (2010)).

### Example 9

### Transcriptomics Data Mining and Analysis of the Microarray

Transcriptomics data mining and analysis of the microarray and RNAseq datasets: The microarray data were originally published in Kim et al., PloS One 6, e26214 (2011) and deposited in the National Center for Biotechnology Information Gene Expression Omnibus database (GEO number: GSE30287). Raw intensities were log 2 and baseline transformed and normalized to their 75th percentile using the R software package, prior to statistical analysis. The raw RNAseq data were processed as described in Pertea et al., Nat Protoc 11, 1650-1667 (2016). Briefly, raw RNAseq reads were first converted to fastQ format. HISAT2 converted fastQ to sam, and SAMtools converted sam files to sorted bam files. StringTie was used to calculate gene expression as fragments per kilobase of transcript per million reads sequenced (FPKM).

Assosciation mapping: The mQTL and eQTL mapping between SNPs and the relative abundance of each compound or transcript using a set of 646 RILs of the MAGIC population was carried out using the R package software GAPIT (Genome Association and Prediction Integrated Tool) (Lipka et al., Bioinformatics 28, 2397-2399 (2012)). General linear models (GLM) were used for association analysis.

### Example 10

### Volatile Measurement

Volatile measurement in the MAGIC RIL population: For the characterization of the herbi-vore-induced volatile emissions, a single replicate of the 650 MAGIC RILs was planted in the glasshouse at the MPI-CE. The third oldest stem-leaf of each plant was treated with wounding and M. sexta oral secretions (W+OS) as described above for the metabolite analysis and the treated leaves were individually enclosed in 500-mL transparent (PET) cups. Volatiles were collected for 24 h on two 5-mm pieces polydimethylsiloxane (PDMS) tubing suspended in the headspace above the leaf in the collection cup (Kallenbach et al., Plant J 78, 1060-1072 (2014)). The PDMS tubing was collected and stored at −202C until analysis.

Volatile were analyzed by thermal desorption-gas chromatography-mass spectrometry (TD-GC-MS). The GC-MS (Shimadzu, GCMS-QP2010Ultra) was equipped with a TD autosampler (Shimadzu TD-20) and a semipolar capillary column (Phenomenex ZB-WAXplus, 30m x 0.25 mm ID, 250 μm film thickness). Compounds were desorbed as previously described (Kallenbach et al., Plant J 78, 1060-1072 (2014)) and separated by applying the following column temperature gradient: 0 to 5 min isothermal at 402C, 5 to 34 min linear ramp to 1852C, 34 to 35.5 min linear ramp to 2302C, 35.5 to 36 min isothermal at 2302C. The MS detector was operated in full scan mode from m/z 33 to 400. Relative quantifications of green leaf volatiles and terpenoids was based on the peak integration of extracted ion chromatograms of characteristic fragment ions. Compounds were identified based on comparisons of retention times and mass spectra with authentic standards and an in-house MS library.

### Example 11

Protein Localization and qRT-PCR Analysis

Protein localization: An A. tumefaciens strain GV3101 harboring 35S::NaPPO1-GFP or 35S::NaPPO2-GFP was infiltrated into N. attenuata leaves. Three days after infiltration, confocal images of epidermal cells of the abaxial side of infiltrated leaves were acquired using a CLSM 510 confocal scanning microscope (Zeiss).

qRT-PCR analysis of JAZ and m/z 347.19 8CHP) biosynthetic genes: N. attenuata wild type leaves were removed from the stem with scissors and 0 h time point (control) tissue samples were collected, flash frozen in liquid nitrogen and stored at −80° C. until use. For treated leaves, one leaf per plant was exposed to herbivory by E. decipiens or M. sexta in the growth chamber (FIGS. 30 and 31). The leaf samples were collected at 0.5, 1, 2, 6, 9, 12 and 24 h after the treat-ment, flash frozen in liquid nitrogen and stored at −80° C. until use.

Primers to specific regions of the targeted genes were designed with amplicon lengths between 70-200 bp using Primer3 (Untergasser et al., Nucleic Acids Res 40, e115 (2012)). After grinding of the leaf tissue sample in liquid nitrogen, total RNA was extracted from an aliquot of 100 mg of powdered leaf material following the protocol of the NucleoSpin RNA Plant kit (MA-CHEREY-NAGEL, Catalogue #: 740949.50). RNA samples were measured on a Nanodrop spectro-photometer (Peqlab, ND-1000). 1 μg RNA was used for synthesizing cDNA following the protocol of the PrimeScript™ RT Reagent Kit (Perfect Real Time, Takara, catalogue #: RR037B). The qPCR experiments were performed using Taykon™ No ROX SYBR® Master Mix (Eurogentec, catalogue #: UF-NSMT-B0701) on the Stratagene MX3005P instrument. The N. attenuata elongation factor1-a gene (EF1-a; accession no. GBGF01000210.1) was used as housekeeping gene for normalization of qPCR results. All qPCR primers are listed in Table 3.

### Example 12

### Yeast Two-Hybrid Assay and VIGS

Yeast two-hybrid assay: Y2H assays were performed by using the Matchmaker Gold Yeast Two-Hybrid System (Clontech) following the manufacturer's instruction. In brief, AD and BD fusions constructed along with their own empty vector as negative control were co-transformed into freshly prepared Y2H gold competent cells with the Yeastmaker™ Yeast Transformation Kit (Clontech, Catalogue #: 630304) and plated on selective dropout medium (SD-Leu/-Trp). The transformations grew on QDO (SD-Leu/-Ade/-His/-Trp) medium in the presence of 2 mM 3-AT at 30° C. for 5-7 days after incubation for recording.

VIGS: Vectors (pTV00) containing fragments of NaAT1 and biosynthesis candidate genes for the CPH metabolite were generated and plant growth and VIGS inoculations were performed as described previously (Saedler and Baldwin, J Exp Bot 55, 151-157 (2004)). Briefly, to silence the NaAT1 gene and m/z 347 biosynthesis candidate genes including NaPPO1, NaPPO2, NaBBL2, a 150-400 bp antisense fragment of these genes was cloned into the polylinker of the pTV00 vector (provided by the laboratory of Sir David Baulcombe) and transformed into Agrobacterium tumefaciens GV3101. The empty cloning vector pTV00 was used as a negative control for non-specific phenotypic effects of the VIGS. The positive control vector, pTVPD, was prepared by cloning a 206 bp fragment of the N. benthamiana phytoene desaturase gene (NtPDS, Ni-ben101Scf01283g02002.1) in antisense orientation into the polylinker of pTV00. 23-25 days post-germination plants were treated by the needleless syringe infiltration method described previously (Saedler and Baldwin, J Exp Bot 55, 151-157 (2004)). Four weeks after potting, silenced leaves were elicited by W+OS treatment. After three days, the treated leaves were flash frozen in liquid nitrogen and stored at −80° C. until use, untreated leaves were collected similarly as controls. The VIGS experiments were repeated at least three times.

### Example 13

### Cloning of Candidate CHP Biosynthetic Genes and Reconstitution of the CHP Pathway

Cloning of candidate CHP biosynthetic genes: Phusion High-Fidelity DNA Polymerase (New England Biolabs, catalogue #: M0530L) was used for all PCR amplification steps according to the manufacturer's instructions. Oligo primers were purchased from Integrated DNA Technologies. DNA fragments were purified from agarose gels using the NucleoSpin Gel and PCR Clean up Kit (Macherey-Nagel, catalogue #: 740609.50). One Shot™ TOP10 chemically competent E. coli (Invi-trogen, catalogue #: C404006) were used for plasmid isolation prior to transformation into other heterologous hosts. Plasmid DNA was isolated from E. coli cultures using the NucleoSpin plasmid Kit (Macherey-Nagel, catalogue #: 740588.50). The sequences were confirmed by Sanger dideoxy sequencing following amplification of the target genes by PCR. The PCR fragments were purified with the DyeEx 2.0 Spin Kit (250) (QIAGEN, catalogue #: 63206). The sequencing was performed on an ABI 3130 Genetic Analyzer at the Department of Department of Molecular Ecology, MPI-CE, Jena, Germany. For a list of primers used for cloning, see Table 3.

Reconstitution of the CHP pathway in Vicia faba, Solanum chilense and Empoasca bioassays: The sequences corresponding to the full-length open reading frames of all selected candidates genes were obtained from the N. attenuata genome release version 2.0 (Xu et al., Proc Natl Acad Sci USA 114, 6133-6138 (2017)). The gene sequences were PCR amplified (for primers, see Table 3) and recombined into the donor vector pDONR207. Sequence-verified entry clones for the candidate genes including NaPPO1, NaPPO2, and NaBBL2 were Gateway recombined into the pEAQ-HT-DEST vector and sequence verified. The resulting pEAQ-HT constructs were transformed into Agrobacterium tumefaciens (GV3101) using the electroporation method. Transformants were grown on yeast extract broth plates supplemented with 20 mg mL⁻¹ gentamicin, 100 mg mL⁻¹ rifampicin, and 25 mg mL⁻¹ kanamycin. A single colony was inoculated into 5 mL of yeast extract broth medium supplemented with 20 mg mL⁻¹ gentamicin, 100 mg mL⁻¹ rifampicin, and 25 mg mL⁻¹ kanamycin. After overnight incubation, bacteria for transient coexpression were mixed, pelleted via centrifugation, and resuspended in infiltration buffer (100 μM acetosyrin-gone, 10 mM MgCl₂, and 10 mM MES, pH 5.7). After a 2 h incubation at room temperature, the bacterial mixtures (OD=0.25 for each strain) were infiltrated into the abaxial sides of fully expanded leaves of 3-4 6-week-old V. faba or S. chilense plants grown in a growth chamber maintained under VIGs conditions. Leaves of the S. chilense plants were treated with MeJA. The infiltrated and MeJA-treated plants were incubated under normal growth conditions for 3 d prior to metabolite analysis. Biological replicates consisted of several leaves from different plants. For substrate infiltration experiments, 500 μM (E)-N-caffeoylputrescine (BOC Sciences, catalogue #: B0005-053482) and 1 mM cis-3-hexenal (Sigma, catalogue #: W256102-SAMPLE-K) dissolved in 0.01% aqueous DMSO in 50 mL acetate buffer (20 mM, pH 4.8) solutions were infiltrated into the abaxial surface of previously PPO1, PPO2 and BBL2 Agrobacterium-infiltrated leaves with a needleless 1-mL syringe, 1 day after Agrobacterium infiltration, 1 mL (E)-N-caffeoylputrescine (500 μM) or cis-3-hexenal (1 mM) were infiltrated in EV plants leaves as negative controls. Leaves disks were harvested after one day, flash frozen and extracted for CPH metabolites profiling on the LC-MS. Four CPH accumulating V. faba or S. chilense leaves were selected for Empoasca bioassays and 25 adult leafhoppers were caged in 50-mL plastic containers (Huhtamaki) on each of the previously Agro-infiltrated leaves. The Empoasca survival rate was recorded every 1 h overnight.

### Example 14

### UHPLC-ESI/gTOF-MS Conditions for Profile Mode Analysis and MS/MS Data Acquisition

UHPLC-ESI/gTOF-MS conditions for profile mode analysis and MS/MS data acquisition: An Acclaim column (150×2.1 mm, particle size 2.2 pm, ThermoFisher Scientific) equipped with a UHPLC SecurityGuard™ ULTRA cartridge (Phenomenex, catalogue #: AJO-8782) was used for the analysis. The following binary gradient was used with a Dionex Ultimate 3000 UHPLC system: 0 to 0.5 min, isocratic 90% A (de-ionized water, 0.1% [v/v]acetonitrile and 0.05% formic acid), 10% B (acetonitrile and 0.05% formic acid); 0.5 to 23.5 min, gradient phase to 10% A, 90% B; 23.5 to 25 min, isocratic 10% A, 90% B. The flow rate was 400 μL min⁻¹. For all MS analyses, the column eluent was infused into an Impact II or Compact (Bruker Daltonics, Bremen, Germany) quadrupole time-of-flight (qTOF) mass spectrometer equipped with an electrospray source operated in positive ionization mode (capillary voltage 4500 V, capillary exit 130 V, dry temperature 200° C., dry gas flow of 10 L min⁻¹).

The indiscriminant MS/MS approach was realized by operating the quadrupole with a very large mass isolation window, which allows all m/z signals to be considered for fragmentation. For this, several independent analyses were performed with increasing CID collision energy values, as neither the Impact II or Compact Bruker instruments are able to perform CE ramping. Briefly, samples were first analyzed by UHPLC-ESI/qTOF-MS using the single MS mode (low fragmentation condition derived from in-source fragmentation) by scanning from m/z 50 to 1500 at a rep-etition rate of 5 Hz. Indiscriminant MS/MS analyses were conducted using nitrogen as a collision gas and included independent measurements at 4 collision-induced dissociation voltages: 20, 30, 40 and 50 eV. The quadrupole was operated throughout the measurement with the largest mass isolation window, from m/z 50 to 1500. This mass range was automatically activated by the operating software of the instrument when the precursor m/z and the isolation width parameters were experimentally set to 200 and 0 Da, respectively. Mass fragments were scanned as described for the single MS mode. Mass calibration was performed using sodium formate (50 mL isopropanol, 200 μL formic acid, 1 mL 1M NaOH in water). Data files were calibrated post-run on the average spectrum of the calibration segment at the beginning of each run, using the Bruker HPC (high-precision calibration) algorithm. Raw data files were converted to the netCDF format using the export function of the Data Analysis v4.0 software (Bruker Daltonics, Bremen, Germany).

IdMS/MS assembly and similarity scoring: Data-independent or indiscriminant MS/MS fragmentation analysis (hereafter referred to as idMS/MS) was conducted in order to gain structural information on the overall detectable metabolic profile. idMS/MS assembly was achieved via correlational analysis between MS1 and MS/MS mass signals for low and high collision energies and newly implemented rules. The correlation analysis for precursor-to-product assignment was implemented using an R script and rules were implemented using a C#script (https://github.com/MPI-DL/indiscriminant-MS-MS-assembly-pipeline) (Li et al., Sci Adv 6, eaaz0381 (2020)).

For MS/MS similarity scoring, idMS/MS spectra were aligned in a pairwise manner and their similarity calculated according to two scores. First, a standard normalized dot product (NDP), also referred to as the cosine correlation method, was used to score fragment similarity among spectra using the following equation:

$$NDP = \frac{{( {\sum }_{i}^{S 1 \& S 2} {W}_{S 1 , i} {W}_{S 2 , i} )}^{2}}{{\sum }_{i} {W}_{S 1 , i}^{2} {\sum }_{i} {W}_{S 2 , i}^{2}}$$

where S1 and S2 correspond, respectively, to spectrum 1 and spectrum 2 and W_{s1,i} and Ws₂,i indicate peak intensity-based weights given to i^{th} common peaks differing by less than 0.01 Da between the two spectra.

Weights were calculated as follows:

$$W = {{[ Peak intensity ]}^{m} [ Mass ]}^{n}$$

$$with m = 0.5 and n = 2 ( Li et al . , Sci Adv 6 , eaaz 0381 ( 2020 ) ) .$$

A second scoring method was implemented involving the analysis of shared neutral losses 10 among individual MS/MS. For this, we used a list of 52 neutral losses (NLs) commonly encoun-tered during tandem MS fragmentation, as well as more specific ones that had been previously annotated for MS/MS spectra of N. attenuata secondary metabolite classes (Li et al., Sci Adv 6, eaaz0381 (2020)), Li et al., Proc Natl Acad Sci USA 113, E7610-E7618, (2016), Li et al., Proc Natl Acad Sci USA 112, E4147-E4155 (2015)). A binary vector of 1 and 0 was created for each MS/MS corresponding to presence and absence of certain NL. NL similarity scores were calculated for each pair of binary NL vectors based on Euclidean distance similarity.

Rules for the assembly of compound-specific idMS/MS: To reduce false positive errors resulting from spurious correlations of background noise due to the fact that some m/z features are only detected in a few samples, we compared data processing results obtained with and without the “fill peaks” function of XCMS (used for background noise correction) and calculated a background noise value from the average correction estimate used by this function to replace “NA” intensity values of undetected peaks. When the “fill peaks” function was used, many “0” intensity values remained in the dataset which influenced the calculations of correlations, and these were replaced with the calculated background value. We also only considered features with intensities that were more than 3 times the background value and considered these as “true peaks”. Only m/z signals with at least eight “true peaks” in the precursor (MS1) and fragment (MS/MS) datasets were considered for PCC calculation.

A precursor mass feature was further defined if its intensity across samples significantly correlated with the decreased intensity of the same mass feature subjected to low or high collision energies and the feature was not annotated as an isotope peak by CAMERA. The correlation analysis was then conducted by calculating all possible precursor-to-product pairs within a 3-s retention time window. m/z values were only considered as fragments if their m/z values were lower than those of the precursor and MS/MS fragmentation occurred in the same sample position within the dataset as the precursor from which it is derived.

Many in-source-fragmentation-generated mass features produced in the MS1 mode can also be selected as candidate precursors, resulting in redundant compound idMS/MS. To reduce such data redundancy, we merged spectra if their NDP similarity exceeded 0.6 and they belonged to the same chromatographic “pcgroup” annotated by CAMERA. Finally, all results from the 4 collision energies used for precursor-to-fragment associations into a final deconvoluted composite spectrum were merged by choosing the highest intensity peak among all candidate peaks of the same m/z value at the different collision energies.

### Example 15

### MS/MS Molecular Networking by Bi-Clustering

MS/MS molecular networking by bi-clustering: To perform the clustering, the R package DiffCoEx was used, which is an extension of the Weighted Gene Coexpression Analysis (WGCNA).

Using NDP and NL-scoring matrices for MS/MS spectra, a comparative correlation matrix was computed using DiffCoEx with the parameters of “cutreeDynamic” set to method=“hybrid”, cutHeight=0.9999, deepSplit=3, minClusterSize=10. The R source code of DiffCoEx was down-loaded from additional file 1 in Tesson et al., BMC bioinformatics 11, 1-9 (2010), the required R WGCNA package can be found at https://horvath.genetics.ucla.edu/html/CoexpressionNet-work/Rpackages/WGCNA/(accessed on Dec. 21, 2021).

Information theory-based calculation of metabolome diversity and specialization and metabolic specificity

Metabolome diversity was calculated using the Shannon entropy of the MS/MS frequency distribution by the following equation as described in Martinez et al., Proc Natl Acad Sci USA 105, 9709-9714 (2008):

$$Hj = - {\sum }_{i = 1}^{m} {P}_{ij} {log}_{2} ( {P}_{ij} )$$

where Pij corresponds to the relative frequency of the i^{th} MS/MS (i=1, 2, . . . , m) in the j^{t}h sample (j=1, 2, . . . , t).

The average frequency of the i^{th} MS/MS among samples was calculated as:

$${P}_{i} = \frac{1}{t} {\sum }_{j = 1}^{t} {P}_{ij}$$

MS/MS specificity was calculated as:

$${S}_{i} = \frac{1}{t} ( {\sum }_{j = 1}^{t} \frac{{P}_{ij}}{{P}_{i}} {log}_{2} \frac{{P}_{ij}}{{P}_{i}} )$$

The metabolome specialization index δj was calculated as the average of the MS/MS specificities for each sample j using the following formula:

$${δ}_{j} = {\sum }_{i = 1}^{m} {P}_{ij} {S}_{i}$$

Selection of 15 RILs for idMS/MS analysis: 15 RILs were selected based on the following two criteria: 1) those which induced the highest phenolamides levels in the MAGIC population, especially for putrescine-containing metabolites; 2) those which produced a diverse set of known phenolamides as well as unknowns consisting of the typical phenolamide fragmentations (e.g., m/z 163.04 with unusual retention times), as evaluated from previous annotations of phenolamides and manual inspections of in-source or MS/MS fragmentations.

### Example 16

### Heterologous Expression and Purification

Heterologous expression and purification of NaPPO1, NaPPo2 and NaBBL2: 50 ng of the pET28a:NaPPO1, pET28a:NaPPO2 and pET28a:NaBBL2 plasmids was transformed into Ro-setta™ (DE3) competent cells (Sigma-Aldrich, catalogue #: 70954) by heat shock for 90s in a 42° C. water bath. Transformants were cultured on LB agar plates containing 50 μg mL⁻¹ kanamycin at 37° C. Five independent colonies were inoculated into 20 mL of LB medium containing 50 μg mL⁻¹ kanamycin and grown for 16 h at 37° C. Selected overnight cultures were inoculated into 500 mL of LB liquid medium containing 50 μg mL⁻¹ kanamycin and grown at 37° C. until OD₆₀₀=0.4, at which point the cultures were cooled on ice for 10 min. Isopropyl p-d-1-thiogalactopyranoside (IPTG) was added to a final concentration of 1 mM. After incubating for 12 h at 16° C. and 160 rpm shaker speed, cells were collected by centrifugation at 5000×g for 10 min at 4° C. The supernatant was discarded and the pellet was resuspended in 9 mL extraction/lysis buffer [50 mM sodium phosphate (pH 8.0), 500 mM NaCl, 10 mM imidazole, Lysozyme (ThermoFisher, catalogue #: 89833), cOmplete™ Protease Inhibitor Cocktail (Merck, catalogue #: 11697498001)], cooled on ice for 1 h, and then disrupted on ice by sonication. The resulting homogenate was centrifuged at 15,000 rpm for 30 min at 4° C. The supernatant was incubated with 1 mL of Ni-NTA agarose resin (Qiagen, catalogue #: 70666-4) pre-equilibrated with 10 mL lysis buffer for 2 h on the rotator at 4° C. The slurry was run on a Poly-Prep® chromatography column (BIO-RAD, catalogue #: 7311550) and proteins were eluted with wash buffer containing increasing imidazole concentrations from 40 mM to 500 mM. Fractions containing purified proteins were confirmed by SDS-PAGE gel analysis. Bradford measurements of protein concentration were performed on a Tecan Infinite M200 plate reader. A calibration curve for protein concentration quantifications was constructed by measuring 0, 10, 20, 40, 60, 80, 100 μg/mL of bovine serum albumin (BSA) with ab-sorbance measured at 595 nm with an optical path of 0.5 cm. The standard curve (y=0.0024×+0.4558, R²=0.99) was used to calculate protein concentrations.

Steady-state kinetics of NaPPO1, NaPPo2 and NaBBL2 enzymes: For steady-state kinetic analysis, the ability to oxidize N-caffeoylputrescine was tested in 84 mM acetate buffer (pH 4.8) at 22° C. water bath, containing 1 mM (Z)-3-hexnal, 1 mM FAD, 32 μg NaPPO1/NaPPO2, 30 μg/90 μg NaBBL2 (total volume 100 μL); assays lacking NaBBL2 enzymes served as negative controls.

The following eight N-caffeoylputrescine final concentrations were each tested at three independent experiments: 10, 20, 40, 60, 80, 120, 140, and 160 μM. Likewise, the ability to catalyze (Z)-3-hexnal was tested in in 84 mM acetate buffer (pH 4.8) at 22° C., containing 80 μM N-caffeoylputrescine, 1 mM FAD, 32 μg NaPPO1/NaPPO2, 30 μg/90 μg NaBBL2 (total volume 100 μL); again, assays lacking NaBBL2 enzymes served as negative controls. The following eight (Z)-3-hexnal final concentrations were each tested in three independent experiments: 500, 600, 700, 800, 1000, 1200, 1600, and 2000 μM. The reactions were initiated by the addition of the corresponding recombinant enzymes and the components were mixed by briefly vortexing, and immediately returned to the 22° C. water bath for incubation. After 20 minutes, all the reactions were briefly vortexed, and 100 μL aliquot of each reaction was transferred to a cold Eppendorf tube (−20° C.) containing 100 μL of MeOH (with 600 ng/mL testosterone as an internal standard) to terminate enzyme activity. Samples were centrifuged at 16,000g, 4° C. for 30 min, and 150 μL supernatants were transferred into to 2 mL glass vials for LC/MS (1 μL injection volume) analysis under the same method as described previously. Standard curves for caffeoylputrescine using authentic standards (with 600 ng/mL testosterone as an internal standard) (FIG. 34) were used to calculate the concentrations of substrate and product in each assay sample. The kinetic constants for caffeoylputrescine and (Z)-3-hexnal, apparent Km and Vmax, were inferred using a nonlinear regression in GraphPad Prism 9 (FIG. 34).

### Example 17

### In Vitro Assays and Production and Purification of CHP

In vitro assays of NaPPO1, NaPPO2, and NaBBL2: NaPPO1, NaPPO2, and NaBBL2 enzyme activity assays were performed by incubating 100 μg of the purified recombinant protein in 84 mM acetate buffer (pH 4.8) containing 20 mM (E)-N-caffeoylputrescine (BOC Sciences, catalogue #: B0005-053482) and 1 M cis-3-hexenal (Sigma, catalogue #: W256102-SAMPLE-K) or trans-2-hexenal (Sigma, catalogue #: W256005-1KG-K). During incubation of the enzyme reactions at 8° C., 1 μL of the reactions buffer was directly injected and analyzed by LC-MS every h to detect products. Assays lacking enzymes or (E)-N-caffeoylputrescine served as negative controls.

Production and purification of CHP: 10 replicates of 3 mL enzyme reactions incubating 100 μg of the purified recombinant protein in 84 mM acetate buffer (pH 4.8) containing (E)-N-caffeoylputrescine and 1 M cis-3-hexenal for 2 days at 8° C. were combined. To remove the salts, the 30 mLenzyme reaction were loaded on a SPE column (CHROMABOND HR-X, 45 μm, 3 mL/200 mg, Macherey-Nagel, catalogue #: 730931P45). The SPE column was dried using argon gas. 3 mL of 100% methanol was used to elute the compound mixture from the SPE column. Further fractionation was conducted on an Agilent 1100 HPLC system equipped with a Nucleodur Sphinx RP18 column (150×4.6 mm, 5 μm particle diameter, Macherey-Nagel, Germany). The following binary mobile phase gradient was applied: 0 to 30 min, gradient phase from 70% A (Milli-Qwater with 0.05% formic acid), 30% B (MeOH with 0.05% formic acid) to 50% A, 50% B; 30 to 35 min, gradient phase from 50% A, 50% B to 100% B; 30 to 35 min, isocratic at 100% B. The flow rate was 900 μL min⁻¹. Fractions were collected throughout the fractionation program with 1 min collection windows; each fraction was examined by LC-MS. The fractions containing the target molecule were pooled, diluted with the 5-fold amount of water and loaded on a SPE column (CHROMABOND HR-X, 45 μm, 3 mL/200 mg). After drying with argon gas, the SPE column was eluted with 700 μL of MeOH-d₃ containing 0.05% formic acid. The eluate was immediately subjected to NMR analysis.

### Example 18

### Empoasca Feeding Assays

Empoasca feeding assays: The survival rate of Empoasca feeding on artificial diet (10% 5 sucrose in water) supplemented with caffeoylputrescine (CP, 100 μM), coumaroylputrescine (CoP, 7 μM), feruloylputrescine (FP, 10 μM), or the m/z 347.19 hexenyl derivative of CP (CPH, 1 μM) was recorded every 1 h overnight. The artificial feeding devices consisted of a 50-mL Falcon tube with a 7-mm hole drilled into the conical bottom through which the leafhoppers were aspi-rated into the tube and which then was covered with Parafilm punctured with needles to create ventilation holes. The large opening of the Falcon tube was covered with a double layer of Parafilm containing the liquid diet (100 μL). The artificial feeding devices were placed upside down with the liquid diet side down on a White Light Box (STRATAGENE) to provide bottom-light illu-mination.

### Example 19

### Phylogenetic Analysis and Statistical Analysis

Phylogenetic analysis: The phylogenetic tree of different species was constructed using 5 Taxonomy Common Tree tool from NCBI. Briefly, species taxonomy names were uploaded to Taxonomy Common Tree to generate a taxonomic tree for the selected group of organisms. The taxonomic tree was saved as a phylip tree file (phy format). The phylogenetic tree was visualized using R package “treeio”.

Statistical analysis: Statistical analysis of the data was performed using R. Statistical significance between multiple groups (>3) of data was evaluated using analysis of variance (ANOVA), followed by Tukey's honestly significant difference (HSD) post hoc tests. For analysis of differences between two groups of data, Student's t tests were used with the two-tailed distribution of two sets of samples with equal variance. For Empoasca choice assay test, a blocking term was included in the analysis of variance to address the comparisons in the separate replicates and Freidman test was used followed by Wilcoxon post hoc tests.

### Example 20

### NMR and Chemicals

NMR: NMR spectra were recorded at 298 K on a 500 MHz Bruker Avance Ill HD spectrometer equipped with a cryoplatform and a 5 mm TCI cryoprobe (Bruker Biospin GmbH, Rheinstet-ten, Germany). The spectra were referenced to the residual solvent signals at⁶c 49.15 and S_{H}3.31. For spectrometer control and data processing Bruker TopSpin ver.3.2 was used. Standard pulse programs as implemented in Bruker TopSpin were used.

Chemicals: N-caffeoylputrescine was purchased from BOC Sciences, Shirley, NY, USA. The synthesis of N-coumaroylputrescine and N-feruloylputrescine performed as described before (Kyselka et al., J Agric Food Chem 66, 11018-11026 (2018). Compounds were assayed for purity by LC-MS.

### Example 21

### BBL2's Role in Biosynthesis of CHP, but not In Vitro

It was found that NaBBL2, while not being required for in vitro synthesis, is required for in vivo CPH biosynthesis. It is assumed that NaBBL2 is playing a role in solving the localization challenge, which could have other possible solutions. For example, plastids and their contents are regularly transferred to vacuoles during stress-induced autophagy (Izumi et al., Plant Cell 29, 377-394 (2017)), and if the biochemical function of PPOs survive their transport to the vacuole, the required constituents for CPH biosynthesis would be all present in the same organelle (FIG. 60).

Most members of the BBE-like protein family contain a bi-covalently attached FAD cofactor, and the physical association of the FAD cofactor to BBL proteins occurs via particular His and Cys residues (Daniel et al., Arch Biochem Biophys 632, 88-103 (2017), Winkler et al., J Biol Chem 281, 21276-21285 (2006)). It was discovered that NaBBL2 lacks the Cys residues involved in co-valent binding of the FAD cofactor; the C to G mutation of BBL2 at the substrate binding site suggests a different biochemical function of NaBBL2 in N. attenuata plants (FIG. 61).

It is proposed that BBL2 may function as a non-catalytic protein in the N. attenuata CPH pathway, such as previously reported for the non-catalytic chalcone isomerase-like (CHIL) in flavonoid metabolism (Ban et al., P Natl Acad Sci USA 115, E5223-E5232 (2018)). In this scenario, BBL2 could interact with the reactive PPO-activated CP to stabilize it in the cell environment and avoid conversion to a by-product that cannot react with (Z)-3-hexenal to form CPH. As such, BBL2 would allow for more efficient channeling of substrates among active enzymes. Many pathways involved in specialized metabolism have been proposed to be organized in protein complexes or metabolons. Metabolons facilitate the channeling of labile and toxic intermediates and increase local substrate concentrations and prevent undesired metabolic cross-talk (Laursen et al., Science, 354, 890-893 (2016); Gou et al., Nature Plants 4, 299-310 (2018)). Such dynamic assembly and disassembly permits rapid reorganization of metabolic profiles in response to environmental challenges and are thought to involve scaffolding proteins (Jorgensen et al., Current opinion in plant biology 8, 280-291 (2005)). Since it was observed that CPH was highly induced in field-grown plants, a scaffolding function devoted to BBL2 could possibly allow for a dynamic assembly and disassembly of a metabolon channeling reaction intermediates and maximizing catalytic efficiency toward CPH production. Consistent with such a mechanism, it was observed that CPH is only formed in plants when local CP concentrations are over 50 μM. The absence of a possible BBL2-dependent stabilization/channeling is probably less of an issue during in vitro CPH assays for which (Z)-3-hexenal is directly accessible to the PPO enzymes. However, when only a small fraction of (Z)-3-hexenal is accessible to the PPO-catalyzed CP micro-environment in vivo, BBL2's stabilization/channeling would be more critical to efficiently produce CPH.

### Example 22

### Tables

TABLE 1
Stability of m/z 347.19 during sample extraction and purification.

| | Stability |
|---|---|
| Leaf extraction conditions | |
| Leaf sample collection using aluminium foil in liquid N₂ | No |
| Leaf sample collection using Eppendorf tubes without | Yes |
| direct contact with liquid N₂ | |
| Sample extraction buffer pH 4.8 | Yes |
| Sample extraction buffer pH 10 | No |
| Purified metabolite conditions | |
| Purifying m/z 347.19 using SPE column with argon flow | Yes |
| Purifying m/z 347.19 using freeze drying | No |
| Purifying m/z 347.19 using rotary evaporator | No |

TABLE 2
Michaelis-Menten kinetics of PPO1, PPO2 and BBL2 on
caffeoylputrescine and (Z)-3-hexenal

| | Caffeoylputrescine | | (Z)-3-hexenal | |
|---|---|---|---|---|
| | Vmax | Km | Vmax | Km |
| PPO1 | 0.21 ± 0.01 | 67.30 ± 10.59 | 0.09 ± 0.01 | 853.22 ± 257.87 |
| PPO1 + | 0.25 ± 0.02 | 86.09 ± 15.34 | 0.10 ± 0.02 | 870.67 ± 381.05 |
| BBL2 | | | | |
| (30 μg) | | | | |
| PPO1 + | 0.26 ± 0.04 | 95.52 ± 28.20 | 0.09 ± 0.02 | 870.67 ± 381.05 |
| BBL2 | | | | |
| (90 μg) | | | | |
| PPO2 | 0.64 ± 0.10 | 204.35 ± 51.37 | 0.023 ± 0.005 | 673.35 ± 393.28 |
| PPO2 + | 0.63 ± 0.09 | 206.18 ± 49.95 | 0.021 ± 0.004 | 662.39 ± 360.69 |
| BBL2 | | | | |
| (30 μg) | | | | |
| PPO2 + | 0.67 ± 0.17 | 207.19 ± 78.07 | 0.024 ± 0.003 | 643.31 ± 217.25 |
| BBL2 | | | | |
| (90 μg) | | | | |

Note.
Calculated Vmax values are given in [μM mg⁻¹min⁻¹]. Km values are given in [μM]. Values are mean ± SEM

TABLE 3
List of oligonucleotides used in the Examples

| | | | SEQ |
|---|---|---|---|
| | | Descrip- | ID |
| Name | Sequence | tion | NO: |
| NaAT1 | GGGGACAAGTTTGTACAAAAAA-GC | Pathway | 47 |
| | AGGCTTAATGAATGTGAAAATTGA- | Elucida- | |
| | GAGCTC | tion | |
| NaAT1 | GGGGACCACTTTGTACAAGAAAGCT | Pathway | 48 |
| | GGG-TATCATTTTGCTTTCAAATCA | Elucida- | |
| | AGAGAATAG | tion | |
| NaPPO1 | GGGGACAAGTTTGTACAAAAAA-GC | Pathway | 49 |
| | AGGCTTAATGGCTTCTTCTTCTACT | Elucida- | |
| | CTAC | tion | |
| NaPPO1 | GGGGACCACTTTGTACAAGAAAGCT | Pathway | 50 |
| | GGG-TAATAAAAAGCCTTGAGTTCA | Elucida- | |
| | ATCAAC | tion | |
| NaPPO2 | GGGGACAAGTTTGTACAAAAAA-GC | Pathway | 51 |
| | AGGCTTAATGGCTTCTTCTTCTTCT | Elucida- | |
| | TCTAC | tion | |
| NaPPO2 | GGGGACCACTTTGTACAAGAAAGCT | Pathway | 52 |
| | GGG-TATTAACAATCGACAAGCTTA | Elucida- | |
| | ATCTC | tion | |
| NaBBL2 | GGGGACAAGTTTGTACAAAAAA-GC | Pathway | 53 |
| | AGGCTTAATGGCTTCTTCTTCTTCT | Elucida- | |
| | TCTAC | tion | |
| NaBBL2 | GGGGACCACTTTGTACAAGAAAGCT | Pathway | 54 |
| | GGG-TATTCACTACTATACTTATGC | Elucida- | |
| | TCTTGC | tion | |
| NaAT1 | ACAAAAGGGGGCTACCAGCA | qPCR | 55 |
| NaAT1 | ATTGGATACGGTCAGGGGCG | qPCR | 56 |
| NaPPO1 | GCGAAGTGGTCAGCATCGAC | qPCR | 57 |
| NaPPO1 | ACGACCCCTCAAGACACACA | qPCR | 58 |
| NaPPO2 | GGTTCCAAAGACAGGTGGCG | qPCR | 59 |
| NaPPO2 | GTGCACACACTGCGTTCAGA | qPCR | 60 |
| NaBBL2 | ACGGAATCTCTCTCAAGGCAGT | qPCR | 61 |
| NaBBL2 | CCTCCTATGCTTGGTTCAACGC | qPCR | 62 |
| NaJAZi | AGTTGAAGGCCAATTCTGTGCT | qPCR | 63 |
| NaJAZi | TGCTGCCATCCACCACCATA | qPCR | 64 |
| NaJAZh | TTCTGCTACGCCGCAAGTACTG | qPCR | 65 |
| NaJAZh | GGTATGGCGCTCTAGCCGTTG | qPCR | 66 |
| NaJAZc | TGCACCATATGGCCTCAGCA | qPCR | 67 |
| NaJAZc | AGCTAGGTCTCCTTACCGGCT | qPCR | 68 |
| NaJAZe | CAATTTGGTCAAGGAGACGTGA | qPCR | 69 |
| NaJAZe | GGCTCTGATCACAATTACAAGG | qPCR | 70 |
| NaJAZg | CACGCATCTACATTGACTTGCCA | qPCR | 71 |
| NaJAZg | AGCAGCTGGAGCTTCTTTGTT | qPCR | 72 |
| NaJAZL | ACTCTTCCACCAGAAAAGTTGCTC | qPCR | 73 |
| NaJAZL | TGAGATGTCCATTGCCAGGAGT | qPCR | 74 |
| NaMYC2a | GGCCCGGAACAACTACTACA | qPCR | 75 |
| NaMYC2a | CCCCGTCGATTAAAGTCTGA | qPCR | 76 |
| NaMYC2b | TCTGGTGCGATGAAGTCAAG | qPCR | 77 |
| NaMYC2b | CTGCTTCGACGTGATTCAAA | qPCR | 78 |
| NaDH29 | ATCAACTAGCCATTAGAATG | qPCR | 79 |
| NaDH29 | CCAAAAATGATTTGCAAGGTC | qPCR | 80 |
| NaCV86 | ATCAAATAGCTGAAGATGTC | qPCR | 81 |
| NaCV86 | CCAACAAAGTAGTGCTGTACT | qPCR | 82 |
| NaIF5α- | GTCGGACGAAGAACACCATT | qPCR | 83 |
| 2-q-F | | | |
| NaIF5α- | CACATCACAGTTGTGGGAGG | qPCR | 84 |
| 2-q-R | | | |
| NaMYB8 | GGGAATTCCATATGATGGGCAGAGC | Y2H | 85 |
| | TCCT | | |
| NaMYB8 | CCG-GAATTCTCATAATTCAGGCAA | Y2H | 86 |
| | TTCTAACATC | | |
| NaMYC2a | CCCAGCATATGATGACGGACTATAG | Y2H | 87 |
| | AATACC | | |
| NaMYC2a | TACAGATCGATGTCGCGATTCAGCA | Y2H | 88 |
| | ATTCTGG | | |
| NaMYC2b | CCCAGCATATGATGAATTTGTGGAA | Y2H | 89 |
| | TACTAG | | |
| NaMYC2b | TACAGATCGATGGCGTGTTTCAGCA | Y2H | 90 |
| | ACTCTGG | | |
| NaAT1 | GCGGCGGTCGACGTTAC-CTATGAT | VIGS | 91 |
| | GCTTTAATGGC | | |
| NaAT1 | GCGGCGGGATCCTGTGACTTGAACT | VIGS | 92 |
| | T-GGATCAAC | | |
| NaPPO1 | GCGGCGGTCGACGATGGGGTATGAT | VIGS | 93 |
| | TAC-GAACC | | |
| NaPPO1 | GCGGCGGGATCCATGTCATCTGCAT | VIGS | 94 |
| | TCACAG-TCT | | |
| NaPPO2 | GCGGCGGTCGACAATGGGCTTCGAT | VIGS | 95 |
| | TACGC | | |
| NaPPO2 | GCGGCGGGATCCAGCTCGTCCGCAT | VIGS | 96 |
| | TC | | |
| NaBBL2 | GCGGCGGTCGACTACTCCTGAGACC | VIGS | 97 |
| | TGCA | | |
| NaBBL2 | GCGGCGGGATCCAGTCCGTCTTGCC | VIGS | 98 |
| | TTTG | | |
| NaPPO1 | ACGCGTCGACCGATGGCTTCTTCTT | Protein | 99 |
| | CTACTC-TAC | Expression | |
| NaPPO1 | CCGCTCGAGCATAAAAAGCCTTGAG | Protein | 100 |
| | -TTCAATCAAC | Expression | |
| NaPPO2 | ACGCGTCGACCGATGGCTTCTTCTT | Protein | 101 |
| | CTTCTTC-TAC | Expression | |
| NaPPO2 | CCGCTCGAGCACAATCGACAAGCTT | Protein | 102 |
| | AATCTCA | Expression | |
| NaBBL2 | CCGGAATTCATGTTTCCAC-TCATA | Protein | 103 |
| | ATTCTGATCAG | Expression | |
| NaBBL2 | CCGAAGCTTCTTCACTACTA-TACT | Protein | 104 |
| | TATGCTCTTGC | Expression | |

1. A compound of general formula (I): 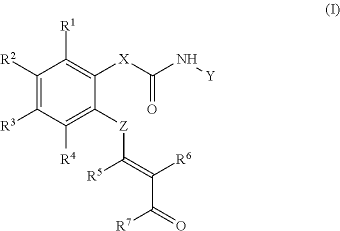
wherein:
R¹, R², R³, and R⁴ are each independently from each other H, OH, (C₁-C₆)-alkyl or (C₁-C₆)-alkoxy;
R⁵, R⁶ and R⁷ are each independently from each other H or (C₁-C₆)-alkyl;
X is straight-chain or branched (C₁-C₈)-alkyl, or straight-chain or branched (C₂-C₅)-alkenyl,
Y is selected from —(CH₂)ₘ—NH₂, —(CH₂)ₙ—NH—(CH₂)ₒ—NH₂, —(CH2)ₚ—NH—(CH₂)_{q}—NH—(CH₂)ᵣ—NH₂ NH—(CH₂)ₘ—NH₂, NH—(CH₂)ₙ—NH—(CH₂)ₒ—NH₂, and NH—(CH₂)ₚ—NH—(CH₂)_{q}—NH—(CH₂)ᵣ—NH2, with m, n, o, p, q, and r each being an integer between 1 and 10, or a tyramine ester; and
Z is straight-chain or branched (C₁-C₈)-alkyl, or straight-chain or branched (C₂-C₅)-alkenyl,
or an enantiomer, diastereomer, stereoisomer, or salt thereof.

2. The compound of claim 1, wherein:
two of R¹, R², R³, and R⁴ are OH;
R⁵, R⁶ and R⁷ are each H; and
X is a straight-chain (C₂-C₈)-alkenyl;
or an enantiomer, diastereomer, stereoisomer, or salt thereof.

3. The compound of claim 2, wherein:
R¹ and R⁴ are H,
R² and R³ are OH, and
X is —CH═CH—,
or an enantiomer, diastereomer, stereoisomer, or salt thereof.

4. The compound of claim 1, which has the following formula (II): 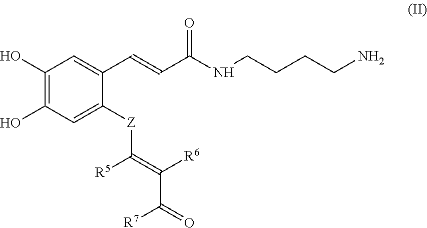
wherein R⁵, R⁶, R⁷ and Z are as defined above;
or an enantiomer, diastereomer, stereoisomer, or salt thereof.

5. The compound of claim 1, which has the following formula (III): 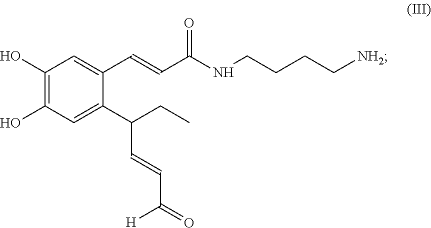 [SMILES: CCC(/C=C/C=O)c1cc(O)c(O)cc1/C=C/C(=O)NCCCCN]
or an enantiomer, diastereomer, stereoisomer, or salt thereof.

6. A method of producing the compound of claim 1, wherein said method is an enzymatic production method using at least a BBL2 berberine bridge enzyme 2) polypeptide, e.g., wherein said BBL2 (berberine bridge enzyme 2) polypeptide is:
(a) encoded by the polynucleotide having the nucleotide sequence of SEQ ID NO: 1;
(b) encoded by a polynucleotide which is a variant of SEQ ID NO: 1;
(c) encoded by a polynucleotide which is an allelic variant of SEQ ID NO: 1;
(d) encoded by a polynucleotide which is a species homologue of SEQ ID NO: 1;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (a) to (d);
(g) represented by the polypeptide of SEQ ID NO: 2;
(h) represented by a polypeptide fragment of SEQ ID NO: 2 having BBL2 (berberine bridge enzyme 2) function;
(i) represented by a polypeptide domain of SEQ ID NO: 2 having BBL2 (berberine bridge enzyme 2) function;
(j) represented by a polypeptide having an amino acid sequence at least 75%, 80%, 90%, 95%, 97%, 98%, or 99% identical to the amino acid sequence of SEQ ID NO: 2 and having BBL2 (berberine bridge enzyme 2) function; or
(k) represented by a polypeptide being encode by any one of the polynucleotides specified in (a) to (f)
wherein enzymatic production optionally is performed with and in a genetically modified cell, tissue or organism, wherein said genetic modification allows for the heterologous expression of the polypeptide.

7. (canceled)

8. (canceled)

9. The method of claim 6, wherein said enzymatic production method additionally uses a PPO (polyphenol oxidase) activity or polypeptide, wherein preferably said PPO (polyphenol oxidase) activity or polypeptide is:
(a) encoded by the polynucleotide having the nucleotide sequence of SEQ ID NO: 3 or 5;
(b) encoded by a polynucleotide which is a variant of SEQ ID NO: 3 or 5;
(c) encoded by a polynucleotide which is an allelic variant of SEQ ID NO: 3 or 5;
(d) encoded by a polynucleotide which is a species homologue of SEQ ID NO: 3 or 5;
(e) encoded by a polynucleotide which is at least 75%, 80%, 90%, 95%, 97%, 98%, or 99% identical to polynucleotide as defined in any one of (a) to (d);
(f) encoded by a polynucleotide capable of hybridizing under stringent conditions to any one of the polynucleotides specified in (a) to (d);
(g) represented by the polypeptide of SEQ ID NO: 4 or 6;
(h) represented by a polypeptide fragment of SEQ ID NO: 4 or 6 having PPO (polyphenol oxidase) activity;
(i) represented by a polypeptide domain of SEQ ID NO: 4 or 6 having PPO (polyphenol oxidase) activity;
(j) represented by a polypeptide having an amino acid sequence at least 75%, 80%, 90%, 95%, 97%, 98%, or 99% identical to the amino acid sequence of SEQ ID NO: 4 or 6 and having PPO (polyphenol oxidase) activity; or
(k) represented by a polypeptide being encoded by any one of the polynucleotides specified in (a) to (f),
wherein said genetic modification optionally results in the expression of the PPO activity of polypeptide.

10. The method of claim 6, wherein said enzymatic production method additionally uses an AT1 (polyamine hydroxycinnamoyltransferase 1) activity or polypeptide, wherein preferably said AT1 (polyamine hydroxycinnamoyltransferase 1) activity or polypeptide is:
(a) encoded by the polynucleotide having the nucleotide sequence of SEQ ID NO: 7;
(b) encoded by a polynucleotide which is a variant of SEQ ID NO: 7;
(c) encoded by a polynucleotide which is an allelic variant of SEQ ID NO: 7;
(d) encoded by a polynucleotide which is a species homologue of SEQ ID NO: 7;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (a) to (d);
(g) represented by the polypeptide of SEQ ID NO: 8;
(h) represented by a polypeptide fragment of SEQ ID NO: 8 having AT1 (polyamine hydroxycinnamoyltransferase 1) activity;
(i) represented by a polypeptide domain of SEQ ID NO: 8 having AT1 (polyamine hydroxycinnamoyltransferase 1) activity;
(j) represented by a polypeptide having an amino acid sequence at least 75%, 80%, 90%, 95%, 97%, 98%, or 99% identical to the amino acid sequence of SEQ ID NO: 8 and having AT1 (polyamine hydroxycinnamoyltransferase 1) activity; or
(k) represented by a polypeptide being encoded by any one of the polynucleotides specified in (a) to (f),
wherein said genetic modification optionally results in the expression of the AT1 activity or polypeptide.

11. The method of claim 6, wherein said enzymatic production method additionally uses an ODC (ornithine decarboxylase) activity or polypeptide and/or an HPL (hydroperoxide lyase) activity or polypeptide, wherein preferably
(A) said ODC (ornithine decarboxylase) activity or polypeptide is:
(a) encoded by a polynucleotide having the nucleotide sequence of SEQ ID NO: 9 or 11;
(b) encoded by a polynucleotide which is a variant of SEQ ID NO: 9 or 11;
(c) encoded by a polynucleotide which is an allelic variant of SEQ ID NO: 9 or 11;
(d) encoded by a polynucleotide which is a species homologue of SEQ ID NO: 9 or 11;
(e) encoded by a polynucleotide which is at least 75%, 80%, 90%, 95%, 97%, 98%, or 99% identical to the polynucleotide as defined in any one of (a) to (d);
(f) encoded by a polynucleotide capable of hybridizing under stringent conditions to any one of the polynucleotides specified in (a) to (d);
(g) represented by a polypeptide of SEQ ID NO: 10 or 12;
(h) represented by a polypeptide fragment of SEQ ID NO: 10 or 12 having ODC (ornithine decarboxylase) activity;
(i) represented by a polypeptide domain of SEQ ID NO: 10 or 12 having ODC (ornithine decarboxylase) activity;
(j) represented by a polypeptide having an amino acid sequence at least 75%, 80%, 90%, 95%, 97%, 98%, or 99% identical to the amino acid sequence of SEQ ID NO: 10 or 12 and having ODC (ornithine decarboxylase) activity; or
(k) represented by a polypeptide being encoded by any one of the polynucleotides specified in (A) (a) to (f); and
(B) said HPL (hydroperoxide lyase) activity or polypeptide is:
(a) encoded by a polynucleotide having the nucleotide sequence of SEQ ID NO: 13, 15 or 17;
(b) encoded by a polynucleotide which is a variant of SEQ ID NO: 13, 15 or 17;
(c) encoded by a polynucleotide which is an allelic variant of SEQ ID NO: 13, 15 or 17;
(d) encoded by a polynucleotide which is a species homologue of SEQ ID NO: 13, 15 or 17;
(e) encoded by a polynucleotide which is at least 75%, 80%, 90%, 95%, 97%, 98%, or 99% identical to tac polynucleotide as defined in any one of (a) to (d);
(f) encoded by a polynucleotide capable of hybridizing under stringent conditions to any one of the polynucleotides specified in (B) (a) to (d);
(g) represented by a polypeptide of SEQ ID NO: 14, 16 or 18;
(h) represented by a polypeptide fragment of SEQ ID NO: 14, 16 or 18 having HPL (hydroperoxide lyase) activity;
(i) represented by a polypeptide domain of SEQ ID NO: 14, 16 or 18 having HPL (hydroperoxide lyase) activity;
(j) represented by a polypeptide having an amino acid sequence at least 75%, 80%, 90%, 95%, 97%, 98%, or 99% identical to the amino acid sequence of SEQ ID NO: 14, 16 or 18 and having HPL (hydroperoxide lyase) activity; or
(k) represented by a polypeptide being encoded by any one of the polynucleotides specified in (B) (a) to (f),
wherein said genetic modification optionally results in the expression of the ODC and/or HPL activity or polypeptide.

12. The method of claim 6, wherein said enzymatic production method additionally uses a PAL (L-phenylalanine ammonia lyase) activity or polypeptide and/or a C4H (trans-cinnamate 4-hydroxylase) activity or polypeptide and/or an 4CL (4-coumarate:coenzyme A ligase) activity or polypeptide and/or an HCT (Hydroxycinnamoyl-transferase) activity or polypeptide and/or a C3H (coumarate 3-hydroxylase) activity or polypeptide, wherein preferably (A) said PAL (L-phenylalanine ammonia lyase) activity or polypeptide is:
(a) encoded by a polynucleotide having the nucleotide sequence of SEQ ID NO: 19, 21, 23 or 25;
(b) encoded by a polynucleotide which is a variant of SEQ ID NO: 19, 21, 23 or 25;
(c) encoded by a polynucleotide which is an allelic variant of SEQ ID NO: 19, 21, 23 or 25;
(d) encoded by a polynucleotide which is a species homologue of SEQ ID NO: 19, 21, 23 or 25;
(e) encoded by a polynucleotide which is at least 75%, 80%, 90%, 95%, 97%, 98%, or 99% identical to the polynucleotide as defined in any one of A (a) to (d);
(f) encoded by a polynucleotide capable of hybridizing under stringent conditions to any one of the polynucleotides specified in A (a) to (d);
(g) represented by a polypeptide of SEQ ID NO: 20, 22, 24 or 26;
(h) represented by a polypeptide fragment of SEQ ID NO: 20, 22, 24 or 26 having PAL (L-phenylalanine ammonia lyase) activity;
(i) represented by a polypeptide domain of SEQ ID NO: 20, 22, 24 or 26 having PAL (L-phenylalanine ammonia lyase) activity;
(j) represented by a polypeptide having an amino acid sequence at least 75%, 80%, 90%, 95%, 97%, 98%, or 99% identical to the amino acid sequence of SEQ ID NO: 20, 22, 24 or 26 and having PAL (L-phenylalanine ammonia lyase) activity; or
(k) represented by a polypeptide being encoded by any one of the polynucleotides specified in A (a) to (f); and
(B) said C4H (trans-cinnamate 4-hydroxylase) activity or polypeptide is:
(a) encoded by a polynucleotide having the nucleotide sequence of SEQ ID NO: 27
(b) encoded by a polynucleotide which is a variant of SEQ ID NO: 27;
(c) encoded by a polynucleotide which is an allelic variant of SEQ ID NO: 27;
(d) encoded by a polynucleotide which is a species homologue of SEQ ID NO: 27;
(e) encoded by a polynucleotide which is at least 75%, 80%, 90%, 95%, 97%, 98%, or 99% identical to the polynucleotide as defined in any one of B (a) to (d);
(f) encoded by a polynucleotide capable of hybridizing under stringent conditions to any one of the polynucleotides specified in B (a) to (d);
(g) represented by the polypeptide of SEQ ID NO: 28;
(h) represented by a polypeptide fragment of SEQ ID NO: 28 having C4H (cinnamate 4-hydroxylase) activity;
(i) represented by a polypeptide domain of SEQ ID NO: 28 having C4H (cinnamate 4-hydroxylase) activity;
(j) represented by a polypeptide having an amino acid sequence at least 75%, 80%, 90%, 95%, 97%, 98%, or 99% identical to the amino acid sequence of SEQ ID NO: 28 and having C4H (cinnamate 4-hydroxylase) activity; or
(k) represented by a polypeptide being encoded by any one of the polynucleotides specified in B (a) to (f); and
(C) said 4CL (4-coumarate:coenzyme A ligase) activity or polypeptide is:
(a) encoded by a polynucleotide having the nucleotide sequence of SEQ ID NO: 29, 105, 107, 109, 111, 113, 115, 117, 119, 121, 123, 125, 127, 129 or 131;
(b) encoded by a polynucleotide which is a variant of SEQ ID NO: 29, 105, 107, 109, 111, 113, 115, 117, 119, 121, 123, 125, 127, 129 or 131;
(c) encoded by a polynucleotide which is an allelic variant of SEQ ID NO: 29, 105, 107, 109, 111, 113, 115, 117, 119, 121, 123, 125, 127, 129 or 131;
(d) encoded by a polynucleotide which is a species homologue of SEQ ID NO: 29, 105, 107, 109, 111, 113, 115, 117, 119, 121, 123, 125, 127, 129 or 131;
(e) encoded by a polynucleotide which is at least 75%, 80%, 90%, 95%, 97%, 98%, or 99% identical to the polynucleotide as defined in any one of C (a) to (d);
(f) encoded by a polynucleotide capable of hybridizing under stringent conditions to any one of the polynucleotides specified in C (a) to (d);
(g) represented by the polypeptide of SEQ ID NO: 30, 106, 108, 110, 112, 114, 116, 118, 120, 122, 124, 126, 128, 130 or 132;
(h) represented by a polypeptide fragment of SEQ ID NO: 30, 106, 108, 110, 112, 114, 116, 118, 120, 122, 124, 126, 128, 130 or 132 having 4CL (4-coumarate:coenzyme A ligase) activity;
(i) represented by a polypeptide domain of SEQ ID NO: 30, 106, 108, 110, 112, 114, 116, 118, 120, 122, 124, 126, 128, 130 or 132 having 4CL (4-coumarate:coenzyme A ligase) activity;
(j) represented by a polypeptide having an amino acid sequence at least 75%, 80%, 90%, 95%, 97%, 98%, or 99% identical to the amino acid sequence of SEQ ID NO: 30, 106, 108, 110, 112, 114, 116, 118, 120, 122, 124, 126, 128, 130 or 132 and having 4CL (4-coumarate:coenzyme A ligase) activity; or
(k) represented by a polypeptide being encoded by any one of the polynucleotides specified in C (a) to (f); and
(D) said HCT (Hydroxycinnamoyl-transferase) activity or polypeptide is:
(a) encoded by a polynucleotide having the nucleotide sequence of SEQ ID NO: 31, 33, 35, 37, 39, 41, or 43;
(b) encoded by a polynucleotide which is a variant of SEQ ID NO: 31, 33, 35, 37, 39, 41, or 43;
(c) encoded by a polynucleotide which is an allelic variant of SEQ ID NO: 31, 33, 35, 37, 39, 41, or 43;
(d) encoded by a polynucleotide which is a species homologue of SEQ ID NO: 31, 33, 35, 37, 39, 41, or 43;
(e) encoded by a polynucleotide which is at least 75%, 80%, 90%, 95%, 97%, 98%, or 99% identical to the polynucleotide as defined in any one of D (a) to (d);
(f) encoded by a polynucleotide capable of hybridizing under stringent conditions to any one of the polynucleotides specified in D (a) to (d);
(g) represented by a polypeptide of SEQ ID NO: 32, 34, 36, 38, 40, 42 or 44;
(h) represented by a polypeptide fragment of SEQ ID NO: 32, 34, 36, 38, 40, 42 or 44 having HCT (Hydroxycinnamoyl-transferase) activity;
(i) represented by a polypeptide domain of SEQ ID NO: 32, 34, 36, 38, 40, 42 or 44 having HCT (Hydroxycinnamoyl-transferase) activity;
(j) represented by a polypeptide having an amino acid sequence at least 75%, 80%, 90%, 95%, 97%, 98%, or 99% identical to the amino acid sequence of SEQ ID NO: 32, 34, 36, 38, 40, 42 or 44 and having HCT (Hydroxycinnamoyl-transferase) activity; or
(k) represented by a polypeptide being encoded by any one of the polynucleotides specified in D (a) to (f); and
(E) said C3H (coumarate 3-hydroxylase) activity or polypeptide is:
(a) encoded by a polynucleotide having the nucleotide sequence of SEQ ID NO: 45;
(b) encoded by a polynucleotide which is a variant of SEQ ID NO: 45;
(c) encoded by a polynucleotide which is an allelic variant of SEQ ID NO: 45;
(d) encoded by a polynucleotide which is a species homologue of SEQ ID NO: 451;
(e) encoded by a polynucleotide which is at least 75%, 80%, 90%, 95%, 97%, 98%, or 99% identical to the polynucleotide as defined in any one of E (a) to (d);
(f) encoded by a polynucleotide capable of hybridizing under stringent conditions to any one of the polynucleotides specified in E (a) to (d);
(g) represented by a polypeptide of SEQ ID NO: 46;
(h) represented by a polypeptide fragment of SEQ ID NO: 46 having C3H (coumarate 3-hydroxylase) activity;
(i) represented by a polypeptide domain of SEQ ID NO: 46 having C3H (coumarate 3-hydroxylase) activity;
(j) represented by a polypeptide having an amino acid sequence at least 75%, 80%, 90%, 95%, 97%, 98%, or 99% identical to the amino acid sequence of SEQ ID NO: 46 and having C3H (coumarate 3-hydroxylase) activity; or
(k) represented by a polypeptide being encoded by any one of the polynucleotides specified in E (a) to (f),
wherein said genetic modification optionally results in the expression of the PAL, C4H, 4CL, HCT, and/or C3H activity or polypeptide.

13-22. (canceled)

23. The method of claim 6, wherein said expression is conveyed by a native, regulated, tissue specific or constitutive promoter

24. The method, organism, tissue or cell of claim 23,
wherein said promoter allows for (i) a polycistronic expression of an activity or polypeptide (ii) an individual expression of an activity or (iii) a group-wise expression of groups of at least two activities.

25. The method, organism, tissue or cell of claim 6, wherein enzymatic production is performed with and in a genetically modified cell, tissue or organism, wherein said genetic modification allows for the heterologous expression of the polypeptide and wherein said expression is an overexpression, e.g., wherein said overexpression is conveyed by a strong regulated or strong constitutive promoter, and/or by the provision of at least a second copy of a genetic element encoding said activity or polypeptide.

26. (canceled)

27. The method of claim 6, wherein said enzymatic activity or polypeptide is derived from an organism belonging to the genus Nicotiana, preferably of the species Nicotiana attenuata.

28-31. (canceled)

32. An expression cassette for heterologous expression in a eukaryotic host cell, preferably a plant cell, wherein said expression cassette comprises a polynucleotide as defined in claim 6.

33. A vector or insertion construct comprising the expression cassette of claim 32.

34. (canceled)

35. (canceled)

36. A composition comprising the compound of claim 2, optionally additionally comprising an acceptable carrier, stabilizer and/or spreading agent.

37-41. (canceled)

42. A method of plant protection comprising contacting a plant or part of a plant with the compound of claim 1.


SEQUENCE LISTING (144 sequences)

SEQ ID NO: 1 - DNA, 1701 bp, Nicotiana attenuata
  source 1..1701: mol_type = other DNA
atgtttccactcataattctgatcagcttttcacttacttccctctctgctactgctactaatggagcagaaggaggagctacaagtctttcaacctgtttaatcaaccacaatatccataacttctctatttactccacaaggaatgatccaaatagtaattactttaacttgctcgacttctcccttcagaatcttcgatttgcagcatcttacatgccaaaaccaaccctcattatcctaccaaacagcaaggaggagctcgtgagcaccattctttgttgcagacaagcatcttatcaaatcagagtaaggtgcggcggacacagctacgaaggaacttcttacgtttcctttgacggttccccattcgtgatcgttgacttgatgaaaatagacgacgtttcagtagatttggattctgaaactgcttgggctcagggcggcgcaacaattggccaaatttattacgccattgccaaggcaagtgacgttcatgcattttcagcaggttcggggccaacagtaggatctggaggtcatatttctggtggcggatttggacttttatctagaaaattcggacttgctgctgataatatagttgatgctcttcttattgatgctgatggacgcttattagaccgaaaagccatgggagaagacgtgttttgggcaatcagaggtggcggcggtggaaattggggaattgtttatgcctggaaaattcgattactcaaagtgcctaaaatcgtaacaacttgtatgatctataggcctggatccaaacaatacgtggctcaaatacttgagaaatggcaaattgttactccaaatttggtcgatgattttactctaggagtactcctgagacctgcagatttgccggcggatatgaaatatggtaatagtactcctattgaaatatttccccaattcaacgcactttatttgggtccaaaaactgaagctctttccatatcaaatgagacatttccggagctaggcgttaagaatgatgaatgcaaggaaatgacttgggtagagtcagcactttttttctctgagttagctgacgttagcggtaactcctctgctgatatctcccgtctgaaagaacgttacatggacggaaaaggtttcttcaaaggcaagacggactatgtgaagaagccagtttcaatggatgggatgctaacatttcttgttgaactcgagaaaaacccgaagggataccttgtcttcgatccttatggcggagccatggacaagattagtgatcaagctattgctttccctcatagaaaaggtaaccttttcgcgattcagtatctagcacagtggaatgaagaggacgattacatgagcaacgtttacatggagtggataagaggattttacaatacaatgacgccatttgtttcaagctcgccaaggggagcttatatcaactacttggatatggatcttggagtgaatatggtcgacaactacttattgcgaaatgctagtagtagttattcttcctctgttgatgctgtggagagagctagagcgtggggtgaaatgtatttcttgcataactatgataggttggttaaagctaagacacaaattgatccactaaatgtttttcgacatgaacagagtattcctcctatgcttggttcaacgcaagagcataagtatagtagtgaatga

SEQ ID NO: 2 - protein, 566 aa, Nicotiana attenuata
MFPLIILISFSLTSLSATATNGAEGGATSLSTCLINHNIHNFSIYSTRNDPNSNYFNLLDFSLQNLRFAASYMPKPTLIILPNSKEELVSTILCCRQASYQIRVRCGGHSYEGTSYVSFDGSPFVIVDLMKIDDVSVDLDSETAWAQGGATIGQIYYAIAKASDVHAFSAGSGPTVGSGGHISGGGFGLLSRKFGLAADNIVDALLIDADGRLLDRKAMGEDVFWAIRGGGGGNWGIVYAWKIRLLKVPKIVTTCMIYRPGSKQYVAQILEKWQIVTPNLVDDFTLGVLLRPADLPADMKYGNSTPIEIFPQFNALYLGPKTEALSISNETFPELGVKNDECKEMTWVESALFFSELADVSGNSSADISRLKERYMDGKGFFKGKTDYVKKPVSMDGMLTFLVELEKNPKGYLVFDPYGGAMDKISDQAIAFPHRKGNLFAIQYLAQWNEEDDYMSNVYMEWIRGFYNTMTPFVSSSPRGAYINYLDMDLGVNMVDNYLLRNASSSYSSSVDAVERARAWGEMYFLHNYDRLVKAKTQIDPLNVFRHEQSIPPMLGSTQEHKYSSE

SEQ ID NO: 3 - DNA, 1788 bp, Nicotiana attenuata
  source 1..1788: mol_type = other DNA
atggcttcttcttctactctacctttatgcaccacaaaaactcccttttcttcctccaccaactcatttttctttgcaaaaccctctcatcttttcctcaatggaaaacgtaaccaaagtttcaaggtttcatgcacgggcgagcatgacggaaaccagcttgacgcaattaaagaaggagctgttgaaagaaggaatgtccttttgggtttaggagggctgtatggcgcagctaatcttgcgccattagcctctgctgctcctgtaccaccccccgatctaaaatcatgtggcccggccacgataacggatggtccaactgtatcatatagttgttgcccacctacaccagatgatatggacagcgttccatattacaagatccctcgcatgtccaagcttcgtaagcggccagctgcccaagacgtgagtgaggagtatatagccaagtaccagttagccactagtaaaatgagggaattagacaaagacccatttgatcctcttggcttcaagcaacaagctaatatccattgtgcttattgtaacgatgcttacacaatgggtgacaaaaagttacaagttcaccaatcgtggcttttcttcccgtttcatagatggtacttgtacttctacgagagaatcttgggctccctcatcgatgatccaacttttgctctgccatattggaactgggaccatccaagcggcatgcgtttgcctgctatgttcgatgtcgaaggttcttcactgtacgatgcaagacgtaatccaaatgtccgtaatggaaccattatcgatcttggttttttcggtaatgaagtcaagactaatgaaatacagatgataactaacaacttaattctaatgtatcgtcaaatgataaccaatgctccaagcccgctgttgttcttcggagagccttacagattcggatctaaacccaatccagggcagggaaccattgagaacatccctcatactccggttcacatttggactggtactgtgcgggatacggatttgggtaatggtgtgaaatcatacggtgaggatatgggtaatttctactcggctggtttagacccagttttttacagccaccacgccaatgtggaccgcatgtggaatgaatggaaagcactaggagggaaaagaagggatctcacagacaaagattggttaaactcggagttctttttctacgatgaaaaccgcgacccatggcgtgtcaaagtccgagactgtttggacagtaagaagatggggtatgattacgaaccgaaagccacaccatggcgtaactttaagccagggaaaaagaccacaccgggcaaggtgaatctacgttcagttaagccagccagcaaggtattcccactctcaaatctggacagagccatttgctttagtatagagaggccagctacatcaaggagtcagcaggagaaagatgaatttgaggaggtgctaacattcaagaatttaaagtatgatgatagcaggtatataaaatttgatgtgttcgtcaatgcagacaagactgtgaatgcagatgacattgacaagacagagtatgcagggagctataccagcttgccacatgttcatggacctaataatgcaactcatgtggttgaagtggaagaattcaagctagccatcactgaactgctagaggactgtggtttggaagatgacgacattattgcggtaactgtggttccaaagacggggggcgaagtggtcagcatcgactgtgtgttgattgaactcaaggctttttat

SEQ ID NO: 4 - protein, 596 aa, Nicotiana attenuata
MASSSTLPLCTTKTPFSSSTNSFFFAKPSHLFLNGKRNQSFKVSCTGEHDGNQLDAIKEGAVERRNVLLGLGGLYGAANLAPLASAAPVPPPDLKSCGPATITDGPTVSYSCCPPTPDDMDSVPYYKIPRMSKLRKRPAAQDVSEEYIAKYQLATSKMRELDKDPFDPLGFKQQANIHCAYCNDAYTMGDKKLQVHQSWLFFPFHRWYLYFYERILGSLIDDPTFALPYWNWDHPSGMRLPAMFDVEGSSLYDARRNPNVRNGTIIDLGFFGNEVKTNEIQMITNNLILMYRQMITNAPSPLLFFGEPYRFGSKPNPGQGTIENIPHTPVHIWTGTVRDTDLGNGVKSYGEDMGNFYSAGLDPVFYSHHANVDRMWNEWKALGGKRRDLTDKDWLNSEFFFYDENRDPWRVKVRDCLDSKKMGYDYEPKATPWRNFKPGKKTTPGKVNLRSVKPASKVFPLSNLDRAICFSIERPATSRSQQEKDEFEEVLTFKNLKYDDSRYIKFDVFVNADKTVNADDIDKTEYAGSYTSLPHVHGPNNATHVVEVEEFKLAITELLEDCGLEDDDIIAVTVVPKTGGEVVSIDCVLIELKAFY

SEQ ID NO: 5 - DNA, 1773 bp, Nicotiana attenuata
  source 1..1773: mol_type = other DNA
atggcttcttcttcttcttctactttacctttatgcgccagtaaaactccctcttcttccttcaccaacaccaactcatctttctttgcaaaaccctctcagcttttccttcatggaaaacgtaaccaaaatttcaagttctcatgcaatgccaacagtggcgagcatgacaaaaaccttgacgctgttgacaggaggaatgtcctgttgggtttaggagggctgtatggcgcagctaatcttgcgccattagctactgctgctcctataccacctcctgatctcaaatcttgtagcaaagcccatataaatgaccaagaggaggtttcatacagttgttgcccccctatcccaagtgatatggacagcgttccatattacaagtttccttctatgcccaaactccgtattcggcctgctgctcatgctgctgatgaggagtacattgctaaataccagttagccactagtcgaatgagggaacttgacaaagacccatttgaccctcttggcttcaagcaacaagccaatatccattgtgcttattgcaacggtgcttacaaaattggtggcaaagagttacaagtccatttctcgtggctttttttcccttttcatagatggtacttgtacttctatgagagaatcttgggctctttaattaatgatcctacttttggtttgccatattggaactgggaccatccaaagggcatgcgtatacctcccatgttcgatcgtgaagggtcttccctttacgatgcaagacgtaaccaaagtcaccgtaatggaaccataatcgatcttggttattttggtaccgaagtccaaacaactcaactgcagcagatgactaataacttaactataatgtatcgtcaaatgataactaatgctccttgcccgttgctcttctttggtcagccttaccctctaggaactgatcccagtccagggatggggactattgaaaacattcctcatactcctgtccacatctgggttggtagtggtcctaacgagaataatgtgcaacacggtgaggatatgggtaatttttactcggccggtttagacccgcttttctattcccatcacgccaatgtggaccggatgtggtccgagtggaaagccttaggagggaaaagaagggatctcacgcacaaagattggttgaactcggagttctttttctacgatgaaaaccgcaacccgttccgtgtgaaagtccgtgactgtttggacagtaagaaaatgggcttcgattacgcaccgatgccaaccccatggcgtaattttaagccaataaggaagagcaatgcagggaaggtgaatctaagttcagttccgccagcaagcagggtgttcccactctcaaagctggacagagccatttcattttccatcgataggccatcttcgtcaaggactcaacaggagaaaaatgaacaagaggagatgctaacgttcaacaacattaagtatgacgatagcaagtatataaggttcgatgtgttcctcaacgtggacaagactgtgaatgcggacgagcttgacaaggcggagtatgcggggagttacaccagcttgccacatgttcatggagataatgcctctcatgttacctctgtaacattccagttggccatcactgaactgttggaggatattgggttggaagatgaagacactattgcggtaactgtggttccaaagacaggtggcgaagagatctccattgaaggtgttgagattaagcttgtcgattgttaa

SEQ ID NO: 6 - protein, 590 aa, Nicotiana attenuata
MASSSSSTLPLCASKTPSSSFTNTNSSFFAKPSQLFLHGKRNQNFKFSCNANSGEHDKNLDAVDRRNVLLGLGGLYGAANLAPLATAAPIPPPDLKSCSKAHINDQEEVSYSCCPPIPSDMDSVPYYKFPSMPKLRIRPAAHAADEEYIAKYQLATSRMRELDKDPFDPLGFKQQANIHCAYCNGAYKIGGKELQVHFSWLFFPFHRWYLYFYERILGSLINDPTFGLPYWNWDHPKGMRIPPMFDREGSSLYDARRNQSHRNGTIIDLGYFGTEVQTTQLQQMTNNLTIMYRQMITNAPCPLLFFGQPYPLGTDPSPGMGTIENIPHTPVHIWVGSGPNENNVQHGEDMGNFYSAGLDPLFYSHHANVDRMWSEWKALGGKRRDLTHKDWLNSEFFFYDENRNPFRVKVRDCLDSKKMGFDYAPMPTPWRNFKPIRKSNAGKVNLSSVPPASRVFPLSKLDRAISFSIDRPSSSRTQQEKNEQEEMLTFNNIKYDDSKYIRFDVFLNVDKTVNADELDKAEYAGSYTSLPHVHGDNASHVTSVTFQLAITELLEDIGLEDEDTIAVTVVPKTGGEEISIEGVEIKLVDC

SEQ ID NO: 7 - DNA, 1317 bp, Nicotiana attenuata
  source 1..1317: mol_type = other DNA
atgaatgtgaaaattgagagctcaagaattatcaagcctttctatgaaggaactccaccttcaaccaacactcacatttccttcaatgtctttgacaacgttacctatgatgctttaatggctttaatttacgcctatcgtccacctactcctccaacttccaccattgaaatgggactcagaaaaaccctagcagtttatagggaatgggcaggtagaataggcagagatgaaaatggcaatcgcgtggtttttctcaacgatgagggtgttagattcatcgaggcatcggtcaatgccacgttagatgaagtattgcccttaaagccctctccttctttgcttaagttgcatcctggtatgaaggatgtagtggagttgatccaagttcaagtcacaaggttcacttgtggctctgtgatggtcgggttcactggccaccacatgatagctgacggccacgcggctagcaactttttcgtcgcgtggggccaagcttgccgtggtgtagaaatcactccacttcctttgcatgatcgtgctattttccatcctcggaaccctccacttatcgaattcaaccacgtgggggcagaattcatgtccaaatcattaaacaagaaggaattcatcaaactagagaatacagaaaaaaacataattgttcacaaagtccatttcacattggagttcctaggcaaactcaaggcaaatgcttctttcatgaatgggaaaaccaagacttatagcacgtttgaaagtcttgttgcgcatttatggagggtcataactaaggcacgcgaactagacgggtcccaaaatacacaaataagaatctcagtggatggtcgcagaagagttgtaccaagagtagcagatgaattttttggtaacatagttctttgggcatttccaacatccaaagtgagagatcttgtaaatgaaccacttcactatgcaacaaaaataattcatgatgctattactaaagtagatgacaagtatttcaagtcatttattgactttgcaaatcataaagtaacagaagatctcatcccaacagcagacatgaagaaggatacactttgtcctaatttggaagttgatagttggttaaggtttccattctatgacttggattttggaacaggttgcccttttgttttcatgccttcttattatccaactgaaggtatgatgtttcttgtgccatctttcattggagatggaagtattgatgctttcattcctctttaccaagacaattcgcctaccttcaagaaaatttgctattctcttgatttgaaagcaaaatga

SEQ ID NO: 8 - protein, 438 aa, Nicotiana attenuata
MNVKIESSRIIKPFYEGTPPSTNTHISFNVFDNVTYDALMALIYAYRPPTPPTSTIEMGLRKTLAVYREWAGRIGRDENGNRVVFLNDEGVRFIEASVNATLDEVLPLKPSPSLLKLHPGMKDVVELIQVQVTRFTCGSVMVGFTGHHMIADGHAASNFFVAWGQACRGVEITPLPLHDRAIFHPRNPPLIEFNHVGAEFMSKSLNKKEFIKLENTEKNIIVHKVHFTLEFLGKLKANASFMNGKTKTYSTFESLVAHLWRVITKARELDGSQNTQIRISVDGRRRVVPRVADEFFGNIVLWAFPTSKVRDLVNEPLHYATKIIHDAITKVDDKYFKSFIDFANHKVTEDLIPTADMKKDTLCPNLEVDSWLRFPFYDLDFGTGCPFVFMPSYYPTEGMMFLVPSFIGDGSIDAFIPLYQDNSPTFKKICYSLDLKAK

SEQ ID NO: 9 - DNA, 1302 bp, Nicotiana attenuata
  source 1..1302: mol_type = other DNA
atggccggccaaacaatcatcgtttccgggttgaacccggcggccattcttcagtccacaattggcggcggagcttctcctacagcggcggcggcggcggaaaacggcaccagaaaagtcatccctctttcaagagatgccttacaagatttcatgttatcaatcataacccaaaaattacaagatgagaaacaacctttttacgtgttagatttgggtgaggttgtttatcttatggaccaatggaaatctgctctcccaaatatccgtccattttacgctgttaaatgtaaccctgaaccgtcgttcctttcaattttatctgctatgggctcaaattttgattgtgctagccgtgctgaaattgagtgcgttttagcccttggcatttcacctgaccgtattgtttttgcaaatccatgcaaaccggaatccgatattatttttgcagctaaagttggggtgaatttgacaacctacgattccgaagatgaggtttacaagatccgaaagcatcacccaaaatccgaactcttgctccgaatcaagcccatgttcgacggcaacgcgagatgtccaatgggcccaaaatacggcgcgcttccagaagaagtcgaaccgctgctccgcgcagctcaggccgcccggctcaccgtctccggcgtctccttccacatcggaagcggagacgccgattcgaacgcttatctcggcgccatagccgcggctaaggaagtgtttgaaacagctgctaaactcgggatgtcgaaaatgactgttctagacgtcggcggcgggtttacatccggccaccagttcacaaccgccgccgctgccgttaaatcagctttaaaacaacgcttcgatgacgaaccggagttgacaatcatagctgaaccgggtcggttttttgcagagacggcgtttactttggcaacgacgattatagggaaaagagtgaggggtgaattgagggagtattggattaacgacggactgtacggttcgatgaactgtgtactttacgaccatgcgacggtgaatgcaacgccgttagctgttcagtcgaatcgtagtaacgtcacctgcggcgggtcgaaaacgtttccgacgactgtgtttgggcccacttgtgatgctcttgatactgttttaagggattaccagttaccggagctgcaggttaatgattggctggtttttcctaatatgggcgcttatactaaagcagctgggtccaattttaatggatttaatacttccgccattgttactcacctcgcttatgcttatccaagctga

SEQ ID NO: 10 - protein, 433 aa, Nicotiana attenuata
MAGQTIIVSGLNPAAILQSTIGGGASPTAAAAAENGTRKVIPLSRDALQDFMLSIITQKLQDEKQPFYVLDLGEVVYLMDQWKSALPNIRPFYAVKCNPEPSFLSILSAMGSNFDCASRAEIECVLALGISPDRIVFANPCKPESDIIFAAKVGVNLTTYDSEDEVYKIRKHHPKSELLLRIKPMFDGNARCPMGPKYGALPEEVEPLLRAAQAARLTVSGVSFHIGSGDADSNAYLGAIAAAKEVFETAAKLGMSKMTVLDVGGGFTSGHQFTTAAAAVKSALKQRFDDEPELTIIAEPGRFFAETAFTLATTIIGKRVRGELREYWINDGLYGSMNCVLYDHATVNATPLAVQSNRSNVTCGGSKTFPTTVFGPTCDALDTVLRDYQLPELQVNDWLVFPNMGAYTKAAGSNFNGFNTSAIVTHLAYAYPS

SEQ ID NO: 11 - DNA, 1197 bp, Nicotiana attenuata
  source 1..1197: mol_type = other DNA
atgttattatctacagctatcggtaagagtcatgttcccaaaataccaaaagatgggatgccagatttaatccgttcaattgcagagaaccatgaagctggccagccattttatctacttgatttggctataattgaaaggcttatggacaaatggaaccattattttccaaatataaaacctttttatgctgtgaaatgcaacactgaacctgctcttcttactaaactagccaaattgggtgctaattttgattgtgctagccaactagaaatagaaaccgtcttaaatctcggaattagcccaaaccaaatcatatttgctaacccatgcaaagctatttcccacatcaaatacgcagccaatgttggggtcaatctcacaacttttgattctaaatttgaaattgacaagatcaagaaatggcacccacattgtcatttgttgctacgaattaaagcccctagtgatagtggcgcgttacgtcccctggggaagaaattcggcgtgttaccagaagaagttgagccactactgcattacgcttataatgtggtagggttaaaagttgtaggcgtttcatttcacgttggatctatagcacaagatcctagcatttatcgcaaggcgattgcagttgctaggaccgtgtttgatgttgttgatcatcttcgaatgccaaaaatgcaaattttaaatattggtggaggatttagatcaacaccattgttcgaagaaatagctagtgtagtaaatgaagcagtccaagattatttttccatgcctaatttaacaatatttgcagaaccaggacggttttttgcagaaacagcctttacgttagtcactcatgtgattggtaaaagagttagaggtgagaaaatagagtattggattgatgaagggatttatggatcatttaggccaacactttataatagttgttttgtgggtataaagccattgttacgtcaggtaacagaaaaatctagtcaaatatgtgagtcaactatttatggaccaagttgtgactcacttgatgcagtggctattgacataaaattgccagagcttcatttggatgatctgatagtgttttataatatgggcgcatattcaatatgtggaggaactaagttcaatggatttgatatgttatctacacctacctatcttgttaactcaaattcaagctaa

SEQ ID NO: 12 - protein, 398 aa, Nicotiana attenuata
MLLSTAIGKSHVPKIPKDGMPDLIRSIAENHEAGQPFYLLDLAIIERLMDKWNHYFPNIKPFYAVKCNTEPALLTKLAKLGANFDCASQLEIETVLNLGISPNQIIFANPCKAISHIKYAANVGVNLTTFDSKFEIDKIKKWHPHCHLLLRIKAPSDSGALRPLGKKFGVLPEEVEPLLHYAYNVVGLKVVGVSFHVGSIAQDPSIYRKAIAVARTVFDVVDHLRMPKMQILNIGGGFRSTPLFEEIASVVNEAVQDYFSMPNLTIFAEPGRFFAETAFTLVTHVIGKRVRGEKIEYWIDEGIYGSFRPTLYNSCFVGIKPLLRQVTEKSSQICESTIYGPSCDSLDAVAIDIKLPELHLDDLIVFYNMGAYSICGGTKFNGFDMLSTPTYLVNSNSS

SEQ ID NO: 13 - DNA, 1491 bp, Nicotiana attenuata
  source 1..1491: mol_type = other DNA
atgtccacactcatggcgaaaatgatgagcggatctactcctactaaccctggtagcactggtacatcctcaccgtcgctaactccgccgccggcttctcttcccgtccgtacaattcccggcggctacggttggcctttgttaggaccaatatctgatagattggactataactggttccagggacctaacactttttttaccaagagaattgaaaagcacaagagcactgtttttagaacaaatgtgcctccttgttttccgttttttctgggtgttaatccgaatgtggtggcggttcttgacgtcaaatcgttttcacatctgtttgatatggagattgtagagaaagctaatgttcttgttggggatttcatgcctagtgttaagtatactggagatatgcgtgtttgtgcttatcttgatacttctgaacctaaacatactcagataaagaacttttcattggacattctaaaaagaagctcaaaaacatgggtgccaacacttgtcaatgagctcaactccatgtttgaaacttttgaatcagatatctcaaaatcaaactcagcttctcttctccctactatgcaaaaattcctcttcaactttttctccctctctctcttaggagccaatccatcagcttcaccagaaatagccaattctggctacgtaatgcttgacacgtggctagctattcaacttgcacctactgttagtattggtctacttcaaccccttgaagaaatatttgtccactcttttaattacccttttttccttgtcaaaggtagttatgaaaaactcatacaatttgtcaaaaatgaagctaaggaagttttaaatagaggaaaatcagagtttggacttactgaacaagaagctatacataaccttttgttcattcttgggttcaatgcttttggtggtttctctattttcttgccaaccctattgggaaatcttggagatgaaaaaaatgcagaattacaagagaagttaagaaatgaagtgagagagaaagttggattaaagacagaaaatttgagttttgagagtgttaaagaaatggaacttgttcaatcttttgtatatgaaacacttagacttagtccacctgtgccaagtcaatatgcaagagcaagaaaagattttaaactaagttcacatgattcagtatatgaaatcaagaaaggtgaacttctttgtggatatcagccattggtaatgagagatccaaaggtgtttgatgatcctgaaaagtttgtgttggaaaggtttacaaaggaaaaagggaaagaattgctgaattatttgttttggtcaaatggaccacagactgggagaccaactgaatcaaacaaacaatgtgcggctaaggatgttgttactcttactgcttctttgattgtggcttatatttttcaaaggtatgattcagtgagtttctcttctggttcactcacatctgtgaaaaaagccagttga

SEQ ID NO: 14 - protein, 496 aa, Nicotiana attenuata
MSTLMAKMMSGSTPTNPGSTGTSSPSLTPPPASLPVRTIPGGYGWPLLGPISDRLDYNWFQGPNTFFTKRIEKHKSTVFRTNVPPCFPFFLGVNPNVVAVLDVKSFSHLFDMEIVEKANVLVGDFMPSVKYTGDMRVCAYLDTSEPKHTQIKNFSLDILKRSSKTWVPTLVNELNSMFETFESDISKSNSASLLPTMQKFLFNFFSLSLLGANPSASPEIANSGYVMLDTWLAIQLAPTVSIGLLQPLEEIFVHSFNYPFFLVKGSYEKLIQFVKNEAKEVLNRGKSEFGLTEQEAIHNLLFILGFNAFGGFSIFLPTLLGNLGDEKNAELQEKLRNEVREKVGLKTENLSFESVKEMELVQSFVYETLRLSPPVPSQYARARKDFKLSSHDSVYEIKKGELLCGYQPLVMRDPKVFDDPEKFVLERFTKEKGKELLNYLFWSNGPQTGRPTESNKQCAAKDVVTLTASLIVAYIFQRYDSVSFSSGSLTSVKKAS

SEQ ID NO: 15 - DNA, 1584 bp, Nicotiana attenuata
  source 1..1584: mol_type = other DNA
atggcatcatcttcttttgctcttccttctcttaagctacaattccccccacataaatcatcatcatcatctcgtaacaagtactcatcctctcttcggcctattaaatcttcggtgtctgaaaagccaccttacatttcgtctccagggacttctccgatccaatctaaacctacaaaactcccgatccgaaaagtccctggagactacgggctaccattagtcggtccgtggaaggaccgacttgatttcttttacaatcaaggtagagatgagtttttcaaatctcgaattcagaaacatcagtctactgtttttcgaaccaacatgccacctggtcctttcatttccttcaatcccaacgttgtcgttttgctggacggcaaaagtttccccaccctttttgatacttctaaagtcgagaaaaaagatcttttcaccggcacttttatgccttctactgagcttactggtggttaccgtgttctctcctatcttgatccttctgaaccaaatcatgccaaattaaagaaactcatgttctttctcctttcatctcgacgcaatgaagtaatcccagaatttcacaacagctattccgagctttttgagacactagaaaatgacttagccactaaaggaaaagctgggttaaacgcagcaaatgatcaagcagcatttaactttcttgctcgatctttttacggagttaatccgttagacaccaatcttggagctgatggacctaagttaattggcaaatgggtgttgtttcagcttcatcctttgctaattcttggccttcctaaagttcttgaagatcttgtccttcatacttttaggctgccttctgctttagtgaaaaaagactaccaaagattgtacaatttcttctacgacaactcaacttccattcttgatgaagctgaaagaactggtatttcacgtgaggaagcttgtcacaatttgctatttgcaacgtgtttcaattcttttggagggatgaaaatattttttcccaatatgttgaaatggattggtcgagctggggttaaactccacactcaattagctcaagagattcggtcaggcattaaatcaaacggtggaaagatcacaatgggtgcaatggaaaacatgccattaatgaaatctgtagtttatgaatccctacgtattgaaccgccagtgacatcacaatacggcagagcaaaacgtgatatggtgattgaatcccacgacggagcatttgagattaaagaaggtgagttactatatgggtttcaaccatttgcaactaaggacccgaaaatttttgaccggtctgaagagtttgtagcggaccggtttattggagaagaaggggagaagttattgaaacatgtgttgtggtctaatggaccggaaaatgaaagcccgtcaataaataacaagcagtgtgtaggaaaagattttgtggttttggtttcaaggttgttgttggttgaattgttcatgagatatgactcgtttgagattgaggttggtgcttctcctttaggtgcttctattaccttaacgtctttgaaaagagccagcttttga

SEQ ID NO: 16 - protein, 527 aa, Nicotiana attenuata
MASSSFALPSLKLQFPPHKSSSSSRNKYSSSLRPIKSSVSEKPPYISSPGTSPIQSKPTKLPIRKVPGDYGLPLVGPWKDRLDFFYNQGRDEFFKSRIQKHQSTVFRTNMPPGPFISFNPNVVVLLDGKSFPTLFDTSKVEKKDLFTGTFMPSTELTGGYRVLSYLDPSEPNHAKLKKLMFFLLSSRRNEVIPEFHNSYSELFETLENDLATKGKAGLNAANDQAAFNFLARSFYGVNPLDTNLGADGPKLIGKWVLFQLHPLLILGLPKVLEDLVLHTFRLPSALVKKDYQRLYNFFYDNSTSILDEAERTGISREEACHNLLFATCFNSFGGMKIFFPNMLKWIGRAGVKLHTQLAQEIRSGIKSNGGKITMGAMENMPLMKSVVYESLRIEPPVTSQYGRAKRDMVIESHDGAFEIKEGELLYGFQPFATKDPKIFDRSEEFVADRFIGEEGEKLLKHVLWSNGPENESPSINNKQCVGKDFVVLVSRLLLVELFMRYDSFEIEVGASPLGASITLTSLKRASF

SEQ ID NO: 17 - DNA, 1560 bp, Nicotiana attenuata
  source 1..1560: mol_type = other DNA
atggcagtagcaacagcaacagcaacattatcttcatcttcttcacttcccttccattctcttcatcaacagtttccttcaaaatacttcactgttcgtcccattacagtctctttatccgaaaaaataccagcagtaacacagtcgtctgagttcacaaaattaccgatccgtacaattcctggcgattatggtcttcctttaattggtccatggaaagatagacaagattatttttataatcaaggtaaagaagaattcttcagatcaagaattcaaaaatacaaatctactgtatttaaaaccaatatgccacctggaaatttcatttcttccaatccaaacgttgtcgttttgctcgacggcaagagttttcccaccctcttcgacgtttctaaagtcgagaaaaaggatctcttcaccggaactttcatgccgtcgactgaactcaccggcggttaccgtgttctttcttatcttgatccttctgaacctacccatgaaaaattaaaaaagctactcttttttcttctttcttctcgtcgtgattatataataccccagtttcacgaaagctatacggagctgtttaaaaccctagaaaaggaaatggagaaaaacggtaaagctgatttaaactcggctaatgatcaagctgcttttaatttcttagctagatcattgtacggagcgaacccagttgagactaagctcggaactgatggtcccacattgatcggcaaatgggtgttgtttcagcttcatcctttgctcactcttggtcttccgaaggttctagacgactttctcctccataatttccggttaccgccagctctggtcaagaaagattaccagagactctacgatttcttctatgagagttccactgctgtgctaaacgaagctggaaattttggtatttcgcgcgacgaagcttgtcataatcttctcttcgctacgtgtttcaattcctttggcgggatgaagattttctttcctaatatgctgaaatggatagctagagcaggagtagagctccatatccggttagcgaacgagatccgatccgccgtcaaatccgccggcgggaagatcacgatgtcggcgatggagaaaatgcctgtgatgaaatcggttgtatacgaggctttacgaattgatccaccagttgcttctcaatacggaagagccaaacgtgaccttatgatcgaatcacacgatggcgtttttgaggtgaaaaaaggcgaaatgttattcgggtaccaaccgtttgcgactaaggatccgaaaatatttgaccgacccgatgagttcgtacccgatcggttcgtaggtgaagaaggggaaaagctattgaaacatgtattatggtctaacgggccggaaacggagagtccgacggtggagaataaacagtgtgctggaaaggatttcgtggtgctggtttcgagattgttggtaacggagtttttcctccgttatgacacgttggatatcgacgtcggcacgtcgcctttaggagctaagattactataacttctttgaagagagcttag

SEQ ID NO: 18 - protein, 519 aa, Nicotiana attenuata
MAVATATATLSSSSSLPFHSLHQQFPSKYFTVRPITVSLSEKIPAVTQSSEFTKLPIRTIPGDYGLPLIGPWKDRQDYFYNQGKEEFFRSRIQKYKSTVFKTNMPPGNFISSNPNVVVLLDGKSFPTLFDVSKVEKKDLFTGTFMPSTELTGGYRVLSYLDPSEPTHEKLKKLLFFLLSSRRDYIIPQFHESYTELFKTLEKEMEKNGKADLNSANDQAAFNFLARSLYGANPVETKLGTDGPTLIGKWVLFQLHPLLTLGLPKVLDDFLLHNFRLPPALVKKDYQRLYDFFYESSTAVLNEAGNFGISRDEACHNLLFATCFNSFGGMKIFFPNMLKWIARAGVELHIRLANEIRSAVKSAGGKITMSAMEKMPVMKSVVYEALRIDPPVASQYGRAKRDLMIESHDGVFEVKKGEMLFGYQPFATKDPKIFDRPDEFVPDRFVGEEGEKLLKHVLWSNGPETESPTVENKQCAGKDFVVLVSRLLVTEFFLRYDTLDIDVGTSPLGAKITITSLKRA

SEQ ID NO: 19 - DNA, 2154 bp, Nicotiana attenuata
  source 1..2154: mol_type = other DNA
atggagtatgccaatggaaattgtaatggtcatgttagtcaagaattatgtattaagggtcatgaacaacaagaccctttgaattggaacatggcagctgatgcactcaagggtagccaccttgatgaagtcaagaaaatggtggaggagtttaggaagccggtggtccggctaggtggagagacgttaacgataggtcaggtggccgcgatagcaggcaggtcgggcacagaggtgatggtggagctggcggaggcggctagggcgggcgtcaaggccagcagtgattgggtcattgaaggcatgaaaaatgggactgatagctatggtgtaactactggttttggtgctacatcacataggagaaccaagcaaggtgcagctctacagaaggagctaataaggttcttgaatgccggaatctttggcaatgggacggagtcttgccacacgctgccacactccgccacgagggccgccatgttggtgaggatcaacaccctactccaaggctactccggcataagatttgagatcttagaagctataacaaaattgctcaacaataacatcaccccatgtttgcctctccgtggcacaatcacagcatctggtgacttggtaccactgtcctacattgctgggcttttaacgggccggcccaattccaaagcaactggacctaatggagagttgttggatgctaacaaggcgtttcaacgggccggtatagacactggattctttgagttgcagcccaaagaaggcttagctttggttaatggcactgctgttggttctggtttggctgctatggttctttttgacgctaacattcttgctgttctttcggaagttttatcagctatttttgctgaagttatgcaaggaaagcctgagtttacagaccatttaacacacaaattgaagcatcaccctggtcaaatagaagctgcagctataatggaacatattttggaaggaagttgttatgttaaggaagctaagaaaatccatgaaatggatcctttacaaaagcctaagcaagatcgttacgctctacgtacatctccacaatggttaggtcctcaaattgaggtcattagatcagcaacaaaaatgattgaaagagagattaactcagttaatgacaaccctttgattgatgtttcaagaaacaaggcattacatggtggaaatttccaagggacaccaataggtgtatcaatggacaacactaggctagcacttgcctcgattgggaaacttatgtttgcacaattttctgaacttgtcaatgactattacaacaatggattaccttcaaatttgaccgggggacggaatcctagcttggattatggtttcaaaggtgctgaaattgccatggcatcttactgttctgaactccaatttcttgcaaatccagtgactaaccatgtccaaagtgctgagcaacacaaccaagatgtcaactctttgggcttaatctcctcaagaaaaacagctgaggcggtcgacattttgaaactcatgtcttctacttatctagtagcactttgccaagccattgacttgagacacttggaagaaaatctcaaggcttcagtgaaaagtgcagtgagtttagtggccaagaaagtgctgataactggtcaaaatggagggcttcaccaatcaagattcagtgagaaagacttgctcacagtggttgatagagaatattgttttgcatatgctgatgacccttgcagtgctaactatccattaatgcagaagttaagacaagtcttagtggatcatgctctatcgaatggcgacgcggagaagaattctagcacttcaattttccataaaatcaaagcatttgaggaagaattgaagattgttttgcctaaggaaattgagagtgctagatgtaacttggagaatggaaagccagcaattcctaataggattcaagaatgcaggtcatatccattgtacaagtttgtgagggaagaattaaagactaattatttgacaggtgagaaagtccaatcacctggagaagaatttgacaaagtattcacagctatgaatggagggaagttagtagatccattgcttaattgcttgaagaaatggaatggtgctcctcttccactttgctag

SEQ ID NO: 20 - protein, 717 aa, Nicotiana attenuata
MEYANGNCNGHVSQELCIKGHEQQDPLNWNMAADALKGSHLDEVKKMVEEFRKPVVRLGGETLTIGQVAAIAGRSGTEVMVELAEAARAGVKASSDWVIEGMKNGTDSYGVTTGFGATSHRRTKQGAALQKELIRFLNAGIFGNGTESCHTLPHSATRAAMLVRINTLLQGYSGIRFEILEAITKLLNNNITPCLPLRGTITASGDLVPLSYIAGLLTGRPNSKATGPNGELLDANKAFQRAGIDTGFFELQPKEGLALVNGTAVGSGLAAMVLFDANILAVLSEVLSAIFAEVMQGKPEFTDHLTHKLKHHPGQIEAAAIMEHILEGSCYVKEAKKIHEMDPLQKPKQDRYALRTSPQWLGPQIEVIRSATKMIEREINSVNDNPLIDVSRNKALHGGNFQGTPIGVSMDNTRLALASIGKLMFAQFSELVNDYYNNGLPSNLTGGRNPSLDYGFKGAEIAMASYCSELQFLANPVTNHVQSAEQHNQDVNSLGLISSRKTAEAVDILKLMSSTYLVALCQAIDLRHLEENLKASVKSAVSLVAKKVLITGQNGGLHQSRFSEKDLLTVVDREYCFAYADDPCSANYPLMQKLRQVLVDHALSNGDAEKNSSTSIFHKIKAFEEELKIVLPKEIESARCNLENGKPAIPNRIQECRSYPLYKFVREELKTNYLTGEKVQSPGEEFDKVFTAMNGGKLVDPLLNCLKKWNGAPLPLC

SEQ ID NO: 21 - DNA, 2154 bp, Nicotiana attenuata
  source 1..2154: mol_type = other DNA
atggcatcaaatggtcatgttaatggaggagaagactttgagttgtgcaagaaatcatcagccactgatccattgaattgggaaatggcgtctgaatctttaagagggagtcatttggatgaagtgaaaaaaatggtgagtgaatttagaaaaccaatggtaaaacttggtggtgaaactttaacagtggcacaagtggctgctattgcagttagggacaaaagtgcaaatggtgttaaagttgaactttctgaagatgcaagagctggtgttaaagctagtagtgattgggttatggatagtatgaataaaggaacagatagttatggtgttactactggttttggtgctacttctcatagaagaaccaagaatggtggtgctcttcaaaaagaacttattaggttcttgaatgctggtgtttttggaaatggaacagaaacaaaccacacattgccacattcagcaacaagggcagcaatgcttgtaaggatcaacacactcttacaaggatactctggcatcagatttgaaatcttggaagcaattacaaaattgattaacagcaacatcactccatgtttacctctccgtggcacgatcactgcctcgggcgatcttgtcccgttatcctacattgctggtttgctcactggtaggcctaattccaaagctgttggtcccaatggtgagacacttaatgctgaagaagcgttccgcgttgctggtgttaatggtggatttttcgagttgcagcctaaggaaggacttgctcttgtaaatggtacagcagttggttctggtatggcatcaatggttctctttgattccaacattcttgctgttatgtctgaagttttatcagcgattttcgctgaagtaatgaatggaaagcccgaattcactgaccatttgacacacaagttgaagcaccaccctggtcaaattgaggctgctgctattatggaacacattttggatggaagctcttatgtgaaggcggctcaaaagctacatgaaatggatcctctccaaaaaccaaagcaagatcgttatgctctccgaacatctccacaatggcttggccctcaaattgaagtcattcgctctgcaactaagatgattgagagggagattaactcagtgaacgataaccccttgatcgatgtttcaagaaacaaggcgttacatggtggcaacttccagggaacccctatcggcgtctccatggataatgcaagattggctcttgcatcaattgggaaattgatgtttgctcaattctcggaacttgtcaacgactattacaataacggtttgccatctaatctcaccgcatcaagaaatccaagcttggactatggtttcaagggagctgaaatcgcgatggcttcttactgctcagaacttcaattcttggcaaatccagtgaccaaccatgtccaaagtgctgagcaacacaaccaagatgtcaactccttaggcttaatctcagcaaggaaaacagctgaagctgtcgatatcttgaagctcatgtcatcaacttatctagtggcactttgccaagctatcgacttgagacatttggaggaaaacttaaagaatgcagtcaagaatacagttagccaagtagctaagagaactcttacaatgggtgctaatggtgaacttcatccagcaagattctgtgaaaaggaattgcttcgagtcgtggatagggaatacttgttcgcctacgctgatgatccttgcagttgcaactaccctttaatgcagaaactgagacaagttcttgttgatcatgcaatgaagaatggtgaaagtgagaagaatgtgaacagctcaatcttccaaaagattggagctttcgaagacgaattaaaggctgttttacctaaagaagtggagaatgcaagagctgcattggagagtggcaaccctgctattgctaacaggattacagaatgcagatcttatccattgtacaggtttgtgagaaaggagcttggaacagaattgttgacaggagaaagagtccgatcaccgggcgaggagtgtgacaaggtgttcacagcaatgtgcaatggacagatcattgatccaatgttggagtgtctcaagagctggaatggtgctcctcttcctatctgttag

SEQ ID NO: 22 - protein, 717 aa, Nicotiana attenuata
MASNGHVNGGEDFELCKKSSATDPLNWEMASESLRGSHLDEVKKMVSEFRKPMVKLGGETLTVAQVAAIAVRDKSANGVKVELSEDARAGVKASSDWVMDSMNKGTDSYGVTTGFGATSHRRTKNGGALQKELIRFLNAGVFGNGTETNHTLPHSATRAAMLVRINTLLQGYSGIRFEILEAITKLINSNITPCLPLRGTITASGDLVPLSYIAGLLTGRPNSKAVGPNGETLNAEEAFRVAGVNGGFFELQPKEGLALVNGTAVGSGMASMVLFDSNILAVMSEVLSAIFAEVMNGKPEFTDHLTHKLKHHPGQIEAAAIMEHILDGSSYVKAAQKLHEMDPLQKPKQDRYALRTSPQWLGPQIEVIRSATKMIEREINSVNDNPLIDVSRNKALHGGNFQGTPIGVSMDNARLALASIGKLMFAQFSELVNDYYNNGLPSNLTASRNPSLDYGFKGAEIAMASYCSELQFLANPVTNHVQSAEQHNQDVNSLGLISARKTAEAVDILKLMSSTYLVALCQAIDLRHLEENLKNAVKNTVSQVAKRTLTMGANGELHPARFCEKELLRVVDREYLFAYADDPCSCNYPLMQKLRQVLVDHAMKNGESEKNVNSSIFQKIGAFEDELKAVLPKEVENARAALESGNPAIANRITECRSYPLYRFVRKELGTELLTGERVRSPGEECDKVFTAMCNGQIIDPMLECLKSWNGAPLPIC

SEQ ID NO: 23 - DNA, 2139 bp, Nicotiana attenuata
  source 1..2139: mol_type = other DNA
atggccggtgttgcacaaaatggtcaccaagaaatggatttttgcgttaaagtggatccattaaactgggaaatggcagctgattcattgaaaggtagccatttagatgaagtgaagataatggtggctgagtataggaaaccagtagtgaaacttggaggtgagactttaacagtggctcaagttgcggctattgctgcaaaagataatgctaaaactgtaacagtggagctttctgaaggggcaagagctggtgttaaagctagtagtgattgggttatggatagtatgagtaaaggaactgatagttatggtgttactactggttttggtgctacttcacatagaaggacaaagaatggtggtgctcttcaaaaagaacttattaggttcttgaatgctggagtttttggtaatggaacagagtcatgtcacacattaccacaatcagggacaagggcagctatgctagttaggatcaacacactcctacaaggatattctggcatcagatttgaaatcttggaagcaatcaccaaattgcttaaccacaacgtcactccatgtttgccccttcgcggcacaatcactgcctctggtgatctcgtccccttgtcctacattgccggtttactcacaggtcggcctaattccaaagccgttggacctaatggtgagaccctcaatgctgaagaagcgttccgtgttgctggagttaacggtggatttttcgagttgcaacctaaggaaggccttgcccttgtgaatggcactgcagttggttctggtttggcctcaatggttctctttgatgctaacattctcgcggtcttttctgaagttctctcagctattttcgctgaggtaatgaatggaaagcccgagttcactgaccacttgacacacaagttgaagcatcaccccggacaaattgaggctgctgctattatggaacatattttggatggtagctcttatgtgaaggcggctcagaagcttcacgaaacggatcctcttcaaaaaccaaagcaagatcgttacgccctcagaacatcaccccagtggctcggccctcaaattgaggtcatccgttctgcaaccaagatgattgagagagagattaactcagtgaacgacaaccctttgatcgatgtttcaagaaacaaggcgttacacggtggtaatttccagggcacaccaattggtgtctctatggacaatgctagattagcccttgcctcaattgggaaattgatgtttgcccaattctccgagcttgtcaacgattactacaacaacgggttgccatctaatctgacagcaggaaggaatccaagcttggactatggtttcaagggatctgagattgccatggcttcatactgttcagaacttcaattcttggcaaatccagtgactaaccacgtacaaagtgccgagcaacacaaccaagatgtgaactccttgggcttaatctcagctagaaaaacagctgaagccgttgacatcttgaagctaatgtcatccacatatctagtcgcactctgccaagcgatagacttgaggcatttggaagaaaatctgaggaatgcagtcaagaacacggtgagccaagtcgctaagaggaccttgacaatgggtgccaatggagaacttcatccatcaagattctgtgaaaaggacctgcttcgagtcgtggacagggaatacgtctttgcatacgctgatgacgcctgcagcgccaactacccactgatgcagaaactgaggcaagtactcgtcgaccacgctttacaaaatggcgaaaatgagaagaacgcgaacagctctatcttccaaaagatactagcttttgaagacgagttaaaggccgtgttgccaaaagaagtcgagagtgcaagaatcgcgcttgaaagcggtaaccctgcaattgccaacaggataaaggaatgcagatcttatccactctacagatttgttagagaagaacttggagctgaattattgacaggagaaaaagtcaggtcaccaggtgaagaatgtgacaaagtgtttacagcaatgtgcaatggacaaataattgattcattgttagaatgtctcaaggaatggaatggtgctcctctaccaatctgttag

SEQ ID NO: 24 - protein, 712 aa, Nicotiana attenuata
MAGVAQNGHQEMDFCVKVDPLNWEMAADSLKGSHLDEVKIMVAEYRKPVVKLGGETLTVAQVAAIAAKDNAKTVTVELSEGARAGVKASSDWVMDSMSKGTDSYGVTTGFGATSHRRTKNGGALQKELIRFLNAGVFGNGTESCHTLPQSGTRAAMLVRINTLLQGYSGIRFEILEAITKLLNHNVTPCLPLRGTITASGDLVPLSYIAGLLTGRPNSKAVGPNGETLNAEEAFRVAGVNGGFFELQPKEGLALVNGTAVGSGLASMVLFDANILAVFSEVLSAIFAEVMNGKPEFTDHLTHKLKHHPGQIEAAAIMEHILDGSSYVKAAQKLHETDPLQKPKQDRYALRTSPQWLGPQIEVIRSATKMIEREINSVNDNPLIDVSRNKALHGGNFQGTPIGVSMDNARLALASIGKLMFAQFSELVNDYYNNGLPSNLTAGRNPSLDYGFKGSEIAMASYCSELQFLANPVTNHVQSAEQHNQDVNSLGLISARKTAEAVDILKLMSSTYLVALCQAIDLRHLEENLRNAVKNTVSQVAKRTLTMGANGELHPSRFCEKDLLRVVDREYVFAYADDACSANYPLMQKLRQVLVDHALQNGENEKNANSSIFQKILAFEDELKAVLPKEVESARIALESGNPAIANRIKECRSYPLYRFVREELGAELLTGEKVRSPGEECDKVFTAMCNGQIIDSLLECLKEWNGAPLPIC

SEQ ID NO: 25 - DNA, 2139 bp, Nicotiana attenuata; identical to SEQ ID NO: 23
  source 1..2139: mol_type = other DNA

SEQ ID NO: 26 - protein, 712 aa, Nicotiana attenuata; identical to SEQ ID NO: 24

SEQ ID NO: 27 - DNA, 1521 bp, Nicotiana attenuata
  source 1..1521: mol_type = other DNA
atggatcttctccttctagagaagacccttataggcttattctttgctatcattgtagcaatagttgtttctaagttacgtagcaagaaatttaagctgcccccaggtccaattcctgtgccaatttttggcaattggcttcaagttggtgatgatttgaatcacagaaacctcactgaatatgccaagaaatttggtgacatgttcttgttaagaatgggacagaggaatcttgtggttgtatcatcccctgaattagcgaaagaagttttgcatacacaaggggttgaatttggatcaagaactaggaatgtggtgtttgatattttcaccggaaaaggccaagatatggtttttacagtatatggtgaacactggaggaagatgaggaggattatgacagtcccattttttacaaacaaagtggtgcagcagtataggcgtgggtgggaagatgaagtggcacatgttgttgaggatgtgaagaaaaatccagagtcagcaactaatgggattgtgttgaggaaaaggttgcagcttatgatgtacaataacatgtacaggattatgtttgataggaggtttgagagtgaggatgatcctttgtttaacaagctcaaggctttgaatggagagaggagtagattggctcagagttttgagtacaattatggtgattttatccctattttgagacctttcttgagaggttacttgaacatctgtaaggaagttaagcagaggaggttgcagcttttcaaagattactttgttgatgaaagaaagaaacttgcaaacacaacgaagagcatggacaatagtgcgctaaagtgtgccattgatcacattcttgaggctgaacagaagggagagatcaatgaggataatgtcctttacattgttgagaacatcaatgttgctgcaatagaaactacactgtggtcaattgagtggggtattgctgaactagtgaaccaccctgaaatccagaagaaactccgtgacgagattgacagtgttcttggagtaggagtgcaaatcactgagccagaactcaacaagcttccttaccttcaggctgtgatcaaggagacccttcgtctccgtatggcaatccctcttttagtcccacacatgaaccttcacgatgcgaagcttgctggatatgacattcccgcggagagcaagatcctggtaaacgcttggtggctggctaacaaccccgctacctggaagaagcccgaagagtttaggccagagaggttcttcgaagaggagaagcacgttgaggctaatggcaatgacttcagatatcttccatttggtgttggtaggaggagctgccctggaattatccttgcattgccaattcttggcattactttgggacgcttggtgcagaactttgagctgttgcctcctccaggacagtcaaagcttgacacaacagagaaaggcgggcaattcagtctgcacattttgaagcattccaccattgtgatgaaaccaagatctttttaa

SEQ ID NO: 28 - protein, 506 aa, Nicotiana attenuata
MDLLLLEKTLIGLFFAIIVAIVVSKLRSKKFKLPPGPIPVPIFGNWLQVGDDLNHRNLTEYAKKFGDMFLLRMGQRNLVVVSSPELAKEVLHTQGVEFGSRTRNVVFDIFTGKGQDMVFTVYGEHWRKMRRIMTVPFFTNKVVQQYRRGWEDEVAHVVEDVKKNPESATNGIVLRKRLQLMMYNNMYRIMFDRRFESEDDPLFNKLKALNGERSRLAQSFEYNYGDFIPILRPFLRGYLNICKEVKQRRLQLFKDYFVDERKKLANTTKSMDNSALKCAIDHILEAEQKGEINEDNVLYIVENINVAAIETTLWSIEWGIAELVNHPEIQKKLRDEIDSVLGVGVQITEPELNKLPYLQAVIKETLRLRMAIPLLVPHMNLHDAKLAGYDIPAESKILVNAWWLANNPATWKKPEEFRPERFFEEEKHVEANGNDFRYLPFGVGRRSCPGIILALPILGITLGRLVQNFELLPPPGQSKLDTTEKGGQFSLHILKHSTIVMKPRSF

SEQ ID NO: 29 - DNA, 1629 bp, Nicotiana attenuata
  source 1..1629: mol_type = other DNA
atggagaaagatacaaaacacggcgatataattttccgatcaaaactccctgatatatacatccctaatcaccttcctttacattcctactgtttcgaaaacatttccgagttcagttctcgtccttgtttaatcaatggagccaacaaacaaatttatacgtatgctgatgttgaactcaattcaagaaaagttgcagctggccttcacaagcaagggattcaacaaaaagatacaatcatgatcctattgcctaactccccagaatttgtgtttgctttcattggtgcatcgtaccttggagctatttctacaatggccaatcctttgtttactcctgctgaggttgtgaagcaagccaaggcttctagtgctaagatcattgtcacacaagcgtgtcatgttaacaaagtgaaagattatgcatttgagaatgatgtgaagatcatatgcatcgactccgccccggagggctgtctccatttctccgtgctaactcaggccgatgagcacgatattccggagatagaaatccaacccgacgatgtggtggcgttgccgtactcctccgggacgacgggattacctaaaggagtgatgttgacgcacaagggacttgtcacaagcttggcacaacaagttgatggtgaaaatccgaatttgtatatccatagcgaggacgtgatgctttgtgtgttgcccttatttcatatctattccctcaactccgttttgctttgtggattaagggttggagcagcgattttgattatgcagaaatttgatattgttccgttcttggagttgatacaaagttacaaggtgacaatagggccgtttgtaccacctattgttttggccattgctaagagtcctatggttgatgattatgatctttcatcagtaagaactgtcatgtctggggctgcaccattaggaaaggagcttgaagatactgttcgagccaaatttcctaatgctaaacttggtcagggttatggtatgacagaagctggaccagtgttggcaatgtgcttggcatttgcaaaagaaccctttgaaataaaatcaggggcatgtggaacagttgtgagaaatgctgaaatgaaaattgtggatcctgaaactggtaattctcttcccaggaatcaatctggagaaatttgcattagaggagaccaaatcatgaaaggctacctgaatgatccagaggccacagcaagaacaatagacaaagaaggatggttatatacaggtgacattggctacattgatgatgacgacgagctttttattgtggatcgattgaaggaactaattaaatacaaaggatttcaagttgcacctgctgagctcgaagccctccttctcaaccatcccaacatttctgatgctgctgttgttcccatgaaagacgagcaagcgggagaagttccagtggcttttgttgttagatccaatgaatccaccattaccgaagatgaagtcaaggattttatttcaaagcaggtgatattttataagaggataaagcgggtatttttcgtggatgctgttcctaaatctccatctggcaaaatccttcgaaaagatttgagagctaaactggctactgggcttccaaattaa

SEQ ID NO: 30 - protein, 542 aa, Nicotiana attenuata
MEKDTKHGDIIFRSKLPDIYIPNHLPLHSYCFENISEFSSRPCLINGANKQIYTYADVELNSRKVAAGLHKQGIQQKDTIMILLPNSPEFVFAFIGASYLGAISTMANPLFTPAEVVKQAKASSAKIIVTQACHVNKVKDYAFENDVKIICIDSAPEGCLHFSVLTQADEHDIPEIEIQPDDVVALPYSSGTTGLPKGVMLTHKGLVTSLAQQVDGENPNLYIHSEDVMLCVLPLFHIYSLNSVLLCGLRVGAAILIMQKFDIVPFLELIQSYKVTIGPFVPPIVLAIAKSPMVDDYDLSSVRTVMSGAAPLGKELEDTVRAKFPNAKLGQGYGMTEAGPVLAMCLAFAKEPFEIKSGACGTVVRNAEMKIVDPETGNSLPRNQSGEICIRGDQIMKGYLNDPEATARTIDKEGWLYTGDIGYIDDDDELFIVDRLKELIKYKGFQVAPAELEALLLNHPNISDAAVVPMKDEQAGEVPVAFVVRSNESTITEDEVKDFISKQVIFYKRIKRVFFVDAVPKSPSGKILRKDLRAKLATGLPN

SEQ ID NO: 31 - DNA, 1194 bp, Nicotiana attenuata
  source 1..1194: mol_type = other DNA
atgatgaaagtcgaggtgaaagaatcaacaatggtgaaaccagcagcagacacaccacagacaaacctatggaactcaaacgtggatttagttgtcccaaatttccacaccccaagtgtatatttctacaggccattatcaagtgcttcaaatttctttgattcgaatgtgcttaaggaggcgttgagtagagcgttagtgccgttttatccaatggctggaagattaaaaagggatgaagattggcgtattgaaattgaatgcagaggacaaggggttttgtttgttgaagcagagagtgacggaatggttgatgattttggtgattttgcgcctactttggagctccggcgtcttattcctgccgttgattattctcagggaatccattcttatgctctcctagttttgcagatcactcatttcaaatgtggaggagtttctctcggtgtaggaatgcaacatcatgctgctgatggtttttctggtcttcacttcatcaacacatggtcggacatggctcgtggtttggacctcacaattccacctttcatagaccgaacacttctccgtgctcgtgatccacccttgcctcagttcccacacgttgaataccagccacctccaactttcaagaacattgccaaaactgaagcagttcctgagactgctgtttccatcttcaagttaacccgtgatcaaatcaatgccttgaaagccaagtctaaggaagatggaaataccgtaaactacagctcctatgaaatgttagcaggacatgtatggcgctctacttgtatggcacgaggacttgaagaagatcaaggaagcaagttgtatatagcaaccgatggacgttctaggctcaggcctactcttccaccaggctactttggtaatgtgatatttactgctactccaattgcagtggctggtgatctaaaatccaagccgatttggtatgctgcaagtaaaattcatgatgcattggctcggatggacaacgactacttaagatcagccctcgattacttggaattacagcccgacctaaaggctcttgttcgcggagcacatacttttagatgcccaaatcttgggatcactagttgggttaggctgcctatacacgatgcagattttggctggggtagaccgatatttatgggacctggattgcttatgaagggttaa

SEQ ID NO: 32 - protein, 397 aa, Nicotiana attenuata
MMKVEVKESTMVKPAADTPQTNLWNSNVDLVVPNFHTPSVYFYRPLSSASNFFDSNVLKEALSRALVPFYPMAGRLKRDEDWRIEIECRGQGVLFVEAESDGMVDDFGDFAPTLELRRLIPAVDYSQGIHSYALLVLQITHFKCGGVSLGVGMQHHAADGFSGLHFINTWSDMARGLDLTIPPFIDRTLLRARDPPLPQFPHVEYQPPPTFKNIAKTEAVPETAVSIFKLTRDQINALKAKSKEDGNTVNYSSYEMLAGHVWRSTCMARGLEEDQGSKLYIATDGRSRLRPTLPPGYFGNVIFTATPIAVAGDLKSKPIWYAASKIHDALARMDNDYLRSALDYLELQPDLKALVRGAHTFRCPNLGITSWVRLPIHDADFGWGRPIFMGPGLLMKG

SEQ ID NO: 33 - DNA, 1308 bp, Nicotiana attenuata
  source 1..1308: mol_type = other DNA
atgaagattgaggtgagagaatcaacgatggtgaagccggcgactgagacgccgcaacagaggctgtggaactcaaatgtggatttggtggtgccgaatttccacacgcctagtgtttacttttacaggccgactggatccccaaatttcttcgacggaaaggtgcttaaggaagctctaagcaaagcacttgtgccgttttatcctatggcagggagactgtgtagggacgaggatggtcgtattgaaattgactgtaaaggtcagggagtgctttttgtggaagctgagtcggatggtgtggtggatgattttggtgattttgcaccgacgttggaactccgtcaactcatccccgccgttgattactcacaaggaattcaatcttatgctctcttagtgttgcagataacacatttcaaatgtgggggagtttcccttggtgtgggcatgcaacatcacgcagcagatggagcttctggtcttcacttcatcaacacatggtccgatatggctcgtggtctggacctcaccatcccaccttttatagaccggaccctcctccgtgctcgtgatccacctcagcctcagtttccccacgtcgagtaccagccacctcccactctcaaagtaactccggaaaacaccaccaaccctgaagttgttcctgaaaccagcgtgtccatcttcaaattaacccgtgatcaaatcaataccctcaaagccaagtccaaggaagatggaaatactgttaactacagctcctatgagatgttggcaggacatgtgtggcgctccacgtgcatggcacgaggactcgctcatgatcaagaaaccaaattgtacatagcaactgatggacgttccaggcttcggccctctctcccaccaggctatttcggtaatgtgatatttactactactcctattgcagtggctggtgatatccaatcaaagcctatttggtatgctgccagtaaattacacgatgcattggctagaatggacaatgactatttaagatcagctcttgattatttggagttgcagcctgacttaaaggctcttgttcgtggtgcacatacatttaagtgccccaatttaggaataactagttggtctaggctgccaattcatgatgctgattttggctggggtaggcctatatttatgggacctggtggtattgcttatgaaggtttaagctttatattaccaagtcctaccaatgatggcagtcaatcagttgccatctcgctacaagcagaacacatgaaactttttgagaagttcttgtatgacttttga

SEQ ID NO: 34 - protein, 435 aa, Nicotiana attenuata
MKIEVRESTMVKPATETPQQRLWNSNVDLVVPNFHTPSVYFYRPTGSPNFFDGKVLKEALSKALVPFYPMAGRLCRDEDGRIEIDCKGQGVLFVEAESDGVVDDFGDFAPTLELRQLIPAVDYSQGIQSYALLVLQITHFKCGGVSLGVGMQHHAADGASGLHFINTWSDMARGLDLTIPPFIDRTLLRARDPPQPQFPHVEYQPPPTLKVTPENTTNPEVVPETSVSIFKLTRDQINTLKAKSKEDGNTVNYSSYEMLAGHVWRSTCMARGLAHDQETKLYIATDGRSRLRPSLPPGYFGNVIFTTTPIAVAGDIQSKPIWYAASKLHDALARMDNDYLRSALDYLELQPDLKALVRGAHTFKCPNLGITSWSRLPIHDADFGWGRPIFMGPGGIAYEGLSFILPSPTNDGSQSVAISLQAEHMKLFEKFLYDF

SEQ ID NO: 35 - DNA, 1428 bp, Nicotiana attenuata
  source 1..1428: mol_type = other DNA
atgggtgccaaaagagatttcaatgtgaaggtgatcaaaactgaggtggtggcagcatcgttgccaatgcaagagcattggctaccacagtcaaatcttgatttgctcttgcctccaatggacgttggagttttcttttgttaccagaaccccattatttctgggatgccaaagtattcatttggatcaatggtcaaagttctaaaggtttctttggctgaaaccttggtttcctattatgcatttgcaggggagtttgttcagaaccttgctggagagcccgaagttctttgcaacaatgccggagtcagtttcacagaagcttgggctgatatagagctcaaagaaatgaacttgtataatccagatgaaagcatagagggtaagcttgtaccaaagaagaagcacggtgtattagccgttcaggctacacaactcaaatgtggaggaatagtggtggcttgtacatttgatcatagagttgctgatgcttattcaaccgacatgtttctcgtatcatgggcagaaatagctcagtccaaaccactctctcagctcccatctttcccacgttcctttctttctccacggcgtccctcactctacgatcccttggtaatagacagcatgtacatacctatctctgccctaaaacaagaaacacctgctgtcacaaatattgatgatgatgatgatcaagttataagtagaatttactatgtcaaggctgaagaaatcagccgcctccaatcattagcaaatagttgtaataacaacatgattagcacaaaattccacaggtttactaagcttgaaactttcagtgctttcctatggaaaactatagccaatgggatgggaaaaaatagtactcctaattacgagaacttcaggtttgggattgtggttaatggcaggagtagattgagcgacggagttgaagaacaagccaaggtactaaaagggtattttgggaatgttctttccattccatttggggagaagaaagtggagaaactaaaggagaaacctttaagttgggtagcaaatgcagttcatgagtttcttgaacctgcaaaaactaaagaacattttcttgggttaataaattgggtggaggatcatcgtccagaaccagctttagcaagaatctacgccacaaatgaagatgcgctggcggtggtggtgtcgtcggggcagcagtttccggtgaggaagattgaatttgggtggggagagccagtgtttgggtcgtatcattttccatgggaagggaaagcaggatatgtgatgccaatgccgagtcctaaaggaaatggagattgggttgtttacatgcacatgctgaaatggcagttggaattggttgaggcttctgcggccaatgtgttcaagcccctgactgcagattatctcaacttgaattaa

SEQ ID NO: 36 - protein, 475 aa, Nicotiana attenuata
MGAKRDFNVKVIKTEVVAASLPMQEHWLPQSNLDLLLPPMDVGVFFCYQNPIISGMPKYSFGSMVKVLKVSLAETLVSYYAFAGEFVQNLAGEPEVLCNNAGVSFTEAWADIELKEMNLYNPDESIEGKLVPKKKHGVLAVQATQLKCGGIVVACTFDHRVADAYSTDMFLVSWAEIAQSKPLSQLPSFPRSFLSPRRPSLYDPLVIDSMYIPISALKQETPAVTNIDDDDDQVISRIYYVKAEEISRLQSLANSCNNNMISTKFHRFTKLETFSAFLWKTIANGMGKNSTPNYENFRFGIVVNGRSRLSDGVEEQAKVLKGYFGNVLSIPFGEKKVEKLKEKPLSWVANAVHEFLEPAKTKEHFLGLINWVEDHRPEPALARIYATNEDALAVVVSSGQQFPVRKIEFGWGEPVFGSYHFPWEGKAGYVMPMPSPKGNGDWVVYMHMLKWQLELVEASAANVFKPLTADYLNLN

SEQ ID NO: 37 - DNA, 1311 bp, Nicotiana attenuata
  source 1..1311: mol_type = other DNA
atgggaagtgaaaaaatcatgaagattaacatcaaagaatcaacattagtaaaaccatcaaaaccaacaccaacaaagagactttggagttctaacttagatttaatagtgggaagaattcatcttttaacagtatatttttataaaccaaatggatcttcaaatttctttgatacaaaaataatgaaagaagcattaagtaatgttcttgtttcattttatccaatggctgggagattaggtagagatgaacaaggaagaattgagataaattgtaatggagaaggagttttatttgttgaagctgaaagtgatgcttttgttgatgattttggtgattttactccaagtttggaacttaggaaacttattcctactgttgatacttctggtgatatttctactttccccctcatcatctttcaggttactcgtttcaaatgtggtggagtttcacttggtggaggagtattccacactttatcagatggtctctcatccattcacttcatcaacacatggtccgatattgcccgaggcctctccgtggccatcccgccgttcattgaccggaccctcctccgtgcacgggacccaccgacatcgtcgttcgagcacattgagtatcatcctcctccatctcttatttcatcatcaaaaaccaaagaatccacaagcccaaagcctagtaccacaaccatgttaaaattctctagtgaccaacttgggcttctaaagtccaagtccaaacatgaaggtagcacatacgaaatcctcgcggcccatatttggcgttgcacgtgcaaagcacgtgcactgtccgacgatcaattgaccaaattacatgttgccactgatggtaggtctagactttgccctcctttgccaccaggttacttaggaaatgttgtgttcacagctacaccaatggcaaaatcaagtgaacttttacaagaaccattgacaaattcagctaagagaattcatagttcattgtcaaaaatggatgacaattacctaagatcagctctcgattacctcgaattacagcccgatttatcggctttaatccgtggcccgacatactttgctagccctaatcttaatattaatagttggactagattgcctgttcatgattcagattttggatggggaagaccaattcatatgggaccagcttgcattttatatgaagggacagtttatatattaccaagtccaaatagtaaagatagaaacttacgtttggctgtttgtttagatgctgatcatatgccactatttgagaagtatttgtatgaattctaa

SEQ ID NO: 38 - protein, 436 aa, Nicotiana attenuata
MGSEKIMKINIKESTLVKPSKPTPTKRLWSSNLDLIVGRIHLLTVYFYKPNGSSNFFDTKIMKEALSNVLVSFYPMAGRLGRDEQGRIEINCNGEGVLFVEAESDAFVDDFGDFTPSLELRKLIPTVDTSGDISTFPLIIFQVTRFKCGGVSLGGGVFHTLSDGLSSIHFINTWSDIARGLSVAIPPFIDRTLLRARDPPTSSFEHIEYHPPPSLISSSKTKESTSPKPSTTTMLKFSSDQLGLLKSKSKHEGSTYEILAAHIWRCTCKARALSDDQLTKLHVATDGRSRLCPPLPPGYLGNVVFTATPMAKSSELLQEPLTNSAKRIHSSLSKMDDNYLRSALDYLELQPDLSALIRGPTYFASPNLNINSWTRLPVHDSDFGWGRPIHMGPACILYEGTVYILPSPNSKDRNLRLAVCLDADHMPLFEKYLYEF

SEQ ID NO: 39 - DNA, 1305 bp, Nicotiana attenuata
  source 1..1305: mol_type = other DNA
atgaagatcaatattaaggaatcaacaatggtgaaaccatcaaagccaactcctacaaaaaggctatggaactcaaatttggatttaatagtgggaagaattcatcttttgacagtatatttctacaaatcaaatggaatttctccaaatttctttgattttagaattatgaaggaggctttaagcaatgttttagtttcattttatccaatggctgggagattggctaaggataatgaaaaagaaagaattgagataaattgtaatggagaaggagttctttttattgaagctgaaattgatgattttattgatgattttggtgattttattccaagtttggaaataaagaagatgttaattcctaatgttgatacttctggtgacatctcttcttttccacttgtcatatttcaggtaactcgattcaagtgtggtggagtctccttagggtgtggcattttccatacattatctgacggcaattcatcccttcacttcatcaacacatggtcggaagttgcccgaggcctctccgtcaccgtctcgccgttcatcgaccggtccctcctccgtgcacgggacccaccgacaccagcttataaacacgtggaatatcatcctcctccgaccctaaaatcatcgtcaaaaaacgttcatgataaagttagttgtccaaaatccagcaccacggccatgtttaagatcacaagtgaccaactgaccctactcaagaccaagtccgagcacgagggtagtacttatgaagtactcgcggcccatatttggcgttgcacgtgcaaagcacgtggcctagatgatgaccaattgacaaagttacatgttgctacagatggtaggtcaagactttttcctcctttaccaccaggctacttaggaaatgttgttttcacagctacaccaattgccaaatctggtgtaattttatcagagccattgatgaacactgcacaaagaattcataaggtgttagcaagaatggacgacgaatacttaagatcagcgatagattatctcgaattacagacagatttatctaaattaatccgaggaccgacgtattttgctagtcctaatcttaatattaacagttggactaggttgcctgttcatgattcagattttggatggggaaaaccaattcatatgggacctgcatgtattttgtatgaagggacagtttacatattgccaagtccaagtaatgacaagaacttgaagttggctgtgtgtttagatgctgaccatatgccactttttgagaagtacttgtatgacttttga

SEQ ID NO: 40 - protein, 434 aa, Nicotiana attenuata
MKINIKESTMVKPSKPTPTKRLWNSNLDLIVGRIHLLTVYFYKSNGISPNFFDFRIMKEALSNVLVSFYPMAGRLAKDNEKERIEINCNGEGVLFIEAEIDDFIDDFGDFIPSLEIKKMLIPNVDTSGDISSFPLVIFQVTRFKCGGVSLGCGIFHTLSDGNSSLHFINTWSEVARGLSVTVSPFIDRSLLRARDPPTPAYKHVEYHPPPTLKSSSKNVHDKVSCPKSSTTAMFKITSDQLTLLKTKSEHEGSTYEVLAAHIWRCTCKARGLDDDQLTKLHVATDGRSRLFPPLPPGYLGNVVFTATPIAKSGVILSEPLMNTAQRIHKVLARMDDEYLRSAIDYLELQTDLSKLIRGPTYFASPNLNINSWTRLPVHDSDFGWGKPIHMGPACILYEGTVYILPSPSNDKNLKLAVCLDADHMPLFEKYLYDF

SEQ ID NO: 41 - DNA, 1119 bp, Nicotiana attenuata
  source 1..1119: mol_type = other DNA
atggcaacttgcaatggaaatggagctgcaaaattttcctccaaattgcaagttgaagcaatccaaactgtgataccaatgaagataaccaatccacggttatcgaggcgaatcgcggtatccgaaaatttttgctcgggcgatttgcaaaggcgatttcacatggtgttgtactataacaaagcatcagaggcggattcaggatggatagtcgcgggttggtttaaagagtcattaggaatggcaatagttgaaagtccattacttggtggaagattaaggaaattagaggataataatgatttggaattggtttcaaatgattctggtgttagattggttgaggctaatgctgaaataaatatggctgattttattgatctcaaaaacaaggaaaatgttgaaactgagcttgttttctgggaagatattcatgaaacaagtcctcagttttctccccttttctatgtccaggtgacaaatttcaagtgtggaggatactcaattgggataagttgcagcctttttatagcagatccttttgtcatgacaagtttcttgaaaaactggtccaaaattcacaacaacttgatctcacaaattgatgcaccaaaaattccagcattttatactccaaatttaagaaaagttggttttttacctactatatctactagcttaaccacaagaaatcaaaccactcaaactttaattttcaaaattcccaaaaaccccttgaatttaaacaaaaatatcctcaaaaatcttgcatcaaaatgtattggagaaacagaagaaaatattggtaaaaatttgtcttcaaaattcactttgtttgtcaaagagattactaattctgagaatgtaattaaggtggaaacttgtattagagaggagataattaaggaggaatgtggattaatatctaaaaatggtggattaattttagcaagttgggatgatttgggaggtgataaagtgagttttaatgaaggaaataaggctgttaatatttcatgttggattattaatgcagaaaatcaagatcttgtgatgattactccatttcctgatgaagatagctctcaactaaacattataattacagttactaattag

SEQ ID NO: 42 - protein, 372 aa, Nicotiana attenuata
MATCNGNGAAKFSSKLQVEAIQTVIPMKITNPRLSRRIAVSENFCSGDLQRRFHMVLYYNKASEADSGWIVAGWFKESLGMAIVESPLLGGRLRKLEDNNDLELVSNDSGVRLVEANAEINMADFIDLKNKENVETELVFWEDIHETSPQFSPLFYVQVTNFKCGGYSIGISCSLFIADPFVMTSFLKNWSKIHNNLISQIDAPKIPAFYTPNLRKVGFLPTISTSLTTRNQTTQTLIFKIPKNPLNLNKNILKNLASKCIGETEENIGKNLSSKFTLFVKEITNSENVIKVETCIREEIIKEECGLISKNGGLILASWDDLGGDKVSFNEGNKAVNISCWIINAENQDLVMITPFPDEDSSQLNIIITVTN

SEQ ID NO: 43 - DNA, 1410 bp, Nicotiana attenuata
  source 1..1410: mol_type = other DNA
atgtgtaaaaaagagataactacaaaaagaaacttcaatgtaaagatgagcaagagtgaagtggtggcagcagttttgccaatacaagagcattggctaccactgtccaatcttgacttgctcttgcctccattggatgttggagttatcttttgctatcaaaatcctatttctgtgacaacaaaaatagaatttggttcaatggttagaattctcaaagcttctttggctgaaacattagttatttactatgcatttgctggtgagatagttcagaacttaggtggagaacctgagcttctttgcaacaataatggagtcaattttacagaagcttttgctgatgttgagctcaaagaaatcaacttttataatccagatgagagtatagagggaaaacttgttccaaataagaagcatggtgtactagctattcaggttacacaactcaaatgtggaggactagttgtgggttgcacatttgatcatcgagttgctgatgcatattcagccaacttgtttcttgtatcatggtctgaattagctcaatccaaacctctcactcagctcccatctttccgtcgatcgttcctttttcctcgacgtcctggttactacgatgactcaatcgatagcctgtacgtaccaataccaaccctaccaccctcaaaacccgaaacccttagccctaacgatcaagccataagtcgaatttactatgtcacgggggagaaaatcagtcaccttcaatcactagccaatcgtgaccaaaaaagtgcaaaattccagaggtctaaacttgaatcattcagcgcttacctgtggaaaactattgcatgtggaattaaagaaacatcaaattccaagaatttcaggtttggtatcgttgttgatggtaggacaagattaagtaatggagatgataatcaagaaaaattactaaaagggtattttggtaatgttctctctatcccatttggaaacaagaaaattgaggagctaaaagagaagccgttgagttgggtagcaaatgcgattcatgagtttatagaaaaagcagtaacacgcgaacattttcttggattgatagattgggttgaggctcatcgtcctgaaccggccgtggcaaaaatatacgccacggacgacgatggacccgccgtggtagtgtcatcagggcaacaatttccagccagagagatagatttcggatggggtgagccagtcttctggtcgtatcattttccatgggcgggaaaatcaggatatgtgatgccaatgccaagtcctaaaggaaatggagattggattgtttatatgcatttgatgaaatggcaattggaactaattgagacttctgcatccaatgtgttcaagcctgtgactgcaaattatctcagcttaatgtga

SEQ ID NO: 44 - protein, 469 aa, Nicotiana attenuata
MCKKEITTKRNFNVKMSKSEVVAAVLPIQEHWLPLSNLDLLLPPLDVGVIFCYQNPISVTTKIEFGSMVRILKASLAETLVIYYAFAGEIVQNLGGEPELLCNNNGVNFTEAFADVELKEINFYNPDESIEGKLVPNKKHGVLAIQVTQLKCGGLVVGCTFDHRVADAYSANLFLVSWSELAQSKPLTQLPSFRRSFLFPRRPGYYDDSIDSLYVPIPTLPPSKPETLSPNDQAISRIYYVTGEKISHLQSLANRDQKSAKFQRSKLESFSAYLWKTIACGIKETSNSKNFRFGIVVDGRTRLSNGDDNQEKLLKGYFGNVLSIPFGNKKIEELKEKPLSWVANAIHEFIEKAVTREHFLGLIDWVEAHRPEPAVAKIYATDDDGPAVVVSSGQQFPAREIDFGWGEPVFWSYHFPWAGKSGYVMPMPSPKGNGDWIVYMHLMKWQLELIETSASNVFKPVTANYLSLM

SEQ ID NO: 45 - DNA, 1536 bp, Nicotiana attenuata
  source 1..1536: mol_type = other DNA
atggctgtttctttagctgttgcaattcctttagctttcattttcacatttatagcttatcatctctactaccgtctccgattcaagcttcctccgggcccaagtccacggcccatcgtcggaaacctgtaccagataaagcccgtgaggtttcgatgcttttacgagtgggcccaaacctacggacctatcatttcggtttggttcgggtcgacccttaacgttgtcgtttccagctcggaattagctaaggaagttctgaaagaaaaggaccagcagttggctgaccggcacaggagccgatcggcggccaagtttagcagagacgggcaggatcttatttgggccgattatgggcctcattatgttaaggttagaaaggtttgtacgattgagctttttacgcccaaaaggcttgaagctcttaggccaatccgagaagatgaggtcactgctatggtggagtccatttacagagattgtactaatcctgataacgtggggaagaatctgctggtgaagaagtaccttggagcagtggcctttaacaacataacaaggcttgcctttgggaagaggtttgtaaactttgagggtgtgatggatgaacaaggaaacgagttcaaggcgattgtcgccaatggattgaagcttggtgcatcccttgccatggccgaacacatcccatggcttcgctggatgttccctattgacgaggatgcctttgctaagcacggagcacgtagagaccgtcttacccgagctattatggaggagcacacccttgctcgccaacaaagtggaggagccaagcaacacttttttgatgcattattgaccctccaacagaaatatgacttaagtgaagacactctcattggcttactttgggatatgatcacagcaggaatggacacaactgcaatctctgttgaatgggcaatggctgaggtgatcaagaacccaagagtgcagcagaaggcccaagaagagctcgaccgagtggtcggctacgagcgtgtgatgaacgaaacagacttccccaacctcccatacctacaatgcgtggccaaggaagcgctaaggctgcaccctccaactcctctaatgctcccacacagagccaacacgaacgtcaagattggtggctacgacattcccaagggctccaacgtgcacgtgaacgtctgggcagttgctcgtgatcccaaggtgtggaaaaacccattggagttcagaccagagaggttccttgaggaagatgtggacatgaagggacatgattttaggctactcccatttggtgctggtagaagagtatgtccaggggcacaattgggtatcaacttggtcacctctatgttgggccatcttttgcaccattttcattgggctccttctaatggattgagcccagaggagattgatatgggtgagaaccctgggcttgttacctatatgaggactccattacaggccgatgccactcccagattgcctgcggagttgtataaacgaattgcagcggacatgtaa

SEQ ID NO: 46 - protein, 511 aa, Nicotiana attenuata
MAVSLAVAIPLAFIFTFIAYHLYYRLRFKLPPGPSPRPIVGNLYQIKPVRFRCFYEWAQTYGPIISVWFGSTLNVVVSSSELAKEVLKEKDQQLADRHRSRSAAKFSRDGQDLIWADYGPHYVKVRKVCTIELFTPKRLEALRPIREDEVTAMVESIYRDCTNPDNVGKNLLVKKYLGAVAFNNITRLAFGKRFVNFEGVMDEQGNEFKAIVANGLKLGASLAMAEHIPWLRWMFPIDEDAFAKHGARRDRLTRAIMEEHTLARQQSGGAKQHFFDALLTLQQKYDLSEDTLIGLLWDMITAGMDTTAISVEWAMAEVIKNPRVQQKAQEELDRVVGYERVMNETDFPNLPYLQCVAKEALRLHPPTPLMLPHRANTNVKIGGYDIPKGSNVHVNVWAVARDPKVWKNPLEFRPERFLEEDVDMKGHDFRLLPFGAGRRVCPGAQLGINLVTSMLGHLLHHFHWAPSNGLSPEEIDMGENPGLVTYMRTPLQADATPRLPAELYKRIAADM

SEQ ID NO: 47 - DNA, 54 bp, synthetic construct
  misc_feature 1..54: note = Oligonucleotide
  source 1..54: mol_type = other DNA
ggggacaagtttgtacaaaaaagcaggcttaatgaatgtgaaaattgagagctc

SEQ ID NO: 48 - DNA, 58 bp, synthetic construct
  misc_feature 1..58: note = Oligonucleotide
  source 1..58: mol_type = other DNA
ggggaccactttgtacaagaaagctgggtatcattttgctttcaaatcaagagaatag

SEQ ID NO: 49 - DNA, 53 bp, synthetic construct
  misc_feature 1..53: note = Oligonucleotide
  source 1..53: mol_type = other DNA
ggggacaagtttgtacaaaaaagcaggcttaatggcttcttcttctactctac

SEQ ID NO: 50 - DNA, 55 bp, synthetic construct
  misc_feature 1..55: note = Oligonucleotide
  source 1..55: mol_type = other DNA
ggggaccactttgtacaagaaagctgggtaataaaaagccttgagttcaatcaac

SEQ ID NO: 51 - DNA, 54 bp, synthetic construct
  misc_feature 1..54: note = Oligonucleotide
  source 1..54: mol_type = other DNA
ggggacaagtttgtacaaaaaagcaggcttaatggcttcttcttcttcttctac

SEQ ID NO: 52 - DNA, 54 bp, synthetic construct
  misc_feature 1..54: note = Oligonucleotide
  source 1..54: mol_type = other DNA
ggggaccactttgtacaagaaagctgggtattaacaatcgacaagcttaatctc

SEQ ID NO: 53 - DNA, 54 bp, synthetic construct; identical to SEQ ID NO: 51
  misc_feature 1..54: note = Oligonucleotide
  source 1..54: mol_type = other DNA

SEQ ID NO: 54 - DNA, 55 bp, synthetic construct
  misc_feature 1..55: note = Oligonucleotide
  source 1..55: mol_type = other DNA
ggggaccactttgtacaagaaagctgggtattcactactatacttatgctcttgc

SEQ ID NO: 55 - DNA, 20 bp, synthetic construct
  misc_feature 1..20: note = Oligonucleotide
  source 1..20: mol_type = other DNA
acaaaagggggctaccagca

SEQ ID NO: 56 - DNA, 20 bp, synthetic construct
  misc_feature 1..20: note = Oligonucleotide
  source 1..20: mol_type = other DNA
attggatacggtcaggggcg

SEQ ID NO: 57 - DNA, 20 bp, synthetic construct
  misc_feature 1..20: note = Oligonucleotide
  source 1..20: mol_type = other DNA
gcgaagtggtcagcatcgac

SEQ ID NO: 58 - DNA, 20 bp, synthetic construct
  misc_feature 1..20: note = Oligonucleotide
  source 1..20: mol_type = other DNA
acgacccctcaagacacaca

SEQ ID NO: 59 - DNA, 20 bp, synthetic construct
  misc_feature 1..20: note = Oligonucleotide
  source 1..20: mol_type = other DNA
ggttccaaagacaggtggcg

SEQ ID NO: 60 - DNA, 20 bp, synthetic construct
  misc_feature 1..20: note = Oligonucleotide
  source 1..20: mol_type = other DNA
gtgcacacactgcgttcaga

SEQ ID NO: 61 - DNA, 22 bp, synthetic construct
  misc_feature 1..22: note = Oligonucleotide
  source 1..22: mol_type = other DNA
acggaatctctctcaaggcagt

SEQ ID NO: 62 - DNA, 22 bp, synthetic construct
  misc_feature 1..22: note = Oligonucleotide
  source 1..22: mol_type = other DNA
cctcctatgcttggttcaacgc

SEQ ID NO: 63 - DNA, 22 bp, synthetic construct
  misc_feature 1..22: note = Oligonucleotide
  source 1..22: mol_type = other DNA
agttgaaggccaattctgtgct

SEQ ID NO: 64 - DNA, 20 bp, synthetic construct
  misc_feature 1..20: note = Oligonucleotide
  source 1..20: mol_type = other DNA
tgctgccatccaccaccata

SEQ ID NO: 65 - DNA, 22 bp, synthetic construct
  misc_feature 1..22: note = Oligonucleotide
  source 1..22: mol_type = other DNA
ttctgctacgccgcaagtactg

SEQ ID NO: 66 - DNA, 21 bp, synthetic construct
  misc_feature 1..21: note = Oligonucleotide
  source 1..21: mol_type = other DNA
ggtatggcgctctagccgttg

SEQ ID NO: 67 - DNA, 20 bp, synthetic construct
  misc_feature 1..20: note = Oligonucleotide
  source 1..20: mol_type = other DNA
tgcaccatatggcctcagca

SEQ ID NO: 68 - DNA, 21 bp, synthetic construct
  misc_feature 1..21: note = Oligonucleotide
  source 1..21: mol_type = other DNA
agctaggtctccttaccggct

SEQ ID NO: 69 - DNA, 22 bp, synthetic construct
  misc_feature 1..22: note = Oligonucleotide
  source 1..22: mol_type = other DNA
caatttggtcaaggagacgtga

SEQ ID NO: 70 - DNA, 22 bp, synthetic construct
  misc_feature 1..22: note = Oligonucleotide
  source 1..22: mol_type = other DNA
ggctctgatcacaattacaagg

SEQ ID NO: 71 - DNA, 23 bp, synthetic construct
  misc_feature 1..23: note = Oligonucleotide
  source 1..23: mol_type = other DNA
cacgcatctacattgacttgcca

SEQ ID NO: 72 - DNA, 21 bp, synthetic construct
  misc_feature 1..21: note = Oligonucleotide
  source 1..21: mol_type = other DNA
agcagctggagcttctttgtt

SEQ ID NO: 73 - DNA, 24 bp, synthetic construct
  misc_feature 1..24: note = Oligonucleotide
  source 1..24: mol_type = other DNA
actcttccaccagaaaagttgctc

SEQ ID NO: 74 - DNA, 22 bp, synthetic construct
  misc_feature 1..22: note = Oligonucleotide
  source 1..22: mol_type = other DNA
tgagatgtccattgccaggagt

SEQ ID NO: 75 - DNA, 20 bp, synthetic construct
  misc_feature 1..20: note = Oligonucleotide
  source 1..20: mol_type = other DNA
ggcccggaacaactactaca

SEQ ID NO: 76 - DNA, 20 bp, synthetic construct
  misc_feature 1..20: note = Oligonucleotide
  source 1..20: mol_type = other DNA
ccccgtcgattaaagtctga

SEQ ID NO: 77 - DNA, 20 bp, synthetic construct
  misc_feature 1..20: note = Oligonucleotide
  source 1..20: mol_type = other DNA
tctggtgcgatgaagtcaag

SEQ ID NO: 78 - DNA, 20 bp, synthetic construct
  misc_feature 1..20: note = Oligonucleotide
  source 1..20: mol_type = other DNA
ctgcttcgacgtgattcaaa

SEQ ID NO: 79 - DNA, 20 bp, synthetic construct
  misc_feature 1..20: note = Oligonucleotide
  source 1..20: mol_type = other DNA
atcaactagccattagaatg

SEQ ID NO: 80 - DNA, 21 bp, synthetic construct
  misc_feature 1..21: note = Oligonucleotide
  source 1..21: mol_type = other DNA
ccaaaaatgatttgcaaggtc

SEQ ID NO: 81 - DNA, 20 bp, synthetic construct
  misc_feature 1..20: note = Oligonucleotide
  source 1..20: mol_type = other DNA
atcaaatagctgaagatgtc

SEQ ID NO: 82 - DNA, 21 bp, synthetic construct
  misc_feature 1..21: note = Oligonucleotide
  source 1..21: mol_type = other DNA
ccaacaaagtagtgctgtact

SEQ ID NO: 83 - DNA, 20 bp, synthetic construct
  misc_feature 1..20: note = Oligonucleotide
  source 1..20: mol_type = other DNA
gtcggacgaagaacaccatt

SEQ ID NO: 84 - DNA, 20 bp, synthetic construct
  misc_feature 1..20: note = Oligonucleotide
  source 1..20: mol_type = other DNA
cacatcacagttgtgggagg

SEQ ID NO: 85 - DNA, 29 bp, synthetic construct
  misc_feature 1..29: note = Oligonucleotide
  source 1..29: mol_type = other DNA
gggaattccatatgatgggcagagctcct

SEQ ID NO: 86 - DNA, 34 bp, synthetic construct
  misc_feature 1..34: note = Oligonucleotide
  source 1..34: mol_type = other DNA
ccggaattctcataattcaggcaattctaacatc

SEQ ID NO: 87 - DNA, 31 bp, synthetic construct
  misc_feature 1..31: note = Oligonucleotide
  source 1..31: mol_type = other DNA
cccagcatatgatgacggactatagaatacc

SEQ ID NO: 88 - DNA, 32 bp, synthetic construct
  misc_feature 1..32: note = Oligonucleotide
  source 1..32: mol_type = other DNA
tacagatcgatgtcgcgattcagcaattctgg

SEQ ID NO: 89 - DNA, 31 bp, synthetic construct
  misc_feature 1..31: note = Oligonucleotide
  source 1..31: mol_type = other DNA
cccagcatatgatgaatttgtggaatactag

SEQ ID NO: 90 - DNA, 32 bp, synthetic construct
  misc_feature 1..32: note = Oligonucleotide
  source 1..32: mol_type = other DNA
tacagatcgatggcgtgtttcagcaactctgg

SEQ ID NO: 91 - DNA, 35 bp, synthetic construct
  misc_feature 1..35: note = Oligonucleotide
  source 1..35: mol_type = other DNA
gcggcggtcgacgttacctatgatgctttaatggc

SEQ ID NO: 92 - DNA, 34 bp, synthetic construct
  misc_feature 1..34: note = Oligonucleotide
  source 1..34: mol_type = other DNA
gcggcgggatcctgtgacttgaacttggatcaac

SEQ ID NO: 93 - DNA, 33 bp, synthetic construct
  misc_feature 1..33: note = Oligonucleotide
  source 1..33: mol_type = other DNA
gcggcggtcgacgatggggtatgattacgaacc

SEQ ID NO: 94 - DNA, 34 bp, synthetic construct
  misc_feature 1..34: note = Oligonucleotide
  source 1..34: mol_type = other DNA
gcggcgggatccatgtcatctgcattcacagtct

SEQ ID NO: 95 - DNA, 30 bp, synthetic construct
  misc_feature 1..30: note = Oligonucleotide
  source 1..30: mol_type = other DNA
gcggcggtcgacaatgggcttcgattacgc

SEQ ID NO: 96 - DNA, 27 bp, synthetic construct
  misc_feature 1..27: note = Oligonucleotide
  source 1..27: mol_type = other DNA
gcggcgggatccagctcgtccgcattc

SEQ ID NO: 97 - DNA, 29 bp, synthetic construct
  misc_feature 1..29: note = Oligonucleotide
  source 1..29: mol_type = other DNA
gcggcggtcgactactcctgagacctgca

SEQ ID NO: 98 - DNA, 29 bp, synthetic construct
  misc_feature 1..29: note = Oligonucleotide
  source 1..29: mol_type = other DNA
gcggcgggatccagtccgtcttgcctttg

SEQ ID NO: 99 - DNA, 34 bp, synthetic construct
  misc_feature 1..34: note = Oligonucleotide
  source 1..34: mol_type = other DNA
acgcgtcgaccgatggcttcttcttctactctac

SEQ ID NO: 100 - DNA, 35 bp, synthetic construct
  misc_feature 1..35: note = Oligonucleotide
  source 1..35: mol_type = other DNA
ccgctcgagcataaaaagccttgagttcaatcaac

SEQ ID NO: 101 - DNA, 35 bp, synthetic construct
  misc_feature 1..35: note = Oligonucleotide
  source 1..35: mol_type = other DNA
acgcgtcgaccgatggcttcttcttcttcttctac

SEQ ID NO: 102 - DNA, 32 bp, synthetic construct
  misc_feature 1..32: note = Oligonucleotide
  source 1..32: mol_type = other DNA
ccgctcgagcacaatcgacaagcttaatctca

SEQ ID NO: 103 - DNA, 35 bp, synthetic construct
  misc_feature 1..35: note = Oligonucleotide
  source 1..35: mol_type = other DNA
ccggaattcatgtttccactcataattctgatcag

SEQ ID NO: 104 - DNA, 35 bp, synthetic construct
  misc_feature 1..35: note = Oligonucleotide
  source 1..35: mol_type = other DNA
ccgaagcttcttcactactatacttatgctcttgc

SEQ ID NO: 105 - DNA, 1671 bp, Nicotiana attenuata
  source 1..1671: mol_type = other DNA
atggcacagtttagcaatggctcaaccgtcgatccaagaaccggctattctccggcggactcaacattctacagcaaacgagacccaattcccttgccagcaaacgaagctctcgacgtgacaactttcatatcatcacgagcacaccgcggcaagatcgctttcatcgatgccacaacagggaagcaactcactttctccgacgtctggaacgccgtcgaatctttagccacgtgtctctctatcgacatgggaattcataagggcaacgtagttcttctcctctcaccaaactccatctatttccccattgtttgcctcgccgtcatgtctctcggcgctattatcaccacaacaaaccccctcaacaccccacgtgaaattaacaaacagatcaaagattcaaaccctgtaatcgccttcacaatccctgaactcctccctaagctcgtcgattctaatctcccgattattcttatcaacaatcaaacaattaatcttactcaaaagggtaatttcaatttcaagattttgggtaatttagaaaaaatgattcagaaaaaacatgttcaaagcagattgaaagagcgagtgacacaagatgatacagctactctgctttattcttcaggaactactggtgcaagtaaaggagttgtttcatctcatagaaatttaattgctatggttcaaacaattgtgggtcggttcaaattagacgaaggagaacaaacttttatttgtacagtacccatgtttcatatttacggtttagcagcttttgcagctgggttattagctagtggatcgacagtcgtggtactatcgaaattcgaaatggacgaaatgttattatcaattcacaaatacaaagcaacttatttacctctcgtgccaccaattcttgttgcgctagtgaataatgctgaggtgattaaaaagaaatatgatttgagcagtttaaagaaagttctttctgggggtgcacctttgagtaaggaagtaattgaagggtttttggagaaatatccaagtgtgacaattttgcaaggttatggcttgactgaatcaacgggaattggggcgtcgacggattcgttagaggagagccggagatatgggacggcggggatgttgtcgccgagcatgtcggcaaaaattgtggatcccgaaagtgggaaggcttttccggtgaataagaccggtgaactttggctaaggggacctaccattatgaaaggttatttcagtaatgaagaagctactgcctcaactttagactcagagggatggttaagaactggagatctctgttatatagatgaggacggtttcatttttgtggttgatagattaaaggagctgatcaaatataagggttatcaggttcctccagcagagttagaggctttgttgctcactcatccagaaatttctgatgctgctgttataccgtttccggataaggatgtcggacagtttcctatggcatatgtagtaagaaaatctggaagtactctgtcagagagtgcagtcatggattttattgccaaacaggtggctccatacaagagaatccggagagttgcatttgtggcttccatacccaagaatccttctggcaaaatactcaggaaggatctgataaagctagcaacatctaaactttga

SEQ ID NO: 106 - protein, 556 aa, Nicotiana attenuata
MAQFSNGSTVDPRTGYSPADSTFYSKRDPIPLPANEALDVTTFISSRAHRGKIAFIDATTGKQLTFSDVWNAVESLATCLSIDMGIHKGNVVLLLSPNSIYFPIVCLAVMSLGAIITTTNPLNTPREINKQIKDSNPVIAFTIPELLPKLVDSNLPIILINNQTINLTQKGNFNFKILGNLEKMIQKKHVQSRLKERVTQDDTATLLYSSGTTGASKGVVSSHRNLIAMVQTIVGRFKLDEGEQTFICTVPMFHIYGLAAFAAGLLASGSTVVVLSKFEMDEMLLSIHKYKATYLPLVPPILVALVNNAEVIKKKYDLSSLKKVLSGGAPLSKEVIEGFLEKYPSVTILQGYGLTESTGIGASTDSLEESRRYGTAGMLSPSMSAKIVDPESGKAFPVNKTGELWLRGPTIMKGYFSNEEATASTLDSEGWLRTGDLCYIDEDGFIFVVDRLKELIKYKGYQVPPAELEALLLTHPEISDAAVIPFPDKDVGQFPMAYVVRKSGSTLSESAVMDFIAKQVAPYKRIRRVAFVASIPKNPSGKILRKDLIKLATSKL

SEQ ID NO: 107 - DNA, 405 bp, Nicotiana attenuata
  source 1..405: mol_type = other DNA
atggccaatcctctgtttacgccagcagaggttgtaaaacaagccaaaacctcaagtgctaagattattataactcaattgtgctttgcaggcaaagtgaaggattacgctagtgaaaatgatgtgaaggttatttgtattgattctgaaccagaaggttgtcttcatttctcagaatggacacaatctgacgagcatgaaattcctgaggcgaaaatccagccggacgacgtcgtatcgctgccgtattcctctggcactacgggtctgccaaagggagttatgttgactcacaagggattaatcacgagcattgcacaacaagttgacggtgaaattgcaaatttgtatatgcacagtgaagatgtattgataaactcgatattgctccgcttttggagttaa

SEQ ID NO: 108 - protein, 134 aa, Nicotiana attenuata
MANPLFTPAEVVKQAKTSSAKIIITQLCFAGKVKDYASENDVKVICIDSEPEGCLHFSEWTQSDEHEIPEAKIQPDDVVSLPYSSGTTGLPKGVMLTHKGLITSIAQQVDGEIANLYMHSEDVLINSILLRFWS

SEQ ID NO: 109 - DNA, 1641 bp, Nicotiana attenuata
  source 1..1641: mol_type = other DNA
atggatacaaagcaaggagacataattttcaaatcaaaactccctgatatatacatccctaatcatcttcctttacattcttattgctttgaaaacatttcagaattcagtactcgtccttgtttgatcaatggagccaatgaccaaatttatacatacgctgatgttgagctcatttcaagaaaagttgcagctggatttaacaagctaggaatcaaacaaaaggacactataatgatcctcttgcctaattctcctgaatttgtattcacctttttaggtgcatcatatcttggagctatttcaactatggcaaatcctttgtttacccaagtagaggtagtaaaacaagccaaggcctcaaatcccaagcttattgttacacaagagtgcaatttggacaaagtgaaaagctatgcttttgagcatgatgtgaaggtcatttgcattgactcgtcgtcagagcgctgcttacacttctctgacttgattttaaatcctggattcgcttctagtatcgaacatgacctttccgaggtggaaatccaaccggacgatatactggcgctgccgttctcgtccgggaccacggggctaccaaaaggggtgatgttgacacataaggggctagtcactagtgtggcacaacaagttgatggtgaaaatccaactttgtatattcatagtgaggatgtgatactttgtgttttgcctttgtttcatatttactctttggattctgttttactttgtggattaagggttggtgcagctattttgattatgcagaaatttgatattgttccatttttggagttgatacaaaagtacaaagtgactattgctccttttgtaccacctattgttttggccattgctaagagtgcaatggttgataattatgatctttcatcagtaaggatagtcatgtgtggggcagctccattaggaaaggatcttgaagatgcagttcgaacaaaatttcctaatgctaaacttggtcagggttatgggatgacggaggctggttcagtgttggcaatgtgtttggcatttgcaaaagagccatttgagataaaatcaggggcctgtggaactgttataaggaatgctgagatgaaaattatggatctaaacactggtagctctcttcctcgaaacaaatctggagagatttgcatacgaggtgaccaaatcatgaaaggttacctgaatgatcctgaggcaactgcacaaacaatagacaaagaaggatggttacacagtggtgacattggctacattgacgacgacgacgagctttttattgtggatcgattgaaggaattgataaaatacaaaggatttcaagtggctcctgctgaacttgaagcacttctcctcaaccttccttatatttctgatgctgctgttgtccccatgaaagatgagcaagcaggagaagttccagtggcttttgttgttaaatccagtggatgcaccattactgaggacgaaatcaaggattttatctcaaagcaggtgatattttataagagaataaagcgagtattttttgtggatgcggttcctaaatctccatctggcaaaatacttcgaaaggacttgagagccagtctagcagatgggcttccaaattaa

SEQ ID NO: 110 - protein, 546 aa, Nicotiana attenuata
MDTKQGDIIFKSKLPDIYIPNHLPLHSYCFENISEFSTRPCLINGANDQIYTYADVELISRKVAAGFNKLGIKQKDTIMILLPNSPEFVFTFLGASYLGAISTMANPLFTQVEVVKQAKASNPKLIVTQECNLDKVKSYAFEHDVKVICIDSSSERCLHFSDLILNPGFASSIEHDLSEVEIQPDDILALPFSSGTTGLPKGVMLTHKGLVTSVAQQVDGENPTLYIHSEDVILCVLPLFHIYSLDSVLLCGLRVGAAILIMQKFDIVPFLELIQKYKVTIAPFVPPIVLAIAKSAMVDNYDLSSVRIVMCGAAPLGKDLEDAVRTKFPNAKLGQGYGMTEAGSVLAMCLAFAKEPFEIKSGACGTVIRNAEMKIMDLNTGSSLPRNKSGEICIRGDQIMKGYLNDPEATAQTIDKEGWLHSGDIGYIDDDDELFIVDRLKELIKYKGFQVAPAELEALLLNLPYISDAAVVPMKDEQAGEVPVAFVVKSSGCTITEDEIKDFISKQVIFYKRIKRVFFVDAVPKSPSGKILRKDLRASLADGLPN

SEQ ID NO: 111 - DNA, 1650 bp, Nicotiana attenuata
  source 1..1650: mol_type = other DNA
atggatccaaggaatggctattgcccacaaactaaaactttccatagcctaagacaaccagtcccattgccaccgttaacggagcctctctctatcatccaatacaccctctctcttctcaactcaccgaccaccggaatcaatgtcaacgccaccactttccttattgacgccaccaccggccaccgtctctcttacgccgacttccttcaaaaaattcaatctcttgcttcttctatccaagcccgttttccttccctttctaaaaacgatgtcgcctttgttctctcccctccttcaccccatattccaattgtatacttcgcactcctctctttagggattgttgtttctccagctaatccactgtcttccactgccgaggtaactcacatgattcagctgtgtaaacccgccattgctttcgcagcttcttcaactgccgttaaactgccgtcgcttccccttggcactattctactcgattcccctgaatttgtctctatgattcaagctcccgtttctactcaaatatcttatccagttattcaccagtcggattcggcagcgattctttactcatctggaactactggaaaagtaaaaggtgttgagttaactcaccggaatttaatcgcgttaacaaccgctttgtattacagcaaattcaataatgttgcggatgaaggtgaaaaggcggagcaagaggtgtcgttcatgacgttgccgatgtttcacgtgtttggattttttatgcttattagattagcggcgttaggggagacggtggtgattattgagagatttgatttcgagaagatgttggcggcggtagagaagtacaaagttacgtacatgccggtgtcgccgccgctggtggtggcaatggcgaagtcggatttggcgttgaagcatgatctcagctcactgaagctgctggcgtgcggcggagcgccgctggggaaggaggtggcagagcggtttaagagcaggttccctaacgtggagataatacagggatatggtctgaccgagactactggaggagcaacagggatgagtggccctgaggaatcagaccggtatgggtctgctggtcgccttgctgtgaatgtggaagctaagattgttgatcctgataatggagagaccttacctcctggccagcgtggagagctttggatacgtgggccaacaatcatgaaaggctatgttggagataaacaagcaacctcagcaacactcgattcagaaggttggctgaaaactggtgatctctgctattttgatacagatgggtttctatatgttgtcgataggttaaaagaactgataaagtacaaggcgtatcaggttccccctgctgaacttgaacatttgcttcaatcaattcctgatgttgctgatgcagcagttattccatatcctgatgaagaagcaggggagattcctatggcttatatagtcagaagaccaggaagcactattagtgagtcacaaattatagatgttatcgcagaacaggttgcaccatacaagaagatacggcgtgttgcattcatcaacgcaataccaaaatctccagcaggaaagattttgagaagagaactcgttaatcatgcctctagtggtgcttctgcaagattatga

SEQ ID NO: 112 - protein, 549 aa, Nicotiana attenuata
MDPRNGYCPQTKTFHSLRQPVPLPPLTEPLSIIQYTLSLLNSPTTGINVNATTFLIDATTGHRLSYADFLQKIQSLASSIQARFPSLSKNDVAFVLSPPSPHIPIVYFALLSLGIVVSPANPLSSTAEVTHMIQLCKPAIAFAASSTAVKLPSLPLGTILLDSPEFVSMIQAPVSTQISYPVIHQSDSAAILYSSGTTGKVKGVELTHRNLIALTTALYYSKFNNVADEGEKAEQEVSFMTLPMFHVFGFFMLIRLAALGETVVIIERFDFEKMLAAVEKYKVTYMPVSPPLVVAMAKSDLALKHDLSSLKLLACGGAPLGKEVAERFKSRFPNVEIIQGYGLTETTGGATGMSGPEESDRYGSAGRLAVNVEAKIVDPDNGETLPPGQRGELWIRGPTIMKGYVGDKQATSATLDSEGWLKTGDLCYFDTDGFLYVVDRLKELIKYKAYQVPPAELEHLLQSIPDVADAAVIPYPDEEAGEIPMAYIVRRPGSTISESQIIDVIAEQVAPYKKIRRVAFINAIPKSPAGKILRRELVNHASSGASARL

SEQ ID NO: 113 - DNA, 1617 bp, Nicotiana attenuata
  source 1..1617: mol_type = other DNA
atggaaaaatctggttatgggcgtgatggaatatacagatcacttagacctcctctgtttcttccaaaaaatcaaaatctttctatggtctcttttctcttcagaaatatttcatcttttcctgaaaaaacagcactctttgatgctgattctggtcaaactttgactttttctcaattcaaaacaattgtgtccaaaatttcccatggtctaattcaccaatttggtatcaagaaaaatgatgttgttcttatttttgcacctaactctattcaattcccttgttgtttctttggtgttattgctttaggtgctattgctacaactgttaatcctatgtacactgtttctgaactttctaagcaaattaaagattgtaaaccaaagcttattattactgtccccgagttgcttgataaaattagtggctttaatttacctattttgcttttgggtcacaagaaaaattctaattttagtggcgtaacgtttactgagttagttgataaagcagggtatgtgaatttggataataatgggataaagcagagtgatactgctgcattgttgtactcttcaggtacaacagggattagtaaaggagtagttttaagtcataggaatttcattgctgcatctttaatggtaactgctgatcaagatttgaagggtgaaatgcacaatgtgtttttgtgtgtgttgcctatgtttcatgtgtttggtcttgctgttattatgtatgcacaattgcaaaggggtagtgctattgtgtctatggctaagtttgatttggaaatgattttgaagactgttgataagtatagggttacgcatttgtggattgtgcctccggttgttttagcattggctaagaatcctgtggtgaagaagtataatttaacgtcattgaggaatattggttcgggggctgcacctttagggaaggaattgatggaggaatgcgctaagaactttcctcaggctgttgttgttcagggatatggtatgacagaaacatgtggaattgcagcactggagagtccatatgttggaccgagacacacaggatcagcggggaccttggttcctggagtggaagctcaaataattagtgttgataaactgaagcctcttcctcctggacaattaggggaaatatgggtgcgtggtcccaacatgatgcaaggctatttcaataatccacaggccacaaagctcactatagataatcagggttgggttcatacgggagatcttggatattttgatgaggaaggaatgctctatgtcgttgataggatcaaagagctgatcaagtacaaaggttttcaggttgcaccagcagagcttgaaggtctacttgtttctcatcctgaaatattagatgctgttgttatcccgtttcctgatgctgaggctggtgaggttccaattgcatacgttgttcgctcgcctaatagctctctaactgaagaagatgttcagaagttcattgcagatcaggttgcacctttcaaaagattgcggaaggtgacattcgtaaacagtgtccccaagtcagcctctgggaaaatcctaagacgagaactgatcgagaaggttcgatcaaaactctaa

SEQ ID NO: 114 - protein, 538 aa, Nicotiana attenuata
MEKSGYGRDGIYRSLRPPLFLPKNQNLSMVSFLFRNISSFPEKTALFDADSGQTLTFSQFKTIVSKISHGLIHQFGIKKNDVVLIFAPNSIQFPCCFFGVIALGAIATTVNPMYTVSELSKQIKDCKPKLIITVPELLDKISGFNLPILLLGHKKNSNFSGVTFTELVDKAGYVNLDNNGIKQSDTAALLYSSGTTGISKGVVLSHRNFIAASLMVTADQDLKGEMHNVFLCVLPMFHVFGLAVIMYAQLQRGSAIVSMAKFDLEMILKTVDKYRVTHLWIVPPVVLALAKNPVVKKYNLTSLRNIGSGAAPLGKELMEECAKNFPQAVVVQGYGMTETCGIAALESPYVGPRHTGSAGTLVPGVEAQIISVDKLKPLPPGQLGEIWVRGPNMMQGYFNNPQATKLTIDNQGWVHTGDLGYFDEEGMLYVVDRIKELIKYKGFQVAPAELEGLLVSHPEILDAVVIPFPDAEAGEVPIAYVVRSPNSSLTEEDVQKFIADQVAPFKRLRKVTFVNSVPKSASGKILRRELIEKVRSKL

SEQ ID NO: 115 - DNA, 1605 bp, Nicotiana attenuata
  source 1..1605: mol_type = other DNA
atggacgcaaacagcggattctgttcaaaaaccaaaactttccacagtctccgtccacctctcttgcttcctccagaagcttctcctctttccgctgcttcttacgcactctctctccaacaatgccccgattccgccgccgccgccttcatcgactccgccaccggccgctgcatctccttctccgagttccgaacacgtgtcatttccctagcttcctcacttcaacacacattccgcttctccaaaaacgacgtcgcatttattctctctccaaattcaactaatgttccggttctctactttgcactcctctccctcggcgttatagtctctcccgctaatcctatgagtacggaatccgagctttttaaacaaattaagttaacgaatcctgtaattgcttttgctacgtcgaggaacttccagaagcttcctatgttgaaacacggaacagttcttattgacttaccggagtttgaatcgatgatgatgaatacggagttgaaattcgagcctgtggaagtgaatcaattagatttggcggcaattatgtattcgtcggggactacaggggaggtaaaaggagtcaagctgagtcatcgtaattttattgcaattattgctaatttttatgctcagagacaagagcggacgtcgccggcgccggtgatgttgtatacggtaccgttgtttcatgtgttcggtttacactatgcgttgaaatcggtagcgttgagtgagactgtagttgtgatggagagatttgatttgaggaaaatgttgaaggcggtggaggagtttaaggtgacgcaattggtggtggctccgccggttgtggtggcaatggctaaaggaagtgttaccagtggctatgatttgagttcgttggtagcggttggtagtggaggtgctccgcttgggaaagatgtgatccaagcattttcagacaagtttccaaataccatagtatttcaaggttatggactaactgagacaagcggggcagcatttcgagctgcaactacagaagaaatgctacgtggggattcagtgggaaggctaatagcaaatactgaagcaaaaattgtagatccagatacggggattgctctgcctcctggtgaacaaggagagctctggattaaaggtccaaccattatgcaaggttacgtgggtgatccgaaggcaacttctgaaactctggtgccgggtggatggttgaggactggtgatctttgctatatcgatcgtcatggttaccttttcattgtagataggctgaaggagctaatcaaatataaaggataccaggttgctcctgctgaactggaacaacttcttcaatcgcacccggacatagtagatgctgctgtcataccatttcctgatgaagaagctggtcaagtacccatggcatttgttgtgagacgtccccagagcgctctggacaaagagcaagtgattgattttatttcgaaacaggttgctccatataagaaaataaggcgtgtggcatttgttagttccatacccaagaatccatctgggaagatattgagaaaagaattaaggaggattcatataccaggatctaagttgtaa

SEQ ID NO: 116 - protein, 534 aa, Nicotiana attenuata
MDANSGFCSKTKTFHSLRPPLLLPPEASPLSAASYALSLQQCPDSAAAAFIDSATGRCISFSEFRTRVISLASSLQHTFRFSKNDVAFILSPNSTNVPVLYFALLSLGVIVSPANPMSTESELFKQIKLTNPVIAFATSRNFQKLPMLKHGTVLIDLPEFESMMMNTELKFEPVEVNQLDLAAIMYSSGTTGEVKGVKLSHRNFIAIIANFYAQRQERTSPAPVMLYTVPLFHVFGLHYALKSVALSETVVVMERFDLRKMLKAVEEFKVTQLVVAPPVVVAMAKGSVTSGYDLSSLVAVGSGGAPLGKDVIQAFSDKFPNTIVFQGYGLTETSGAAFRAATTEEMLRGDSVGRLIANTEAKIVDPDTGIALPPGEQGELWIKGPTIMQGYVGDPKATSETLVPGGWLRTGDLCYIDRHGYLFIVDRLKELIKYKGYQVAPAELEQLLQSHPDIVDAAVIPFPDEEAGQVPMAFVVRRPQSALDKEQVIDFISKQVAPYKKIRRVAFVSSIPKNPSGKILRKELRRIHIPGSKL

SEQ ID NO: 117 - DNA, 1677 bp, Nicotiana attenuata
  source 1..1677: mol_type = other DNA
atggaaatggtgaaaaacaaagaaaatcacaatgatcaatacatttttcgatcaaaacttcctgatatttacattccaaatcatctccctcttcatacttattgctttgaaaatatctcccaattcagttcacgtacttgcttgatcaatagtgccacgggtgcaacatatacctatgccgatgttgaactcacctccaaaagagttgctatcggactttacaaacttggaattcaacaaagagatgttatcatgcttttacttccgaattcgcccgaattcgtgttcgcgttcctcggagcatcgttccgaggagcgataagcacgacggcgaatccgttctacacgccgtcggagatcacaaagcaagcaaaagcatcaaatgtgaagcttattattacacaatcttgttacgttgataaagtcaagagtttcgctgaggaaaacgaggtgaaaattatgtgtacggataactcaccaccagaatatggttgtcttagtttcgctgagttaattactgatcaatcagaagaagataatcataattatgattataataccattttatctgatatagaaatcttttctgacgatgtcgtggcgttgccttattcgtcgggcacgacggggttgcccaaaggagtaatgttaacacataaagggctagtaacaagtatagcacaacaagttgatggtgaaaaccctaatctttatttcaatagtgaagatgtgattatgtgtgttttgccattgtttcatatttattcactcaattctgtgttgctttgtgggctaagagttggggcaacaattttaataatgcaaaagtttgagattaaaggattaatggagctagttgagaagtatagggtgacaattgcaccatttgtgccgcctatagttttggccatagcaaaaagtccattggttgataaatatgatttgtcaagtataaggatgataatgtcaggagctgcaccaatggggaaagagcttgaagatactgttagagctaaattgccgaatgccatactaggacagggatatggaatgactgaagcaggtccagtgttatcaatgtgtctagcctttgcaaaacaacagtttgaagtcaagtcaggttcctgtgggacagttgttcggaatgctgagatgaagatcgtcgacaccaacaccggtgcgtcgcttccccggaatcatgccggagaaatctgcattagaggtgatcagattatgaaaggatatctgaataatccaaaagcgacagaggaaacaatagacaaagaaggatggttacacactggtgacattggatacatagatgatgatgatcaagtatttattgtggatagattaaaagaattaatcaaatacaaaggatatcaagttgctcctgctgaattagaagctctactaatttctaatccaaatataactgatgctgctgttgtaccgatgaaagatgaggttgcaggagaagttcctgttgcttttgtagtgagagcaaatggtagcaacatttcagaggaagagatcaaaaaatatatatccgaacaggttatattttacaagcgaatcaatcgagtatttttcacagaagcaatcccaaaagctccatcagggaaaatacttcgaaaagatttaagagccaagcttgcgacaggtttagcttgttaa

SEQ ID NO: 118 - protein, 558 aa, Nicotiana attenuata
MEMVKNKENHNDQYIFRSKLPDIYIPNHLPLHTYCFENISQFSSRTCLINSATGATYTYADVELTSKRVAIGLYKLGIQQRDVIMLLLPNSPEFVFAFLGASFRGAISTTANPFYTPSEITKQAKASNVKLIITQSCYVDKVKSFAEENEVKIMCTDNSPPEYGCLSFAELITDQSEEDNHNYDYNTILSDIEIFSDDVVALPYSSGTTGLPKGVMLTHKGLVTSIAQQVDGENPNLYFNSEDVIMCVLPLFHIYSLNSVLLCGLRVGATILIMQKFEIKGLMELVEKYRVTIAPFVPPIVLAIAKSPLVDKYDLSSIRMIMSGAAPMGKELEDTVRAKLPNAILGQGYGMTEAGPVLSMCLAFAKQQFEVKSGSCGTVVRNAEMKIVDTNTGASLPRNHAGEICIRGDQIMKGYLNNPKATEETIDKEGWLHTGDIGYIDDDDQVFIVDRLKELIKYKGYQVAPAELEALLISNPNITDAAVVPMKDEVAGEVPVAFVVRANGSNISEEEIKKYISEQVIFYKRINRVFFTEAIPKAPSGKILRKDLRAKLATGLAC

SEQ ID NO: 119 - DNA, 1698 bp, Nicotiana attenuata
  source 1..1698: mol_type = other DNA
atggcaacattcatgaaaccccatttgaactcccatgccacagagggtgaagttgtgaaaaacccttctaaaacccccttttggtactgtgctgaaacaggagtatacaatagtaagcacccttttcttaaactgccagaagacccttttcttgatgttgtttccttcattttctcacataagcatggtggattctcagctcttgttgattccatttctggcatttcaattccctattcagagctttactcaatggttaagtccatggctagtagtcttcaccaaaggggtgtctcacaaggtgatgttgtgttaattctcctcccaaattccatttgtttccctgttattttgttgggtgttttgagtcttggtgcaattgcaaccactatgaatcccttcagtagttggttagagattaaaaaacaagcccttgattgtggtgttattctggcatttactactgatgataaagttgataaattggctgcattaggttttcctgtaataggggtgtcagaaagtttggtttctaattcaaatggtagtcaaagttctggttttcatgagctgatttcatgtgatcccaattgggattcaaggcctaaaataagtcagcaagatacagcagcaattctatactcgtcgggtactactggtactggcaaaggtgttgtcttgactcatgcgaactttatagccatggtacaactttttgtgaggtttgaagcttcacaatatgaatatttgagtactgagaatgtgtacttggctgttgtaccgatgtttcatgtgtacgggctctctctctttgtgatgggattgttgtcattggggtctaccgttgttgtgatgggaaaatttgatgctgatgagatggtgaaagctattgaaagatatggtgtcacccacttccccacggtcccaccattattgatggcattgactagaagagcaaaggatggtgcttcgagcagtatgaagagcttgaaacaggtttcatgtggcgcagctcccttaaacacaaaatctattgaagaatttgttcgcactctcccagatgttgatttcattcagggatatggcatgactgagtcaactgctattggaacacgtggttacagtactgaaaagcttcgtaattattcttcggtagggcttctagctccaaacatgcaagctaaagtagtggattggatcacaggttcacccttacctcccaactgcatgggcgagctctggctttgtggacctggtgtcatgaaagggtacttgaataactctgaggctactaagtatacgattgatggaaatggttggctccacactggtgatattgcttattttgatgaagaaggatatctttatgtcattgaccgactgaaagagattatcaaatataagggctttcagatagctcctgctgatttagaagctgtattggtctctcatccagatatcgttgatgctgcagtaacgggagccagagatgaggaagctggagaaattccagtggcatttgtcgtcaagagagatgggtgtgcactttcccaaacagatgttatcgactttgtttgtaagcaggttgcaccatacaagaagattagaagggtttacttcaaagcttcaatacctaggtctgcagctggaaagattcttagaaaggagttaaggaacttattaacttccaaattgtag

SEQ ID NO: 120 - protein, 565 aa, Nicotiana attenuata
MATFMKPHLNSHATEGEVVKNPSKTPFWYCAETGVYNSKHPFLKLPEDPFLDVVSFIFSHKHGGFSALVDSISGISIPYSELYSMVKSMASSLHQRGVSQGDVVLILLPNSICFPVILLGVLSLGAIATTMNPFSSWLEIKKQALDCGVILAFTTDDKVDKLAALGFPVIGVSESLVSNSNGSQSSGFHELISCDPNWDSRPKISQQDTAAILYSSGTTGTGKGVVLTHANFIAMVQLFVRFEASQYEYLSTENVYLAVVPMFHVYGLSLFVMGLLSLGSTVVVMGKFDADEMVKAIERYGVTHFPTVPPLLMALTRRAKDGASSSMKSLKQVSCGAAPLNTKSIEEFVRTLPDVDFIQGYGMTESTAIGTRGYSTEKLRNYSSVGLLAPNMQAKVVDWITGSPLPPNCMGELWLCGPGVMKGYLNNSEATKYTIDGNGWLHTGDIAYFDEEGYLYVIDRLKEIIKYKGFQIAPADLEAVLVSHPDIVDAAVTGARDEEAGEIPVAFVVKRDGCALSQTDVIDFVCKQVAPYKKIRRVYFKASIPRSAAGKILRKELRNLLTSKL

SEQ ID NO: 121 - DNA, 1656 bp, Nicotiana attenuata
  source 1..1656: mol_type = other DNA
atgggaactcatgctgtagaaagctcacagcaacaagaatgtgagcacattttccggagtagatatccaccagttcaagtaccggataatgtgactctcccagattttgtgcttcacaatgtagagttatacaccgacaaaatggcatttgtggatgctaccactggcaaaggctacacttatggtgaagttgcaagagacataaggaggttcgccaaggccttgagatcccttggcttaaggaaaggacgggtggtggtggtagttcttccaaatgtaccagaatatgctattgttgctctcggaatcatggctgctggtggcgtcttctccggtgcaaatccagctgctcattcatcagaaattatgaaacaagttgaatctgctgatggcaagcttattgtctctgatctaccaacctatcacaaggttaaagattgtgggctgccagtaataatactaggtgaagaacatgtagaaggaacaattcattgggatgaattgcttgaagctgcagagcgtgccggttcaagaactgatcacataacaaaccaagaagatgaaatggtgcagcaaaatgatttatgtgcactgcccttctcatcaggcactacggggctgtccaagggagtgatgttaactcacagaaatctagtagcaaatctctgctctacactcttcagtgttagcccagaaatgataggccaagttacaacactaggtttgataccatttttccacatttatgggataactggaatctgttgtgcaaccattagaaacaaagggaaagtggtagtcatgcgtaggtacgaactaagggcatttctaaatgcactcatcgcgcatgaagtcacatttgcgccgattgtgccacctatcatcttggcacttgttaagaaccctattgtggatgaatttgatctcaataagcttaagcttagatccattatgacagcggcagcaccacttgcccctgagattcttaatgaatttgagaagaaatttccggatgttcaggtccaagaggcatatgggatgactgagcacagctgcattactctctctcatagtgaccagcatactgctaaaagaaattctgttggtttcattctacctaatttggaggtaaagttcgttgatcctgatacgggtagatctctccccaaaaacacaccaggcgagatatgtgtcaaaagccaatgtgttatgaagggttactacaaaaatgaatttgagacttgccttaccattgataaagatggatggcttcacactggtgacattggctacatagacgatgatggagatatcttcctagtcgatcgtatcaaagagcttatcaagtacaagggattccaagttgctccagctgagttagaggggatccttctcacacatccttcagtagaagatgctgcagtagttgggctgcctgatgaagaagcaggagagataccagcggcatgggtagtcttgaactcaaaagcaaaagaaagcgaagaggacattatcaactacattgcatcgactgttgcacagtataaacgagtgagagtggtgcagttcgtcgatagtattccaaaatctccttctggaaaaataatgagaagacttatcaaggataagatgctagagagacttaagaatgcatag

SEQ ID NO: 122 - protein, 551 aa, Nicotiana attenuata
MGTHAVESSQQQECEHIFRSRYPPVQVPDNVTLPDFVLHNVELYTDKMAFVDATTGKGYTYGEVARDIRRFAKALRSLGLRKGRVVVVVLPNVPEYAIVALGIMAAGGVFSGANPAAHSSEIMKQVESADGKLIVSDLPTYHKVKDCGLPVIILGEEHVEGTIHWDELLEAAERAGSRTDHITNQEDEMVQQNDLCALPFSSGTTGLSKGVMLTHRNLVANLCSTLFSVSPEMIGQVTTLGLIPFFHIYGITGICCATIRNKGKVVVMRRYELRAFLNALIAHEVTFAPIVPPIILALVKNPIVDEFDLNKLKLRSIMTAAAPLAPEILNEFEKKFPDVQVQEAYGMTEHSCITLSHSDQHTAKRNSVGFILPNLEVKFVDPDTGRSLPKNTPGEICVKSQCVMKGYYKNEFETCLTIDKDGWLHTGDIGYIDDDGDIFLVDRIKELIKYKGFQVAPAELEGILLTHPSVEDAAVVGLPDEEAGEIPAAWVVLNSKAKESEEDIINYIASTVAQYKRVRVVQFVDSIPKSPSGKIMRRLIKDKMLERLKNA

SEQ ID NO: 123 - DNA, 1572 bp, Nicotiana attenuata
  source 1..1572: mol_type = other DNA
atggcgagtttcacactcactggattgttgaaacatgttgccgaaaaattcccttctcatcgtgcgatttccatttctgggaagtttgatattactcatgcacggcttcaagaactcgttgaacgtgctgcttctcaacttgtatctgtcggtgttaagcacggcgatgtcgttgcactcacttttcccaacactatcgagttggtgatcatgtttctagctgtaattcgagtacgagccacggcggcgccactcaattcagcttacatgaccgaagaattcgagttctatttatccgattcagaatcgaaactcttaataactggaaaagaaggaaatgaagcggctcaaaccgcagcttccaagctgaaaatccgtcatattactgcaactctgcctcaagccgactctgatatcaccttttcccctgctccttccgggtcggaccttgaatcagtgtccaaaatcgttaacgacccgtcggatgttggactttttcttcatacatcgggcaccacgagccgaccaaaaggtgttcctctgacccagctaaatttggcatcttcagtgaataatatcaaatcggtttataaactcagtgattcggattcaacggtgatcgtgttgccgttatttcacgttcacgggttaattgccggattactgagttcactgggagccggagcatccgtaacacttccttccgctgggagattttctgcttcaactttctggccagatatgaaaaaatacaacgccacgtggtacacggctgtacctacaattcaccagattcttttggattaccacctcagcaaacccgaatccaattacccgaagctccggctcattcggagttgcagtgcagcactagctccatcagtgatggcccggttagaggaagcattcggggctcctgttttggaggcgtatgcgatgacagaggcgacccatttgatggcttcgaacccgttacccgaaaatggcccgcatatacccgggtcggttgggaaacccgtgggtcaagagatggctatattggatgagaatggtgtggtacaaggtgctaatgctaagggagaggtttgcattagaggtccaaatgtaacaaaagggtataagaataatcctgaggctaataaatcagctttccagtttggttggtttcacactggtgatgtggggtatttggactctgatggatatttgcatttggttggcagaattaaggagttaatcaaccgtggaggggagaaaatatcacctattgaagtggatgcagtactggtttctcatgcggaaattgctcaggcagttgcttttggcgttcctgacgacaagtatggtgaagagataaactgtgcagttattccaagagaagggtcaaacattgatgaggcagaggtgctgagattttgtaagaagaatctcgcagcttttaaggtccctaagaaggttttcatgactgactctctaccaaagactgcaacaggaaaaattcaacgacgacttgttgcagagcacttcctagcacagatttcaactgctaaagtccccaagttcggagcttag

SEQ ID NO: 124 - protein, 523 aa, Nicotiana attenuata
MASFTLTGLLKHVAEKFPSHRAISISGKFDITHARLQELVERAASQLVSVGVKHGDVVALTFPNTIELVIMFLAVIRVRATAAPLNSAYMTEEFEFYLSDSESKLLITGKEGNEAAQTAASKLKIRHITATLPQADSDITFSPAPSGSDLESVSKIVNDPSDVGLFLHTSGTTSRPKGVPLTQLNLASSVNNIKSVYKLSDSDSTVIVLPLFHVHGLIAGLLSSLGAGASVTLPSAGRFSASTFWPDMKKYNATWYTAVPTIHQILLDYHLSKPESNYPKLRLIRSCSAALAPSVMARLEEAFGAPVLEAYAMTEATHLMASNPLPENGPHIPGSVGKPVGQEMAILDENGVVQGANAKGEVCIRGPNVTKGYKNNPEANKSAFQFGWFHTGDVGYLDSDGYLHLVGRIKELINRGGEKISPIEVDAVLVSHAEIAQAVAFGVPDDKYGEEINCAVIPREGSNIDEAEVLRFCKKNLAAFKVPKKVFMTDSLPKTATGKIQRRLVAEHFLAQISTAKVPKFGA

SEQ ID NO: 125 - DNA, 1644 bp, Nicotiana attenuata
  source 1..1644: mol_type = other DNA
atgccgatggagagtactaacgaaacaaagcaatcaggagatttaatcttccgatcaaaactccctgatatttacatccctaagcatttacctttacattcttattgtttcgaaaacatttcggagttcagttctcgtccctgtttgatcaatggagccaacgatcaaatttatacttatgctgaagttgagctcacttgcagaaaagttgcagctggtcttaacaagttagggatccaacaaaaggacacgattatgatccttttgccgaattcccctgaatttgtgtttgcttttatgggcgcttcatatttaggagccatctctacaatggccaatcctctgtttacgccagcagaggttgtaaaacaagccaaagcctcaagtgctaagattattataacgcaatcgtgctttgtgggcaaagtgaaggattacgctagcgaaaatgatgtgaaggttatttgtattgattctgcaccagaaggttgtcttcatttctctgaattgacacaatctgatgagcatgaaattcctgaggtgaaaatccaatcggacgacgtcgttgcgctgccgtattcctctggcactacagggctgccgaaaggtgtgatgttgacacacaagggattagtcacgagcgttgcacaacaagttgacggtgaaaatgcaaatttgtatatgcacagtgaagatgtgttgatgtgtgtgttgcctttgttccatatttactcactcaattccattttgctttgtggattgagagtcggtgcagcgattttgattatgcagaaattcgacattgctccgttcctggagttaatacagaagtacaaggtgtcaattgggccatttgtgccgcctattgttctggccattgctaagagtccaattgttgatagctatgatctttcatcagtaaggactgtcatgtctggggctgcaccgttaggaaaagaactcgaagacgctgtgcgaaccaaattccctaaggctaaacttggccagggttatggaatgacggaagctgggccggtgttggcaatgtgtctggcatttgcaaaagaaccgtttgatataaaatcaggggcatgtggtactgttgtgaggaacgccgagatgaaaattgtagatccggatacgggttgctctcttcctcgtaaccaacccggtgagatttgcattagaggtgaccagatcatgaaaggttacctgaatgatcccgaggccacaacgagaacaatagacaaagaaggatggttacacactggcgacattgggttcattgacgaggatgacgagcttttcattgtggatcgattgaaggaattgatcaaatacaaaggatttcaagtggcacctgcagaaattgaagcccttctcctcaaccatcccaacatttctgatgctgctgttgtccccatgaaagacgagcaagcgggagaagttccggtagcttttgttgttaggtcaaatggatctgctattactgaagatgaagtcaaggatttcgtctcgaaacaggtgatattttataagagagtaaagcgcgtatttttcgtggagacggtacccaaatctccgtcgggaaaaattcttagaaaggacttgagagctagactggctgctggtgttccaaattaa

SEQ ID NO: 126 - protein, 547 aa, Nicotiana attenuata
MPMESTNETKQSGDLIFRSKLPDIYIPKHLPLHSYCFENISEFSSRPCLINGANDQIYTYAEVELTCRKVAAGLNKLGIQQKDTIMILLPNSPEFVFAFMGASYLGAISTMANPLFTPAEVVKQAKASSAKIIITQSCFVGKVKDYASENDVKVICIDSAPEGCLHFSELTQSDEHEIPEVKIQSDDVVALPYSSGTTGLPKGVMLTHKGLVTSVAQQVDGENANLYMHSEDVLMCVLPLFHIYSLNSILLCGLRVGAAILIMQKFDIAPFLELIQKYKVSIGPFVPPIVLAIAKSPIVDSYDLSSVRTVMSGAAPLGKELEDAVRTKFPKAKLGQGYGMTEAGPVLAMCLAFAKEPFDIKSGACGTVVRNAEMKIVDPDTGCSLPRNQPGEICIRGDQIMKGYLNDPEATTRTIDKEGWLHTGDIGFIDEDDELFIVDRLKELIKYKGFQVAPAEIEALLLNHPNISDAAVVPMKDEQAGEVPVAFVVRSNGSAITEDEVKDFVSKQVIFYKRVKRVFFVETVPKSPSGKILRKDLRARLAAGVPN

SEQ ID NO: 127 - DNA, 1719 bp, Nicotiana attenuata
  source 1..1719: mol_type = other DNA
atgttgagtgtagctagtgttgaagcccagaaggcagagttatcttcttctgtgattcctccttcaaatcaatcttctgaagagttacatgtgtttagatcaagattaccagatataccaatttccaataatgtccctcttcatgtttacttgtttgaaaggctctctgaatttcaagataggacatgtcttatatcaggtagcagtggaaaatcctacacttttgctgaaactcatctcatttgccagaaaatagctgctggtttaaacaacatcggaatcaaaaagggagatgtaattatgacttttctccagaactgcgcggaatttgtgtttacttttctctcggcttctatgattggcgctgttataactacagctaatccattttacacaaaagctgaggcgtttaagcaattaaaagcgtcgaacgcgaaacttatcgttactcaatctcagtacgtggataaatttcgtgattccggagagaatgacccgaaaattggcgaagatttttcagttattacaatcgacgacccgcctgaaaattgcttacatttctctgtactttctgaagctaacgaagagaaaatgccaaaaggaattgttatccaacccgatgatccagtagctttgccattttcatcaggaacaacagggttaccaaaaggtgtgattttaactcacaaaagtttaatcacaggagtagctcaattagtcgacggagacaatccaaatttgtacttgaaaaacgatgacgtggtgctatgcgttctacctctgtttcacatatttgcgttaaattcagtgcttttagtctcgttaagagcaggagctagtgttttattaatgcaaaaattcgaaattggtgcattgctggagctgatacaacaccaccgcgtgtcagttgctgctgtagttccgcccttggttcttgcgttggctaaaaatccaatggttgattcgttcgatttgagttcaattaggctcgtgttgtccggggcggcgccgctggggaaagagttggaggaagcgctacatcgtagagtcccgcaagctatatttggacaggggtatggtatgacagaggcaggaccagtagtaacaatgtgcccagcatttgcaaagcaaccattttcaaccaaatctggctcatgtggttcagtagttcgaaatgcagacctcaaagtggtcgaccctgaaactggtggctcccttggccgcaaccaacctggcgaaatttgcatccgcggttcacaaatcatgaaaggttatttgaatgatgatgaagccactgcacggaccattgatgtcgatggatggctccacactggtgatataggatatgtagatgacgatgatgaaatatacatcgtcgatagagtgaaagagctcatcaagttcaaaggtttccaggtgccaccagctgagcttgagtctcttttagtaagccatccagatattgcagatgctgctgttgtgccgcaaaaggatgatgcggcaggggaagtcccagttgcatttgtggtccgctccgccaatggttttgaaattactgaagaagctataaaggaatttattgccaaacaggtgatattctataaaagattgcacaaggtgtactttattcactctattccaaagtctccgtctggaaagatactgaggaaagaactgagagccaaactagctgcaccaccagcctcctccaccgagtga

SEQ ID NO: 128 - protein, 572 aa, Nicotiana attenuata
MLSVASVEAQKAELSSSVIPPSNQSSEELHVFRSRLPDIPISNNVPLHVYLFERLSEFQDRTCLISGSSGKSYTFAETHLICQKIAAGLNNIGIKKGDVIMTFLQNCAEFVFTFLSASMIGAVITTANPFYTKAEAFKQLKASNAKLIVTQSQYVDKFRDSGENDPKIGEDFSVITIDDPPENCLHFSVLSEANEEKMPKGIVIQPDDPVALPFSSGTTGLPKGVILTHKSLITGVAQLVDGDNPNLYLKNDDVVLCVLPLFHIFALNSVLLVSLRAGASVLLMQKFEIGALLELIQHHRVSVAAVVPPLVLALAKNPMVDSFDLSSIRLVLSGAAPLGKELEEALHRRVPQAIFGQGYGMTEAGPVVTMCPAFAKQPFSTKSGSCGSVVRNADLKVVDPETGGSLGRNQPGEICIRGSQIMKGYLNDDEATARTIDVDGWLHTGDIGYVDDDDEIYIVDRVKELIKFKGFQVPPAELESLLVSHPDIADAAVVPQKDDAAGEVPVAFVVRSANGFEITEEAIKEFIAKQVIFYKRLHKVYFIHSIPKSPSGKILRKELRAKLAAPPASSTE

SEQ ID NO: 129 - DNA, 1638 bp, Nicotiana attenuata
  source 1..1638: mol_type = other DNA
ataaacccaaaaaatggctactctactcataccaaaactttccacagcctaaggtcatcactcccattgccacctttatcacagcccatttccataattcagtacacactctctctcctcaactccacaaccaccgccaccacctctaacaccactttcctcatcgactccaccacaggccgccgcctctcttactccgaattccttcacaaaacccaatcccttgcctcttctctcaagattcaattcccgtccctttccaaaaacgacgtcgcctttatcctgtctcctccatcgcttcatgttccaatcctctacttcgcccttctctcccttggcattgttgtttcccctgctaacccactctcttcttctaccgagttgactcacatgatccaactcagtaaacccaccattgcttttactacttcatcgatttcccataaactcatcccttcatttcccctcggcaccattttaattgactcccccgaattccttgaaccccccattgctacgcctatctccaacttcaactcaattgttcaccaaactgattcggcagctattctttactcctctggaactactggccgagtcaagggagtcgagttgactcaccggaatttgattgccttaactgccggcttatacaataacaaattcaccagcgacgaaagtgaaaatgcagagcaagacgtggcgattttgacgttgccgttgtttcacgtttttggattcttcatgctgattaggttagcggcgatgggggaaacggcggtgataatggagagattcgatttcgagaagatgttggaggcggtggagaagtataaggtgacgtacatgcctgtgtctccgcccttggtggtggcgatggcgaagtcggagttggtactgaagtatgatctgacgtcgctgaagctggtggcgtgcggcggagcgccgctaggaagggaggtggctgagcggttcaaggcgaggttctctaacgtggagattgtgcagggatatggtctaaccgagagtacgggagcaggtacagggatgataagacctgaggaaacagcccgatatggatctgctggtcgccttgcagataatttggaagctaagatagtcgatcctgatagtgaggaggccttacctcccgggaagcgtggagagctttggatacgcgggccaacaatcatgaaaggttatgttggagataaacaagaaacatcagccactcttaattcagaaggttggctaaaaactggtgatctctgctattttgactcggatgggttcctgtttattgttgataggctaaaagaactgataaagtacaaggcatatcaggtaccccctgctgaactggaacagttgcttcaatcaattcctgaaattgctgatgcagcagttattccatatcctgatgaagaagcagggcagattccaatggcttatgttgtcagaaaacctgggagcactattagtgagtgtcacattgtggattcaattgcaaaacaggttgcaccatacaaaaagatacggcgtgttgcattcattaactcaataccaagatctccagcaggaaagatcttgagaagagaactcattgatcaggccatttctggtgcttcagctagatta

SEQ ID NO: 130 - protein, 546 aa, Nicotiana attenuata
INPKNGYSTHTKTFHSLRSSLPLPPLSQPISIIQYTLSLLNSTTTATTSNTTFLIDSTTGRRLSYSEFLHKTQSLASSLKIQFPSLSKNDVAFILSPPSLHVPILYFALLSLGIVVSPANPLSSSTELTHMIQLSKPTIAFTTSSISHKLIPSFPLGTILIDSPEFLEPPIATPISNFNSIVHQTDSAAILYSSGTTGRVKGVELTHRNLIALTAGLYNNKFTSDESENAEQDVAILTLPLFHVFGFFMLIRLAAMGETAVIMERFDFEKMLEAVEKYKVTYMPVSPPLVVAMAKSELVLKYDLTSLKLVACGGAPLGREVAERFKARFSNVEIVQGYGLTESTGAGTGMIRPEETARYGSAGRLADNLEAKIVDPDSEEALPPGKRGELWIRGPTIMKGYVGDKQETSATLNSEGWLKTGDLCYFDSDGFLFIVDRLKELIKYKAYQVPPAELEQLLQSIPEIADAAVIPYPDEEAGQIPMAYVVRKPGSTISECHIVDSIAKQVAPYKKIRRVAFINSIPRSPAGKILRRELIDQAISGASARL

SEQ ID NO: 131 - DNA, 1470 bp, Nicotiana attenuata
  source 1..1470: mol_type = other DNA
aaagttgcactaattgactctgcaaataaccagaaattaacttatgctcaactccaacgttctattgcttcacttgccacaggattgtaccatgcactaggtgttagaaaaggtgatgttgtctttgttttgtcaccaaattcacttttgtatccaataatatgtctagctgttctttcagttggtgctatattatctcctgccaatccacttaacactgaatcagaaatcgggaaacaagtacgtttatcgggtgcaaaattagctattgctgcaccagaagaagtgcataaattagtccacactagtgtgccaactcttctcacaactcgacccaagaacgatgaaaattcagtttcagtagaggatttgattgagaattgtgatcctttagaaatgccaaaggacagaccaatgctttcagatacagcagcaatactatactcttcaggaactactggagttagcaaaggtgtagtgttaactcatgctaatttttttgcaattatgaaacttcttaagttctacgtcgatgtaacatcgtctcaagatgatattttcctctgtttcataccgacgtttcatgtatacggtctagtctttttcggtctgggattattttgctcaggtgttacaactgtgttgatgcaaaaatttgatttccaagctatgttggaagcaattcagacacacaaagtcagtaacattccagcagttccacctgtgattctcgggctagttaagtataacaaaggtggttatgatttatcatcgttgcggaaggtttcctcaggggctgccactgagcaaagaggtattgtagcagatgcattccgggagaagttcccatgggtggagctcaagcaaggctatggtctaactgagacaactggtgcggctacatttttcattaccaatgaagaggcaaaggcgcgtccatgctcagcaggaatgttgttcccgaccttctccgctaaggtggttgatcacgagacaggggaggcgatgccaccatttaaagaaggtgaactgtggttaaaaggtccaggggtgatgaaagagtatttcggaaatgaggaatcaactgctgctacaatcactgctgatggctggctccgaactggtgatctttgttactttgacgacgagggatatttgtacatcgttgatcgaataaaggaactcatcaagcataatggttatcaggtagctccagcagaacttgaagcaatactattgagtcaccatcagatacttgatgcagcagttgtcccactagaagacgaggaagcaggacaaataccagtggcatatgttgtaaaagcagctggttctgagctcacagaagaccaggtcattcaatttgtttctagccaagtagccccatacaagaaagtcagaagagttaattttatcagtggtataccaagatcagctgcagggaaaatattgcgaaaggagttagtgcaa

SEQ ID NO: 132 - protein, 490 aa, Nicotiana attenuata
KVALIDSANNQKLTYAQLQRSIASLATGLYHALGVRKGDVVFVLSPNSLLYPIICLAVLSVGAILSPANPLNTESEIGKQVRLSGAKLAIAAPEEVHKLVHTSVPTLLTTRPKNDENSVSVEDLIENCDPLEMPKDRPMLSDTAAILYSSGTTGVSKGVVLTHANFFAIMKLLKFYVDVTSSQDDIFLCFIPTFHVYGLVFFGLGLFCSGVTTVLMQKFDFQAMLEAIQTHKVSNIPAVPPVILGLVKYNKGGYDLSSLRKVSSGAATEQRGIVADAFREKFPWVELKQGYGLTETTGAATFFITNEEAKARPCSAGMLFPTFSAKVVDHETGEAMPPFKEGELWLKGPGVMKEYFGNEESTAATITADGWLRTGDLCYFDDEGYLYIVDRIKELIKHNGYQVAPAELEAILLSHHQILDAAVVPLEDEEAGQIPVAYVVKAAGSELTEDQVIQFVSSQVAPYKKVRRVNFISGIPRSAAGKILRKELVQ

SEQ ID NO: 133 - protein, 607 aa, Vitis vinifera
MASLPWSLTTSTAIANTTNISAFPPSPLFQRASHVPVARNRSRRFAPSKVSCNSANGDPNSDSTSDVRETSSGKLDRRNVLLGIGGLYGAAGGLGATKPLAFGAPIQAPDISKCGTATVPDGVTPTNCCPPVTTKIIDFQLPSSGSPMRTRPAAHLVSKEYLAKYKKAIELQKALPDDDPRSFKQQANVHCTYCQGAYDQVGYTDLELQVHASWLFLPFHRYYLYFNERILAKLIDDPTFALPYWAWDNPDGMYMPTIYASSPSSLYDEKRNAKHLPPTVIDLDYDGTEPTIPDDELKTDNLAIMYKQIVSGATTPKLFLGYPYRAGDAIDPGAGTLEHAPHNIVHKWTGLADKPSEDMGNFYTAGRDPIFFGHHANVDRMWNIWKTIGGKNRKDFTDTDWLDATFVFYDENKQLVKVKVSDCVDTSKLRYQYQDIPIPWLPKNTKAKAKTTTKSSKSGVAKAAELPKTTISSIGDFPKALNSVIRVEVPRPKKSRSKKEKEDEEEVLLIKGIELDRENFVKFDVYINDEDYSVSRPKNSEFAGSFVNVPHKHMKEMKTKTNLRFAINELLEDLGAEDDESVIVTIVPRAGGDDVTIGGIEIEFVSD

SEQ ID NO: 134 - protein, 586 aa, Phytolacca americana
MASLSSPPTSTTTSATTLATNISNPLFPKTSQFSLPGCRNRRHVMPKISCKVDKDELYPKTTNDDGNTNKLDRRNMLIGLGSIYGAGTTLGGGPATLAAPLITPNPTEFGASDVGLNCCPPTARNIVDFQLPPQPTTLRVRPPAHLVDDQYLQKFSRAISLMKALPANDPRNFSQQANVHCAYCDGGYTQTGFPNLELQVHFSWLFFPFHRFYLYFFERILGKLINDPSFAIPFWNWDTPAGMQIPGIYTSPNSPLYDPRRDRAHQPPRVIDLNYDGQDDPNDLTGQDLISANLTLMYRQMVSNSTTTRLFLGQPFRVGNRPSPGAGSIENVPHGPVHVWTGDPNQPNGEDMGTFYSAARVPIFYAHHAIVDRMWAIWNALGNDRQDFTDSDWLNASCIVYGGNGQAVRVPSRDSLDKARLGYTYQGVDLPEWPSSPTPQRSKLNTTSRIPMSPTLSTVKFPRALDSVIRTDVKRSKKSRGRREKEVEEEILVIEIELEKDIYVKFDVHVNDEDEVPFKKTRVLTEFAGSFVNVPHKHHKEGQHGMKTALRLGLTALIEELGADDDETVTITLVPRAGNDVVVTRM

SEQ ID NO: 135 - protein, 562 aa, Antirrhinum majus
MFKNPNIRYHKLSSKSNDNDQESSHRCKHILLFIITLFLLIVGLYIANSLAYARFASTSTGPIAAPDVTKCGQPDLPPGTAPINCCPPIPAKIIDFELPPPSTTMRVRRAAHLVDDAYIAKFKKAVELMRALPEDDPRSFKQQANVHCAYCAGAYNQAGFTNLKLQIHRSWLFFPFHRYYIYFFERILGKLINDTTFALPFWNYDSPGGMTIPSMFIDTNSSLYDSLRDSNHQPPTIVDLNYAFSDSDNTTTPEEQMIINLKIVYRQMVSSAKTPQLFFGRPYRRGDQEFPGVGSIELVPHGMIHLWTGSENTPYGENMGAFYSTARDPIFFAHHSNVDRMWSIWKTLGGPRRTDLTDPDFLDASFVFYDENAEMVRVKVRDCLDEKKLGYVYQDVEIPWLNTRPTPKVSPSLLKKFHRTNTANPRQVFPAILDRVLKVIVTRPKKTRSRKEKDELEEILVIEGIELERDHGHVKFDVYINADEDDLAVISPENAEFAGSFVSLWHKPIKGKRTKTQLLTLSICDILEDLDADEDDYVLVTLVPRNAGDAIKIHNVKIELDG

SEQ ID NO: 136 - protein, 590 aa, Nicotiana attenuata; identical to SEQ ID NO: 6

SEQ ID NO: 137 - protein, 596 aa, Nicotiana attenuata; identical to SEQ ID NO: 4

SEQ ID NO: 138 - protein, 599 aa, Solanum tuberosum
MASVCNSSSTTTTLKTPFISSNTSLGSTPKPSQLFLHGKRNKTFKVSCKVSNSGDQNQNIETNSVDRRNVLLGLGGLYGVANAIPLAASATPIPSPDLKTCGRATISDGPLVPYTCCPPPMPTNFDTIPYYKFPSMTKLRIRPPAHAVDEEYIAKYNLAISRMKDLDKTDPLNPLGFKQQANIHCAYCNGAYIIGGKELQVHNSWLFFPFHRWYLYFYERILGKLIDDPTFALPYWNWDHPKGMRLPPMFDREGTSIYDERRNQQVRNGTVMDLGSFGDKVQTTQLQLMSNNLTLMYRQMVTNAPCPLLFFGAPYVLGNNVEAPGTIENIPHIPVHIWAGTVRGSTFPNGDTSYGEDMGNFYSAGLDPVFYCHHGNVDRMWNEWKAIGGKRRDLSEKDWLNSEFFFYDENKKPYRVKVRDCLDAKKMGYDYAPMPTPWRNFKPKTKASVGKVNTTTLPPVNKVFPLTKMDKAISFSINRPASSRTQQEKNEQEEMLTFDNIKYDNRGYIRFDVFLNVDNNVNANELDKAEFAGSYTSLPHVHRVGENDHTATVTFQLAITELLEDIGLEDEETIAVTLVPKKGGEGISIENVEIKLLDC

SEQ ID NO: 139 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 140 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 141 - protein, 540 aa, Camellia japonica
MMGISSLVVFPFLVFIAIANSLVPLASSSSEYEGFLQCLDLRSNSSIPIYNPSSTSYTSILHASIYNLRFISPTTPKPNFIITPMRESHVQATVVCSREHGLLIRTRSGGHDFEGSSFVATVPFVLLDLIHLRTISVNIEDETAWVQTGATIGELYYRIAEKSRTHAFPAGLCPSVGVGGHISGAGYGILMRKYGLSADHVIDARLVDVNGRILDKESMGEDYFWAIRGGGGASFGVILAWKIRLVPVPPTVTTFVVPRVLEQGATALVHKWQIIADKLDDDLFLGLSVQALHLDPDHPEKKTIVISFLGFYLGAPEKTLQLMEESFPELGLMKEDCIEMSWIKSALYFGIFQLETDLSLLLERKNPIPPKNRYMSKSDYVHEPVSEAVLEGIWKRFNEVDEPEIIMSPYGGKMNEIEESAIAFPHRKGNMYKINYLVSWKELGEEAEKKHASWIRELYNYMTPYVSKNPRCSYLNFKDNDLGHKKNGTATYLEAKVWGSKYYKNNFDRLVSVKTKVDPDDFFTNEQSIPPLRSLAEAKK

SEQ ID NO: 142 - protein, 566 aa, Nicotiana attenuata; identical to SEQ ID NO: 2

SEQ ID NO: 143 - protein, 541 aa, Papaver somniferum
MRLSARGFVWSALLACTASALSEAAATASNSSQTLRTCVSQALVAGDVNTRIIDPSNDTYTDARLGEKIQFNEFPALIAYAKKAEEVASLVRCAQRSGFKAVPRSGGHHFEAWSALNGTLVIDLSHINHVNVSADTTTANVGAGIRQGALYLALDEHNVTFPGGICPTVALGGLVSSGGFSLQMRALGLAAEYVQSARVVLADGSLVTASSSSHEDLFWAIRGGGGGTYGIIVDFDLQLMQFPTSAMVAISWNASSDRYPVAQRFFDWAPVQIPAFTSQVNVYKSSINFLGQYLGGTENELRKLINESGLLNIGTPTVYISGNCDTDNSRLFGYTSYECVPANETNRQIMNVLPEPFSQYSDYPQYQYENEPEDPSIPIAEPWARFNRISKSFFMQKDNILPAADLKTVIDMMGQLDTDSEIWGEWHAWNISSATKADYAFPWREQAYAHLEFQVHGSLTNSTKQATYEKWFADLETYLRPKIGVASYSGEMDAHISTNPFESYYGDSVCRLVEVKKAYDPDNFFTNPDAITPTVPEGISC

SEQ ID NO: 144 - protein, 473 aa, Eschscholzia californica
MGNTTSIAGRDCLISALGGNSALAVFPNELLWTADVHEYNLNLPVTPAAITYPETAAQIAGVVKCASDYDYKVQARSGGHSFGNYGLGGADGAVVVDMKHFTQFSMDDETYEAVIGPGTTLNDVDIELYNNGKRAMAHGVCPTIKTGGHFTIGGLGPTARQWGLALDHVEEVEVVLANSSIVRASNTQNQDVFFAVKGAAANFGIVTEFKVRTEPAPGLAVQYSYTFNLGSTAEKAQFVKDWQSFISAKNLTRQFYNNMVIFDGDIILEGLFFGSKEQYDALGLEDHFAPKNPGNILVLTDWLGMVGHALEDTILKLVGNTPTWFYAKSLGFRQDTLIPSAGIDEFFEYIANHTAGTPAWFVTLSLEGGAINDVAEDATAYAHRDVLFWVQLFMVNPLGPISDTTYEFTDGLYDVLARAVPESVGHAYLGCPDPRMEDAQQKYWRTNLPRLQELKEELDPKNTFHHPQGVMPA